(19) United States
(12) Patent Application Publication
(10) Pub. No.: US 2023/0126711 A1
(43) Pub. Date: Apr. 27, 2023

(54) ENTERIC NITRERGIC NEURONS AND METHODS OF USING THE SAME

(71) Applicant: The Regents of the University of California, Oakland, CA (US)

(72) Inventor: Faranak FATTAHI, San Francisco, CA (US)

(21) Appl. No.: 17/914,246

(22) PCT Filed: Mar. 25, 2021
(86) PCT No.: PCT/US21/24244

§ 371 Date: Sep. 23, 2022

Related U.S. Application Data
(60) Provisional application No. 62/994,837, filed on Mar. 25, 2020.
(60) Provisional application No. 63/036,492, filed on Jun. 9, 2020.

(51) Int. Cl. A61K 35/30; C12N 5/0793; C12N 5/0735

(52) U.S. Cl. CPC A61K 35/30; C12N 5/0619; C12N 5/0606; C12Y 114/13039; C12N 2501/115; C12N 2501/155; C12N 2501/727; C12N 2506/45; C12N 2533/90

(57) ABSTRACT

The present disclosure relates generally to methods of inducing differentiation of pluripotent stem cells into enteric nitrergic neurons, and enteric nitrergic neurons produced by such methods. Also provided are used of such enteric nitrergic neurons for screening potential therapeutic agents suitable for preventing and/or treating enteric nervous system disorders, such as gastroparesis, esophageal achalasia, chronic intestinal pseudo-obstruction, and hypertrophic pyloric stenosis, and applications of such enteric nitrergic neurons in regenerative medicine, such as cell transplantation therapy, for preventing and/or treating enteric nervous system disorders.

### CROSS-REFERENCE TO RELATED APPLICATIONS

This application claims priority to U.S. Provisional Application No. 62/994,837 filed on Mar. 25, 2020, and U.S. Provisional Application No. 63/036,492 filed on Jun. 9, 2020, the entire contents of each of the aforementioned applications are incorporated by reference herein in its entirety.

### TECHNOLOGY FIELD

The present disclosure generally relates to compositions comprising enteric nitrergic neurons and methods of inducing differentiation of stem cells into enteric nitrergic neurons, and cell culture systems comprising enteric nitrergic neurons. Also provided are used of such enteric nitrergic neurons for screening potential therapeutic agents suitable for preventing and/or treating enteric nervous system disorders, such as gastroparesis, esophageal achalasia, chronic intestinal pseudo-obstruction, and hypertrophic pyloric stenosis, and applications of such enteric nitrergic neurons in regenerative medicine, such as cell transplantation therapy, for preventing and/or treating gastrointestinal disorders.

### BACKGROUND

During embryogenesis, neural crest (NC) induction occurs at the interface of the non-neuronal ectoderm and the folding neural plate as a result of bone morphogenic protein (BMP), fibroblast growth factor (FGF), and WNT signaling pathway activity (1). During neurulation, dorsally localized NC cells delaminate and migrate away from the newly formed neural tube. Migratory NC cells proliferate and act as progenitors for a remarkable diversity of cell types including various populations of peripheral neurons and glia, melanocytes, endocrine cells and mesenchymal precursor cells (1-3). In the developing embryo, the neural crest shows an anterior-posterior spatial organization associated with the expression of regionally specific HOX genes. Distinct functional regions include the cranial NC, vagal NC, trunk NC and sacral NC located anteriorly to posteriorly respectively.

While the enteric nervous system (ENS) is generated from both the vagal and sacral NC, vagal NC lineages positive for HOXB3 (4) and HOXB5 (5) migrate most extensively to colonize the entire length of the bowel (6). Upon inclusion into the foregut, vagal NC cells display enteric neural crest (ENC) identity characterized by the expression of SOX10, PHOX2B, EDNRB, and ASCL1. Colonization of the intestinal tract by the ENC has been depicted as a rostrocaudally moving wave of proliferative multipotent ENS progenitors (7). By week seven of embryogenesis in humans, migratory ENC cells will reach the terminal hindgut (8). Failure of ENC migration to the caudal regions of the bowel can result in congenital aganglionosis of the colon, a disorder known as Hirschsprung's disease.

Post migratory ENC cells will commit to neuronal fates, a differentiation step associated with the downregulation of SOX10, sustained expression of EDNRB, ASCL1 and PHOX2B, and upregulation of pan neuronal markers such as TUJ1 (9). ENC progenitors further differentiate to establish ganglia located between the circular and longitudinal layers of enteric smooth muscle, forming the myenteric plexus. Recent spatiotemporal analysis of the murine ENS has shown that ENC progenitors within the myenteric plexus proliferate along the serosa-mucosal axis to subsequently form the ganglia of the submucosal plexus (10). Together, the myenteric and submucosal plexi will establish the neuronal circuitry of the functional ENS.

Due to the capacity of the NC to undergo an extensive range of cell fate decisions, methods seeking to optimize NC induction and subtype specification from human pluripotent stem cells (hPSCs) have been an important focus of research (11-13). Such hPSC-based NC induction methods commonly rely on a variation of the dual SMAD signaling inhibition protocol for neural induction, combined with the temporal activation of WNT signaling (12-14). However, such methods often involve the use of poorly defined culture components such as serum, BSA fractions, and other animal-derived products, that may affect the reliability and reproducibility of NC induction. Accordingly, methods use fully defined, xeno-free culture conditions for more reliable induction of cranial NC from hPSCs have been previously developed (15, 16).

The spatial and temporal transience of the ENC has been a major factor in limiting access to primary cells, particularly from human embryonic or fetal tissue samples. As a result, studying the developing ENS has largely relied upon studies in murine models. Work with such murine models resulted in the discovery of growth factors involved in the proliferation and differentiation of EN precursors, such as Neurotrophin-3 (NT-3) and glial cell line-derived neurotrophic factor (GDNF) (17, 18) among others. More recent single cell transcriptomics analysis of the developing murine ENS have revealed novel molecular states of lineally and functionally related ENS progenitors (10). An appreciable conservation of the transcriptional processes underpinning ENS development across mammals (19) supports the application of these factors to direct hPSC-derived ENC cells towards neurogenic commitments and may help further guide the identification, characterization and derivation of human enteric neuronal subtype lineages.

Small molecule-based methods for derivation of central nervous system (CNS) and peripheral nervous system (PNS) lineages from hPSCs including NC were established previously (12, 20-22). Methods for derivation of enteric neurons (ENs) from hPSCs have also been previously described. See e.g., PCT application No. PCT/US2019/068447 filed on Dec. 23, 2019, incorporated by reference herein in its entirety. However, despite extensive efforts and the important medical implications of GI disorders, the in vitro derivation of enteric nitrergic neurons, inhibitory neurons of the GI system that regulate gut motility and involve in a broad range of GI disorders, remains elusive.

### SUMMARY OF EMBODIMENTS

The disclosure relates to a method of differentiating at least one or a plurality of stem cells into at least one or a plurality of enteric nitrergic neurons, as well as compositions comprising such enteric nitrergic neurons and their uses thereof The disclosure further relates to a composition comprising a plurality of enteric neurons, wherein at least about 30% of the enteric neurons express nitric oxide synthase (NOS1). In some embodiments, at least about 30% of the enteric neurons express human NOS1. In some embodiments, at least about 60% of the enteric neurons express NOS1. In some embodiments, at least about 80% of the enteric neurons express NOS1. In some embodiments, the NOS1 expressed in the NO neurons of the disclosure comprises at least about 70% sequence identity to SEQ ID NO: 10, SEQ ID NO: 11 or SEQ ID NO: 12, or a functional fragment thereof In some embodiments, the nitric oxide synthase comprises SEQ ID NO: 10, SEQ ID NO: 11 or SEQ ID NO: 12, or a functional fragment thereof. In some embodiments, the enteric neurons are derived from one or a plurality of pluripotent stem cells. In some embodiments, the one or plurality of pluripotent stem cells are human inducible pluripotent stem cells.

In some embodiments, the composition of the disclosure further comprises neurons that express human CHAT. In some embodiments, the composition of the disclosure further comprises neurons that express human GABA. In some embodiments, the composition of the disclosure further comprises neurons that express human 5HT. In some embodiments, at least about 40% of the enteric neurons are neurons that express human CHAT. In some embodiments, at least about 9% of the enteric neurons are neurons that express human GABA. In some embodiments, at least about 6% of the enteric neurons are neurons that express human 5HT. In some embodiments, the plurality of cells expressing NOS1 are deficient in expression of any one or combination of: CHAT, 5HT, and GABA. In some embodiments, the plurality of cells expressing NOS1 are at least about 45 days in culture. In some embodiments, the plurality of cells are derived from cells that express human CD49 and SOX10 in culture from about 12 to about 15 days. In some embodiments, the plurality of cells are derived from cells that express human TRKC, PHOX2B and EDNRB in culture from about 15 to about 30 days. In some embodiments, the plurality of cells are derived from cells that express human TRKC and TUJ1 in culture from about 30 days to about 45 days. In some embodiments, the plurality of cells expressing NOS1 are derived from a combination of two or more of: human inducible pluripotent stem cells that are in culture at least about 12 days; cells that express human CD49 and SOX10 in culture from about 12 to about 15 days; cells that express human TRKC, PHOX2B and EDNRB in culture from about 15 to about 30 days; cells that express human TRKC and TUJ1 in culture from about 30 days to about 45 days.

In some embodiments the disclosure relates to a composition or a system comprising a cell culture vessel comprising a plurality of enteric neurons supported in a culture medium, wherein at least about 20% of the enteric neurons express nitric oxide synthase. In some embodiments, from about 20 to about 60% of the enteric neurons comprised in the system express nitric oxide synthase. In some embodiments, the cell culture comprises any of the aforementioned compositions. In some embodiments, the cell culture vessel further comprises a hydrogel. In some embodiments, the cell culture vessel further comprises smooth muscle cells proximate to or adjacent to the plurality of enteric neurons.

The disclosure further relates to a pharmaceutical composition comprising: a) a therapeutically effective amount of one or a plurality of enteric neurons; and b) a pharmaceutically acceptable carrier. In some embodiments, the composition or system comprises from about 20% to about 100% of the enteric neurons comprising NOS1. In some embodiments, at least about 80% of the enteric neurons express NOS1. In some embodiments, the NOS1 expressed in the cells comprises at least about 70% sequence identity to SEQ ID NO: 10, SEQ ID NO: 11 or SEQ ID NO: 12, or a functional fragment thereof. In some embodiments, the NOS1 comprises SEQ ID NO: 10, SEQ ID NO: 11 or SEQ ID NO: 12, or a functional fragment thereof.

In some embodiments, the enteric neurons are derived from one or a plurality of pluripotent stem cells. In some embodiments, the one or plurality enteric neurons are derived from human inducible pluripotent stem cells. In some embodiments, the enteric neurons are derived from a combination of two or more of: human inducible pluripotent stem cells that are in culture at least about 12 days; cells that express human CD49 and SOX10 in culture from about 12 to about 15 days; cells that express human TRKC, PHOX2B and EDNRB in culture from about 15 to about 30 days; cells that express human TRKC and TUJ1 in culture from about 30 days to about 45 days. In some embodiments, the plurality of cells expressing NOS1 are deficient in expression of any one or combination of: ChAT, 5HT, and GABA.

The disclosure further relates to a method of producing a plurality of enteric neurons, the method comprising exposing one or more nitrergic agents to a plurality of enteric neural crest cells, wherein at least about 30% of the enteric neurons express NOS1. In some embodiments, at least about 60% of the enteric neurons produced express NOS1. In some embodiments, from about 60% to about 80% of the enteric neurons express NOS1. In some embodiments, the NOS1 expressed in the cells comprises at least 70% sequence identity to SEQ ID NO: 10, SEQ ID NO: 11 or SEQ ID NO: 12, or a functional fragment thereof In some embodiments, the NOS1 comprises SEQ ID NO: 10, SEQ ID NO: 11 or SEQ ID NO: 12, or a functional fragment thereof.

In some embodiments, the method of the disclosure further comprises differentiating one or a plurality of stem cells into one or a plurality of enteric neural crest cells prior to the step of exposing the neural crest cells to the one or more nitrergic agents. In some embodiments, the one or plurality of stem cells are human inducible pluripotent stem cells or embryonic stem cells. In some embodiments, the one or more nitrergic agents used in the methods of the disclosure are receptor tyrosine kinase (RTK) inhibitors. In some embodiments, the nitrergic agent is selected from the group consisting of: PP121, afatinib, ibrutinib, mizoribine, donepezil, cilostazol, RG108, prucalopride, PluriSIn #1, L-Arginine, AMG-458, OG-L002, GSK2801, GSK J4, GSK591, and sodium orthovanadate, or a salt of any of the foregoing. In some embodiments, the nitrergic agent is PP121.

The disclosure additionally relates to a method of producing nitric oxide synthase (NOS)-expressing enteric neurons, the method comprising exposing one or more nitrergic agents to one or a plurality of enteric neural crest cells. In some embodiments, the enteric neurons express NOS1 at least about 70% sequence identity to SEQ ID NO: 10, SEQ ID NO: 11 or SEQ ID NO: 12, or a functional fragment thereof. In some embodiments, NOS1 comprises SEQ ID NO: 10, SEQ ID NO: 11 or SEQ ID NO: 12, or a functional fragment thereof. In some embodiments, the plurality of enteric neural crest cells are differentiated from one or a plurality of pluripotent stem cells. In some embodiments, the one or plurality of pluripotent stem cells are human pluripotent stem cells. In some embodiments, the one or more nitrergic agents used are receptor tyrosine kinase (RTK) inhibitors. In some embodiments, the nitrergic agents are selected from one or a combination of: PP121, afatinib, ibrutinib, mizoribine, donepezil, cilostazol, RG108, prucalopride, PluriSIn #1, L-Arginine, AMG-458, OG-L002, GSK2801, GSK J4, GSK591, and sodium orthovanadate, or a salt of any of the foregoing. In some embodiments, the RTK inhibitor is PP121.

In some embodiments, the nitrergic agent has a structure represented by a formula:

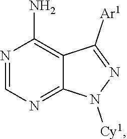

wherein Cy¹ is selected from C3-C8 cycloalkyl and C2-C9 heterocycloalkyl, and is substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹;

wherein R¹, when present, is selected from hydrogen, C1-C4 alkyl, C2-C4 alkenyl, and C1-C4 haloalkyl;

wherein Ar¹ is selected from C6-C10 aryl and C2-C9 heteroaryl, and is substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr²;

wherein Ar², when present, is selected from C6-C10 aryl and C2-C9 heteroaryl, and is substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, and (C1-C4)(C1-C4) dialkylamino,

or a pharmaceutically acceptable salt thereof.

In some embodiments, the nitrergic agent has a structure represented by the aforementioned formula, wherein Cy¹ is unsubstituted C3-C8 cycloalkyl. In some embodiments, the nitrergic agent has a structure represented by the aforementioned formula, wherein Cy¹ is unsubstituted cyclopentyl. In some embodiments, the nitrergic agent has a structure represented by the aforementioned formula, wherein Cy¹ is C2-C9 heterocycloalkyl substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹. In some embodiments, the nitrergic agent has a structure represented by the aforementioned formula, wherein Cy¹ is C2-C9 heterocycloalkyl substituted with a group selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹. In some embodiments, the nitrergic agent has a structure represented by the aforementioned formula, wherein Cy¹ is C2-C9 heterocycloalkyl substituted with a —C(O)R¹ group. In some embodiments, the nitrergic agent has a structure represented by the aforementioned formula, wherein Cy¹ is piperidinyl substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹. In some embodiments, the nitrergic agent has a structure represented by the aforementioned formula, wherein Cy¹ is piperidinyl substituted with a group selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹. In some embodiments, the nitrergic agent has a structure represented by the aforementioned formula, wherein Cy¹ is piperidinyl substituted with a —C(O)R¹ group. In some embodiments, the nitrergic agent has a structure represented by the aforementioned formula, wherein R¹, when present, is C2-C4 alkenyl. In some embodiments, the nitrergic agent has a structure represented by the aforementioned formula, wherein R¹, when present, is ethenyl. In some embodiments, the nitrergic agent has a structure represented by the aforementioned formula, wherein Ar¹ is unsubstituted C2-C9 heteroaryl. In some embodiments, the nitrergic agent has a structure represented by the aforementioned formula, wherein Ar¹ is unsubstituted pyrrolopyridinyl. In some embodiments, the nitrergic agent has a structure represented by the aforementioned formula, wherein Ar¹ is selected from C6-C10 aryl substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr². In some embodiments, the nitrergic agent has a structure represented by the aforementioned formula, wherein Ar¹ is selected from C6-C10 aryl substituted with a group selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr². In some embodiments, the nitrergic agent has a structure represented by the aforementioned formula, wherein Ar¹ is selected from C6-C10 aryl substituted with —OAr². In some embodiments, the nitrergic agent has a structure represented by the aforementioned formula, wherein Ar¹ is selected from C6 aryl substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr². In some embodiments, the nitrergic agent has a structure represented by the aforementioned formula, wherein Ar¹ is selected from C6 aryl substituted with a group selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr². In some embodiments, the nitrergic agent has a structure represented by the aforementioned formula, wherein Ar¹ is selected from C6 aryl substituted with a —OAr². In some embodiments, the nitrergic agent has a structure represented by the aforementioned formula, wherein Ar², when present, is unsubstituted C6-C10 aryl. In some embodiments, the nitrergic agent has a structure represented by the aforementioned formula, wherein Ar², when present, is unsubstituted C6 aryl.

In some embodiments, the nitrergic agent has a structure represented by a formula selected from:

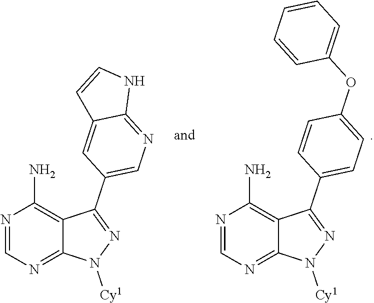

In some embodiments, the nitrergic agent is selected from:

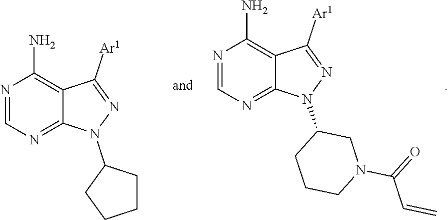

In some embodiments, the nitrergic agent is selected from:

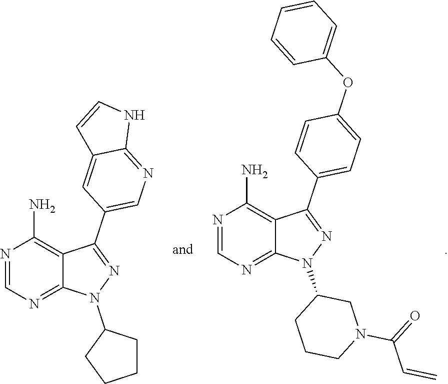

In some embodiments, the nitrergic agent is selected from:

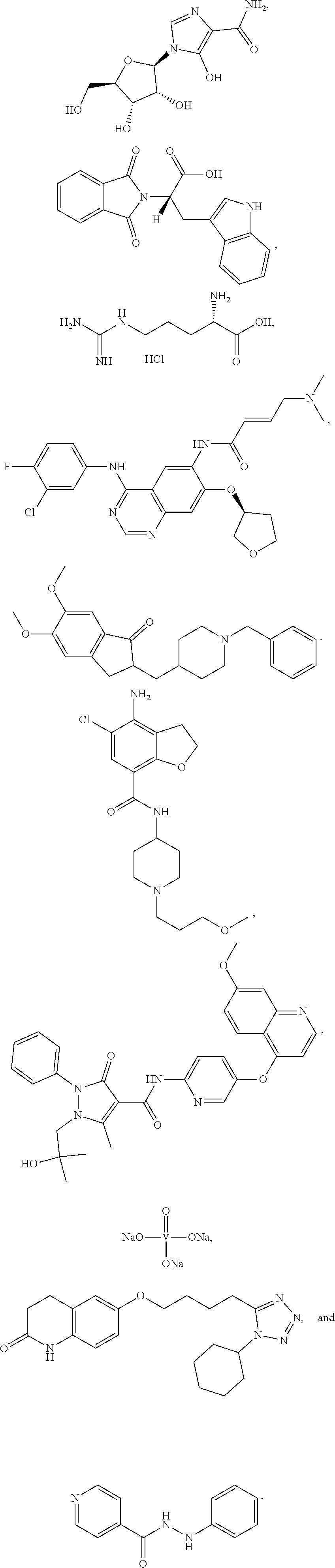

or a pharmaceutically acceptable salt thereof.

In some embodiments, the method of the disclosure further comprises isolating the NOS-expressing enteric neurons using one or more surface antigens specific for the NOS-expressing enteric neurons.

The discourse also relates to a method of evaluating a neuromodulatory effect of an agent, the method comprising: a) culturing a plurality of enteric neurons in the presence or absence of the agent, wherein at least about 30% of the enteric neurons express nitric oxide synthase; and b) detecting and/or measuring nitric oxide released by the agent; wherein a detectable level of nitric oxide in the presence of the agent is indicative of a neuromodulatory effect, and no detectable level of nitric oxide in the presence of the agent is indicative of the agent not conferring a neuromodulatory effect. In some embodiments, at least about 60% of the enteric neurons express nitric oxide synthase. In some embodiments, at least about 80% of the enteric neurons express nitric oxide synthase. In some embodiments, the nitric oxide synthase comprises at least about 70% sequence identity to SEQ ID NO: 10, SEQ ID NO: 11 or SEQ ID NO: 12, or a functional fragment thereof. In some embodiments, the nitric oxide synthase comprises SEQ ID NO: 10, SEQ ID NO: 11 or SEQ ID NO: 12, or a functional fragment thereof In some embodiments, the enteric neurons are differentiated from one or a plurality of stem cells. In some embodiments, the one or plurality of stem cells are human pluripotent stem cells.

The disclosure further relates to a method for screening an agent capable of modulating calcium influx, the method comprising: a) culturing a plurality of enteric neurons in the presence or absence of the agent, wherein at least about 30% of the enteric neurons express nitric oxide synthase; and b) detecting and/or measuring nitric oxide released by the agent; wherein a detectable level of nitric oxide in the presence of the agent is indicative of the agent capable of modulating calcium influx, and no detectable level of nitric oxide in the presence of the agent is indicative of the agent not conferring ability to modulate calcium influx. In some embodiments, at least about 60% of the enteric neurons express NOS1. In some embodiments, at least about 80% of the enteric neurons express NOS1. In some embodiments, the nitric oxide synthase comprises at least about 70% sequence identity to SEQ ID NO: 10, SEQ ID NO: 11 or SEQ ID NO: 12, or a functional fragment thereof. In some embodiments, the NOS1 comprises SEQ ID NO: 10, SEQ ID NO: 11 or SEQ ID NO: 12, or a functional fragment thereof.

The disclosure further relates to a method of measuring or quantifying a neuromodulatory effect of an agent, the method comprising: a) culturing one or a plurality of enteric neurons in the presence or absence of the agent, wherein at least about 30% of the enteric neurons express NOS1; and b) detecting and/or measuring nitric oxide released by the agent; wherein a detectable level of nitric oxide in the presence of the agent is indicative of a neuromodulatory effect, and no detectable level of nitric oxide in the presence of the agent is indicative of the agent not conferring a neuromodulatory effect. In some embodiments, at least about 60% of the enteric neurons express nitric oxide synthase. In some embodiments, at least about 80% of the enteric neurons express nitric oxide synthase. In some embodiments, the nitric oxide synthase comprises at least about 70% sequence identity to SEQ ID NO: 10, SEQ ID NO: 11 or SEQ ID NO: 12, or a functional fragment thereof In some embodiments, the nitric oxide synthase comprises SEQ ID NO: 10, SEQ ID NO: 11 or SEQ ID NO: 12, or a functional fragment thereof.

The disclosure also relates to a method of isolating a population of enteric neurons expressing NOS1, wherein the population is: a) culturing one or a plurality of enteric neurons; b) exposing the enteric neurons to one or a plurality of nitrergic agents; c) isolating one or a plurality of enteric neurons by exposing the neurons to a solid support comprising an antibody specific for one or a combination of: CD47, CD58, CD59, CD90, CD181, CD235a and/or NOS1. In some embodiments, the step of culturing comprises: i) culturing stem cells for about 12 days; ii) differentiating the stem cells into cells that express human CD49 and SOX10; iii) culturing cells that express human CD49 and SOX10 from about 1 to about 4 days after step (i) and prior to step (b). In some embodiments, the method further comprises differentiating cells that express human CD49 and SOX10 into cells that express human TRKC, PHOX2B and EDNRB for about 15 days; and differentiating cells that express cells that express human TRKC, PHOX2B and EDNRB into cells that express human TRKC and TUJ1 for about 15 days.

### BRIEF DESCRIPTION OF THE DRAWINGS

FIG. 1 depicts a schematic of directed derivation of enteric nitrergic neurons (enteric NO neurons) from human pluripotent stem cells (hPSCs).

FIG. 2 depicts a schematic for the generation of enteric nitrergic neurons from pluripotent stem cells.

FIG. 3A-3C depict the high-throughput chemical screening for identifying compounds that enhance nitrergic neuron induction. FIG. 3A is a schematic illustration of high-throughput small molecule screen searching for compounds that promote NO neuron induction. FIG. 3B depicts the primary screen data and cut off for identification of hit compounds. FIG. 3C is a list of hit compounds that promote more than about 8-fold increase in percentage of NO neurons in differentiated cultures.

FIG. 4 depicts the chemical structure of some compounds identified from the screening as having the potential to enhance nitrergic neuron induction.

FIG. 5 depicts the dose-response analysis of hit compounds PP121 and GSK591 to define optimal concentration.

FIG. 6 depicts the optimization of treatment window for PP121. Day 15-20 appears to be the optimal PP121 treatment window.

FIG. 7A-7D depict PP121 enhances NOS1 induction efficiency through RTK inhibition. FIG. 7A shows the percentage of NO neurons in cultures treated with PP121 compared to no treatment (NT) measured by flow cytometry for NOS1. FIG. 7B shows the immunofluorescence staining of NOS1 in PP121 treated culture and no treatment controls. FIG. 7C shows tyrosine kinase receptor (RTK) ligands reduce the percentage of NO neurons and block the effect of PP121. NO neurons quantified using flow cytometry for NOS1. FIG. 7D shows PDGF treatment reduced the percentage of NO neurons in differentiated cultures based on quantification of flow cytometry analysis. Sunitinib which is an RTK inhibitor selective for PDGFR increases NO neurons similar to PP121.

FIG. 8A-8D depicts prospective isolation of enteric nitrergic neurons. Top of FIG. 8A is a schematic illustration of NOS1::GFP reporter construct generated to facilitate FACS purification of NO neurons. Bottom of FIG. 8A depicts co-expression of GFP and NOS1 shown using immunofluorescence staining. FIG. 8B depicts FACS purification of GFP positive neurons. It is discovered that CD24 is a pan enteric neuron marker that labels all neurons in the differentiated cultures. FIG. 8C depicts the results of q-RT PCR showing that FACS purified GFP+ neurons express higher levels of NOS1 and similar levels of neuronal marker TUJ1 compared to other neurons. FIG. 8D depicts the flowcytometry analysis confirming co-expression of GFP with NOS1 in four independent differentiations.

FIG. 9 depicts that modulators of WNT, BMP and GDNF signaling are differentially expressed in NO neurons. Top 50 upregulated and downregulated transcripts in NO neurons compared to other enteric neurons. This list can serve as a panel of specific markers for NO neurons.

FIG. 10 depicts other signaling modulators differentially expressed in NO neurons, including additional upregulated and downregulated transcripts related to various signaling pathways.

FIG. 11 depicts transcription factors and NT genes differentially expressed in NO neurons, including additional upregulated and downregulated transcripts related to specific transcription factor families and NO biosynthesis pathway.

FIG. 12A-12B depict that antibody screening identifies novel surface antigens specific for NO neurons, which can be used for FACS purification. FIG. 12A is a schematic illustration of surface marker screening experiment aimed at identifying markers that facilitate FACS purification of NO neurons. FIG. 12B shows the identification of several surface antibodies that label cells in enteric neuron cultures.

FIG. 13 depicts a list of antibodies that bind to neurons in the differentiated culture. CD24 μlabels all neurons. Values above CD24 represent markers specific for NO neurons.

FIG. 14A-14B depicts that transcription profiling identifies candidate surface antigens specific for NO neurons. FIG. 14A is a schematic illustration of transcription profiling. FIG. 14B depicts a list of upregulated and downregulated transcripts for surface markers in NO neurons compared to other neurons.

FIG. 15 depicts key follow-up applications for hPSC-derived enteric NO neurons for high-throughput screening for drugs that enhance NO neuron activity and transplantation of NO neurons for cell therapy in GI motility disorders.

FIG. 16A-16B depict development of NO release assay to perform high-throughput screening.

FIG. 17A-17C depict NO release assay for high-throughput compounds screening to identify NO neuron modulators. FIG. 17A is a schematic illustration of the NO release assay. FIG. 17B depicts a primary screen data and cut off for identification of hit compounds. FIG. 17C is a list of hit compounds that promote NO release in the NO release assay.

FIG. 18A-18B depicts transplanted hPSC-derived NO neurons engraft extensively in NOS1−/− mouse colon. FIG. 18A is a schematic illustration of the transplantation of enteric NO neurons into NOS1−/− mouse colon. FIG. 18B depicts an immunofluorescence staining of STEM121 in different segments of mouse colon tissue 8 weeks after transplantation. NO neurons were transplanted in a mouse model of GI motility disorder and showed great capacity to engraft and survive. STEM121 is a marker for a human-specific cytoplasmic protein. Immunofluorescence staining of STEM121 shows a large number of human cells in different segments of mouse colon tissue 8 weeks after transplantation.

FIG. 19 depicts transplanted hPSC-derived NO neurons engraft extensively in NOS1−/− mouse colon. Immunofluorescence staining of STEM121, TUJ1 and NOS1 shows a large number of human NO neurons in mouse colon tissue 8 weeks after transplantation. Given the remarkable potential of cells to engraft and survive in intestinal tissue, they show promise for cell therapy in GI motility disorders such as achalasia and gastroparesis.

FIG. 20 depicts a summary of efficient derivation of enteric NO neurons for drug discovery and cell therapy in GI motility disorders.

### DETAILED DESCRIPTION OF EMBODIMENTS

Before the present methods are described, it is to be understood that the present disclosure is not limited to the particular processes, compositions, or methodologies described, as these may vary. It is also to be understood that the terminology used in the description is for the purposes of describing the particular versions or embodiments only, and is not intended to limit the scope of the present disclosure. Unless defined otherwise, all technical and scientific terms used herein have the same meanings as commonly understood by one of ordinary skill in the art. For example, Singleton et al., Dictionary of Microbiology and Molecular Biology 2^{nd} Ed., J. Wiley & Sons (New York, N.Y. 1994), provide one skilled in the art with a general guide to many of the terms used in the present disclosure.

Moreover, the practice of the present disclosure will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual,” 2^{nd} Ed. (Sambrook et al., 1989); “Oligonucleotide Synthesis” (M. J. Gait, Ed., 1984); “Animal Cell Culture” (R. I. Freshney, Ed., 1987); “Methods in Enzymology” (Academic Press, Inc.); “Handbook of Experimental Immunology,” 4^{th} Ed. (D. M. Weir & C. C. Blackwell, Eds., Blackwell Science Inc., 1987); “Gene Transfer Vectors for Mammalian Cells” (J. M. Miller & M. P. Calos, Eds., 1987); “Current Protocols in Molecular Biology” (F. M. Ausubel et al., Eds., 1987); and “PCR: The Polymerase Chain Reaction,” (Mullis et al., Eds., 1994). Although any methods and materials similar or equivalent to those described herein can be used in the practice or testing of embodiments of the present disclosure, the methods, devices, and materials in some embodiments are now described. All publications mentioned herein are incorporated by reference in their entirety. Nothing herein is to be construed as an admission that the present disclosure is not entitled to antedate such disclosure by virtue of prior invention.

### Definitions

Unless defined otherwise, all technical and scientific terms used herein have the same meaning as commonly understood by one of ordinary skill in the art to which the invention pertains.

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The term “about” or “approximately” is used herein to mean within the typical ranges of tolerances in the art. For example, “about” can be understood as about 2 standard deviations from the mean. According to certain embodiments, when referring to a measurable value such as an amount and the like, “about” is meant to encompass variations of +20%, +10%, +5%, ±1%, ±0.9%, ±0.8%, ±0.7%, ±0.6%, +0.5%, ±0.4%, ±0.3%, ±0.2% or +0.1% from the specified value as such variations are appropriate to perform the disclosed methods. When “about” is present before a series of numbers or a range, it is understood that “about” can modify each of the numbers in the series or range.

The term “activator” refers to compounds that increase, induce, stimulate, activate, facilitate, or enhance activation the signaling function of the molecule or pathway, e.g., Wnt signaling.

“Analogues” of the compounds disclosed herein are pharmaceutically acceptable salts, prodrugs, deuterated forms, radio-actively labeled forms, isomers, solvates and combinations thereof. The “combinations” mentioned in this context refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like. The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include, e.g., sodium, potassium, calcium, ammonium, organic amino, or magnesium salt. As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “antibody,” as used herein, broadly refers to any immunoglobulin (Ig) molecule comprised of four polypeptide chains, two heavy (H) chains and two light (L) chains, or any functional fragment, mutant, variant, or derivative thereof, which retains the essential epitope binding features of an Ig molecule. Such mutant, variant, or derivative antibody formats are known in the art. Non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H1}; (ii) V_{H}-C_{H2}; (iii) VH-CH3; (iv) V_{H}-C_{H1}-C_{H2}; (v) V_{H}-C_{H1}-C_{H2}-C_{H3}; (vi) V_{H}-C_{H2}-C_{H3}; (vii) V_{H}-C_{L}; (viii) V_{L}-C_{H1}; (ix) V_{L}-C_{H2}; (x) V_{L}-C_{H3}; (xi) V_{L}-C_{H1}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antigen binding portion” or “antigen binding fragment” of an antibody (or simply “antibody portion” or “antibody fragment”), as used herein, refers to one or more fragments of an antibody that retain the ability to specifically bind to an antigen (e.g., hCD40). It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” or “antigen binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

Full length antibodies comprise immunoglobulin constant regions of one or more immunoglobulin classes. Immunoglobulin classes include IgG, IgM, IgA, IgD, and IgE isotypes and, in the case of IgG and IgA, their subtypes. In some embodiments, a full length antibody of the disclosure has a constant domain structure of an IgG type antibody.

The term “antigen” refers to a polypeptide that can stimulate the production of antibodies or a T cell response in an animal, including polypeptides that are injected or absorbed into an animal. An antigen reacts with the products of specific humoral or cellular immunity.

As used in the specification and in the claims, the term “comprising” can include the aspects “consisting of” and “consisting essentially of” Comprising can also mean “including but not limited to.”

The terms “biologically effective amount” as used herein is any amount of an agent, chemical, biological molecule, protein or ligand sufficient to cause a biological effect. In some embodiments, the biologically effective amount of a nitrergic agent is an amount sufficient to case induction of NOS1.

The term “culture vessel” as used herein is defined as any vessel suitable for growing, culturing, cultivating, proliferating, propagating, or otherwise similarly manipulating cells. A culture vessel may also be referred to herein as a “culture insert.” In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. In some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel is a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of enteric nitrergic neurons. In some embodiments, the culture vessel comprises a hydrogel and one or a plurality of isolated enteric nitrergic neurons, to which one or a plurality of muscle cells are seeded.

The terms “deficient in expression” as used herein refers to a cell that is free of biologically effective amounts of a nucleic acid molecule to protein that confers a particular response. In some embodiments, the cells expresses a basal level of protein but not enough to confer a specific biological response. In some embodiments, deficient in expression means that the cell is unable to express biologically active protein, or, in spite of basal expression, is incapable of expressing biologically active isoforms.

The term “derivative” as used herein refers to a chemical compound with a similar core structure.

The term “embryonic stem cell” as used herein refers to a primitive (undifferentiated) cell that is derived from preimplantation-stage embryo, capable of dividing without differentiating for a prolonged period in culture, and are known to develop into cells and tissues of the three primary germ layers. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

The term “embryonic stem cell line” as used herein refers to a population of embryonic stem cells which have been cultured under in vitro conditions that allow proliferation without differentiation for up to days, months to years. In some embodiments, an “embryonic stem cell line” refers to a population of cells derived from the inner cell mass of the pre-implantation blastocyst capable of self-renewal and differentiation into the three primary germ layers. In some embodiments, embryonic stem cell lines are those listed in the NIH Human Embryonic Stem Cell Registry, such as CHB-1, CHB-2, CHB-3, CHB-4, CHB-5, CHB-6, CHB-8, CHB-9, CHB-10, CHB-11, CHB-12, RUES1, RUES2, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 26, HUES 27, HUES 28, CyT49, RUES3, WA01 (H1), UCSF4, NYUES1, NYUES2, NYUES3, NYUES4, NYUES5, NYUES6, NYUES7, MFS5, HUES 48, HUES 49, HUES 53, HUES 65, HUES 66, UCLA 1, UCLA 2, UCLA 3, WA07 (H7), WA09 (H9), WA13 (H13), WA14 (H14), HUES 62, HUES 63, HUES 64, CT1, CT2, CT3, CT4, MA135, Endeavour-2, WIBR1, WIBR2, HUES 45, Shef 3, Shef 6, WIBR3, WIBR4, WIBR5, WIBR6, BJNhem19, BJNhem20, SA001, SA002, UCLA 4, UCLA 5, UCLA 6, HUES PGD 13, HUES PGD 3, ESI-014, ESI-017, HUES PGD 11, HUES PGD 12, WA15, WA16, WA17, WA18, WA19, etc. In some embodiments, embryonic stem cells comprise gene(s) associated with diseases or disorders.

The term “enteric nervous system precursor,” “ENS precursor,” “enteric neural crest precursor,” “enteric NC precursor” or “ENC precursor” as used herein refers to a cell expressing one or more enteric neural crest lineage marker. An ENS precursor is a cell with the ability to mature into an enteric neuron. A human ENS precursor refers to an ENS precursor that is from a human. Non-limiting examples of enteric neural crest lineage markers include PAX3, EDNRB, RET, PHOX2A, PHOX2B, NTRK-3, HAND2, HOXB3, HOXB5 and ASCL1.

The term “enteric neural crest cell” as used herein means a cell produced by inducing differentiation of a pluripotent stem cell, wherein the enteric neural crest cell expresses at least one or more of: SOX10, PHOX2B, EDNRB, TFAP2A, BRN3A, ISL1 and/or ASCL1. In some embodiments, the neural crest cell is present in an embryonic body or neural rosette. In some embodiments, the neural crest cell expresses vagal markers HOXB2, HOXB3, and/or HOXB5. In some embodiments, neural crest cells express p75 and HNK1. In some embodiments, neural crest cells express HOXB2, HOXB3, HAND2 and EDNRB.

The term “enteric neuron” as used herein refers to a cell produced by inducing differentiation of an enteric neural crest cell and expressing one or more enteric neuron marker. Non-limiting examples of enteric neuron markers include TUJ1, MAP2, PHOX2A, PHOX2B, TRKC, ASCL1, HAND2, EDNRB, 5HT, GABA, NOS, SST, TH, CHAT, DBH, Substance P, VIP, NPY, GnRH, and CGRP. In some embodiments, enteric neurons exhibit downregulation of SOX10, sustained expression of EDNRB, ASCL1 and PHOX2B, and upregulation of TUJ1 and TRKC. In some embodiments, enteric neurons express neuronal subtype specific markers including the cholinergic neuronal marker Choline Acetyl Transferase (CHAT), serotonin (5-HT) receptor, gamma-Aminobutyric acid (GABA), and neuronal nitric oxide synthase (nNOS). In some embodiments, CHAT expression indicates the presence of cholinergic neurons. In some embodiments, expression of NOS1 indicates the presence of nitrergic neurons. In some embodiments, enteric neurons include glial cells expressing glial fibrillary acidic protein (GFAP) and SOX10.

The term “enteric nitrergic neuron” as used herein refers to an enteric neuron that expresses neuronal nitric oxide synthase. Enteric nitrergic neurons are inhibitory motor neurons that play crucial roles in regulating gastrointestinal motility and thus, loss or damage of these neurons can contribute to developing gastrointestinal motility disturbances suffered by patients worldwide.

The term “exposing” as used herein refers to bringing a disclosed compound and a cell, target receptor, or other biological entity together in such a manner that the compound can affect the activity of the cell (e.g., receptor, cell, etc.), either directly (i.e., by interacting with the target or cell itself) or indirectly (i.e., by interacting with another molecule, such as co-factor, factor, or protein on which the activity of the cell is dependent). In some embodiments, the activity of cell is differentiation. In some embodiments, the compound is one or more differentiation factors.

By “fragment” is meant a portion of a polypeptide or nucleic acid molecule. This portion contains, preferably, at least about about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence identity to the wild-type sequence upon which the sequence is derived.

The term “hydrogel” as used herein is defined as any water-insoluble, crosslinked, three-dimensional network of polymer chains with the voids between polymer chains filled with or capable of being filled with water. The term “hydrogel matrix” as used herein is defined as any three-dimensional hydrogel construct, system, device, or similar structure. In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof. In some embodiments, the hydrogel or hydrogel matrix comprises Matrigel® or vitronectin. In some embodiments, the hydrogel or hydrogel matrix can be solidified into various shapes, for example, a bifurcating shape designed to mimic a neuronal tract. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, two or more hydrogels or hydrogel matrixes are used simultaneously cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed tissue culture vessel, it is possible for some embodiments to include any number of aforementioned wells or independently addressable location within the cell culture vessel such that a hydrogel matrix in one well or location is different or the same as the hydrogel matrix in another well or location of the cell culture vessel.

The term “induced pluripotent stem cell,” or iPSC, means a type of pluripotent cell made by reprogramming a somatic cell to have the same properties as embryonic stem cells, namely, the ability to self-renew and differentiate into the three primary germ layers. In some embodiments, iPSCs include mammalian cells, such cells from human, mouse, rat, monkey, horse, goat, sheep, dog, cat, etc., reprogrammed to express Oct4, Nanog, Sox2, and optionally c-Myc. In some embodiments, iPSCs comprise reprogrammed primary cell lines. In some embodiments, iPSCs are obtained from a repository, such as the Coriell Institute for Medical Research (e.g., Catalog ID GM25256 (WTC-11), GM25430, GM23392, GM23396, GM24666, GM27177, GM24683), California Institute for Regenerative Medicine: California's Stem Cell Agency (e.g., CW60261, CW60354, CW60359, CW60480, CW60335, CW60280, CW60594, CW60083, CW60086, CW60087, CW60167, CW60186), and the American Type Culture Collection (ATCC®) (e.g., ATCC-DYR0530 Human Induced Pluripotent Stem (IPS) Cells (ATCC® ACS-1012™, ATCC® ACS-1011™, ATCC® ACS-1024™, ATCC® ACS-1028™, ATCC® ACS-1031™, ATCC® ACS-1004™, ATCC® ACS-1029™, ATCC® ACS-1020™, ATCC® ACS-1007™, ATCC® ACS-1030™). Induced pluripotent stem cells may be derived from cell types such as fibroblasts taken from the skin, lung, or vein of subjects that are apparently healthy or diseased.

As used herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “inhibitor” as used herein refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway. An inhibitor can be any compound or molecule that changes any activity of a named protein (signaling molecule, any molecule involved with the named signaling molecule, a named associated molecule, such as a glycogen synthase kinase 33 (GSK3P)) (e.g., including, but not limited to, the signaling molecules described herein), for one example, via directly contacting SMAD signaling, contacting SMAD mRNA, causing conformational changes of SMAD, decreasing SMAD protein levels, or interfering with SMAD interactions with signaling partners (e.g., including those described herein), and affecting the expression of SMAD target genes (e.g. those described herein). Inhibitors also include molecules that indirectly regulate SMAD biological activity by intercepting upstream signaling molecules (e.g., within the extracellular domain). Examples of a signaling molecule and an effect include: Noggin which sequesters bone morphogenic proteins, inhibiting activation of ALK receptors 1, 2, 3, and 6, thus preventing downstream SMAD activation. Likewise, Chordin, Cerberus, Follistatin, similarly sequester extracellular activators of SMAD signaling. Bambi, a transmembrane protein, also acts as a pseudo-receptor to sequester extracellular TGFb signaling molecules. Antibodies that block activins, nodal, TGFb, and BMPs are contemplated for use to neutralize extracellular activators of SMAD signaling, and the like. Inhibitors are described in terms of competitive inhibition (binds to the active site in a manner as to exclude or reduce the binding of another known binding compound) and allosteric inhibition (binds to a protein in a manner to change the protein conformation in a manner which interferes with binding of a compound to that protein's active site) in addition to inhibition induced by binding to and affecting a molecule upstream from the named signaling molecule that in turn causes inhibition of the named molecule. An inhibitor can be a “direct inhibitor” that inhibits a signaling target or a signaling target pathway by actually contacting the signaling target.

The term “ligands” as used herein refers to molecules and proteins that bind to receptors, such as transforming growth factor-beta (TFGP), Activin, Nodal, bone morphogenic proteins (BMPs), etc.

The term “Matrigel®” means a solubilized basement membrane preparation extracted from the Engelbreth-Holm-Swarm (EHS) mouse sarcoma comprising ECM proteins including laminin, collagen IV, heparin sulfate proteoglycans, entactin/nidogen, and other growth factors. In some embodiments, Cultrex® BME (Trevigen, Inc.) or Geltrex® (Thermo-Fisher Inc.) may be substituted for Matrigel®.

The term “nitrergic agent” as used herein refers to an agent, when exposed to a call, causes or induces the cell to express nitric oxide synthase (NOS1). In some embodiments, the nitrergic agent is a RTK inhibitor. The term “RTK inhibitor” as used herein, includes, but is not limited to, protein tyrosine kinase and/or serine and/or threonine kinase inhibitors or lipid kinase inhibitors. In some embodiments, the cells are exposed a biologically effective amount of one or more nitrergic agents. In some embodiments, the biologically amount is from about 1 picogram to about 1000 nanograms. In some embodiments, the biologically amount is from about 1 nanogram to about 100 micrograms.

“Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. In some embodiments, the disclosed compositions are administered with at least one pharmaceutically acceptable carrier.

The term “pharmaceutically acceptable carrier” is art recognized and includes a pharmaceutically acceptable material, composition or vehicle, suitable for administering compositions of the present disclosure to subjects. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but not limited to, sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. In some embodiments, the pharmaceutically acceptable carrier is sterile and pyrogen-free water. In some embodiments, the pharmaceutically acceptable carrier is Ringer's Lactate, sometimes known as lactated Ringer's solution. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The term “pluripotent stem cell” as used herein is defined as a cell that is self-replicating capable of developing into cells and tissues of the three primary germ layers. Pluripotent stem cells include embryonic and induced pluripotent cells as defined herein. Contemplated pluripotent stem cells originate from mammals, such as human, mouse, rat, monkey, horse, goat, sheep, dog, cat, etc. A human pluripotent stem cell, or hPSC, refers to a pluripotent stem cell that is from a human.

The term “preventing” or “prevention” or “prevent” as used herein refers to prophylactic or preventative measures that prevent or slow the development of a targeted pathologic condition or disorder. Those in need of treatment include those already diagnosed with the disorder; those prone to have the disorder; and those in whom the disorder is to be prevented.

The term “rho kinase inhibitor” means a compound that decreases the activity of rho kinase. In some embodiments, the rho kinase inhibitor is N-[(3-Hydroxyphenyl)methyl]-N′-[4-(4-pyridinyl)-2-thiazolyl]urea dihydrochloride (RKI-1447), (+)-(R)-trans-4-(1-aminoethyl)-N-(4-pyridyl)cyclohexanecarboxamide dihydrochloride (Y-27632), Fasudil (HA-1077), Hydroxyfasudil (HA 1100 hydrochloride), Thiazovivin, GSK429286A, Narciclasine, and/or (+)-(R)-trans4-(1-aminoethyl)-N-(1H-pyrrolo[2,3-b]pyridin-4-yl)cyclohexanecarboxamide dihydrochloride (Y-30141).

As used herein, the term “signaling” in reference to a “signal transduction protein” refers to a protein that is activated or otherwise affected by ligand binding to a membrane receptor protein or some other stimulus. Examples of signal transduction protein include, but are not limited to, a SMAD, a wingless (WNT) complex protein, including beta-catnin, NOTCH, transforming growth factor beta (TGFP), Activin, Nodal and glycogen synthase kinase 33 (GSK3P) proteins. For many cell surface receptors or internal receptor proteins, ligand-receptor interactions are not directly linked to the cell's response. The ligand activated receptor can first interact with other proteins inside the cell before the ultimate physiological effect of the ligand on the cell's behavior is produced. Often, the behavior of a chain of several interacting cell proteins is altered following receptor activation or inhibition. The entire set of cell changes induced by receptor activation is called a signal transduction mechanism or signaling pathway.

The term “signals” as used herein refer to internal and external factors that control changes in cell structure and function. They can be chemical or physical in nature.

As used herein, a “spheroid” or “cell spheroid” means any grouping of cells in a three-dimensional shape that generally corresponds to an oval or circle rotated about one of its principal axes, major or minor, and includes three-dimensional egg shapes, oblate and prolate spheroids, spheres, and substantially equivalent shapes.

The term “subject” as used herein refers to any animal (e.g., a mammal), including, but not limited to, humans, non-human primates, canines, felines, rodents, and the like. Preferably, the subject is a human subject. The terms “subject,” “individual,” and “patient” are used interchangeably herein. The terms “subject,” “individual,” and “patient” thus encompass individuals having cancer (e.g., breast cancer), including those who have undergone or are candidates for resection (surgery) to remove cancerous tissue.

A “therapeutically effective amount” of a therapeutic agent, or combinations thereof, is an amount sufficient to treat disease in a subject.

The terms “treating” or “treatment” or “treat” as used herein refer to therapeutic measures that cure, slow down, lessen symptoms of, and/or halt progression of a diagnosed pathologic condition or disorder.

The term “two-dimensional culture” as used herein is defined as cultures of cells on flat hydrogels, including Matrigel® and vitronectin, disposed in culture vessels.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

The term “vitronectin” means a protein encoded by the VTN gene. A non-limiting example of vitronectin is the vitronectin from human (Homo sapiens, UniProt accession No. P04004) having the following sequence:

(SEQ ID NO: 1)
MAPLRPLLILALLAWVALADQESCKGRCTEGFNVDKKCQCDELCSYYQS
CCTDYTAECKPQVTRGDVFTMPEDEYTVYDDGEEKNNATVHEQVGGPSL
TSDLQAQSKGNPEQTPVLKPEEEAPAPEVGASKPEGIDSRPETLHPGRP
QPPAEEELCSGKPFDAFTDLKNGSLFAFRGQYCYELDEKAVRPGYPKLI
RDVWGIEGPIDAAFTRINCQGKTYLFKGSQYWRFEDGVLDPDYPRNISD
GFDGIPDNVDAALALPAHSYSGRERVYFFKGKQYWEYQFQHQPSQEECE
GSSLSAVFEHFAMMQRDSWEDIFELLFWGRTSAGTRQPQFISRDWHGVP
GQVDAAMAGRIYISGMAPRPSLAKKQRFRHRNRKGYRSQRGHSRGRNQN
SRRPSRATWLSLFSSEESNLGANNYDDYRMDWLVPATCEPIQSVFFFSG
DKYYRVNLRTRRVDTVDPPYPRSIAQYWLGCPAPGHL.

Another non-limiting example of vitronectin is the vitronectin from rat (Rattus norvegicus; GenBank accession No. NP 062029) having the following sequence:

(SEQ ID NO: 2)
MASLRPFFILALLALVSLADQESCKGRCTQGFMASKKCQCDELCTYYQS
CCVDYMEQCKPQVTRGDVFTMPEDEYWSYDYPEETKNSTSTGVQSENTS
LHFNLKPRAEETIKPTTPDPQEQSNTQEPEVGQQGVAPRPDTTDEGTSE
FPEEELCSGKPFDAFTDLKNGSLFAFRGEYCYELDETAVRPGYPKLIQD
VWGIEGPIDAAFTRINCQGKTYLFKGSQYWRFEDGVLDPDYPRNISEGF
SGIPDNVDAALALPAHSYSGRERVYFFKGKQYWEYEFQQQPSQEECEGS
SLSAVFEHFALLQRDSWENIFELLFWGRSSDGAKGPQFISRDWHGVPGK
VDAAMAGRIYITGSTFRSVQAKKQKSGRRSRKRYRSRRGRGHSRSRSRS
MSSRRPSRSVWFSLLSSEESGLGTYNYDYDMNWRIPATCEPIQSVYFFS
GDKYYRVNLRTRRVDSVNPPYPRSIAQYWLGCPTSEK.

A further non-limiting example of vitronectin is the vitronectin from mouse (Mus musculus; UniProt accession No. P29788) having the following sequence:

(SEQ ID NO: 3)
MAPLRPFFILALVAWVSLADQESCKGRCTQGFMASKKCQCDELCTYYQS
CCADYMEQCKPQVTRGDVFTMPEDDYWSYDYVEEPKNNTNTGVQPENTS
PPGDLNPRTDGTLKPTAFLDPEEQPSTPAPKVEQQEEILRPDTTDQGTP
EFPEEELCSGKPFDAFTDLKNGSLFAFRGQYCYELDETAVRPGYPKLIQ
DVWGIEGPIDAAFTRINCQGKTYLFKGSQYWRFEDGVLDPGYPRNISEG
FSGIPDNVDAAFALPAHRYSGRERVYFFKGKQYWEYEFQQQPSQEECEG
SSLSAVFEHFALLQRDSWENIFELLFWGRSSDGAREPQFISRNWHGVPG
KVDAAMAGRIYVTGSLSHSAQAKKQKSKRRSRKRYRSRRGRGHRRSQSS
NSRRSSRSIWFSLFSSEESGLGTYNNYDYDMDWLVPATCEPIQSVYFFS
GDKYYRVNLRTRRVDSVNPPYPRSIAQYWLGCPTSEK.

In some embodiments therefore, the vitronectin comprises at least about 70%, 75%, 80%, 85%, 90%, 95%, 96%, 97%, 98% or 99% sequence identity to SEQ ID NO: 1 or a functional fragment thereof In some embodiments therefore, the vitronectin comprises at least about 70%, 75%, 80%, 85%, 90%, 95%, 96%, 97%, 98% or 99% sequence identity to SEQ ID NO: 2 or a functional fragment thereof. In some embodiments therefore, the vitronectin comprises at least about 70%, 75%, 80%, 85%, 90%, 95%, 96%, 97%, 98% or 99% sequence identity to SEQ ID NO: 3 or a functional fragment thereof In some embodiments, vitronectin comprises at least about 75%, 80%, 85%, 90%, 95%, 96%, 97%, 98% or 99% sequence identity to SEQ ID NO: 1, SEQ ID NO: 2 or SEQ ID NO: 3, or a functional fragment thereof. In some embodiments, the vitronectin comprises SEQ ID NO: 1, SEQ ID NO: 2 or SEQ ID NO: 3, or a functional fragment thereof.

Methods

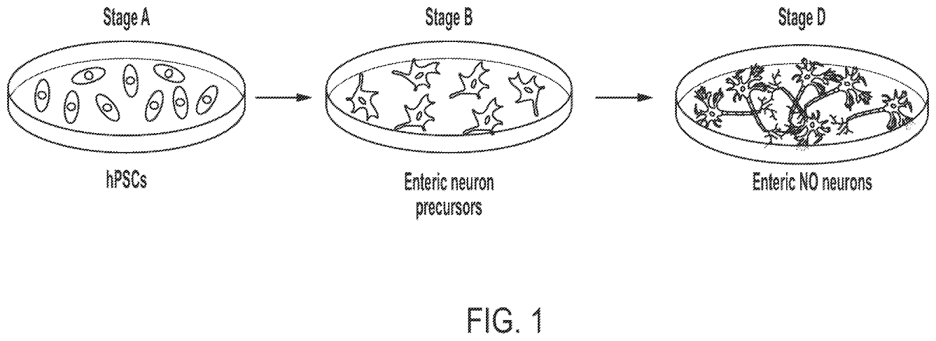

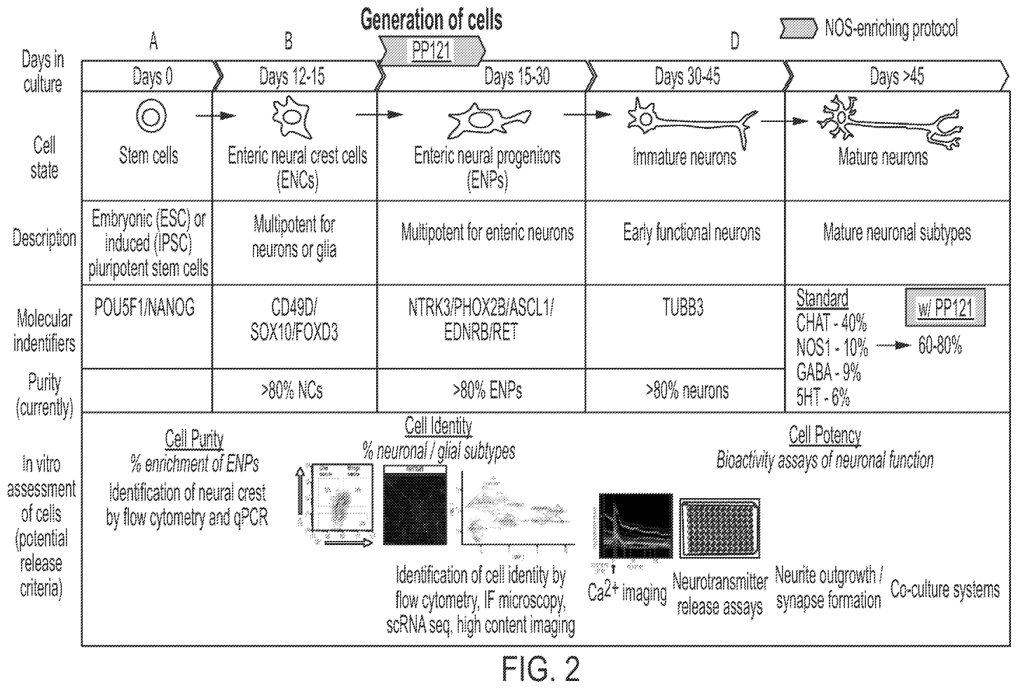

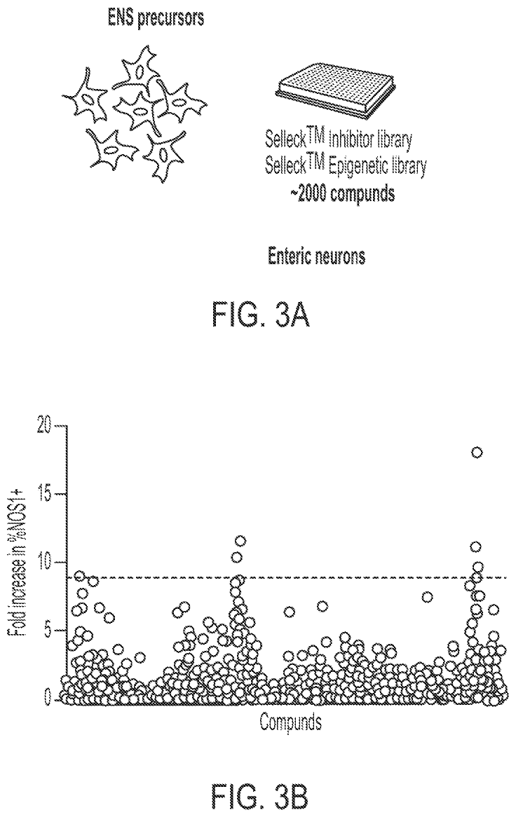

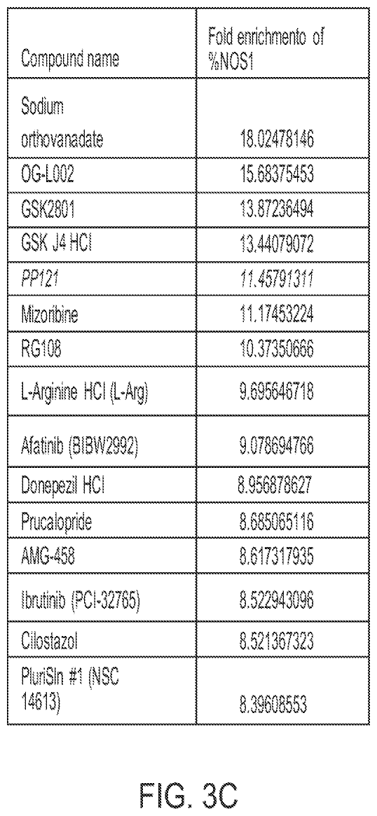

The disclosure provides novel methods for directed derivation of enteric nitrergic neurons from hPSCs (FIG. 1). Currently, there is no comparable methods for directed differentiation of enteric nitrergic neurons. Enteric nitrergic neurons are inhibitory neurons of the gastrointestinal (GI) system that regulate gut motility and are involved in a broad range of GI disorders. The methods of the present disclosure provide a scalable platform that make possible to produce unlimited number of hPSC-derived enteric nitrergic neurons on demand and enables high-throughput screening (HTS) assays for drug discovery targeting a broad range of GI disorders. The enteric nitrergic neurons generated by the methods of the disclosure also find applications in regenerative medicine, such as cell transplantation therapy, for motility disorders such as achalasia and gastroparesis. It should further be appreciated that the enteric nitrergic neurons generated by the methods of the disclosure may be used to replace damaged or absent cells relevant to enteric neuropathies. Moreover, the enteric nitrergic neurons generated by the methods of the disclosure provide translational applications that present a rational approach for preclinical development and as research tools.

The disclosure first provide improved methods for the derivation of enteric neural progenitors from human pluripotent stem cells (22). Many labs in the stem cell field no longer rely on the support of feeder cells and have adopted the use of defined basal media, such as mTeSR™1 (Stemcell Tech, 85850) or Essential 8 (Life Technologies, A2858501) for the maintenance of hPSC lines. Nevertheless, previous ENC induction methods commonly involve media containing serum replacement factors, namely knockout serum replacement (KSR), as is also the case in Comparative Example 2 (14, 20). In an effort to reduce the inconsistencies and quality control measures that undefined conditions may introduce to a protocol, optimizing ENC induction in minimal, chemically defined conditions, was pursued.

Recent studies have implemented alternative strategies for general NC induction using hPSCs, namely free floating embryoid body based approaches (23, 24). The migratory cells that come as a result of embryoid body and subsequent neural rosette formations have been shown to be positive for neural crest specific markers SOX10, TFAP2A, BRN3A, ISL1 and ASCL1, and a subset found to be positive for regionally specific vagal markers HOXB2 and HOXB5, even without the inclusion of RA (23). Overall neural crest induction efficiency was assessed by FACS of p75 and HNK1 double positive cells, a strategy used to isolate NC cells in previous protocols (Lee et al. 2007). Results showed >60% induction efficiency in ES cell line H9 and across independent hiPSC lines (23). Enriched NC populations were then co-cultured with primary gut explants in a Transwell system to promote ENC identities enriched for HOXB2, HOXB3, HAND2 and EDNRB. Notably, this method incorporates brain-derived neurotrophic factor (BDNF), glial cell line-derived neurotrophic factor (GDNF), nerve growth factor (NGF), neurotrophin-3 (NT3) into culture conditions. How these factors affect commitments of EN precursors, namely identities positive for VIP and calretinin (23), remains an interesting point of inquiry. A similar embryoid body approach incorporated brief exposure to RA during NC induction before eventually combining hPSC-derived NC cells with hPSC-derived intestinal organoids (HIOs) (24). In terms of the potential in ENC induction efficiency, comparative data between monolayer and embryoid body strategies remains limited. Indeed, the appropriate use of each strategy for a given application should be explored further.

The disclosure provides a method for the derivation of enteric nitrergic neuron (or enteric NO neuron) lineages from hPSCs. Methods for derivation of enteric neurons (ENs) from hPSCs have been previously described in, for example, PCT application No. PCT/US2019/068447 filed on Dec. 23, 2019, incorporated by reference herein in its entirety. However, despite extensive efforts and the important medical implications of GI disorders, the in vitro derivation of enteric nitrergic neurons, inhibitory neurons of the GI system that regulate gut motility and involve in a broad range of GI disorders, remains elusive. In fact, there is currently no alternative method to generate enteric NO neurons from stem cells.

The disclosure relates to a method of differentiating at least one or a plurality of stem cells into at least one or a plurality of enteric nitrergic neurons (or enteric NO neurons), the method comprising exposing one or more nitrergic agents to a plurality of enteric neural crest cells, wherein at least about 30% of the enteric neurons express NOS1. In some embodiments, the method further comprises differentiating one or a plurality of stem cells into one or a plurality of enteric neural crest cells prior to the step of exposing the neural crest cells. In some embodiments, the disclosure relates to a method of differentiating a stem cell into an enteric neuronal cell by exposing the cells to a first differentiation medium, a second differentiation medium, a third differentiation medium, wherein the first differentiation medium comprises: BMP4 (about 1 ng ml⁻¹), SB431542 (about 10 μM), CHIR 99021 (about 600 nM), with Essential 6™ Medium; wherein the second differentiation medium comprises: SB431542 (about 10 μM), CHIR 99021 (about 1.5 μM), with Essential 6™ medium; wherein the third differentiation medium comprises SB431542 (about 10 μM), CHIR 99021 (about 1.5 μM), Retinoic Acid (about 1 μM), with Essential 6™ medium. In some embodiments, the methods comprise an exposure step comprising exposing the cells in culture to a fourth differentiation medium comprising: GDNF (about 10 ng ml⁻¹), Ascorbic Acid (about 100 μM), N2 Supplement (about 10 μl ml⁻¹), B27 Supplement (about 20 μl ml⁻¹), Glutagro (about 10 μl ml⁻¹), MEM Nonessential Amino Acids (about 10 μl ml⁻¹), with Neurobasal® Medium. In some embodiments, the cells are further exposed to a biologically effective amount of one or a plurality of nitrergic agents during exposure to the first, second, third or fourth differentiation medium. In some embodiments, the cells are further exposed to a biologically effective amount of one or a plurality of nitrergic agents during exposure to the fourth differentiation medium. In some embodiments, the cells are exposed to the following maintenance medium between exposure to the third or fourth differentiation mediums:

FGF2 (about 10 ng ml⁻¹), CHIR 99021 (about 3 μM), N2 Supplement (about 10 μl ml⁻¹), B27 Supplement (about 20 μl ml⁻¹), Glutagro (about 10 μl ml⁻¹), MEM Nonessential Amino Acids (about 10 μl ml⁻¹), with Neurobasal® Medium.

In some embodiments, the disclosure relates to a method of differentiating cells into enteric neurons or creating a library of enteric neurons comprising:
(a) culturing human pleuripotent stem cells from about 5 to about 7 days;
(b) inducing the human pluripotent stem cells into enteric neural crest cells for about 12 to about 15 days;
(c) exposing the neural crest cells to the fourth differentiation medium for a period of time sufficient to differentiate the neural crest cells to enteric neurons.
In some embodiments, the neural crest cells are exposed to the fourth differentiation medium for a period of from about 1 to about 3 days.
In some embodiments, the step of inducing the human pluripotent stem cells into neural crest cells is from about 12 to about 15 days. In some embodiments the human pluripotent stem cells are differentiated into neural crest cells by sequential exposure to the first, second and third differentiation medium. In some embodiments, the method further comprises the step of exposing the cells to a biologically effective amount of one or a plurality of nitrergic agents.

In some embodiments, the disclosure relates to a method of differentiating neural crest cells into entric neurons by exposing the cells to the fourth differentiation medium. In some embodiments, the step of exposing the neural crest cells is from about 8 hours to about 48 hours. In some embodiments, the method further comprises the step of exposing the cells to a biologically effective amount of one or a plurality of nitrergic agents.

The disclosure also relates to a method of producing nitric oxide synthase (NOS)-expressing enteric neurons, the method comprising exposing one or more nitrergic agents to one or a plurality of enteric neural crest cells. In some embodiments, the plurality of enteric neural crest cells are differentiated from one or a plurality of pluripotent stem cells.

In some embodiments, the one or plurality of stem cells comprises an embryonic stem cell. In some embodiments, the one or plurality of stem cells comprises a pluripotent stem cell. In some embodiments, the one or plurality of stem cells comprises a human embryonic stem cell. In some embodiments, the one or plurality of stem cells comprises a human pluripotent stem cell. In some embodiments, the one or plurality of stem cells comprises an induced human pluripotent stem cell. In some embodiments, the one or plurality of stem cells comprises human inducible pluripotent stem cells. In some embodiments, the one or plurality of stem cells comprises hematopoetic stem cells, neural stem cells, adipose derived stem cells, bone marrow derived stem cells, induced pluripotent stem cells, astrocyte derived induced pluripotent stem cells, fibroblast derived induced pluripotent stem cells, renal epithelial derived induced pluripotent stem cells, keratinocyte derived induced pluripotent stem cells, peripheral blood derived induced pluripotent stem cells, hepatocyte derived induced pluripotent stem cells, mesenchymal derived induced pluripotent stem cells, neural stem cell derived induced pluripotent stem cells, adipose stem cell derived induced pluripotent stem cells, preadipocyte derived induced pluripotent stem cells, chondrocyte derived induced pluripotent stem cells, and/or skeletal muscle derived induced pluripotent stem cells.

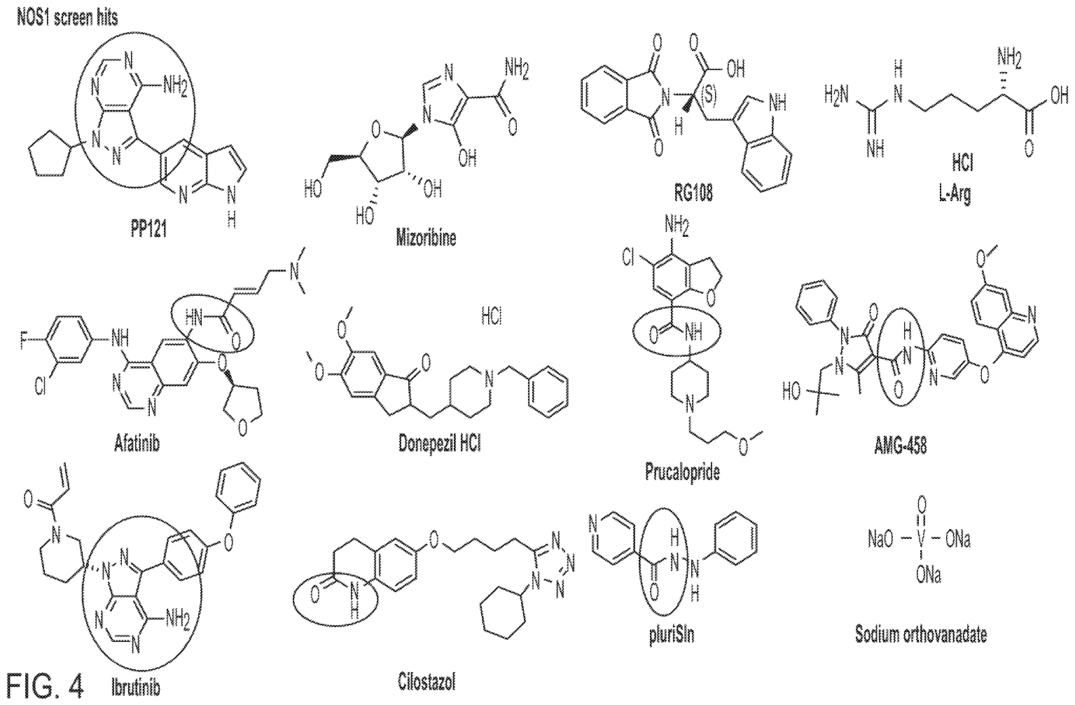

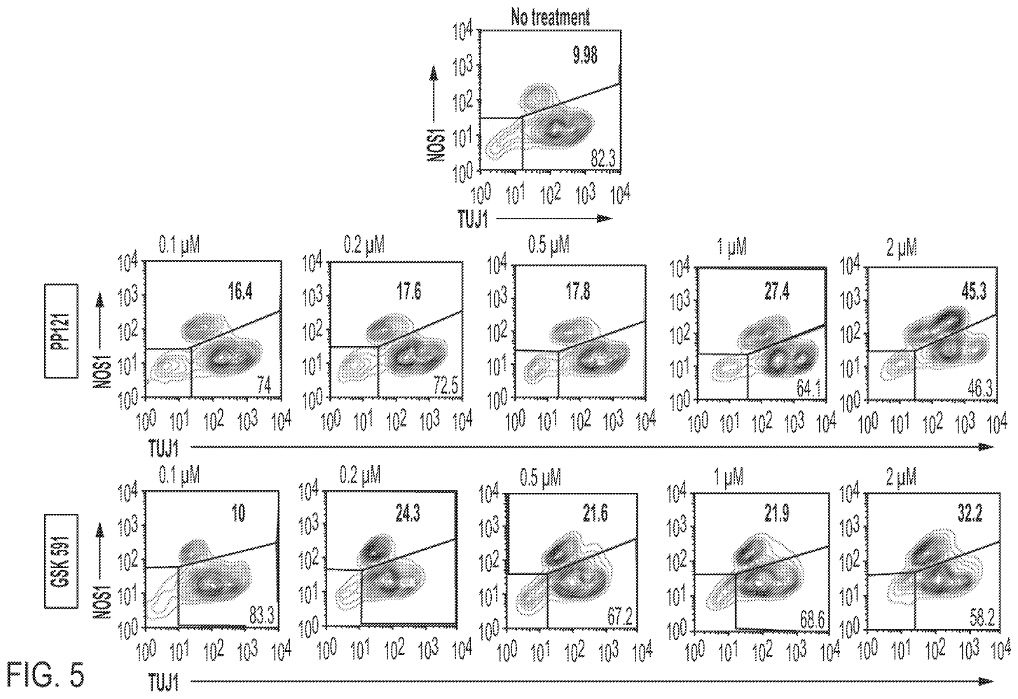

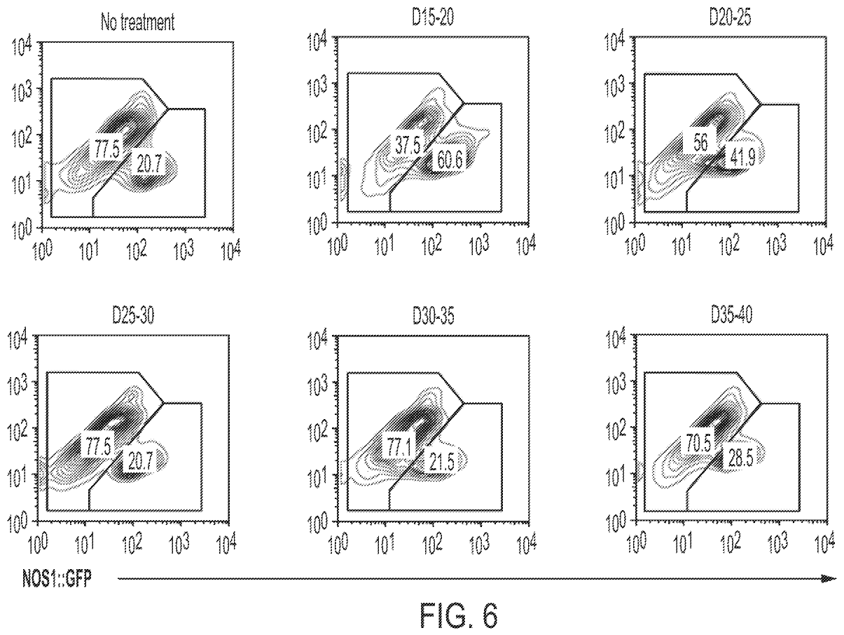

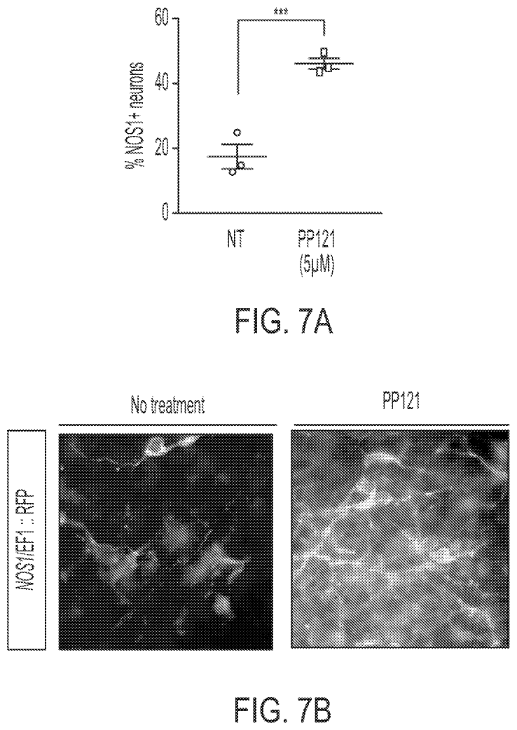

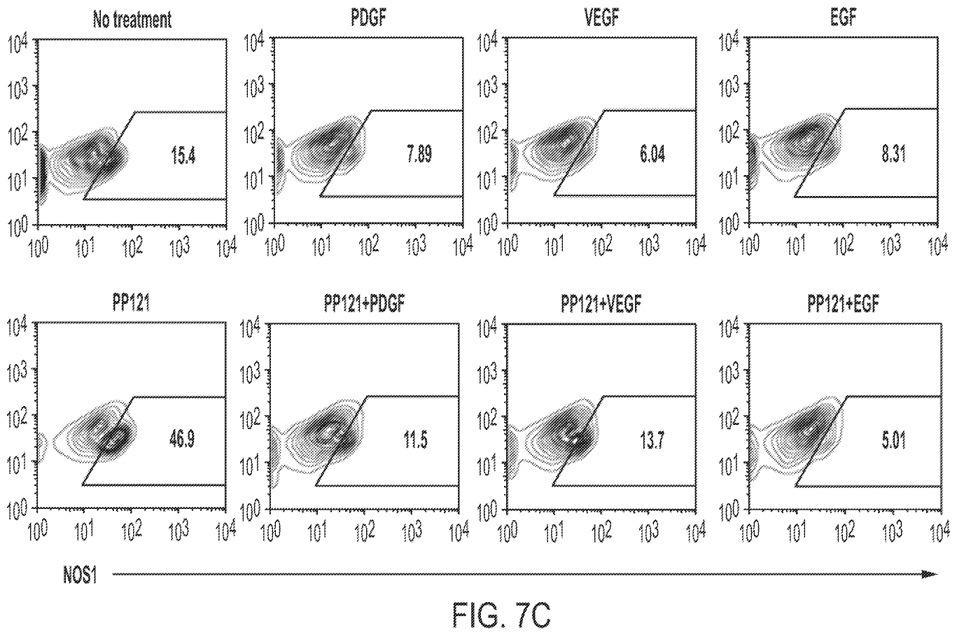

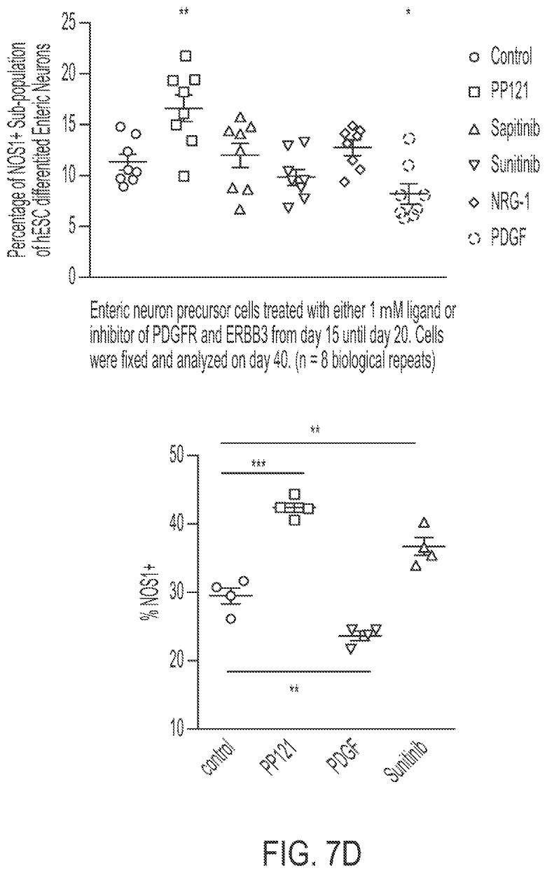

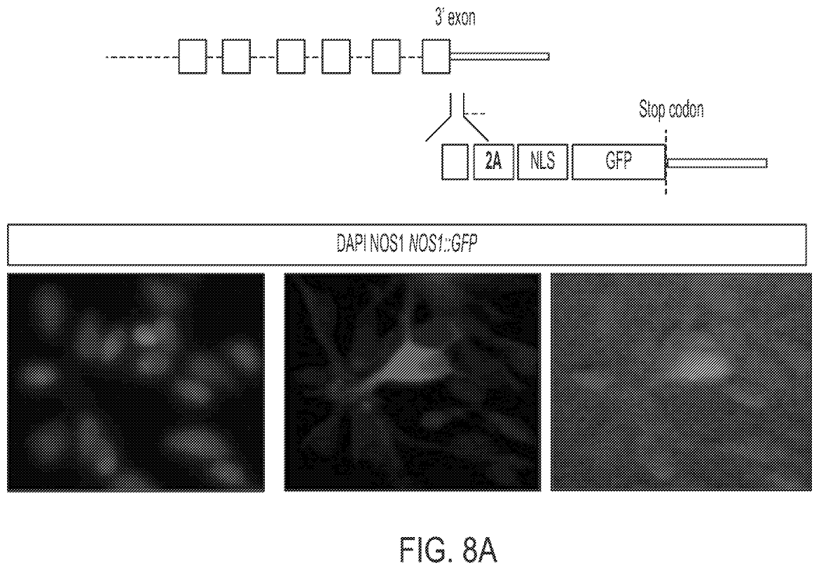

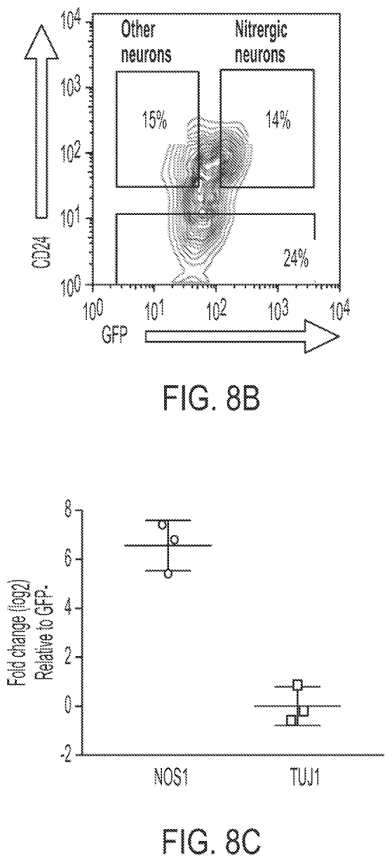

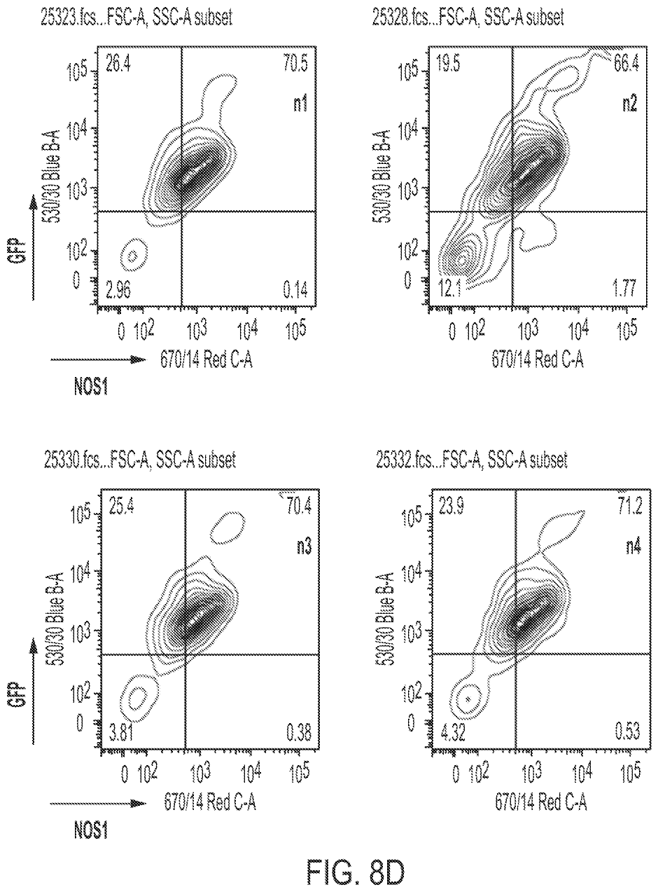

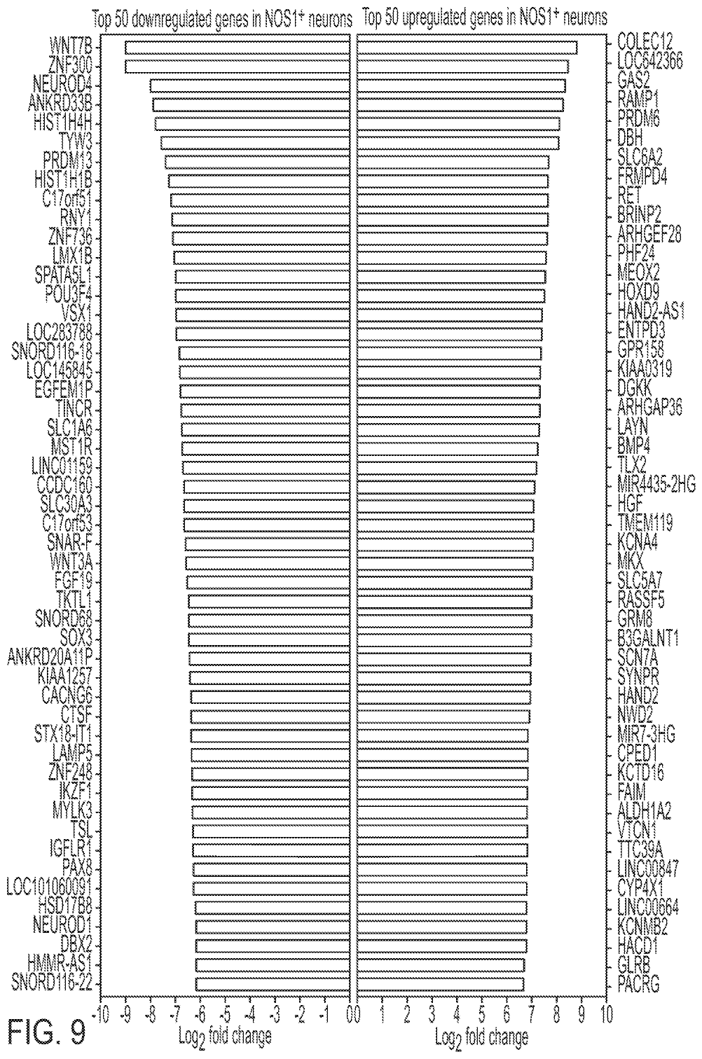

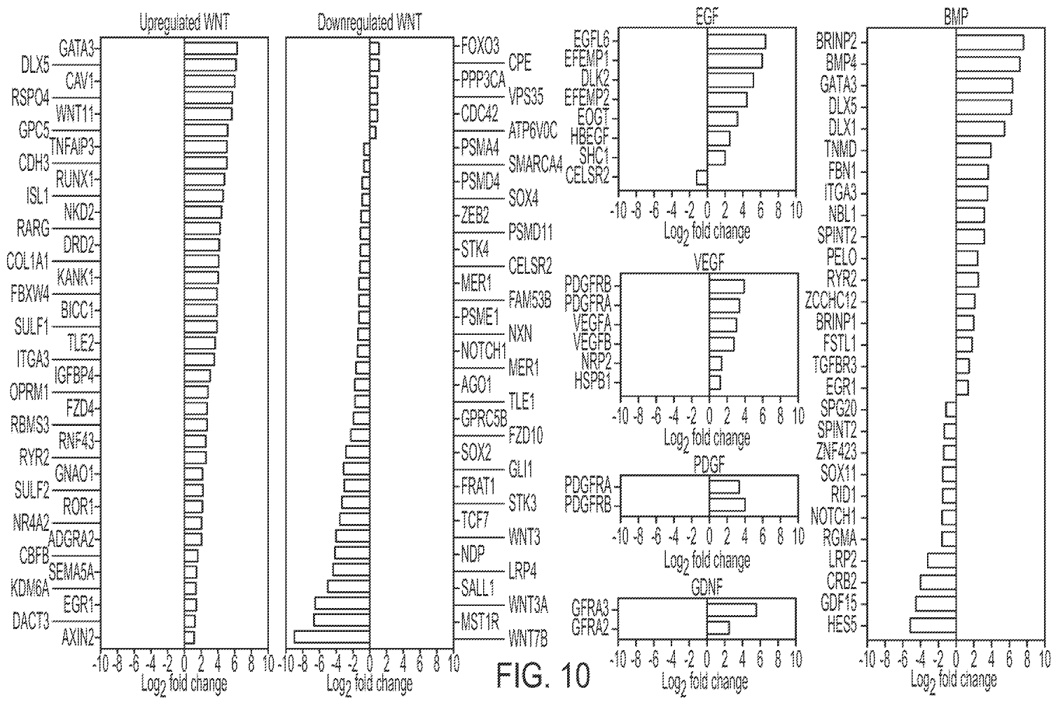

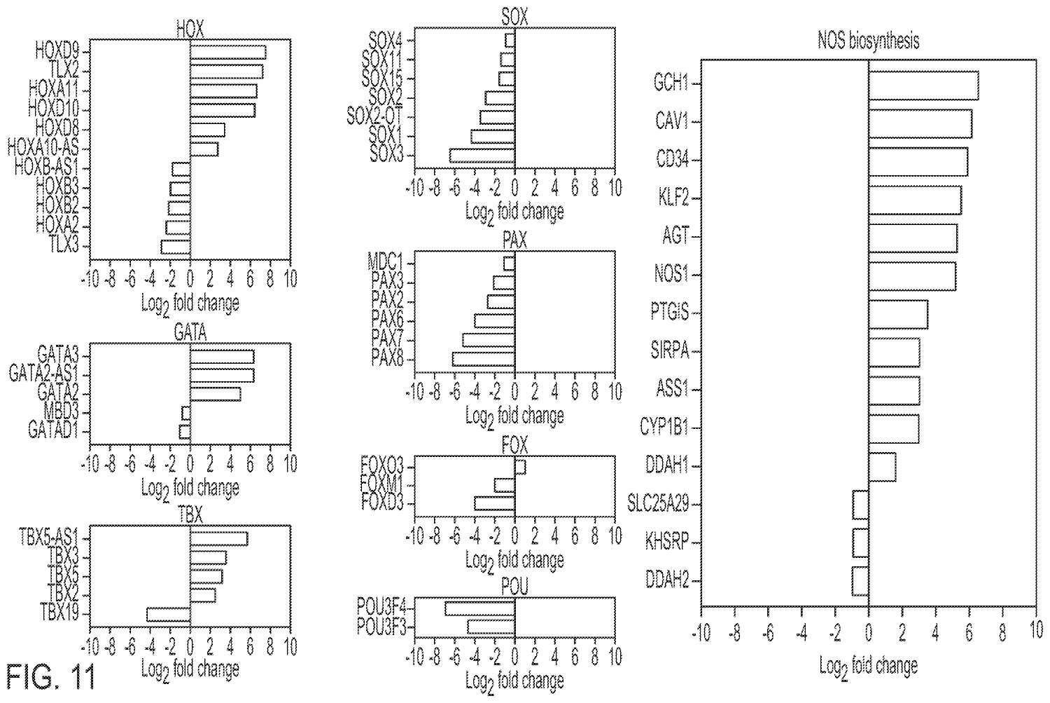

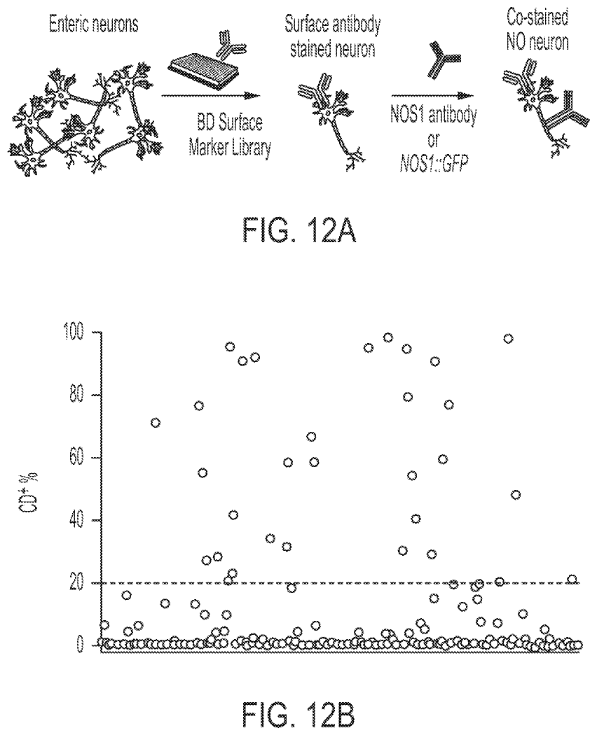

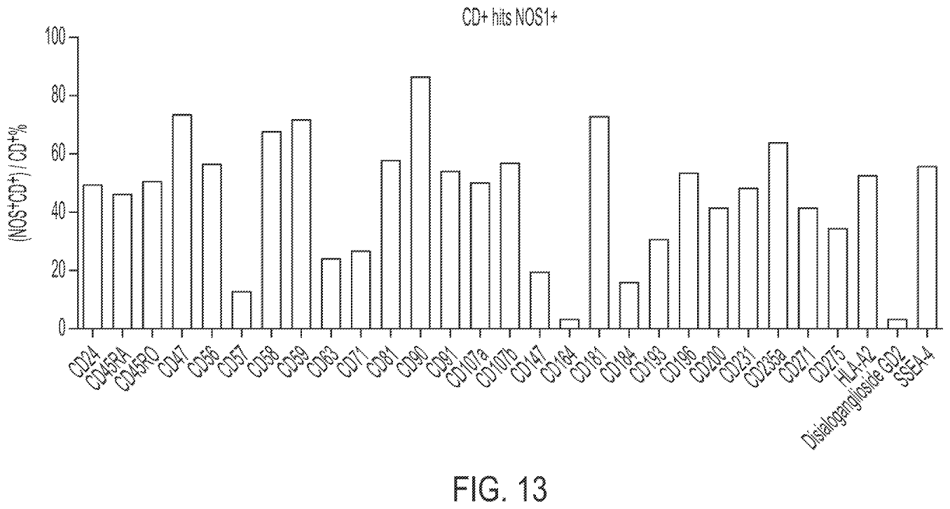

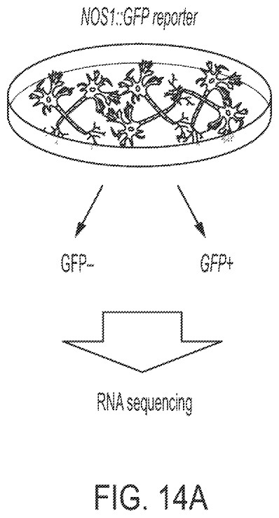

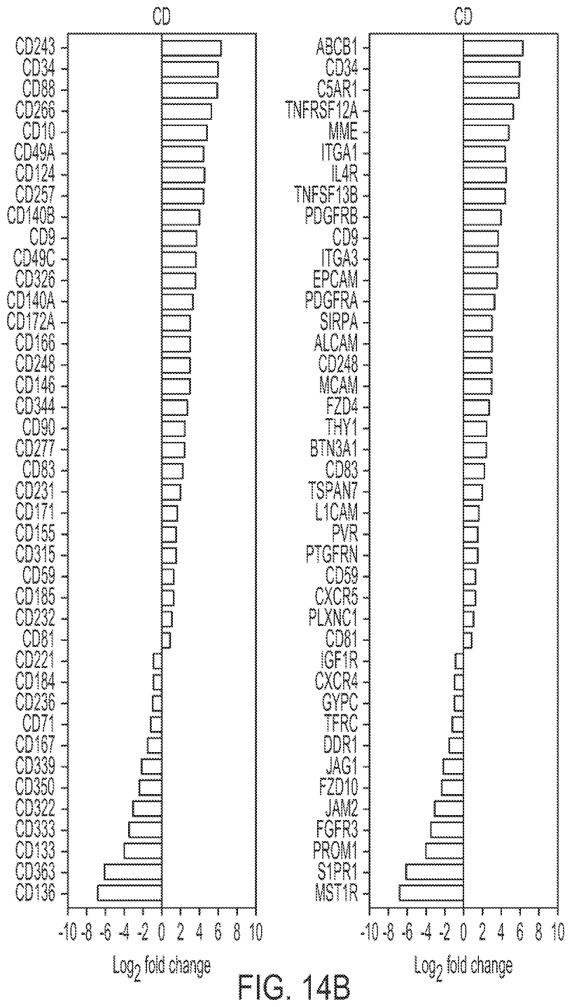

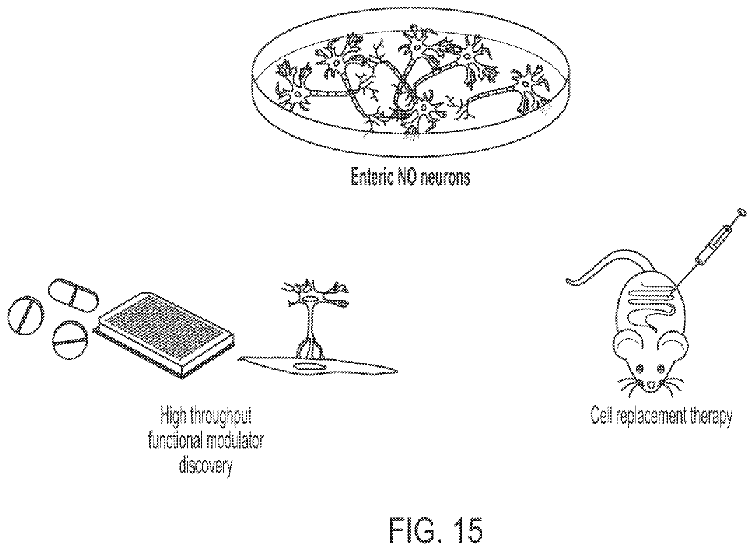

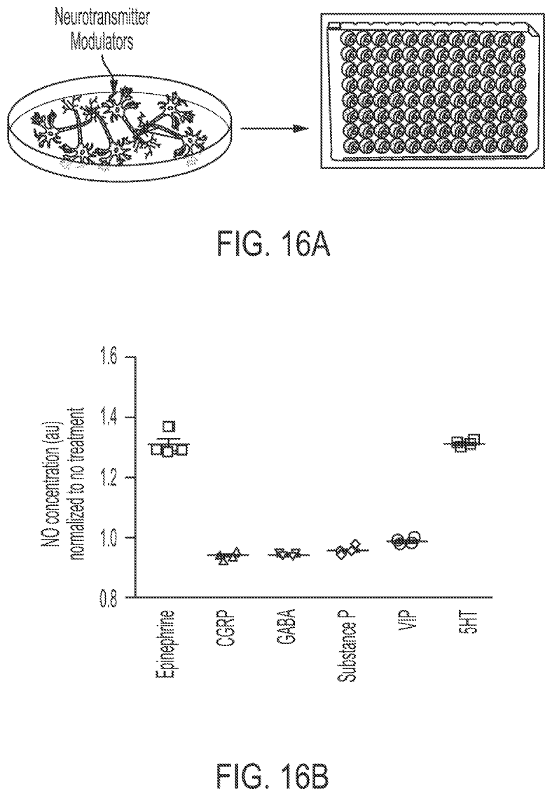

The term “nitrergic agent” as used herein refers to an agent, when exposed to a call, causes or induces the cell to express nitric oxide synthase (NOS1). In some embodiments, the nitrergic agent is a RTK inhibitor. The term “RTK inhibitor” as used herein, includes, but is not limited to, protein tyrosine kinase and/or serine and/or threonine kinase inhibitors or lipid kinase inhibitors. In some embodiments therefore, the nitrergic agent is a protein tyrosine kinase. In some embodiments therefore, the nitrergic agent is a serine and/or threonine kinase inhibitor. In some embodiments therefore, the nitrergic agent is a lipid kinase inhibitor. In some embodiments, the nitrergic agent is a compound selected from the group of PP121, afatinib, ibrutinib, mizoribine, donepezil, cilostazol, RG108, prucalopride, PluriSIn #1, L-Arginine, AMG-458, OG-L002, GSK2801, GSK J4, GSK591, and sodium orthovanadate, or a salt of any of the foregoing. In some embodiments, the nitrergic agent is a compound selected from the group of compounds provided in FIG. 4. In some embodiments, the nitrergic agent is PP121, which is a dual inhibitor of tyrosine and phosphoinositide kinases, or a salt thereof. In some embodiments, the nitrergic agent is afatinib, or a salt thereof. In some embodiments, the nitrergic agent is ibrutinib, or a salt thereof. In some embodiments, the nitrergic agent is mizoribine, or a salt thereof. In some embodiments, the nitrergic agent is donepezil, or a salt thereof. In some embodiments, the nitrergic agent is cilostazol, or a salt thereof. In some embodiments, the nitrergic agent is RG108, or a salt thereof. In some embodiments, the nitrergic agent is prucalopride, or a salt thereof. In some embodiments, the nitrergic agent is PluriSIn #1, or a salt thereof. In some embodiments, the nitrergic agent is L-Arginine, or a salt thereof. In some embodiments, the nitrergic agent is AMG-458, or a salt thereof. In some embodiments, the nitrergic agent is OG-L002, or a salt thereof. In some embodiments, the nitrergic agent is GSK2801, or a salt thereof. In some embodiments, the nitrergic agent is GSK J4, or a salt thereof. In some embodiments, the nitrergic agent is GSK591, or a salt thereof. In some embodiments, the nitrergic agent is sodium orthovanadate, or a salt thereof.

In some embodiments, the nitrergic agent has a structure represented by a formula:

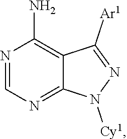

wherein Cy¹ is selected from C3-C8 cycloalkyl and C2-C9 heterocycloalkyl, and is substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹;

wherein R₁, when present, is selected from hydrogen, C1-C4 alkyl, C2-C4 alkenyl, and C1-C4 haloalkyl;

wherein Ar¹ is selected from C6-C10 aryl and C2-C9 heteroaryl, and is substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr²;

wherein Ar², when present, is selected from C6-C10 aryl and C2-C9 heteroaryl, and is substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, and (C1-C4)(C1-C4) dialkylamino,

or a pharmaceutically acceptable salt thereof.

In some embodiments, the nitrergic agent has a structure represented by the aforementioned formula, wherein Cy¹ is unsubstituted C3-C8 cycloalkyl. In some embodiments, the nitrergic agent has a structure represented by the aforementioned formula, wherein Cy¹ is unsubstituted cyclopentyl. In some embodiments, the nitrergic agent has a structure represented by the aforementioned formula, wherein Cy¹ is C2-C9 heterocycloalkyl substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹. In some embodiments, the nitrergic agent has a structure represented by the aforementioned formula, wherein Cy¹ is C2-C9 heterocycloalkyl substituted with a group selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹. In some embodiments, the nitrergic agent has a structure represented by the aforementioned formula, wherein Cy¹ is C2-C9 heterocycloalkyl substituted with a —C(O)R¹ group. In some embodiments, the nitrergic agent has a structure represented by the aforementioned formula, wherein Cy¹ is piperidinyl substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹. In some embodiments, the nitrergic agent has a structure represented by the aforementioned formula, wherein Cy¹ is piperidinyl substituted with a group selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹. In some embodiments, the nitrergic agent has a structure represented by the aforementioned formula, wherein Cy¹ is piperidinyl substituted with a —C(O)R¹ group. In some embodiments, the nitrergic agent has a structure represented by the aforementioned formula, wherein R¹, when present, is C2-C4 alkenyl. In some embodiments, the nitrergic agent has a structure represented by the aforementioned formula, wherein R¹, when present, is ethenyl. In some embodiments, the nitrergic agent has a structure represented by the aforementioned formula, wherein Ar¹ is unsubstituted C2-C9 heteroaryl. In some embodiments, the nitrergic agent has a structure represented by the aforementioned formula, wherein Ar¹ is unsubstituted pyrrolopyridinyl. In some embodiments, the nitrergic agent has a structure represented by the aforementioned formula, wherein Ar¹ is selected from C6-C10 aryl substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr². In some embodiments, the nitrergic agent has a structure represented by the aforementioned formula, wherein Ar¹ is selected from C6-C10 aryl substituted with a group selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr². In some embodiments, the nitrergic agent has a structure represented by the aforementioned formula, wherein Ar¹ is selected from C6-C10 aryl substituted with —OAr². In some embodiments, the nitrergic agent has a structure represented by the aforementioned formula, wherein Ar¹ is selected from C6 aryl substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr². In some embodiments, the nitrergic agent has a structure represented by the aforementioned formula, wherein Ar¹ is selected from C6 aryl substituted with a group selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr². In some embodiments, the nitrergic agent has a structure represented by the aforementioned formula, wherein Ar¹ is selected from C6 aryl substituted with a —OAr². In some embodiments, the nitrergic agent has a structure represented by the aforementioned formula, wherein Ar², when present, is unsubstituted C6-C10 aryl. In some embodiments, the nitrergic agent has a structure represented by the aforementioned formula, wherein Ar², when present, is unsubstituted C6 aryl.

In some embodiments, the nitrergic agent has a structure represented by a formula selected from:

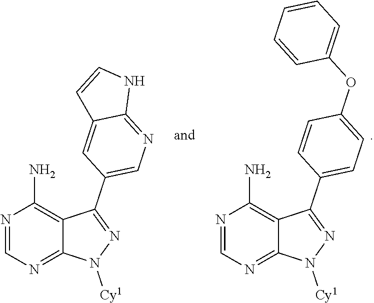

In some embodiments, the nitrergic agent is selected from:

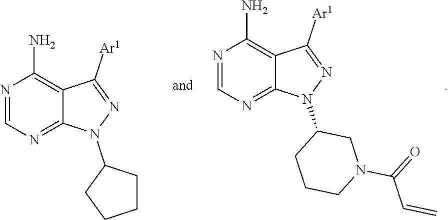

In some embodiments, the nitrergic agent is selected from:

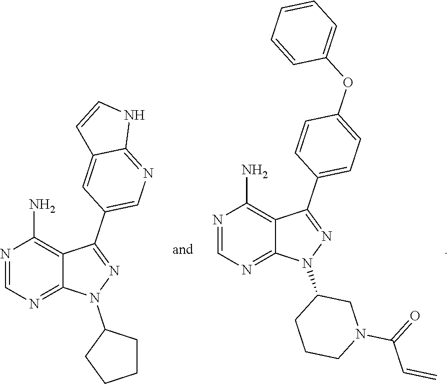

In some embodiments, the nitrergic agent is selected from:

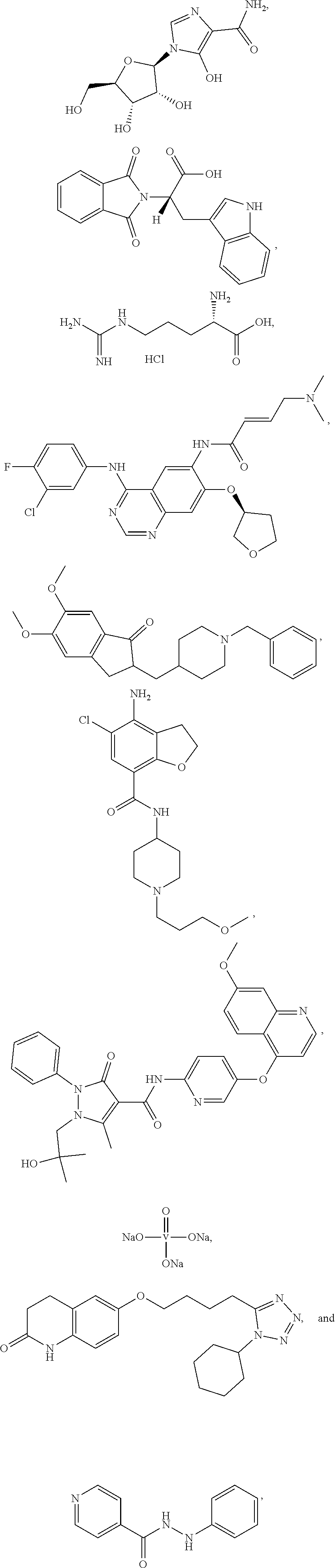

or a pharmaceutically acceptable salt thereof.

In some embodiments, portion of the enteric nitrergic neurons obtained by the methods of the disclosure express neuronal nitric oxide synthase 1, also known as NOS or NOS1. NOS1 is an enzyme that produces nitric oxide (NO) in the central and peripheral nervous systems. A non-limiting example of NOS1 is the NOS1 from human (Homo sapiens, UniProt accession No. P29475) having the following sequence:

(SEQ ID NO: 10)
MEDHMFGVQQIQPNVISVRLFKRKVGGLGFLVKERVSKPPVIISDLIRG
GAAEQSGLIQAGDIILAVNGRPLVDLSYDSALEVLRGIASETHVVLILR
GPEGFTTHLETTFTGDGTPKTIRVTQPLGPPTKAVDLSHQPPAGKEQPL
AVDGASGPGNGPQHAYDDGQEAGSLPHANGLAPRPPGQDPAKKATRVSL
QGRGENNELLKEIEPVLSLLTSGSRGVKGGAPAKAEMKDMGIQVDRDLD
GKSHKPLPLGVENDRVFNDLWGKGNVPVVLNNPYSEKEQPPTSGKQSPT
KNGSPSKCPRFLKVKNWETEVVLTDTLHLKSTLETGCTEYICMGSIMHP
SQHARRPEDVRTKGQLFPLAKEFIDQYYSSIKRFGSKAHMERLEEVNKE
IDTTSTYQLKDTELIYGAKHAWRNASRCVGRIQWSKLQVFDARDCTTAH
GMFNYICNHVKYATNKGNLRSAITIFPQRTDGKHDFRVWNSQLIRYAGY
KQPDGSTLGDPANVQFTEICIQQGWKPPRGRFDVLPLLLQANGNDPELF
QIPPELVLEVPIRHPKFEWFKDLGLKWYGLPAVSNMLLEIGGLEFSACP
FSGWYMGTEIGVRDYCDNSRYNILEEVAKKMNLDMRKTSSLWKDQALVE
INIAVLYSFQSDKVTIVDHHSATESFIKHMENEYRCRGGCPADWVWIVP
PMSGSITPVFHQEMLNYRLTPSFEYQPDPWNTHVWKGTNGTPTKRRAIG
FKKLAEAVKFSAKLMGQAMAKRVKATILYATETGKSQAYAKTLCEIFKH
AFDAKVMSMEEYDIVHLEHETLVLVVTSTFGNGDPPENGEKFGCALMEM
RHPNSVQEERKSYKVRFNSVSSYSDSQKSSGDGPDLRDNFESAGPLANV
RFSVFGLGSRAYPHFCAFGHAVDTLLEELGGERILKMREGDELCGQEEA
FRTWAKKVFKAACDVFCVGDDVNIEKANNSLISNDRSWKRNKFRLTFVA
EAPELTQGLSNVHKKRVSAARLLSRQNLQSPKSSRSTIFVRLHTNGSQE
LQYQPGDHLGVFPGNHEDLVNALIERLEDAPPVNQMVKVELLEERNTAL
GVISNWTDELRLPPCTIFQAFKYYLDITTPPTPLQLQQFASLATSEKEK
QRLLVLSKGLQEYEEWKWGKNPTIVEVLEEFPSIQMPATLLLTQLSLLQ
PRYYSISSSPDMYPDEVHLTVAIVSYRTRDGEGPIHHGVCSSWLNRIQA
DELVPCFVRGAPSFHLPRNPQVPCILVGPGTGIAPFRSFWQQRQFDIQH
KGMNPCPMVLVFGCRQSKIDHIYREETLQAKNKGVFRELYTAYSREPDK
PKKYVQDILQEQLAESVYRALKEQGGHIYVCGDVTMAADVLKAIQRIMT
QQGKLSAEDAGVFISRMRDDNRYHEDIFGVTLRTYEVTNRLRSESIAFI
EESKKDTDEVFSS.

Another non-limiting example of NOS1 is the NOS1 from rat (Rattus norvegicus; UniProt accession No. P29476) having the following sequence:

(SEQ ID NO: 11)
MEENTFGVQQIQPNVISVRLFKRKVGGLGFLVKERVSKPPVIISDLIR
GGAAEQSGLIQAGDIILAVNDRPLVDLSYDSALEVLRGIASETHVVLI
LRGPEGFTTHLETTFTGDGTPKTIRVTQPLGPPTKAVDLSHQPSASKD
QSLAVDRVTGLGNGPQHAQGHGQGAGSVSQANGVAIDPTMKSTKANLQ
DIGEHDELLKEIEPVLSILNSGSKATNRGGPAKAEMKDTGIQVDRDLD
GKSHKAPPLGGDNDRVFNDLWGKDNVPVILNNPYSEKEQSPTSGKQSP
TKNGSPSRCPRFLKVKNWETDVVLTDTLHLKSTLETGCTEHICMGSIM
LPSQHTRKPEDVRTKDQLFPLAKEFLDQYYSSIKRFGSKAHMDRLEEV
NKEIESTSTYQLKDTELIYGAKHAWRNASRCVGRIQWSKLQVFDARDC
TTAHGMFNYICNHVKYATNKGNLRSAITIFPQRTDGKHDFRVWNSQLI
RYAGYKQPDGSTLGDPANVQFTEICIQQGWKAPRGRFDVLPLLLQANG
NDPELFQIPPELVLEVPIRHPKFDWFKDLGLKWYGLPAVSNMLLEIGG
LEFSACPFSGWYMGTEIGVRDYCDNSRYNILEEVAKKMDLDMRKTSSL
WKDQALVEINIAVLYSFQSDKVTIVDHHSATESFIKHMENEYRCRGGC
PADWVWIVPPMSGSITPVFHQEMLNYRLTPSFEYQPDPWNTHVWKGTN
GTPTKRRAIGFKKLAEAVKFSAKLMGQAMAKRVKATILYATETGKSQA
YAKTLCEIFKHAFDAKAMSMEEYDIVHLEHEALVLVVTSTFGNGDPPE
NGEKFGCALMEMRHPNSVQEERKSYKVRFNSVSSYSDSRKSSGDGPDL
RDNFESTGPLANVRFSVFGLGSRAYPHFCAFGHAVDTLLEELGGERIL
KMREGDELCGQEEAFRTWAKKVFKAACDVFCVGDDVNIEKPNNSLISN
DRSWKRNKFRLTYVAEAPDLTQGLSNVHKKRVSAARLLSRQNLQSPKF
SRSTIFVRLHTNGNQELQYQPGDHLGVFPGNHEDLVNALIERLEDAPP
ANHVVKVEMLEERNTALGVISNWKDESRLPPCTIFQAFKYYLDITTPP
TPLQLQQFASLATNEKEKQRLLVLSKGLQEYEEWKWGKNPTMVEVLEE
FPSIQMPATLLLTQLSLLQPRYYSISSSPDMYPDEVHLTVAIVSYHTR
DGEGPVHHGVCSSWLNRIQADDVVPCFVRGAPSFHLPRNPQVPCILVG
PGTGIAPFRSFWQQRQFDIQHKGMNPCPMVLVFGCRQSKIDHIYREET
LQAKNKGVFRELYTAYSREPDRPKKYVQDVLQEQLAESVYRALKEQGG
HIYVCGDVTMAADVLKAIQRIMTQQGKLSEEDAGVFISRLRDDNRYHE
DIFGVTLRTYEVTNRLRSESIAFIEESKKDADEVFSS.

A further non-limiting example of NOS1 is the NOS1 from mouse (Mus musculus; UniProt accession No. Q9Z0J4) having the following sequence:

(SEQ ID NO: 12)
MEEHTFGVQQIQPNVISVRLFKRKVGGLGFLVKERVSKPPVIISDLIR
GGAAEQSGLIQAGDIILAVNDRPLVDLSYDSALEVLRGIASETHVVLI
LRGPEGFTTHLETTFTGDGTPKTIRVTQPLGTPTKAVDLSRQPSASKD
QPLAVDRVPGPSNGPQHAQGRGQGAGSVSQANGVAIDPTMKNTKANLQ
DSGEQDELLKEIEPVLSILTGGGKAVNRGGPAKAEMKDTGIQVDRDLD
GKLHKAPPLGGENDRVFNDLWGKGNVPVVLNNPYSENEQSPASGKQSP
TKNGSPSRCPRFLKVKNWETDVVLTDTLHLKSTLETGCTEQICMGSIM
LPSHHIRKSEDVRTKDQLFPLAKEFLDQYYSSIKRFGSKAHMDRLEEV
NKEIESTSTYQLKDTELIYGAKHAWRNASRCVGRIQWSKLQVFDARDC
TTAHGMFNYICNHVKYATNKGNLRSAITIFPQRTDGKHDFRVWNSQLI
RYAGYKQPDGSTLGDPANVEFTEICIQQGWKPPRGRFDVLPLLLQANG
NDPELFQIPPELVLEVPIRHPKFDWFKDLGLKWYGLPAVSNMLLEIGG
LEFSACPFSGWYMGTEIGVRDYCDNSRYNILEEVAKKMDLDMRKTSSL
WKDQALVEINIAVLYSFQSDKVTIVDHHSATESFIKHMENEYRCRGGC
PADWVWIVPPMSGSITPVFHQEMLNYRLTPSFEYQPDPWNTHVWKGTN
GTPTKRRAIGFKKLAEAVKFSAKLMGQAMAKRVKATILYATETGKSQA
YAKTLCEIFKHAFDAKAMSMEEYDIVHLEHEALVLVVTSTFGNGDPPE
NGEKFGCALMEMRHPNSVQEERKSYKVRFNSVSSYSDSRKSSGDGPDL
RDNFESTGPLANVRFSVFGLGSRAYPHFCAFGHAVDTLLEELGGERIL
KMREGDELCGQEEAFRTWAKKVFKAACDVFCVGDDVNIEKANNSLISN
DRSWKRNKFRLTYVAEAPELTQGLSNVHKKRVSAARLLSRQNLQSPKS
SRSTIFVRLHTNGNQELQYQPGDHLGVFPGNHEDLVNALIERLEDAPP
ANHVVKVEMLEERNTALGVISNWKDESRLPPCTIFQAFKYYLDITTPP
TPLQLQQFASLATNEKEKQRLLVLSKGLQEYEEWKWGKNPTMVEVLEE
FPSIQMPATLLLTQLSLLQPRYYSISSSPDMYPDEVHLTVAIVSYHTR
DGEGPVHHGVCSSWLNRIQADDVVPCFVRGAPSFHLPRNPQVPCILVG
PGTGIAPFRSFWQQRQFDIQHKGMNPCPMVLVFGCRQSKIDHIYREET
LQAKNKGVFRELYTAYSREPDRPKKYVQDVLQEQLAESVYRALKEQGG
HIYVCGDVTMAADVLKAIQRIMTQQGKLSEEDAGVFISRLRDDNRYHE
DIFGVTLRTYEVTNRLRSESIAFIEESKKDTDEVFSS.

In some embodiments therefore, the NOS1 expressed in the enteric neurons produced by the disclosed methods comprises at least about 70%, 75%, 80%, 85%, 90%, 95%, 96%, 97%, 98% or 99% sequence identity to SEQ ID NO: 10 or a functional fragment thereof. In some embodiments therefore, NOS1 comprises at least about 70%, 75%, 80%, 85%, 90%, 95%, 96%, 97%, 98% or 99% sequence identity to SEQ ID NO: 11 or a functional fragment thereof. In some embodiments therefore, NOS1 comprises at least about 70%, 75%, 80%, 85%, 90%, 95%, 96%, 97%, 98% or 99% sequence identity to SEQ ID NO: 12 or a functional fragment thereof. In some embodiments, the NOS1 comprises SEQ ID NO: 10 or a functional fragment thereof In some embodiments, the NOS1 comprises SEQ ID NO: 11 or a functional fragment thereof In some embodiments, the NOS1 comprises SEQ ID NO: 12 or a functional fragment thereof.

In some embodiments, at least about 20% to about 100% of the enteric neurons produced by the disclosed methods express NOS1. In some embodiments, at least about 25% to about 90% of the enteric neurons produced by the disclosed methods express NOS1. In some embodiments, at least about 30% to about 85% of the enteric neurons produced by the disclosed methods express NOS1. In some embodiments, at least about 35 to about 80% of the enteric neurons produced by the disclosed methods express NOS1. In some embodiments, at least about 40% to about 80% of the enteric neurons produced by the disclosed methods express NOS1. In some embodiments, at least about 45% to about 80% of the enteric neurons produced by the disclosed methods express NOS1. In some embodiments, at least about 50% to about 80% of the enteric neurons produced by the disclosed methods express NOS1. In some embodiments, at least about 60% to about 80% of the enteric neurons produced by the disclosed methods express NOS1.

In some embodiments, at least about 20% of the enteric neurons produced by the disclosed methods express NOS1. In some embodiments, at least about 25% of the enteric neurons produced by the disclosed methods express NOS1. In some embodiments, at least about 30% of the enteric neurons produced by the disclosed methods express NOS1. In some embodiments, at least about 35% of the enteric neurons produced by the disclosed methods express NOS1. In some embodiments, at least about 40% of the enteric neurons produced by the disclosed methods express NOS1. In some embodiments, at least about 45% of the enteric neurons produced by the disclosed methods express NOS1. In some embodiments, at least about 50% of the enteric neurons produced by the disclosed methods express NOS1. In some embodiments, at least about 55% of the enteric neurons produced by the disclosed methods express NOS1. In some embodiments, at least about 60% of the enteric neurons produced by the disclosed methods express NOS1. In some embodiments, at least about 65% of the enteric neurons produced by the disclosed methods express NOS1. In some embodiments, at least about 70% of the enteric neurons produced by the disclosed methods express NOS1. In some embodiments, at least about 75% of the enteric neurons produced by the disclosed methods express NOS1. In some embodiments, at least about 80% of the enteric neurons produced by the disclosed methods express NOS1. In some embodiments, at least about 85% of the enteric neurons produced by the disclosed methods express NOS1. In some embodiments, at least about 90% of the enteric neurons produced by the disclosed methods express NOS1. In some embodiments, at least about 95% of the enteric neurons produced by the disclosed methods express NOS1. In some embodiments, at least about 100% of the enteric neurons produced by the disclosed methods express NOS1.

The enteric neurons produced by any of the disclosed methods can be identified by any known enteric neuron markers, which include, but not limited to, CHAT, 5HT, GABA, TUJ1, MAP2, PHOX2A, PHOX2B, TRKC, ASCL1, HAND2, EDNRB, NOS, SST, TH, DBH, Substance P, VIP, NPY, GnRH, and CGRP. In some embodiments, enteric neurons can be identified by any plurality of enteric neuron markers chosen from combinations of two or more of: CHAT, 5HT, GABA, TUJ1, MAP2, PHOX2A, PHOX2B, TRKC, ASCL1, HAND2, EDNRB, NOS, SST, TH, DBH, Substance P, VIP, NPY, GnRH, and CGRP. In such embodiments, antibodies to these proteins may be used together or in sequence to isolate or identify the cells.

CHAT, or choline acetyl transferase, is an enzyme that catalyzes the transfer of an acetyl group from the coenzyme acetyl-CoA to choline, yielding acetylcholine (ACh). A non-limiting example of CHAT is the CHAT from human (Homo sapiens, UniProt accession No. P28329) having the following sequence:

(SEQ ID NO: 4)
MGLRTAKKRGLGGGGKWKREEGGGTRGRREVRPACFLQSGGRGDPGDV
GGPAGNPGCSPHPRAATRPPPLPAHTPAHTPEWCGAASAEAAEPRRAG
PHLCIPAPGLTKTPILEKVPRKMAAKTPSSEESGLPKLPVPPLQQTLA
TYLQCMRHLVSEEQFRKSQAIVQQFGAPGGLGETLQQKLLERQEKTAN
WVSEYWLNDMYLNNRLALPVNSSPAVIFARQHFPGTDDQLRFAASLIS
GVLSYKALLDSHSIPTDCAKGQLSGQPLCMKQYYGLFSSYRLPGHTQD
TLVAQNSSIMPEPEHVIVACCNQFFVLDVVINFRRLSEGDLFTQLRKI
VKMASNEDERLPPIGLLTSDGRSEWAEARTVLVKDSTNRDSLDMIERC
ICLVCLDAPGGVELSDTHRALQLLHGGGYSKNGANRWYDKSLQFVVGR
DGTCGVVCEHSPFDGIVLVQCTEHLLKHVTQSSRKLIRADSVSELPAP
RRLRWKCSPEIQGHLASSAEKLQRIVKNLDFIVYKFDNYGKTFIKKQK
CSPDAFIQVALQLAFYRLHRRLVPTYESASIRRFQEGRVDNIRSATPE
ALAFVRAVTDHKAAVPASEKLLLLKDAIRAQTAYTVMAITGMAIDNHL
LALRELARAMCKELPEMFMDETYLMSNRFVLSTSQVPTTTEMFCCYGP
VVPNGYGACYNPQPETILFCISSFHSCKETSSSKFAKAVEESLIDMRD
LCSLLPPTESKPLATKEKATRPSQGHQP.

Another non-limiting example of CHAT is the CHAT from rat (Rattus norvegicus; UniProt accession No. P32738) having the following sequence:

(SEQ ID NO: 5)
MPILEKAPQKMPVKASSWEELDLPKLPVPPLQQTLATYLQCMQHLVPEE
QFRKSQAIVKRFGAPGGLGETLQEKLLERQEKTANWVSEYWLNDMYLNN
RLALPVNSSPAVIFARQHFQDTNDQLRFAACLISGVLSYKTLLDSHSLP
TDWAKGQLSGQPLCMKQYYRLFSSYRLPGHTQDTLVAQKSSIMPEPEHV
IVACCNQFFVLDVVINFRRLSEGDLFTQLRKIVKMASNEDERLPPIGLL
TSDGRSEWAKARTVLLKDSTNRDSLDMIERCICLVCLDGPGTGELSDTH
RALQLLHGGGCSLNGANRWYDKSLQFVVGRDGTCGVVCEHSPFDGIVLV
QCTEHLLKHMMTSNKKLVRADSVSELPAPRRLRLKCSPETQGHLASSAE
KLQRIVKNLDFIVYKFDNYGKTFIKKQKYSPDGFIQVALQLAYYRLYQR
LVPTYESASIRRFQEGRVDNIRSATPEALAFVQAMTDHKAAMPASEKLQ
LLQTAMQAHKQYTVMAITGMAIDNHLLALRELARDLCKEPPEMFMDETY
LMSNRFVLSTSQVPTTMEMFCCYGPVVPNGNGACYNPQPEAITFCISSF
HSCKETSSVEFAEAVGASLVDMRDLCSSRQPADSKPPAPKEKARGPSQA
KQS.

A further non-limiting example of CHAT is the CHAT from mouse (Mus musculus; UniProt accession No. Q03059) having the following sequence:

(SEQ ID NO: 6)
MPILEKVPPKMPVQASSCEEVLDLPKLPVPPLQQTLATYLQCMQHLVPE
EQFRKSQAIVKRFGAPGGLGETLQEKLLERQEKTANWVSEYWLNDMYLN
NRLALPVNSSPAVIFARQHFQDTNDQLRFAASLISGVLSYKALLDSQSI
PTDWAKGQLSGQPLCMKQYYRLFSSYRLPGHTQDTLVAQKSSIMPEPEH
VIVACCNQFFVLDVVINFRRLSEGDLFTQLRKIVKMASNEDERLPPIGL
LTSDGRSEWAKARTVLLKDSTNRDSLDMIERCICLVCLDGPGTGDLSDT
HRALQLLHGGGCSLNGANRWYDKSLQFVVGRDGTCGVVCEHSPFDGIVL
VQCTEHLLKHMMTGNKKLVRVDSVSELPAPRRLRWKCSPETQGHLASSA
EKLQRIVKNLDFIVYKFDNYGKTFIKKQKCSPDGFIQVALQLAYYRLYQ
RLVPTYESASIRRFQEGRVDNIRSATPEALAFVQAMTDHKAAVLASEKL
QLLQRAIQAQTEYTVMAITGMAIDNHLLALRELARDLCKEPPEMFMDET
YLMSNRFILSTSQVPTTMEMFCCYGPVVPNGYGACYNPHAEAITFCISS
FHGCKETSSVEFAEAVGASLVDMRDLCSSRQPADSKPPTAKERARGPSQ
AKQS.

In some embodiments therefore, the enteric neuron marker CHAT used in the disclosed methods comprises at least about 70%, 75%, 80%, 85%, 90%, 95%, 96%, 97%, 98% or 99% sequence identity to SEQ ID NO: 4 or a functional fragment thereof In some embodiments therefore, the CHAT comprises at least about 70%, 75%, 80%, 85%, 90%, 95%, 96%, 97%, 98% or 99% sequence identity to SEQ ID NO: 5 or a functional fragment thereof In some embodiments therefore, the CHAT comprises at least about 70%, 75%, 80%, 85%, 90%, 95%, 96%, 97%, 98% or 99% sequence identity to SEQ ID NO: 6 or a functional fragment thereof In some embodiments, the CHAT comprises SEQ ID NO: 4, SEQ ID NO: 5 or SEQ ID NO: 6, or a functional fragment thereof.

5-HT, or serotonin receptor or 5-hydroxytryptamine receptor, is a G protein-coupled receptor and ligand-gated ion channel found in the central and peripheral nervous systems. Serotonin activates the serotonin receptors, mediating both excitatory and inhibitory neurotransmission. A non-limiting example of serotonin receptor is the 5-hydroxytryptamine receptor 1A from human (Homo sapiens, UniProt accession No. P08908) having the following sequence:

(SEQ ID NO: 7)
MDVLSPGQGNNTTSPPAPFETGGNTTGISDVTVSYQVITSLLLGTLIFC
AVLGNACVVAAIALERSLQNVANYLIGSLAVTDLMVSVLVLPMAALYQV
LNKWTLGQVTCDLFIALDVLCCTSSILHLCAIALDRYWAITDPIDYVNK
RTPRRAAALISLTWLIGFLISIPPMLGWRTPEDRSDPDACTISKDHGYT
IYSTFGAFYIPLLLMLVLYGRIFRAARFRIRKTVKKVEKTGADTRHGAS
PAPQPKKSVNGESGSRNWRLGVESKAGGALCANGAVRQGDDGAALEVIE
VHRVGNSKEHLPLPSEAGPTPCAPASFERKNERNAEAKRKMALARERKT
VKTLGIIMGTFILCWLPFFIVALVLPFCESSCHMPTLLGAIINWLGYSN
SLLNPVIYAYFNKDFQNAFKKIIKCKFCRQ.

Another non-limiting example of serotonin receptor is the 5-hydroxytryptamine receptor 1A from rat (Rattus norvegicus; UniProt accession No. P19327) having the following sequence:

(SEQ ID NO: 8)
MDVFSFGQGNNTTASQEPFGTGGNVTSISDVTFSYQVITSLLLGTLIFC
AVLGNACVVAAIALERSLQNVANYLIGSLAVTDLMVSVLVLPMAALYQV
LNKWTLGQVTCDLFIALDVLCCTSSILHLCAIALDRYWAITDPIDYVNK
RTPRRAAALISLTWLIGFLISIPPMLGWRTPEDRSDPDACTISKDHGYT
IYSTFGAFYIPLLLMLVLYGRIFRAARFRIRKTVRKVEKKGAGTSLGTS
SAPPPKKSLNGQPGSGDWRRCAENRAVGTPCTNGAVRQGDDEATLEVIE
VHRVGNSKEHLPLPSESGSNSYAPACLERKNERNAEAKRKMALARERKT
VKTLGIIMGTFILCWLPFFIVALVLPFCESSCHMPALLGAIINWLGYSN
SLLNPVIYAYFNKDFQNAFKKIIKCKFCRR.

A further non-limiting example of serotonin receptor is the 5-hydroxytryptamine receptor 1A from mouse (Mus musculus; UniProt accession No. Q64264) having the following sequence:

(SEQ ID NO: 9)
MDMFSLGQGNNTTTSLEPFGTGGNDTGLSNVTFSYQVITSLLLGTLIFCA
VLGNACVVAAIALERSLQNVANYLIGSLAVTDLMVSVLVLPMAALYQVLN
KWTLGQVTCDLFIALDVLCCTSSILHLCAIALDRYWAITDPIDYVNKRTP
RRAAALISLTWLIGFLISIPPMLGWRTPEDRSNPNECTISKDHGYTIYST
FGAFYIPLLLMLVLYGRIFRAARFRIRKTVKKVEKKGAGTSFGTSSAPPP
KKSLNGQPGSGDCRRSAENRAVGTPCANGAVRQGEDDATLEVIEVHRVGN
SKGHLPLPSESGATSYVPACLERKNERTAEAKRKMALARERKTVKTLGII
MGTFILCWLPFFIVALVLPFCESSCHMPELLGAIINWLGYSNSLLNPVIY
AYFNKDFQNAFKKIIKCKFCR.

In some embodiments therefore, the enteric neuron marker 5-HT used in the disclosed methods comprises at least about 70%, 75%, 80%, 85%, 90%, 95%, 96%, 97%, 98% or 99% sequence identity to SEQ ID NO: 7 or a functional fragment thereof In some embodiments therefore, serotonin receptor comprises at least about 70%, 75%, 80%, 85%, 90%, 95%, 96%, 97%, 98% or 99% sequence identity to SEQ ID NO: 8 or a functional fragment thereof. In some embodiments therefore, serotonin receptor comprises at least about 70%, 75%, 80%, 85%, 90%, 95%, 96%, 97%, 98% or 99% sequence identity to SEQ ID NO: 9 or a functional fragment thereof. In some embodiments, the serotonin receptor comprises SEQ ID NO: 7, SEQ ID NO: 8 or SEQ ID NO: 9, or a functional fragment thereof.

Gamma-aminobutyric acid, or γ-aminobutyric acid, or GABA, acts as a trophic factor to modulate several essential developmental processes including neuronal proliferation, migration, and differentiation. GABA is the chief inhibitory neurotransmitter in the developmentally mature mammalian central nervous system. Its principal role is reducing neuronal excitability throughout the nervous system.

Glial fibrillary acidic protein (GFAP) is a class-III intermediate filament. During the development of the central nervous system, GFAP is a cell-specific marker that distinguishes astrocytes from other glial cells. A non-limiting example of GFAP is the GFAP from human (Homo sapiens, UniProt accession No. P14136) having the following sequence:

(SEQ ID NO: 13)
MERRRITSAARRSYVSSGEMMVGGLAPGRRLGPGTRLSLARMPPPLPTRV
DFSLAGALNAGFKETRASERAEMMELNDRFASYIEKVRFLEQQNKALAA
ELNQLRAKEPTKLADVYQAELRELRLRLDQLTANSARLEVERDNLAQDL
ATVRQKLQDETNLRLEAENNLAAYRQEADEATLARLDLERKIESLEEEIR
FLRKIHEEEVRELQEQLARQQVHVELDVAKPDLTAALKEIRTQYEAMASS
NMHEAEEWYRSKFADLTDAAARNAELLRQAKHEANDYRRQLQSLTCDL
ESLRGTNESLERQMREQEERHVREAASYQEALARLEEEGQSLKDEMARH
LQEYQDLLNVKLALDIEIATYRKLLEGEENRITIPVQTFSNLQIRETSLD
TKSVSEGHLKRNIVVKTVEMRDGEVIKESKQEHKDVM.

Another non-limiting example of GFAP is the GFAP from rat (Rattus norvegicus; UniProt accession No. P47819) having the following sequence:

(SEQ ID NO: 14)
MERRRITSARRSYASSETMVRGHGPTRHLGTIPRLSLSRMTPPLPARVDF
SLAGALNAGFKETRASERAEMMELNDRFASYIEKVRFLEQQNKALAAELN
QLRAKEPTKLADVYQAELRELRLRLDQLTTNSARLEVERDNLTQDLGTL
RQKLQDETNLRLEAENNLAVYRQEADEATLARVDLERKVESLEEEIQFLR
KIHEEEVRELQEQLAQQQVHVEMDVAKPDLTAALREIRTQYEAVATSNM
QETEEWYRSKFADLTDVASRNAELLRQAKHEANDYRRQLQALTCDLESL
RGTNESLERQMREQEERHARESASYQEALARLEEEGQSLKEEMARHLQE
YQDLLNVKLALDIEIATYRKLLEGEENRITIPVQTFSNLQIRETSLDTKS
VSEGHLKRNIVVKTVEMRDGEVIKESKQEHKDVM.

A further non-limiting example of GFAP is the GFAP from mouse (Mus musculus; UniProt accession No. P03995) having the following sequence:

(SEQ ID NO: 15)
MERRRITSARRSYASETVVRGLGPSRQLGTMPRFSLSRMTPPLPARVDFS
LAGALNAGFKETRASERAEMMELNDRFASYIEKVRFLEQQNKALAAELNQ
LRAKEPTKLADVYQAELRELRLRLDQLTANSARLEVERDNFAQDLGTLR
QKLQDETNLRLEAENNLAAYRQEADEATLARVDLERKVESLEEEIQFLRK
IYEEEVRELREQLAQQQVHVEMDVAKPDLTAALREIRTQYEAVATSNMQ
ETEEWYRSKFADLTDAASRNAELLRQAKHEANDYRRQLQALTCDLESLR
GTNESLERQMREQEERHARESASYQEALARLEEEGQSLKEEMARHLQEY
QDLLNVKLALDIEIATYRKLLEGEENRITIPVQTFSNLQIRETSLDTKSV
SEGHLKRNIVVKTVEMRDGEVIKDSKQEHKDVVM.

In some embodiments therefore, the GFAP comprises at least about 70%, 75%, 80%, 85%, 90%, 95%, 96%, 97%, 98% or 99% sequence identity to SEQ ID NO: 13 or a functional fragment thereof In some embodiments therefore, the GFAP comprises at least about 70%, 75%, 80%, 85%, 90%, 95%, 96%, 97%, 98% or 99% sequence identity to SEQ ID NO: 14 or a functional fragment thereof. In some embodiments therefore, the GFAP comprises at least about 70%, 75%, 80%, 85%, 90%, 95%, 96%, 97%, 98% or 99% sequence identity to SEQ ID NO: 15 or a functional fragment thereof. In some embodiments, GFAP comprises SEQ ID NO: 13, SEQ ID NO: 14 or SEQ ID NO: 15, or a functional fragment thereof.

Enteric neural crest cells express transcription factor SOX10, which directs the activity of other genes that signal neural crest cells to become more specific cell types including enteric nerves. A non-limiting example of SOX10 is the SOX10 from human (Homo sapiens, UniProt accession No. P56693) having the following sequence:

(SEQ ID NO: 16)
MAEEQDLSEVELSPVGSEEPRCLSPGSAPSLGPDGGGGGSGLRASPGPGE
LGKVKKEQQDGEADDDKFPVCIREAVSQVLSGYDWTLVPMPVRVNGASK
SKPHVKRPMNAFMVWAQAARRKLADQYPHLHNAELSKTLGKLWRLLN
ESDKRPFIEEAERLRMQHKKDHPDYKYQPRRRKNGKAAQGEAECPGGEA
EQGGTAAIQAHYKSAHLDHRHPGEGSPMSDGNPEHPSGQSHGPPTPPTTP
KTELQSGKADPKRDGRSMGEGGKPHIDFGNVDIGEISHEVMSNMETFDV
AELDQYLPPNGHPGHVSSYSAAGYGLGSALAVASGHSAWISKPPGVALPT
VSPPGVDAKAQVKTETAGPQGPPHYTDQPSTSQIAYTSLSLPHYGSAFPS
ISRPQFDYSDHQPSGPYYGHSGQASGLYSAFSYMGPSQRPLYTAISDPSP
SGPQSHSPTHWEQPVYTTLSRP.

Another non-limiting example of SOX10 is the SOX10 from rat (Rattus norvegicus; UniProt accession No. 055170) having the following sequence:

(SEQ ID NO: 17)
MAEEQDLSEVELSPVGSEEPRCLSPSSAPSLGPDGGGGGSGLRASPGPGE
LGKVKKEQQDGEADDDKFPVCIREAVSQVLSGYDWTLVPMPVRVNGASK
SKPHVKRPMNAFMVWAQAARRKLADQYPHLHNAELSKTLGKLWRLLN
ESDKRPFIEEAERLRMQHKKDHPDYKYQPRRRKNGKAAQGEAECPGGET
DQGGAAAIQAHYKSAHLDHRHPEEGSPMSDGNPEHPSGQSHGPPTPPTTP
KTELQSGKADPKRDGRSLGEGGKPHIDFGNVDIGEISHEVMSNMETFDVT
ELDQYLPPNGHPGHVGSYSAAGYGLSSALAVASGHSAWISKPPGVALPTV
SPPAVDAKAQVKTETTGPQGPPHYTDQPSTSQIAYTSLSLPHYGSAFPSI
SRPQFDYSDHQPSGPYYGHAGQASGLYSAFSYMGPSQRPLYTAISDPSPS
GPQSHSPTHWEQPVYTTLSRP.

A further non-limiting example of SOX10 is the SOX10 from mouse (Mus musculus; UniProt accession No. Q04888) having the following sequence:

(SEQ ID NO: 18)
MAEEQDLSEVELSPVGSEEPRCLSPGSAPSLGPDGGGGGSGLRASPGPGE
LGKVKKEQQDGEADDDKFPVCIREAVSQVLSGYDWTLVPMPVRVNGASK
SKPHVKRPMNAFMVWAQAARRKLADQYPHLHNAELSKTLGKLWRLLN
ESDKRPFIEEAERLRMQHKKDHPDYKYQPRRRKNGKAAQGEAECPGGEA
EQGGAAAIQAHYKSAHLDHRHPEEGSPMSDGNPEHPSGQSHGPPTPPTTP
KTELQSGKADPKRDGRSLGEGGKPHIDFGNVDIGEISHEVMSNMETFDVT
ELDQYLPPNGHPGHVGSYSAAGYGLGSALAVASGHSAWISKPPGVALPTV
SPPGVDAKAQVKTETTGPQGPPHYTDQPSTSQIAYTSLSLPHYGSAFPSI
SRPQFDYSDHQPSGPYYGHAGQASGLYSAFSYMGPSQRPLYTAISDPSPS
GPQSHSPTHWEQPVYTTLSRP.

In some embodiments therefore, the SOX10 comprises at least about 70%, 75%, 80%, 85%, 90%, 95%, 96%, 97%, 98% or 99% sequence identity to SEQ ID NO: 16 or a functional fragment thereof In some embodiments, SOX10 comprises at least about 70%, 75%, 80%, 85%, 90%, 95%, 96%, 97%, 98% or 99% sequence identity to SEQ ID NO: 17 or a functional fragment thereof In some embodiments, the SOX10 comprises at least about 70%, 75%, 80%, 85%, 90%, 95%, 96%, 97%, 98% or 99% sequence identity to SEQ ID NO: 18 or a functional fragment thereof. In some embodiments, SOX10 comprises SEQ ID NO: 16, SEQ ID NO: 17 or SEQ ID NO: 18, or a functional fragment thereof.

Additional molecular identifiers may be used during the process of generating enteric nitrergic neurons depending on the stage of differentiation include, but not limited to, the following.

Day 0 (hESCs and hiPSCs)—POU5F1

(Isoform 2) NM_001173531.2
(SEQ ID NO: 19)
GGAAAAAAGGAAAGTGCACTTGGAAGAGATCCAAGTGGGCAACTTGAAG
AACAAGTGCCAAATAGCACTTCTGTCATGCTGGATGTCAGGGCTCTTTG
TCCACTTTGTATAGCCGCTGGCTTATAGAAGGTGCTCGATAAATCTCTT
GAATTTAAAAATCAATTAGGATGCCTCTATAGTGAAAAAGATACAGTAA
AGATGAGGGATAATCAATTTAAAAAATGAGTAAGTACACACAAAGCACT
TTATCCATTCTTATGACACCTGTTACTTTTTTGCTGTGTTTGTGTGTAT
GCATGCCATGTTATAGTTTGTGGGACCCTCAAAGCAAGCTGGGGAGAGT
ATATACTGAATTTAGCTTCTGAGACATGATGCTCTTCCTTTTTAATTAA
CCCAGAACTTAGCAGCTTATCTATTTCTCTAATCTCAAAACATCCTTAA
ACTGGGGGTGATACTTGAGTGAGAGAATTTTGCAGGTATTAAATGAACT
ATCTTCTTTTTTTTTTTTCTTTGAGACAGAGTCTTGCTCTGTCACCCAG
GCTGGAGTGCAGTGGCGTGATCTCAGCTCACTGCAACCTCCGCCTCCCG
GGTTCAAGTGATTCTCCTGCCTCAGCCTCCTGAGTAGCTGGGATTACAG
TCCCAGGACATCAAAGCTCTGCAGAAAGAACTCGAGCAATTTGCCAAGC
TCCTGAAGCAGAAGAGGATCACCCTGGGATATACACAGGCCGATGTGGG
GCTCACCCTGGGGGTTCTATTTGGGAAGGTATTCAGCCAAACGACCATC
TGCCGCTTTGAGGCTCTGCAGCTTAGCTTCAAGAACATGTGTAAGCTGC
GGCCCTTGCTGCAGAAGTGGGTGGAGGAAGCTGACAACAATGAAAATCT
TCAGGAGATATGCAAAGCAGAAACCCTCGTGCAGGCCCGAAAGAGAAAG
CGAACCAGTATCGAGAACCGAGTGAGAGGCAACCTGGAGAATTTGTTCC
TGCAGTGCCCGAAACCCACACTGCAGCAGATCAGCCACATCGCCCAGCA
GCTTGGGCTCGAGAAGGATGTGGTCCGAGTGTGGTTCTGTAACCGGCGC
CAGAAGGGCAAGCGATCAAGCAGCGACTATGCACAACGAGAGGATTTTG
AGGCTGCTGGGTCTCCTTTCTCAGGGGGACCAGTGTCCTTTCCTCTGGC
CCCAGGGCCCCATTTTGGTACCCCAGGCTATGGGAGCCCTCACTTCACT
GCACTGTACTCCTCGGTCCCTTTCCCTGAGGGGGAAGCCTTTCCCCCTG
TCTCCGTCACCACTCTGGGCTCTCCCATGCATTCAAACTGAGGTGCCTG
CCCTTCTAGGAATGGGGGACAGGGGGAGGGGAGGAGCTAGGGAAAGAAA
ACCTGGAGTTTGTGCCAGGGTTTTTGGGATTAAGTTCTTCATTCACTAA
GGAAGGAATTGGGAACACAAAGGGTGGGGGCAGGGGAGTTTGGGGCAAC
TGGTTGGAGGGAAGGTGAAGTTCAATGATGCTCTTGATTTTAATCCCAC
ATCATGTATCACTTTTTTCTTAAATAAAGAAGCCTGGGACACAGTAGAT
AGACACACTTAAAAAAAAAAA
(Isoform 2) NP_001 167002.1
(SEQ ID NO: 20)
MGVLFGKVFSQTTICRFEALQLSFKNMCKLRPLLQKWVEEADNNENLQE
ICKAETLVQARKRKRTSIENRVRGNLENLFLQCPKPTLQQISHIAQQLG
LEKDVVRVWFCNRRQKGKRSSSDYAQREDFEAAGSPFSGGPVSFPLAPG
PHFGTPGYGSPHFTALYSSVPFPEGEAFPPVSVTTLGSPMHSN

Isoform/Variant includes: (Isoform 4) NM_001285986.1 and NP_001272915.1; (Isoform 3) NM_001285987.1 and NP_001272916.1; (Isoform 1) NM_002701.6 and NP_002692.2; (Isoform 2) NM_203289.5 and NP_976034.4
Day 0 (hESCs and hiPSCs)—NANOG

(Isoform 2) NM_001297698.2
(SEQ ID NO: 21)
ATAAATCTAGAGACTCCAGGATTTTAACGTTCTGCTGGACTGAGCTGGTTGCCTCATGTTATTATGCAGG
CAACTCACTTTATCCCAATTTCTTGATACTTTTCCTTCTGGAGGTCCTATTTCTCTAACATCTTCCAGAA
AAGTCTTAAAGCTGCCTTAACCTTTTTTCCAGTCCACCTCTTAAATTTTTTCCTCCTCTTCCTCTATACT
AACATGAGTGTGGATCCAGCTTGTCCCCAAAGCTTGCCTTGCTTTGAAGCATCCGACTGTAAAGAATCTT
CACCTATGCCTGTGATTTGTGGGCCTGAAGAAAACTATCCATCCTTGCAAATGTCTTCTGCTGAGATGCC
TCACACGGAGACTGTCTCTCCTCTTCCTTCCTCCATGGATCTGCTTATTCAGGACAGCCCTGATTCTTCC
ACCAGTCCCAAAGGCAAACAACCCACTTCTGCAGAGAAGAGTGTCGCAAAAAAGGAAGACAAGGTCCCGG
TCAAGAAACAGAAGACCAGAACTGTGTTCTCTTCCACCCAGCTGTGTGTACTCAATGATAGATTTCAGAG
ACAGAAATACCTCAGCCTCCAGCAGATGCAAGAACTCTCCAACATCCTGAACCTCAGCTACAAACAGGTG
AAGACCTGGTTCCAGAACCAGAGAATGAAATCTAAGAGGTGGCAGAAAAACAACTGGCCGAAGAATAGCA
ATGGTGTGACGCAGGGATGCCTGGTGAACCCGACTGGGAACCTTCCAATGTGGAGCAACCAGACCTGGAA
CAATTCAACCTGGAGCAACCAGACCCAGAACATCCAGTCCTGGAGCAACCACTCCTGGAACACTCAGACC
TGGTGCACCCAATCCTGGAACAATCAGGCCTGGAACAGTCCCTTCTATAACTGTGGAGAGGAATCTCTGC
AGTCCTGCATGCAGTTCCAGCCAAATTCTCCTGCCAGTGACTTGGAGGCTGCCTTGGAAGCTGCTGGGGA
AGGCCTTAATGTAATACAGCAGACCACTAGGTATTTTAGTACTCCACAAACCATGGATTTATTCCTAAAC
TACTCCATGAACATGCAACCTGAAGACGTGTGAAGATGAGTGAAACTGATATTACTCAATTTCAGTCTGG
ACACTGGCTGAATCCTTCCTCTCCCCTCCTCCCATCCCTCATAGGATTTTTCTTGTTTGGAAACCACGTG
TTCTGGTTTCCATGATGCCCATCCAGTCAATCTCATGGAGGGTGGAGTATGGTTGGAGCCTAATCAGCGA
GGTTTCTTTTTTTTTTTTTTTCCTATTGGATCTTCCTGGAGAAAATACTTTTTTTTTTTTTTTTTTTGAA
ACGGAGTCTTGCTCTGTCGCCCAGGCTGGAGTGCAGTGGCGCGGTCTTGGCTCACTGCAAGCTCCGTCTC
CCGGGTTCACGCCATTCTCCTGCCTCAGCCTCCCGAGCAGCTGGGACTACAGGCGCCCGCCACCTCGCCC
GGCTAATATTTTGTATTTTTAGTAGAGACGGGGTTTCACTGTGTTAGCCAGGATGGTCTCGATCTCCTGA
CCTTGTGATCCACCCGCCTCGGCCTCCCTAACAGCTGGGATTTACAGGCGTGAGCCACCGCGCCCTGCCT
AGAAAAGACATTTTAATAACCTTGGCTGCCGTCTCTGGCTATAGATAAGTAGATCTAATACTAGTTTGGA
TATCTTTAGGGTTTAGAATCTAACCTCAAGAATAAGAAATACAAGTACAAATTGGTGATGAAGATGTATT
CGTATTGTTTGGGATTGGGAGGCTTTGCTTATTTTTTAAAAACTATTGAGGTAAAGGGTTAAGCTGTAAC
ATACTTAATTGATTTCTTACCGTTTTTGGCTCTGTTTTGCTATATCCCCTAATTTGTTGGTTGTGCTAAT
CTTTGTAGAAAGAGGTCTCGTATTTGCTGCATCGTAATGACATGAGTACTGCTTTAGTTGGTTTAAGTTC
AAATGAATGAAACAACTATTTTTCCTTTAGTTGATTTTACCCTGATTTCACCGAGTGTTTCAATGAGTAA
ATATACAGCTTAAACATAA
(Isoform 2) NP_001284627.1
(SEQ ID NO: 22)
MSVDPACPQSLPCFEASDCKESSPMPVICGPEENYPSLQMSSAEMPHTETVSPLPSSMDLLIQDSPDSST
SPKGKQPTSAEKSVAKKEDKVPVKKQKTRTVFSSTQLCVLNDRFQRQKYLSLQQMQELSNILNLSYKQVK
TWFQNQRMKSKRWQKNNWPKNSNGVTQGCLVNPTGNLPMWSNQTWNNSTWSNQTQNIQSWSNHSWNTQTW
CTQSWNNQAWNSPFYNCGEESLQSCMQFQPNSPASDLEAALEAAGEGLNVIQQTTRYFSTPQTMDLFLNY
SMNMQPEDV

Isoform/Variant includes: (Isoform 1) NM_024865.4 and NP_079141.2

### Day 12-15 (ENCs)—CD49D (ITGA4)

(Isoform 1 Preproprotein) NM_000885.6
(SEQ ID NO: 23)
GACTATCACACAACTATTTCCTTGGATGTAATTCTTTGTTACCCTTTACAAGTATAAGTGTTACCTTACA
TGGAAACGAAGAAACAAAATTCATAAATTTAAATTCATAAATTTAGCTGAAAGATACTGATTCAATTTGT
ATACAGTGAATATAAATGAGACGACAGCAAAATTTTCATGAAATGTAAAATATTTTTATAGTTTGTTCAT
ACTATATGAGGTTCTATTTTAAATGACTTTCTGGATTTTAAAAAATTTCTTTAAATACAATCATTTTTGT
AATATTTATTTTATGCTTATGATCTAGATAATTGCAGAATATCATTTTATCTGACTCTGCCTTCATAAGA
GAGCTGTGGCCGAATTTTGAACATCTGTTATAGGGAGTGATCAAATTAGAAGGCAATGTGGAAAAACAAT
TCTGGGAAAGATTTCTTTATATGAAGTCCCTGCCACTAGCCAGCCATCCTAATTGATGAAAGTTATCTGT
TCACAGGCCTGCAGTGATGGTGAGGAATGTTCTGAGATTTGCGAAGGCATTTGAGTAGTGAAATGTAAGC
ACAAAACCTCCTGAACCCAGAGTGTGTATACACAGGAATAAACTTTATGACATTTATGTATTTTTAAAAA
ACTTTGTATCGTTATAAAAAGGCTAGTCATTCTTTCAGGAGAACATCTAGGATCATAGATGAAAAATCAA
GCCCCGATTTAGAACTGTCTTCTCCAGGATGGTCTCTAAGGAAATTTACATTTGGTTCTTTCCTACTCAG
AACTACTCAGAAACAACTATATATTTCAGGTTATCTGAGCACAGTGAAAGCAGAGTACTATGGTTGTCCA
ACACAGGCCTCTCAGATACAAGGGGAACACAATTACATATTGGGCTAGATTTTGCCCAGTTCAAAATAGT
ATTTGTTATCAACTTACTTTGTTACTTGTATCATGAATTTTAAAACCCTACCACTTTAAGAAGACAGGGA
TGGGTTATTCTTTTTTGGCAGGTAGGCTATATAACTATGTGATTTTGAAATTTAACTGCTCTGGATTAGG
GAGCAGTGAATCAAGGCAGACTTATGAAATCTGTATTATATTTGTAACAGAATATAGGAAATTTAACATA
ATTGATGAGCTCAAATCCTGAAAAATGAAAGAATCCAAATTATTTCAGAATTATCTAGGTTAAATATTGA
TGTATTATGATGGTTGCAAAGTTTTTTTGTGTGTCCAATAAACACATTGTAAAAAAAAGAATTTGAATTG
ATATCTAAAAACAGAATTTGAATTGATATTTCATCTTGACTTTTAAAGCCCTAGAGGCTAATTGTTAGTA
ACATCAATTTCTATTAGGATATCCGTTTGGCCACACAGCAGGAGGTTAGAGCAATGGAGCATTACTGAGT
TCCTCCCCCTGTCAGATCAGCAGCAGCATTAGATTCTCATAGAAGTGCGAACCATATGGTGAACTGGTAT
GTGAGGGATCTAGAGTGCCATGTTCCTCAAGAGAATCTAATGCCTGATGATCTGAGGTGGAACAGTTCAT
CCTGAAACCATTCCCCCATCCACGGAAAAATTGTCTTCCATGAAACTGGTCCCAAAAAGGGTGGGGACCA
CAGGTTTAAAGCATGGCCACATTTCTTTATATTAAAATTCTAGTTTGTACATTTCTTTTAGAAACAATTA
CATGTTACTTTGGAATCATTTCTTCCATGCTTCCTCCATAAAGACTGATAAGTCTTGGATGCAATCTGTA
AAGAAAATACATTATTTCATCAACTTATTTTGTTGTTTTTCACATACACCTAATAAGTATGGTACACAAT
GCCAATGCCAAATACAAATTGATAACAAACACAGCATTCCCAACAGAGCTGTAATCTAGAAAACTGAGAA
GGTCTGATTGATAAATCATCAACAACAATAATTGCTCTAAAACCTCCTTAACTGACTTCCTTGATTGTCC
AATGCTCTCCATTACCTCTGTAAAACAGTCAGTTATGCCTCTAGAACACCCATGTCTAGTGGGCACCCCT
GCATGCTTCTTCTAACCACTGAGTGTCACAATGCCTACCAAGAATGCGTTTGCAGGTTCCTAAACCTGTT
TATACCAGTTGCTATGTAAAATTGTTCCCAAGGGAAGTTGAATGCTCTGTAAAGGCCTAATAAAAGCAAA
TTACTGAACAAAACATGTTACAGTAATTATGAGTGAGAGGAAACTAAGATGGAAGGATAAAAATCTAACA
CTTTACTATTCAGATGGCTCCACTAAAAGATTTAAGATCTTGATCCATTTTTAAAAATCCAAAATGGAAG
TTGTAGACATTATCTGTAGTTTATGCACAACAATAAATTAGAAAGCCAATGTAGACACGCATAACCAAAG
AAAATGCCTTGGGTCTACATAACAGTTGAATAAATGTAAAGTTGCTTTTTATTTATTGAAA
(Isoform 1 Preproprotein) NP_000876.3
(SEQ ID NO: 24)
MAWEARREPGPRRAAVRETVMLLLCLGVPTGRPYNVDTESALLYQGPHNTLFGYSVVLHSHGANRWLLVG
APTANWLANASVINPGAIYRCRIGKNPGQTCEQLQLGSPNGEPCGKTCLEERDNQWLGVTLSRQPGENGS
IVTCGHRWKNIFYIKNENKLPTGGCYGVPPDLRTELSKRIAPCYQDYVKKFGENFASCQAGISSFYTKDL
IVMGAPGSSYWTGSLFVYNITTNKYKAFLDKQNQVKFGSYLGYSVGAGHFRSQHTTEVVGGAPQHEQIGK
AYIFSIDEKELNILHEMKGKKLGSYFGASVCAVDLNADGFSDLLVGAPMQSTIREEGRVFVYINSGSGAV
MNAMETNLVGSDKYAARFGESIVNLGDIDNDGFEDVAIGAPQEDDLQGAIYIYNGRADGISSTFSQRIEG
LQISKSLSMFGQSISGQIDADNNGYVDVAVGAFRSDSAVLLRTRPVVIVDASLSHPESVNRTKFDCVENG
WPSVCIDLTLCFSYKGKEVPGYIVLFYNMSLDVNRKAESPPRFYFSSNGTSDVITGSIQVSSREANCRTH
QAFMRKDVRDILTPIQIEAAYHLGPHVISKRSTEEFPPLQPILQQKKEKDIMKKTINFARFCAHENCSAD
LQVSAKIGFLKPHENKTYLAVGSMKTLMLNVSLFNAGDDAYETTLHVKLPVGLYFIKILELEEKQINCEV
TDNSGVVQLDCSIGYIYVDHLSRIDISFLLDVSSLSRAEEDLSITVHATCENEEEMDNLKHSRVTVAIPL
KYEVKLTVHGFVNPTSFVYGSNDENEPETCMVEKMNLTFHVINTGNSMAPNVSVEIMVPNSFSPQTDKLF
NILDVQTTTGECHFENYQRVCALEQQKSAMQTLKGIVRFLSKTDKRLLYCIKADPHCLNFLCNFGKMESG
KEASVHIQLEGRPSILEMDETSALKFEIRATGFPEPNPRVIELNKDENVAHVLLEGLHHQRPKRYFTIVI
ISSSLLLGLIVLLLISYVMWKAGFFKRQYKSILQEENRRDSWSYINSKSNDD

Isoform/Variant includes: (Isoform 2 precursor) NM_001316312.1 and NP_001303241.1

### Day 12-15 (ENCs)—SOX10

NM_006941.4
(SEQ ID NO: 25)
GACGATGACAAGTTCCCCGTGTGCATCCGCGAGGCCGTCAGCCAGGTGCTCAGCGGCTACGACTGGACGC
TGGTGCCCATGCCCGTGCGCGTCAACGGCGCCAGCAAAAGCAAGCCGCACGTCAAGCGGCCCATGAACGC
CTTCATGGTGTGGGCTCAGGCAGCGCGCAGGAAGCTCGCGGACCAGTACCCGCACCTGCACAACGCTGAG
CTCAGCAAGACGCTGGGCAAGCTCTGGAGGCTGCTGAACGAAAGTGACAAGCGCCCCTTCATCGAGGAGG
CTGAGCGGCTCCGTATGCAGCACAAGAAAGACCACCCGGACTACAAGTACCAGCCCAGGCGGCGGAAGAA
CGGGAAGGCCGCCCAGGGCGAGGCGGAGTGCCCCGGTGGGGAGGCCGAGCAAGGTGGGACCGCCGCCATC
CAGGCCCACTACAAGAGCGCCCACTTGGACCACCGGCACCCAGGAGAGGGCTCCCCCATGTCAGATGGGA
ACCCCGAGCACCCCTCAGGCCAGAGCCATGGCCCACCCACCCCTCCAACCACCCCGAAGACAGAGCTGCA
GTCGGGCAAGGCAGACCCGAAGCGGGACGGGCGCTCCATGGGGGAGGGCGGGAAGCCTCACATCGACTTC
GGCAACGTGGACATTGGTGAGATCAGCCACGAGGTAATGTCCAACATGGAGACCTTTGATGTGGCTGAGT
TGGACCAGTACCTGCCGCCCAATGGGCACCCAGGCCATGTGAGCAGCTACTCAGCAGCCGGCTATGGGCT
GGGCAGTGCCCTGGCCGTGGCCAGTGGACACTCCGCCTGGATCTCCAAGCCACCAGGCGTGGCTCTGCCC
ACGGTCTCACCACCTGGTGTGGATGCCAAAGCCCAGGTGAAGACAGAGACCGCGGGGCCCCAGGGGCCCC
CACACTACACCGACCAGCCATCCACCTCACAGATCGCCTACACCTCCCTCAGCCTGCCCCACTATGGCTC
AGCCTTCCCCTCCATCTCCCGCCCCCAGTTTGACTACTCTGACCATCAGCCCTCAGGACCCTATTATGGC
CACTCGGGCCAGGCCTCTGGCCTCTACTCGGCCTTCTCCTATATGGGGCCCTCGCAGCGGCCCCTCTACA
CGGCCATCTCTGACCCCAGCCCCTCAGGGCCCCAGTCCCACAGCCCCACACACTGGGAGCAGCCAGTATA
TACGACACTGTCCCGGCCCTAAAGGGGGCCCTGTCGCCACCACCCCCCGCCCAGCCCCTGCCCCCAGCCT
GTGTGCCCTGTTCCTTGCCCACCTCAGGCCTGGTGGTGGCAGTGGAGGAGGCTGAGGAGGCTGAAGAGGC
TGACAGGTCGGGGGGCTTTCTGTCTGGCTCACTGCCCTGATGACCCACCCGCCCCATCCAGGCTCCAGCA
GCAAAGCCCCAGGAGAACAGGCTGGACAGAGGAGAAGGAGGTTGACTGTTGCACCCACACTGAAAGATGA
GGGGCTGCACCTTCCCCCAGGAATGACCCTCTATCCCAGGACCTGAGAAGGGCCTGCTCACCCTCCTCGG
GGAGGGGAAGCACCAGGGTTGGTGGCATCGGAGGCCTTACCACTCCTATGACTCCTGTTTTCTCTCTCAC
AGATAGTGAGGGTCTGACATGCCCATGCCACCTATGCCACAGTGCCTAAGGGCTAGGCCACCCAGAGACT
GTGCCCGGAGCTGGCCGTGTCTCCCACTCAGGGGCTGAGAGTAGCTTTGAGGAGCCTCATTGGGGAGTGG
GGGGTTCGAGGGACTTAGTGGAGTTCTCATCCCTTCAATGCCCCCTCCCTTTCTGAAGGCAGGAAGGAGT
TGGCACAGAGGCCCCCTGATCCAATTCTGTGCCAATAACCTCATTCTTTGTCTGAGAAACAGCCCCCAGT
CCTCCTCCACTACAACCTCCATGACCTTGAGACGCATCCCAGGAGGTGACGAGGCAGGGGCTCCAGGAAA
GGAATCAGAGACAATTCACAGAGCCTCCCTCCCTGGGCTCCTTGCCAGCTCCCTCTTCCCTTACTAGGCT
CTATGGCCCCTGCTCAGTCAGCCCCACTCCCTGGGCTTCCCAGAGAGTGACAGCTGCTCAGGCCCTAACC
CTTGGCTCCAGGAGACACAGGGCCCAGCACCCAGGTTGCTGTCGGCAGGCTGAAGACACTAGAATCCTGA
CCTGTACATTCTGCCCTTGCCTCTTACCCCTTGCCTCCCAGTGGTATTTGAATAAAGTATGTAGCTATAT
CTGCCCCTATTTTCCTGTTCTGCAGCCCCCCAAATCCACATGTAACTCATTACTGTCTCCTGTTATTTAT
CTCAGTAGTCCCCTCTCCTAGCCACTCTAGCCCCTATTAACTCTGCATTAAGCATTCCACATAATAAAAT
TAAAGGTTCCGGTTA
NP_008872.1
(SEQ ID NO: 26)
MAEEQDLSEVELSPVGSEEPRCLSPGSAPSLGPDGGGGGSGLRASPGPGELGKVKKEQQDGEADDDKFPV
CIREAVSQVLSGYDWTLVPMPVRVNGASKSKPHVKRPMNAFMVWAQAARRKLADQYPHLHNAELSKTLGK
LWRLLNESDKRPFIEEAERLRMQHKKDHPDYKYQPRRRKNGKAAQGEAECPGGEAEQGGTAAIQAHYKSA
HLDHRHPGEGSPMSDGNPEHPSGQSHGPPTPPTTPKTELQSGKADPKRDGRSMGEGGKPHIDFGNVDIGE
ISHEVMSNMETFDVAELDQYLPPNGHPGHVSSYSAAGYGLGSALAVASGHSAWISKPPGVALPTVSPPGV
DAKAQVKTETAGPQGPPHYTDQPSTSQIAYTSLSLPHYGSAFPSISRPQFDYSDHQPSGPYYGHSGQASG
LYSAFSYMGPSQRPLYTAISDPSPSGPQSHSPTHWEQPVYTTLSRP

### Day 12-15 (ENCs)—FOXD3

NM_012183.3
(SEQ ID NO: 27)
CCCACGCCAGGGCCAGAGGCCGAGGAAGGCGGGCTAAGTGAGGGGGCGCGGCGTGGAGAACCGCCGGGGC
CGGGAGCGGTAGCGAGCGCCTAGTACCGAGCGCCAGGGACGGCAGGAGTTCGCGGAGCGCGGCCGCTGGG
GGCGGACGGCAGAGCCCGCGCCACGCGATGCGGGGCCGCCGAGTGTGAGCTGAGCCCAGCGGGCCCCAAG
CCACCTGCGGCCCCCTCCCCTCTCCCTGCCCCCCATCTTTCGGGGGCACTCAAACCCTCTTCCCCTGAGC
TCCGTGGCAGCCCCCGAACACCCTCATCGCCCGCTGCCCCCTCCCCGCCGCCGCTACCAACCCCGAGGAG
GGATGACCCTCTCCGGCGGCGGCAGCGCCAGCGACATGTCCGGCCAGACGGTGCTGACGGCCGAGGACGT
GGACATCGATGTGGTGGGCGAGGGCGACGACGGGCTGGAAGAGAAGGACAGCGACGCAGGTTGCGATAGC
CCCGCGGGGCCGCCGGAGCTGCGCCTGGACGAGGCGGACGAGGTGCCCCCGGCGGCACCCCATCACGGAC
AGCCTCAGCCGCCCCACCAGCAGCCCCTGACATTGCCCAAGGAGGCGGCCGGAGCCGGGGCCGGACCGGG
GGGCGACGTGGGCGCGCCGGAGGCGGACGGCTGCAAGGGCGGTGTTGGCGGCGAGGAGGGCGGCGCGAGC
GGCGGCGGGCCTGGCGCGGGCAGCGGTTCGGCGGGAGGCCTGGCCCCGAGCAAGCCCAAGAACAGCCTAG
TGAAGCCGCCTTACTCGTACATCGCGCTCATCACCATGGCCATCCTGCAGAGCCCGCAGAAGAAGCTGAC
CCTGAGCGGCATCTGCGAGTTCATCAGCAACCGCTTCCCCTACTACAGGGAGAAGTTCCCCGCCTGGCAG
AACAGCATCCGCCACAACCTCTCACTCAACGACTGCTTCGTCAAGATCCCCCGCGAGCCGGGCAACCCGG
GCAAGGGCAACTACTGGACCCTGGACCCGCAGTCCGAGGACATGTTCGACAACGGCAGCTTCCTGCGGCG
CCGGAAACGCTTCAAGCGCCACCAGCAGGAGCACCTGCGCGAGCAGACGGCGCTCATGATGCAGAGCTTC
GGCGCTTACAGCCTGGCGGCGGCGGCCGGCGCCGCGGGACCCTACGGCCGCCCCTACGGCCTGCACCCTG
CGGCGGCGGCCGGTGCCTATTCGCACCCGGCAGCGGCGGCGGCCGCGGCTGCTGCGGCGGCGCTCCAGTA
CCCGTACGCGCTGCCGCCGGTGGCACCGGTGCTGCCTCCCGCTGTGCCGCTGCTGCCCTCGGGCGAGCTG
GGCCGCAAAGCGGCCGCCTTCGGCTCACAGCTCGGCCCGGGCCTGCAGCTGCAGCTCAATAGCCTGGGCG
CCGCCGCGGCCGCTGCGGGCACAGCGGGCGCCGCGGGCACCACCGCGTCGCTCATCAAGTCCGAGCCAAG
CGCGCGGCCGTCGTTCAGCATCGAGAACATCATAGGTGGGGGCCCCGCGGCTCCTGGGGGCTCGGCGGTG
GGCGCTGGGGTCGCCGGCGGCACTGGGGGTTCAGGGGGCGGCAGCACGGCGCAGTCGTTTCTGCGGCCAC
CCGGGACCGTGCAGTCGGCAGCGCTCATGGCCACCCACCAACCGCTGTCGCTGAGCCGGACGACTGCCAC
CATCGCGCCCATTCTTAGCGTGCCACTCTCCGGACAGTTTCTGCAGCCCGCAGCCTCGGCCGCCGCCGCT
GCTGCGGCCGCCGCTCAAGCCAAATGGCCGGCGCAATAGGGACGCGCCAATGGCCGGGACCCAGGGTCCG
GCGGCGGCCTCGAGCAACAAATGCACCTCCAGGCTGCGCGCCCTGTCCCAAGCCCGGTCCCGGTCCCGCT
GCCCAATCCTGGACTCTGCCTCTCCCCAATTTCCTTTCCCCTGAGCCCCCAACGCCTACCTTCCGCGGCC
TCCATCCCCTCGCGCACACCTAAGCTGGTCGAGCAAACTCACCGCGCGCCCGCCGGGGATAGCTTTCCAT
ACAGGTAAAACCGAAAACCGAATTTTCCAAAAATGCACCCCGACGGCGCCTGCTCTTAGTACCGTGGGGA
TGGGAGGGAAATTCTTTGTATATATTTGTAAAAAAATTATTGACTTTCCTTTTGGGGTTTTTATTTTTTT
AAGAAAAAACAAATTCCGTAGATTTAGAGCTCTGAACTTTCATTTTTTTTGAAGGTTCACTCTCCGAAGT
TTTATCTGAGAAAAGAATGTATAGAGACGTTGGGAGATTTTAAATATAAAAAATTTTCAAAAAGGCAAAA
AGTGTCATTCTATTATAAAAGTCTGTTTATATATGAATGAATATATATGGTATTCTAAATGTTATTCCAT
CGTGTTGTACACAACTTTGTAAATAAATTTTTAAAATGCCCA
NP_036315.1
(SEQ ID NO: 28)
MTLSGGGSASDMSGQTVLTAEDVDIDVVGEGDDGLEEKDSDAGCDSPAGPPELRLDEADEVPPAAPHHGQ
PQPPHQQPLTLPKEAAGAGAGPGGDVGAPEADGCKGGVGGEEGGASGGGPGAGSGSAGGLAPSKPKNSLV
KPPYSYIALITMAILQSPQKKLTLSGICEFISNRFPYYREKFPAWQNSIRHNLSLNDCFVKIPREPGNPG
KGNYWTLDPQSEDMFDNGSFLRRRKRFKRHQQEHLREQTALMMQSFGAYSLAAAAGAAGPYGRPYGLHPA
AAAGAYSHPAAAAAAAAAAALQYPYALPPVAPVLPPAVPLLPSGELGRKAAAFGSQLGPGLQLQLNSLGA
AAAAAGTAGAAGTTASLIKSEPSARPSFSIENIIGGGPAAPGGSAVGAGVAGGTGGSGGGSTAQSFLRPP
GTVOSAALMATHQPLSLSRTTATIAPILSVPLSGQFLQPAASAAAAAAAAAQAKWPAQ

### Day 15-30 (ENPs)—TRKC (NTRK3)

(Isoform c precursor) NM_001007156.2
(SEQ ID NO: 29)
ACCACATCAACCACGGCATCACCACGCCCTCGTCACTGGATGCCGGGCCCGACACTGTGGTCATTGGCAT
GACTCGCATCCCTGTCATTGAGAACCCCCAGTACTTCCGTCAGGGACACAACTGCCACAAGCCGGACACG
TGGGTCTTTTCAAACATAGACAATCATGGGATATTAAACTTGAAGGACAATAGAGATCATCTAGTCCCAT
CAACTCACTATATATATGAGGAACCTGAGGTCCAGAGTGGGGAAGTGTCTTACCCAAGGTCACATGGTTT
CAGAGAAATTATGTTGAATCCAATAAGCCTTCCCGGACATTCCAAGCCTCTTAACCATGGCATCTATGTT
GAGGATGTCAATGTTTATTTCAGCAAAGGACGTCATGGCTTTTAAAAACTCCTTTTAAGCCTCCTTGTTT
TGATGTCACCTTGGTAGGCTGGGCCCTCTGAGAGGTTGGAAGCTCTAGGCATTGTTCTCTTTGGATCCAG
GGATGCTAAGTAGAAACTGCATGAGCCACCAGTGCCCCGGCACCCTTTAACACCACCAGATGGGTGTTTT
CCCCCATCCACCACTGGCAGGGTTGCCCCTTCCCTCCAATCATCACTGTGCTCCTTTTTTCCCGGCCTAC
GAGGCAGCTCCTGCCACTATCTTTAGAGCCAATAAAGAGAATTAAAAACCTGTGCACCAGGAGCATCTTT
TAAATACACTAGCCATTCTCTTGCTTTACAAAAACAACCTAACCATCACAAGAAAGCCTGATGAAGTCCA
GCCGTGCTCCAGCCTCACTTTCCCTGCTTGGAAGCGTGGGGTCTCCCTGGCTCTCCCAGGATACCATGCT
GTCCTCTTAGTGACCTCGTCGCCCTGCAACCTCCAGTGGGGAAGAGTCACAGAGAGCACCTAAGCAGAGG
TGGAGACGGCGCGGTAAGAGGAGGGGGAGCCAGGCTCAAGTATTGGCACCAAGTTAGGTCTCAGAGGAAA
GAATGGAAACCAATCACTTTACATTTTTATTTTTATTTTCGGTGGAAAAATCATCCTTTTTTGGGACATA
CTTGCCCCCTACTTCCTCTTCTCTCTGGAACGGCTCACAATGAGTGTGACATTAGAAAACTCCTTGCAGA
GGAGAGTTTCTCCAGGCTCTTCCTGGGCCCTTAGATCTGCAGTTCCGACAAGCTTTGGCTGCAGGAGGTT
TTACCCATGAACTGGCCATCCTACTAGGACCACAAGGGACCAAGGGAATCAGGGACAAAGGCCCTTCCTG
CCAGCCCATGATCCCGGGATTGGCTCTCTTCCCCTACTTCCACTTATTCTTGACTCTGAGAACTTTTGGA
ACCCAATGGAATCAGCATTTCAAGGTCAAGATGAACTGAAGGGGAAGAGAAGTAAAACTTGGCCTCCTCC
AGCCCCTCTCATGGCACCAATGGAAGTGTCCTCCTGTTCTCTGGTCAATATGTGTGTTTACTTTGCTTGC
TTTGACTCATGCCTTACTCCATGGCCACCCTCTCCCAAAGAGGGGGCTCGCTTCCCCCATTTTCAACTTG
ATCCACTGAGGAGAGGGAAGGGGGTGACTTTCCCTTCTTCAGTAGGAAAGGCACATTTGTAGGGCCTGAA
ACTCTCCCGTATTTGCTGACTCATTGGTGGAGTAGACTTCTGGCTCCCAGCTCCACTGGCCCATGGGGCC
TCCATTGTATGAAGTCAGCATAGGCTGCCCACCTAATGGTGGAGAGCATGAAACTGGGAGCATCCTGTGG
GGGGCTTGTGGGGGAAAAAGGTGGTTGTTTTAACCCACCGTTGTTTTGGGGTGGTGTTGCACACTAGTAG
AGAATAGAGTCTATGCCTTTGGCAAATTTAACTGGGAGTTTGGATTCCCACTTAAGGGTTTTACTTCTTG
GGTCCTGTGGATGGTGGTTCTTCGTGTCAGGATCCCAGCCCGATTCTGCAAATGCCTCCATGGGGTTTAA
AAACATGAGGCTTTCCAAGTTCTTGCCCAGTATCTGGGGCAGCCTCCAGAGTATCACCTGGGAGTTCAGG
TTCTCTCCAGGGCTCCAGGTGTGTGTTTATCTCGCCCCCTCCAGCTCTCCTCATCCTGCTCCCCATTGCT
CCATGTCAGGCTGTTCCCCATTGTGCCCTGCTGATGCTTTGGGTCCAGGGCCTCCTCCCAAGTGTGGCTT
TAAGGAGTAAGCTTGAGGATGATGTTTTTTAATTATTGTAAATCATTACCTCATTTCCAGCCTCCCAGGC
TCCATCCATCCCAGCATCTTTTATTCTGCCATTTTCCTCACCTTGTGCTATGACAATGGGGCGTTGTGTT
TCCACAGAGACTTATAGGAGTGTTCAGTGTATAGTTTCTTAATAAACACTTTATTTTCTAATGAAAAAAA
AAAAAAAAAAA
(Isoform c precursor) NP_001007157.1
(SEQ ID NO: 30)
MDVSLCPAKCSFWRIFLLGSVWLDYVGSVLACPANCVCSKTEINCRRPDDGNLFPLLEGQDSGNSNGNAS
INITDISRNITSIHIENWRSLHTLNAVDMELYTGLQKLTIKNSGLRSIQPRAFAKNPHLRYINLSSNRLT
TLSWQLFQTLSLRELQLEQNFFNCSCDIRWMQLWQEQGEAKLNSQNLYCINADGSQLPLFRMNISQCDLP
EISVSHVNLTVREGDNAVITCNGSGSPLPDVDWIVTGLQSINTHQTNLNWTNVHAINLTLVNVTSEDNGF
TLTCIAENVVGMSNASVALTVYYPPRVVSLEEPELRLEHCIEFVVRGNPPPTLHWLHNGQPLRESKIIHV
EYYQEGEISEGCLLFNKPTHYNNGNYTLIAKNPLGTANQTINGHFLKEPFPESTDNFILFDEVSPTPPIT
VTHKPEEDTFGVSIAVGLAAFACVLLVVLFVMINKYGRRSKFGMKGPVAVISGEEDSASPLHHINHGITT
PSSLDAGPDTVVIGMTRIPVIENPQYFRQGHNCHKPDTWVFSNIDNHGILNLKDNRDHLVPSTHYIYEEP
EVQSGEVSYPRSHGFREIMLNPISLPGHSKPLNHGIYVEDVNVYFSKGRHGF

Isoform/Variant includes: (Isoform a precursor) NM_001012338.2 and NP_001012338.1; (Isoform d precursor) NM_001243101.1 and NP_001230030.1; (Isoform e precursor) NM_001320134.1 and NP_001307063.1; (Isoform f precursor) NM_001320135.1 and NP_001307064.1; (Isoform a precursor) NM_001375810.1 and NP_001362739.1; (Isoform b precursor) NM_00137581.1 and NP_001362740.1; (Isoform d precursor) NM_001375812.1 and NP_001362741.1; (Isoform c precursor) NM_001375813.1 and NP_001362742.1; (Isoform g precursor) NM_001375814.1 and NP_001362743.1; (Isoform b precursor) NM_002530.4 and NP_002521.2

### Day 15-30 (ENPs)—PHOX2B

NM_003924.4
(SEQ ID NO: 31)
CCGCAGTTCCTTACAAACTCTTCACGGACCACGGCGGCCTCAACGAGAAGCGCAAGCAGCGGCGCATCCG
CACCACTTTCACCAGTGCCCAGCTCAAAGAGCTGGAAAGGGTCTTCGCGGAGACTCACTACCCCGACATC
TACACTCGGGAGGAGCTGGCCCTGAAGATCGACCTCACAGAGGCGCGAGTCCAGGTGTGGTTCCAGAACC
GCCGCGCCAAGTTTCGCAAGCAGGAGCGCGCAGCGGCAGCCGCAGCGGCCGCGGCCAAGAACGGCTCCTC
GGGCAAAAAGTCTGACTCTTCCAGGGACGACGAGAGCAAAGAGGCCAAGAGCACTGACCCGGACAGCACT
GGGGGCCCAGGTCCCAATCCCAACCCCACCCCCAGCTGCGGGGCGAATGGAGGCGGCGGCGGCGGGCCCA
GCCCGGCTGGAGCTCCGGGGGCGGCGGGGCCCGGGGGCCCGGGAGGCGAACCCGGCAAGGGCGGCGCAGC
AGCAGCGGCGGCGGCCGCGGCAGCGGCGGCGGCGGCAGCGGCAGCGGCGGCAGCTGGAGGCCTGGCTGCG
GCTGGGGGCCCTGGACAAGGCTGGGCTCCCGGCCCCGGCCCCATCACCTCCATCCCGGATTCGCTTGGGG
GTCCCTTCGCCAGCGTCCTATCTTCGCTCCAAAGACCCAACGGTGCCAAAGCCGCCTTAGTGAAGAGCAG
TATGTTCTGATCTGGAATCCTGCGGCGGCGGCGGCGGCGGCGACAGCGGGCGAGCCAGGGCCCGGGCGGG
CGAGTGGGCGAGCGGGTAGGCCCAAGGCTATTGTCGTCGCTGCTGCCATGGCTTTTTCATTGAGGGCCTA
AAGTAATCGCGCTAAGAATAAAGGGAAAACGGCGTCGCCCTCATTTCAACCCCACTCCTACCCCCTTCCT
CAACCCCCAAACAAAACAAACAAACTTCCCTGGCTTCGCACCTGCCTGGGGCCTCGCAGCGGGGCCAGGG
CTCCGCCTGCTGATCGGGGGTTGTGAGCAGCGCGGCCTGGACGCGGGGCACTCTCAGGGGGCTGTGTCTG
CGTGTCAGTTTGTGTCTGTCTCGGGGAATGTGTGTCTGTGGCCCAAGCAGGTGACAGGAAGAGATGGGGG
GCCTCAACCAACTTAGTGACTTGTTTAGAAAAAAAAGACAAAAAAGTAAAAATAAAAACAAAAAAGTTGG
ACAAATTAAAGGAGGGGCTAGGGGAGAAGCTGCAGCTGGAGCTGAAGGCTCGATCTTGTGAACCCCTAAA
TCCGCTCCCTCCTAACAGCACGGATTCTCTTGGGGCTCTTCTTCAGGGAAGAGTAGGGACGCCGTTCCAG
CCCCCCTTCCTATCGTGTCCTTGGGTTCGGGTCACTGCGGCGACGACTTGCTCAGACTGTCCCGGCGGCC
GGAGTGACTTTCTCGCACCCCCTTGCCTGTCCCACCTCGCTGAACACCATCCCGCCATTAGCGCATCGGA
ACCCCACACAGTTGCAACTCCCAACCCCGAATCTTTGCAGCCGTTCGGCCCTGAAAGATGCCCTATCCAT
GAGATGCCTTTTCATCTGCAAACTCTGCAAAATGTGTCTCATGTTTCGCAACTCTTTTTTTCCCCCTCGC
TCCCGCCTACCCCGTCGGCATTTTCTTCTTCCACCAGCTTTTACTGAACTTTTTGGCACTGCTTTGGATT
GGGGTCAATTGCAGTCCACGTAACTGGCTGCAGAGAAATCTACCGAGCAAGGAAAAGGCACACACACACG
TTTGCAGGGGTGTCTCGGTTTGCATTTCTGTTGGAATGATCCGAACTGGACTCACATCCTGTATGGTGGA
TGGACTGTATATTGAGGGTTCCATTCTTCGCGCAGTTTAGACATCTCTGTTTTGATTCTTTGTTGTTGTT
TTTATTTTAAAAGGCACAAACTCTAGATATTAGTTGAATGTTGAGGCTTTAACTTTTTCGGTGTCTTTCT
ACAACTGTGTTCTGTGACTCAATTGTATCGTGTTAATATCAGTGCAGACTGTCTCCTCTACGTGACCGTA
TAATGTTTTTCTCTTCTTGTAGTCTCTATGGCGTGTCTTTATGGTGTAATAAGGTTCTCACGGGTTCAAT
CTTTTGTGTTTAGAGAGGCCACGGTTCAGACAATGGTATATATTTTTGTTATCAGGTGCATGTCTGTCTG
ATTTCTTTTTTTTTCCTGTTGGACTATGTTTGTGAACATAATTGTCATAAGTTATGTTTCAGATTTTTGA
ATTTATTTATATGTGTTATAATGAATGCTTCTATTTAAAAGGGAAATATTTCTACATGTGCATATAGTTT
TCCAAGAGTGTACCATTAACTTGATTGTTGATAATAAAAACAAAAAGCAAGTCTA
NP_003915.2
(SEQ ID NO: 32)
MYKMEYSYLNSSAYESCMAGMDTSSLASAYADFSSCSQASGFQYNPIRTTFGATSGCPSLTPGSCSLGTL
RDHQSSPYAAVPYKLFTDHGGLNEKRKQRRIRTTFTSAQLKELERVFAETHYPDIYTREELALKIDLTEA
RVQVWFQNRRAKFRKQERAAAAAAAAAKNGSSGKKSDSSRDDESKEAKSTDPDSTGGPGPNPNPTPSCGA
NGGGGGGPSPAGAPGAAGPGGPGGEPGKGGAAAAAAAAAAAAAAAAAAAAGGLAAAGGPGQGWAPGPGP1
TSIPDSLGGPFASVLSSLQRPNGAKAALVKSSMF

### Day 15-30 (ENPs)—ASCL1

NM_004316.4
(SEQ ID NO: 33)
GAAAGAAGGAAATCAGAAAGGAAGGGAGTTAACAAAATAATAAAAACAGCCTGAGCCACGGCTGGAGAGA
CCGAGACCCGGCGCAAGAGAGCGCAGCCTTAGTAGGAGAGGAACGCGAGACGCGGCAGAGCGCGTTCAGC
ACTGACTTTTGCTGCTGCTTCTGCTTTTTTTTTTCTTAGAAACAAGAAGGCGCCAGCGGCAGCCTCACAC
GCGAGCGCCACGCGAGGCTCCCGAAGCCAACCCGCGAAGGGAGGAGGGGAGGGAGGAGGAGGCGGCGTGC
AGGGAGGAGAAAAAGCATTTTCACTTTTTTTGCTCCCACTCTAAGAAGTCTCCCGGGGATTTTGTATATA
TTTTTTAACTTCCGTCAGGGCTCCCGCTTCATATTTCCTTTTCTTTCCCTCTCTGTTCCTGCACCCAAGT
TCTCTCTGTGTCCCCCTCGCGGGCCCCGCACCTCGCGTCCCGGATCGCTCTGATTCCGGGACTCCTTGGC
CGCCGCTGCGCATGGAAAGCTCTGCCAAGATGGAGAGCGGCGGCGCCGGCCAGCAGCCCCAGCCGCAGCC
CCAGCAGCCCTTCCTGCCGCCCGCAGCCTGTTTCTTTGCCACGGCCGCAGCCGCGGGGGCCGCAGCCGCC
GCAGCGGCAGCGCAGAGCGCGCAGCAGCAGCAGCAGCAGCAGCAGCAGCAGCAGCAGGCGCCGCAGCTGA
GACCGGCGGCCGACGGCCAGCCCTCAGGGGGCGGTCACAAGTCAGCGCCCAAGCAAGTCAAGCGACAGCG
CTCGTCTTCGCCCGAACTGATGCGCTGCAAACGCCGGCTCAACTTCAGCGGCTTTGGCTACAGCCTGCCG
CAGCAGCAGCCGGCCGCCGTGGCGCGCCGCAACGAGCGCGAGCGCAACCGCGTCAAGTTGGTCAACCTGG
GCTTTGCCACCCTTCGGGAGCACGTCCCCAACGGCGCGGCCAACAAGAAGATGAGTAAGGTGGAGACACT
GCGCTCGGCGGTCGAGTACATCCGCGCGCTGCAGCAGCTGCTGGACGAGCATGACGCGGTGAGGGCCGCC
TTCCAGGCAGGCGTCCTGTCGCCCACCATCTCCCCCAACTACTCCAACGACTTGAACTCCATGGCCGGCT
CGCCGGTCTCATCCTACTCGTCGGACGAGGGCTCTTACGACCCGCTCAGCCCCGAGGAGCAGGAGCTTCT
CGACTTCACCAACTGGTTCTGAGGGGCTCGGCCTGGTCAGGCCCTGGTGCGAATGGACTTTGGAAGCAGG
GTGATCGCACAACCTGCATCTTTAGTGCTTTCTTGTCAGTGGCGTTGGGAGGGGGAGAAAAGGAAAAGAA
CTAAGCGAGGCATGCCTGAGAGACATGGCTTTCAGAAAACGGGAAGCGCTCAGAACAGTATCTTTGCACT
CCAATCATTCACGGAGATATGAAGAGCAACTGGGACCTGAGTCAATGCGCAAAATGCAGCTTGTGTGCAA
AAGCAGTGGGCTCCTGGCAGAAGGGAGCAGCACACGCGTTATAGTAACTCCCATCACCTCTAACACGCAC
AGCTGAAAGTTCTTGCTCGGGTCCCTTCACCTCCTCGCCCTTTCTTAAAGTGCAGTTCTTAGCCCTCTAG
AAACGAGTTGGTGTCTTTCGTCTCAGTAGCCCCCACCCCAATAAGCTGTAGACATTGGTTTACAGTGAAA
CTATGCTATTCTCAGCCCTTTGAAACTCTGCTTCTCCTCCAGGGCCCGATTCCCAAACCCCATGGCTTCC
CTCACACTGTCTTTTCTACCATTTTCATTATAGAATGCTTCCAATCTTTTGTGAATTTTTTATTATAAAA
AATCTATTTGTATCTATCCTAACCAGTTCGGGGATATATTAAGATATTTTTGTACATAAGAGAGAAAGAG
AGAGAAAAATTTATAGAAGTTTTGTACAAATGGTTTAAAATGTGTATATCTTGATACTTTAACATGTAAT
GCTATTACCTCTGCATATTTTAGATGTGTAGTTCACCTTACAACTGCAATTTTCCCTATGTGGTTTTGTA
AAGAACTCTCCTCATAGGTGAGATCAAGAGGCCACCAGTTGTACTTCAGCACCAATGTGTCTTACTTTAT
AGAAATGTTGTTAATGTATTAATGATGTTATTAAATACTGTTCAAGAAGAACAAAGTTTATGCAGCTACT
GTCCAAACTCAAAGTGGCAGCCAGTTGGTTTTGATAGGTTGCCTTTTGGAGATTTCTATTACTGCCTTTT
TTTTTCTTACTGTTTTATTACAAACTTACAAAAATATGTATAACCCTGTTTTATACAAACTAGTTTCGTA
ATAAAACTTTTTCCTTTTTTTAAAATGAAAA
NP_004307.2
(SEQ ID NO: 34)
MESSAKMESGGAGQQPQPQPQQPFLPPAACFFATAAAAAAAAAAAAAQSAQQQQQQQQQQQQAPQLRPAA
DGQPSGGGHKSAPKQVKRQRSSSPELMRCKRRLNFSGFGYSLPQQQPAAVARRNERERNRVKLVNLGFAT
LREHVPNGAANKKMSKVETLRSAVEYIRALQQLLDEHDAVSAAFQAGVLSPTISPNYSNDLNSMAGSPVS
SYSSDEGSYDPLSPEEQELLDFTNWF

### Day 15-30 (ENPs)—EDNRB

(Isoform 1 precursor) NM_000115.5
(SEQ ID NO: 35)
CTTTCTAAAAAGAGATTTATTTTTAAATCAATGGGACTCTGATATAAAGGAAGAATAAGTCACTGTAAAA
CAGAACTTTTAAATGAAGCTTAAATTACTCAATTTAAAATTTTAAAATCCTTTAAAACAACTTTTCAATT
AATATTATCACACTATTATCAGATTGTAATTAGATGCAAATGAGAGAGCAGTTTAGTTGTTGCATTTTTC
GGACACTGGAAACATTTAAATGATCAGGAGGGAGTAACAGAAAGAGCAAGGCTGTTTTTGAAAATCATTA
CACTTTCACTAGAAGCCCAAACCTCAGCATTCTGCAATATGTAACCAACATGTCACAAACAAGCAGCATG
TAACAGACTGGCACATGTGCCAGCTGAATTTAAAATATAATACTTTTAAAAAGAAAATTATTACATCCTT
TACATTCAGTTAAGATCAAACCTCACAAAGAGAAATAGAATGTTTGAAAGGCTATCCCAAAAGACTTTTT
TGAATCTGTCATTCACATACCCTGTGAAGACAATACTATCTACAATTTTTTCAGGATTATTAAAATCTTC
TTCTTTCACTATCGTAGCTTAAACTCTGTTTGGTTTTGTCATCTGTAAATACTTACCTACATACACTGCA
TGTAGATGATTAAATGAGGGCAGGCCCTGTGCTCATAGCTTTACGATGGAGAGATGCCAGTGACCTCATA
ATAAAGACTGTGAACTGCCTGGTGCAGTGTCCACATGACAAAGGGGCAGGTAGCACCCTCTCTCACCCAT
GCTGTGGTTAAAATGGTTTCTAGCATATGTATAATGCTATAGTTAAAATACTATTTTTCAAAATCATACA
GATTAGTACATTTAACAGCTACCTGTAAAGCTTATTACTAATTTTTGTATTATTTTTGTAAATAGCCAAT
AGAAAAGTTTGCTTGACATGGTGCTTTTCTTTCATCTAGAGGCAAAACTGCTTTTTGAGACCGTAAGAAC
CTCTTAGCTTTGTGCGTTCCTGCCTAATTTTTATATCTTCTAAGCAAAGTGCCTTAGGATAGCTTGGGAT
GAGATGTGTGTGAAAGTATGTACAAGAGAAAACGGAAGAGAGAGGAAATGAGGTGGGGTTGGAGGAAACC
CATGGGGACAGATTCCCATTCTTAGCCTAACGTTCGTCATTGCCTCGTCACATCAATGCAAAAGGTCCTG
ATTTTGTTCCAGCAAAACACAGTGCAATGTTCTCAGAGTGACTTTCGAAATAAATTGGGCCCAAGAGCTT
TAACTCGGTCTTAAAATATGCCCAAATTTTTACTTTGTTTTTCTTTTAATAGGCTGGGCCACATGTTGGA
AATAAGCTAGTAATGTTGTTTTCTGTCAATATTGAATGTGATGGTACAGTAAACCAAAACCCAACAATGT
GGCCAGAAAGAAAGAGCAATAATAATTAATTCACACACCATATGGATTCTATTTATAAATCACCCACAAA
CTTGTTCTTTAATTTCATCCCAATCACTTTTTCAGAGGCCTGTTATCATAGAAGTCATTTTAGACTCTCA
ATTTTAAATTAATTTTGAATCACTAATATTTTCACAGTTTATTAATATATTTAATTTCTATTTAAATTTT
AGATTATTTTTATTACCATGTACTGAATTTTTACATCCTGATACCTTTTCCTTCTCCATGTCAGTATCAT
GTTCTCTAATTATCTTGCCAAATTTTGAAACTACACACAAAAAGCATACTTGCATTATTTATAATAAAAT
TGCATTCAGTGGCTTTTTAAAAAAATGTTTGATTCAAAACTTTAACATACTGATAAGTAAGAAACAATTA
TAATTTCTTTACATACTCAAAACCAAGATAGAAAAAGGTGCTATCGTTCAACTTCAAAACATGTTTCCTA
GTATTAAGGACTTTAATATAGCAACAGACAAAATTATTGTTAACATGGATGTTACAGCTCAAAAGATTTA
TAAAAGATTTTAACCTATTTTCTCCCTTATTATCCACTGCTAATGTGGATGTATGTTCAAACACCTTTTA
GTATTGATAGCTTACATATGGCCAAAGGAATACAGTTTATAGCAAAACATGGGTATGCTGTAGCTAACTT
TATAAAAGTGTAATATAACAATGTAAAAAATTATATATCTGGGAGGATTTTTTGGTTGCCTAAAGTGGCT
ATAGTTACTGATTTTTTATTATGTAAGCAAAACCAATAAAAATTTAAGTTTTTTTAACAACTACCTTATT
TTTCACTGTACAGACACTAATTCATTAAATACTAATTGATTGTTTAAAAGAAATATAAATGTGACAAGTG
GACATTATTTATGTTAAATATACAATTATCAAGCAAGTATGAAGTTATTCAATTAAAATGCCACATTTCT
GGTC
(Isoform 1 precursor) NP_000106.1
(SEQ ID NO: 36)
MQPPPSLCGRALVALVLACGLSRIWGEERGFPPDRATPLLQTAEIMTPPTKTLWPKGSNASLARSLAPAE
VPKGDRTAGSPPRTISPPPCQGPIEIKETFKYINTVVSCLVFVLGIIGNSTLLRIIYKNKCMRNGPNILI
ASLALGDLLHIVIDIPINVYKLLAEDWPFGAEMCKLVPFIQKASVGITVLSLCALSIDRYRAVASWSRIK
GIGVPKWTAVEIVLIWVVSVVLAVPEAIGFDIITMDYKGSYLRICLLHPVQKTAFMQFYKTAKDWWLFSF
YFCLPLAITAFEYTLMTCEMLRKKSGMQIALNDHLKQRREVAKTVFCLVLVFALCWLPLHLSRILKLTLY
NQNDPNRCELLSFLLVLDYIGINMASLNSCINPIALYLVSKRFKNCFKSCLCCWCQSFEEKQSLEEKQSC
LKFKANDHGYDNFRSSNKYSSS

Isoform/Variant includes: (Isoform 1 precursor) NM_001122659.3 and NP_001116131.1; (Isoform 3) NM_001201397.1 and NP_001188326.1; (Isoform 2 precursor) NM_003991.4 and NP_003982.1

### Day 15-30 (ENPs)—RET

(Isoform d) NM_001355216.1
(SEQ ID NO: 37)
TCCACCTGCTCTCCCAGCACCAAGACCTGCCCCGACGGCCACTGCGATGTTGTGGAGACCCAAGACATCA
ACATTTGCCCTCAGGACTGCCTCCGGGGCAGCATTGTTGGGGGACACGAGCCTGGGGAGCCCCGGGGGAT
TAAAGCTGGCTATGGCACCTGCAACTGCTTCCCTGAGGAGGAGAAGTGCTTCTGCGAGCCCGAAGACATC
CAGGATCCACTGTGCGACGAGCTGTGCCGCACGGTGATCGCAGCCGCTGTCCTCTTCTCCTTCATCGTCT
CGGTGCTGCTGTCTGCCTTCTGCATCCACTGCTACCACAAGTTTGCCCACAAGCCACCCATCTCCTCAGC
TGAGATGACCTTCCGGAGGCCCGCCCAGGCCTTCCCGGTCAGCTACTCCTCTTCCGGTGCCCGCCGGCCC
TCGCTGGACTCCATGGAGAACCAGGTCTCCGTGGATGCCTTCAAGATCCTGGAGGATCCAAAGTGGGAAT
TCCCTCGGAAGAACTTGGTTCTTGGAAAAACTCTAGGAGAAGGCGAATTTGGAAAAGTGGTCAAGGCAAC
GGCCTTCCATCTGAAAGGCAGAGCAGGGTACACCACGGTGGCCGTGAAGATGCTGAAAGAGAACGCCTCC
CCGAGTGAGCTGCGAGACCTGCTGTCAGAGTTCAACGTCCTGAAGCAGGTCAACCACCCACATGTCATCA
AATTGTATGGGGCCTGCAGCCAGGATGGCCCGCTCCTCCTCATCGTGGAGTACGCCAAATACGGCTCCCT
GCGGGGCTTCCTCCGCGAGAGCCGCAAAGTGGGGCCTGGCTACCTGGGCAGTGGAGGCAGCCGCAACTCC
AGCTCCCTGGACCACCCGGATGAGCGGGCCCTCACCATGGGCGACCTCATCTCATTTGCCTGGCAGATCT
CACAGGGGATGCAGTATCTGGCCGAGATGAAGCTCGTTCATCGGGACTTGGCAGCCAGAAACATCCTGGT
AGCTGAGGGGCGGAAGATGAAGATTTCGGATTTCGGCTTGTCCCGAGATGTTTATGAAGAGGATTCCTAC
GTGAAGAGGAGCCAGGGTCGGATTCCAGTTAAATGGATGGCAATTGAATCCCTTTTTGATCATATCTACA
CCACGCAAAGTGATGTATGGTCTTTTGGTGTCCTGCTGTGGGAGATCGTGACCCTAGGGGGAAACCCCTA
TCCTGGGATTCCTCCTGAGCGGCTCTTCAACCTTCTGAAGACCGGCCACCGGATGGAGAGGCCAGACAAC
TGCAGCGAGGAGATGTACCGCCTGATGCTGCAATGCTGGAAGCAGGAGCCGGACAAAAGGCCGGTGTTTG
CGGACATCAGCAAAGACCTGGAGAAGATGATGGTTAAGAGGAGAGACTACTTGGACCTTGCGGCGTCCAC
TCCATCTGACTCCCTGATTTATGACGACGGCCTCTCAGAGGAGGAGACACCGCTGGTGGACTGTAATAAT
GCCCCCCTCCCTCGAGCCCTCCCTTCCACATGGATTGAAAACAAACTCTATGGTAGAATTTCCCATGCAT
TTACTAGATTCTAGCACCGCTGTCCCCTTTGCACTATCCTTCCTCTCTGTGATGCTTTTTAAAAATGTTT
CTGGTCTGAACAAAACCAAAGTCTGCTCTGAACCTTTTTATTTGTAAATGTCTGACTTTGCATCCAGTTT
ACATTTAGGCATTATTGCAACTATGTTTTTCTAAAAGGATGTGAAAATAAGTGTAATTACCACATTGCCC
AGCAACTTAGGATGGTAGAGGAAAAAACAGATCAGGGCGGAACTCTCAGGGGAGACCAAGAACAGGTTGA
ATAAGGCGCTTCTGGGGTGGGAATCAAGTCATAGTACTTCTACTTTAACTAAGTGGATAAATATACAAAT
CTGGGGAGGTATTCAGTTGAGAAAGGAGCCACCAGCACCACTCAGCCTGCACTGGGAGCACAGCCAGGTT
CCCCCAGACCCCTCCTGGGCAGGCAGGTGCCTCTCAGAGGCCACCCGGCACTGGCGAGCAGCCACTGGCC
AAGCCTCAGCCCCAGTCCCAGCCACATGTCCTCCATCAGGGGTAGCGAGGTTGCAGGAGCTGGCTGGCCC
TGGGAGGACGCACCCCCACTGCTGTTTTCACATCCTTTCCCTTACCCACCTTCAGGACGGTTGTCACTTA
TGAAGTCAGTGCTAAAGCTGGAGCAGTTGCTTTTTGAAAGAACATGGTCTGTGGTGCTGTGGTCTTACAA
TGGACAGTAAATATGGTTCTTGCCAAAACTCCTTCTTTTGTCTTTGATTAAATACTAGAAATTTTTTCTG
TTTCCTAACTTCATCATTGATTGTTTGAAATCTTGGAGTTTCAAGCATTTTTTTCAAGCTGAGACGGTTC
CTTTTGCATGCCTCCTGACTCACTGACTCCTCAC
(Isoform d) NP_001342145.1
(SEQ ID NO: 38)
MVPFPVTVYDEDDSAPTFPAGVDTASAVVEFKRKEDTVVATLRVFDADVVPASGELVRRYTSTLLPGDTW
AQQTFRVEHWPNETSVQANGSFVRATVHDYRLVLNRNLSISENRTMQLAVLVNDSDFQGPGAGVLLLHFN
VSVLPVSLHLPSTYSLSVSRRARRFAQIGKVCVENCQAFSGINVQYKLHSSGANCSTLGVVTSAEDTSGI
LFVNDTKALRRPKCAELHYMVVATDQQTSRQAQAQLLVTVEGSYVAEEAGCPLSCAVSKRRLECEECGGL
GSPTGRCEWRQGDGKGITRNFSTCSPSTKTCPDGHCDVVETQDINICPQDCLRGSIVGGHEPGEPRGIKA
GYGTCNCFPEEEKCFCEPEDIQDPLCDELCRTVIAAAVLFSFIVSVLLSAFCIHCYHKFAHKPPISSAEM
TFRRPAQAFPVSYSSSGARRPSLDSMENQVSVDAFKILEDPKWEFPRKNLVLGKTLGEGEFGKVVKATAF
HLKGRAGYTTVAVKMLKENASPSELRDLLSEFNVLKQVNHPHVIKLYGACSQDGPLLLIVEYAKYGSLRG
FLRESRKVGPGYLGSGGSRNSSSLDHPDERALTMGDLISFAWQISQGMQYLAEMKLVHRDLAARNILVAE
GRKMKISDFGLSRDVYEEDSYVKRSQGRIPVKWMAIESLFDHIYTTQSDVWSFGVLLWEIVTLGGNPYPG
IPPERLFNLLKTGHRMERPDNCSEEMYRLMLQCWKQEPDKRPVFADISKDLEKMMVKRRDYLDLAASTPS
DSLIYDDGLSEEETPLVDCNNAPLPRALPSTWIENKLYGRISHAFTRF

Isoform/Variant includes: (Isoform c precursor) NM_020630.5 and NP_065681.1; (Isoform a precursor) NM_020975.6 and NP_066124.1
Day >35 (enteric neurons)—TUJ1 (TUBB3)

(Isoform 2) NM_001197181.2
(SEQ ID NO: 39)
ACCAGACCCCTCTGAGGATGGAGCAGGAGCTGGCTGCCCTGAGGCTGCAAAACTTCTTCCCTCGTGGAGA
CAGGGAGGCACCTCAGACACTCACCCCGGACTCCCTTGAACAGGGACAGGGAGGAACCCCAGGCAGCTAG
ACCCCAGCAGCAGCCACACGAGCACACTGTGGGGCAGGGAGGGGCATCTCTTGAGAACAAAAGATCCATT
TCTCGACTTTCCAAACTGGAGAGCTTCTTGAGAGAAAAGAGAGAGACAGGTACAGGTCCACGCCACCCAC
ACACAGCCCTGTGCACACAGACCGGACACAGGCGTCCACAGTTCTGGGAAGTCATCAGTGATGAGCATGG
CATCGACCCCAGCGGCAACTACGTGGGCGACTCGGACTTGCAGCTGGAGCGGATCAGCGTCTACTACAAC
GAGGCCTCTTCTCACAAGTACGTGCCTCGAGCCATTCTGGTGGACCTGGAACCCGGAACCATGGACAGTG
TCCGCTCAGGGGCCTTTGGACATCTCTTCAGGCCTGACAATTTCATCTTTGGTCAGAGTGGGGCCGGCAA
CAACTGGGCCAAGGGTCACTACACGGAGGGGGCGGAGCTGGTGGATTCGGTCCTGGATGTGGTGCGGAAG
GAGTGTGAAAACTGCGACTGCCTGCAGGGCTTCCAGCTGACCCACTCGCTGGGGGGCGGCACGGGCTCCG
GCATGGGCACGTTGCTCATCAGCAAGGTGCGTGAGGAGTATCCCGACCGCATCATGAACACCTTCAGCGT
CGTGCCCTCACCCAAGGTGTCAGACACGGTGGTGGAGCCCTACAACGCCACGCTGTCCATCCACCAGCTG
GTGGAGAACACGGATGAGACCTACTGCATCGACAACGAGGCGCTCTACGACATCTGCTTCCGCACCCTCA
AGCTGGCCACGCCCACCTACGGGGACCTCAACCACCTGGTATCGGCCACCATGAGCGGAGTCACCACCTC
CTTGCGCTTCCCGGGCCAGCTCAACGCTGACCTGCGCAAGCTGGCCGTCAACATGGTGCCCTTCCCGCGC
CTGCACTTCTTCATGCCCGGCTTCGCCCCCCTCACAGCCCGGGGCAGCCAGCAGTACCGGGCCCTGACCG
TGCCCGAGCTCACCCAGCAGATGTTCGATGCCAAGAACATGATGGCCGCCTGCGACCCGCGCCACGGCCG
CTACCTGACGGTGGCCACCGTGTTCCGGGGCCGCATGTCCATGAAGGAGGTGGACGAGCAGATGCTGGCC
ATCCAGAGCAAGAACAGCAGCTACTTCGTGGAGTGGATCCCCAACAACGTGAAGGTGGCCGTGTGTGACA
TCCCGCCCCGCGGCCTCAAGATGTCCTCCACCTTCATCGGGAACAGCACGGCCATCCAGGAGCTGTTCAA
GCGCATCTCCGAGCAGTTCACGGCCATGTTCCGGCGCAAGGCCTTCCTGCACTGGTACACGGGCGAGGGC
ATGGACGAGATGGAGTTCACCGAGGCCGAGAGCAACATGAACGACCTGGTGTCCGAGTACCAGCAGTACC
AGGACGCCACGGCCGAGGAAGAGGGCGAGATGTACGAAGACGACGAGGAGGAGTCGGAGGCCCAGGGCCC
CAAGTGAAGCTGCTCGCAGCTGGAGTGAGAGGCAGGTGGCGGCCGGGGCCGAAGCCAGCAGTGTCTAAAC
CCCCGGAGCCATCTTGCTGCCGACACCCTGCTTTCCCCTCGCCCTAGGGCTCCCTTGCCGCCCTCCTGCA
GTATTTATGGCCTCGTCCTCCCCACCTAGGCCACGTGTGAGCTGCTCCTGTCTCTGTCTTATTGCAGCTC
CAGGCCTGACGTTTTACGGTTTTGTTTTTTACTGGTTTGTGTTTATATTTTCGGGGATACTTAATAAATC
TATTGCTGTCAGATACCCTT
(Isoform 2) NP_001 184110.1
(SEQ ID NO: 40)
MDSVRSGAFGHLFRPDNFIFGQSGAGNNWAKGHYTEGAELVDSVLDVVRKECENCDCLQGFQLTHSLGGG
TGSGMGTLLISKVREEYPDRIMNTFSVVPSPKVSDTVVEPYNATLSIHQLVENTDETYCIDNEALYDICF
RTLKLATPTYGDLNHLVSATMSGVTTSLRFPGQLNADLRKLAVNMVPFPRLHFFMPGFAPLTARGSQQYR
ALTVPELTQQMFDAKNMMAACDPRHGRYLTVATVFRGRMSMKEVDEQMLAIQSKNSSYFVEWIPNNVKVA
VCDIPPRGLKMSSTFIGNSTAIQELFKRISEQFTAMFRRKAFLHWYTGEGMDEMEFTEAESNMNDLVSEY
QQYQDATAEEEGEMYEDDEEESEAQGPK

Isoform/Variant includes: (Isoform 1) NM_006086.4 and NP_006077.2
Day >35 (enteric neurons)—CHAT

(Isoform 1)NM_001142929.1
(SEQ ID NO: 41)
TTGCCCGAGTTCCTCCGGGAAGCGCTCCGGGTAGATTCTGGGGGCCGGGAGCTGAGATCCCTGGGCGGGG
AGCTGGGGAAGGGATGGGGCTGAGGACAGCGAAGAAGAGGGGGCTTGGGGGAGGGGGGAAATGGAAGAGA
GAGGAGGGAGGAGGTACAAGAGGAAGGAGAGAAGTGCGGCCAGCTTGCTTTCTCCAGTCGGGTGGCCGCG
GGGACCCGGGCGACGTCGGAGGCCCTGCCGGGAACCCAGGCTGCAGCCCCCACCCCCGCGCTGCGACACG
CCCCCCACCCCTTCCGGCTCACACCCCCGCCCACACTCCTGAGTGGTGCGGTGCAGCGTCGGCCGAGGCA
GCAGAGCCGAGGAGAGCAGGTCCACACCTCTGCATCCCTGCACCAGGACTCACCAAGACGCCCATCCTGG
AAAAGGTCCCCCGTAAGATGGCAGCAAAAACTCCCAGCAGTGAGGAGTCTGGGCTGCCCAAACTGCCCGT
GCCCCCGCTGCAGCAGACCCTGGCCACGTACCTGCAGTGCATGCGACACTTGGTGTCTGAGGAGCAGTTC
AGGAAGAGCCAGGCCATTGTGCAGCAGTTTGGGGCCCCTGGTGGCCTCGGCGAGACCCTGCAGCAGAAAC
TCCTGGAGCGGCAGGAGAAGACAGCCAACTGGGTGTCTGAGTACTGGCTGAATGACATGTATCTCAACAA
CCGCCTGGCCCTGCCTGTCAACTCCAGCCCTGCCGTGATCTTTGCTCGGCAGCACTTCCCTGGCACCGAT
GACCAGCTGAGGTTTGCAGCCAGCCTCATCTCTGGTGTACTCAGCTACAAGGCCCTGCTGGACAGCCACT
CCATTCCCACTGACTGTGCCAAAGGCCAGCTGTCAGGGCAGCCCCTTTGCATGAAGCAATACTATGGGCT
CTTCTCCTCCTACCGGCTCCCCGGCCATACCCAGGACACGCTGGTGGCTCAGAACAGCAGCATCATGCCG
GAGCCTGAGCACGTCATCGTAGCCTGCTGCAATCAGTTCTTTGTCTTGGATGTTGTCATTAATTTCCGCC
GTCTCAGTGAGGGGGATCTGTTCACTCAGTTGAGAAAGATAGTCAAAATGGCTTCCAACGAGGACGAGCG
TTTGCCTCCAATTGGCCTGCTGACGTCTGACGGGAGGAGCGAGTGGGCCGAGGCCAGGACGGTCCTCGTG
AAAGACTCCACCAACCGGGACTCGCTGGACATGATTGAGCGCTGCATCTGCCTTGTATGCCTGGACGCGC
CAGGAGGCGTGGAGCTCAGCGACACCCACAGGGCACTCCAGCTCCTTCACGGCGGAGGCTACAGCAAGAA
CGGGGCCAATCGCTGGTACGACAAGTCCCTGCAGTTTGTGGTGGGCCGAGACGGCACCTGCGGTGTGGTG
TGCGAACACTCCCCATTCGATGGCATCGTCCTGGTGCAGTGCACTGAGCATCTGCTCAAGCACATGACGC
AGAGCAGCAGGAAGCTGATCCGAGCAGACTCCGTCAGCGAGCTCCCCGCCCCCCGGAGGCTGCGGTGGAA
ATGCTCCCCGGAAATTCAAGGCCACTTAGCCTCCTCGGCAGAAAAACTTCAACGAATAGTAAAGAACCTT
GACTTCATTGTCTATAAGTTTGACAACTATGGGAAAACATTCATTAAGAAGCAGAAATGCAGCCCTGATG
CCTTCATCCAGGTGGCCCTCCAGCTGGCCTTCTACAGGCTCCATCGAAGACTGGTGCCCACCTACGAGAG
CGCGTCCATCCGCCGATTCCAGGAGGGACGCGTGGACAACATCAGATCGGCCACTCCAGAGGCACTGGCT
TTTGTGAGAGCCGTGACTGACCACAAGGCTGCTGTGCCAGCTTCTGAGAAGCTTCTGCTCCTGAAGGATG
CCATCCGTGCCCAGACTGCATACACAGTCATGGCCATAACAGGGATGGCCATTGACAACCACCTGCTGGC
ACTGCGGGAGCTGGCCCGGGCCATGTGCAAGGAGCTGCCCGAGATGTTCATGGATGAAACCTACCTGATG
AGCAACCGGTTTGTCCTCTCCACTAGCCAGGTGCCCACAACCACGGAGATGTTCTGCTGCTATGGTCCTG
TGGTCCCAAATGGGTATGGTGCCTGCTACAACCCCCAGCCAGAGACCATCCTTTTCTGCATCTCTAGCTT
TCACAGCTGCAAAGAGACTTCTTCTAGCAAGTTTGCAAAAGCTGTGGAAGAAAGCCTCATTGACATGAGA
GACCTCTGCAGTCTGCTGCCGCCTACTGAGAGCAAGCCATTGGCAACAAAGGAAAAAGCCACGAGGCCCA
GCCAGGGACACCAACCTTGACTCCTGCCACTAGGTTTCACCTCCCAAACCCAGCCTCTAGAACAGCCAGA
CCCTGCAG
(Isoform 1)NP_001136401.1
(SEQ ID NO: 42)
MAAKTPSSEESGLPKLPVPPLQQTLATYLQCMRHLVSEEQFRKSQAIVQQFGAPGGLGETLQQKLLERQE
KTANWVSEYWLNDMYLNNRLALPVNSSPAVIFARQHFPGTDDQLRFAASLISGVLSYKALLDSHSIPTDC
AKGQLSGQPLCMKQYYGLFSSYRLPGHTQDTLVAQNSSIMPEPEHVIVACCNQFFVLDVVINFRRLSEGD
LFTQLRKIVKMASNEDERLPPIGLLTSDGRSEWAEARTVLVKDSTNRDSLDMIERCICLVCLDAPGGVEL
SDTHRALQLLHGGGYSKNGANRWYDKSLQFVVGRDGTCGVVCEHSPFDGIVLVQCTEHLLKHMTQSSRKL
IRADSVSELPAPRRLRWKCSPEIQGHLASSAEKLQRIVKNLDFIVYKFDNYGKTFIKKQKCSPDAFIQVA
LQLAFYRLHRRLVPTYESASIRRFQEGRVDNIRSATPEALAFVRAVTDHKAAVPASEKLLLLKDAIRAQT
AYTVMATTGMAIDNHLLALRELARAMCKELPEMFMDETYLMSNRFVLSTSQVPTTTEMFCCYGPVVPNGY
GACYNPQPETILFCISSFHSCKETSSSKFAKAVEESLIDMRDLCSLLPPTESKPLATKEKATRPSQGHQP

Isoform/Variant includes: (Isoform 3) NM_001142933.1 and NP_001136405.1; (Isoform 1) NM_001142934.1 and NP_001136406.1; (Isoform 2) NM_020549.4 and NP_065574.3; (Isoform 1) NM_020984.3 and NP_066264.3; (Isoform 1) NM_020985.3 and NP_066265.3; (Isoform 1) NM_020986.3 and NP_066266.3
Day >35 (enteric neurons)—SLC18A3

NM_003055.3
(SEQ ID NO: 43)
AAGAGCCGACGCGAGGGGAGGGGAGCGCAGCGGCGGGGCTAACGGGCGGGCAAGCGGGCGGGCGGCAACA
GCATGTCCCTCGGCCAGCGCGGGCGGCCTCTTAGCGCGGCGGGGGCTGCTCTGGGCGCGCCCCGGGCGAA
GTGCGCCCAGTCTCCGGCCCCGGCCCCTCGGCGCGCCCGACTTCCCGGCCGCCCCTGAGCCCAGCAGCCG
CGGGTCCCGGGATCGGCTAAGAGTAGCTGCAACGCCTCGCCGGACGGAGTCCTTTCCTTTCCCGGGACGC
TGGGCCATGAGCTCCGCGGCCACCTGAGGCACAGGGGAGTCTGCTCGGCCAGGACAGCCTCCCCGAAGTC
CCGTGCCCTCGCCTCTGCACTGCGGGACGCCAGCGCTCGGCCCTGGCGGAGGCGTCCTCGGAAGAGCATC
GGGGTGGGGGCATGGAATCCGCGGAACCTGCGGGCCAGGCCCGGGCGGCGGCCACCAAGCTGTCGGAGGC
TGTGGGCGCGGCGCTGCAGGAGCCCCGGCGGCAGAGGCGCCTGGTGCTTGTTATCGTGTGCGTGGCGCTG
TTACTGGACAACATGCTGTACATGGTCATCGTGCCCATAGTGCCCGACTACATCGCCCACATGCGCGGGG
GCGGCGAGGGCCCCACCCGGACTCCCGAGGTGTGGGAGCCCACCCTGCCGCTGCCCACTCCGGCCAATGC
CAGCGCCTACACGGCCAACACCTCGGCGTCCCCGACAGCTGCGTGGCCAGCGGGCTCAGCCCTTCGGCCC
CGCTACCCTACGGAGAGCGAAGACGTGAAGATCGGGGTGCTGTTTGCTTCCAAGGCTATCCTGCAGCTGC
TAGTGAACCCCTTGAGCGGGCCCTTCATCGACCGCATGAGCTACGACGTGCCGCTGCTGATCGGCCTGGG
CGTCATGTTCGCCTCTACAGTCCTGTTCGCCTTCGCCGAGGACTACGCCACGCTGTTCGCGGCGCGCAGC
CTGCAGGGCCTGGGCTCAGCCTTCGCCGACACGTCTGGCATAGCCATGATCGCCGATAAGTACCCGGAGG
AGCCGGAGCGCAGTCGTGCACTGGGCGTGGCGCTGGCCTTCATTAGCTTCGGAAGCCTAGTGGCCCCGCC
CTTCGGGGGCATCCTCTATGAGTTCGCCGGCAAGCGCGTGCCCTTCTTGGTGCTAGCTGCCGTGTCGCTC
TTTGACGCGCTGTTGCTGCTGGCAGTGGCCAAACCCTTCTCGGCGGCTGCACGGGCTCGGGCCAACCTGC
CAGTGGGCACTCCCATCCACCGCCTCATGCTAGACCCCTACATTGCCGTGGTGGCCGGCGCGCTCACCAC
CTGTAACATTCCCCTCGCCTTCCTCGAACCCACCATTGCCACGTGGATGAAGCATACGATGGCGGCTTCC
GAGTGGGAGATGGGCATGGCCTGGCTGCCGGCCTTCGTGCCTCATGTGCTGGGCGTCTACCTCACCGTGC
GCCTGGCGGCGCGCTACCCACACCTGCAGTGGCTGTACGGCGCGCTTGGGCTGGCTGTGATCGGCGCCAG
CTCGTGCATCGTGCCCGCCTGCCGCTCCTTCGCGCCGCTAGTGGTCTCACTATGCGGCCTCTGTTTTGGC
ATAGCCCTAGTCGACACAGCACTGCTGCCCACGCTCGCCTTCCTGGTGGACGTGCGCCATGTCTCAGTCT
ATGGCAGCGTCTACGCCATCGCCGACATCTCCTATTCGGTGGCCTACGCGCTCGGGCCCATAGTGGCAGG
CCACATTGTGCACTCGCTGGGCTTTGAGCAGCTCAGCCTTGGCATGGGACTGGCCAACCTGCTCTATGCT
CCCGTCTTGCTGCTGCTCCGCAACGTGGGCCTCCTGACGCGCTCCCGTTCCGAGCGCGATGTGCTGCTTG
ATGAGCCACCGCAAGGTCTGTACGATGCGGTGCGCCTGCGTGAGCGTCCTGTGTCTGGCCAGGACGGCGA
GCCTCGCAGCCCGCCTGGCCCTTTTGATGCGTGCGAGGACGACTACAACTACTACTACACCCGCAGCTAG
CATCCCCACTCCTCCTCCAGCCCACCCAACCGCCTTGGGTCAAGGGGGCTGCTCTGCAAGCCCACTGGCC
AGCTCTGGCTCAGGGCCCACCTCCTCCAGCGAGTACCCCAGCCACTCCTCAACCTTGACTTCTGCCCAAA
TCCCCTCCCTGTGACCCGTTCCATATCCCTTTCTCTCTTGTCCAATGGGGCTTGGAGCACCGAGGCCAGC
GAAGCCATCGCGCTCCTTGCGGAGGTGAAGAGGACCCTGAGTCCCCACCTGCGGCTCCCCTGTGTAGAGC
CTGCATCTGTCTGTCCTTCCTTCCATTGCTCCCAGTGCCAAACTTGGGCCGCTGCACCGCGGCGCCTCCG
CCCAAATCAATAAACTGTGTCTGTCCCAGGA
NP_003046.2
(SEQ ID NO: 44)
MESAEPAGQARAAATKLSEAVGAALQEPRRQRRLVLVIVCVALLLDNMLYMVIVPIVPDYIAHMRGGGEG
PTRTPEVWEPTLPLPTPANASAYTANTSASPTAAWPAGSALRPRYPTESEDVKIGVLFASKAILQLLVNP
LSGPFIDRMSYDVPLLIGLGVMFASTVLFAFAEDYATLFAARSLQGLGSAFADTSGIAMIADKYPEEPER
SRALGVALAFISFGSLVAPPFGGILYEFAGKRVPFLVLAAVSLFDALLLLAVAKPFSAAARARANLPVGT
PIHRLMLDPYIAVVAGALTTCNIPLAFLEPTIATWMKHTMAASEWEMGMAWLPAFVPHVLGVYLTVRLAA
RYPHLQWLYGALGLAVIGASSCIVPACRSFAPLVVSLCGLCFGIALVDTALLPTLAFLVDVRHVSVYGSV
YAIADISYSVAYALGPIVAGHIVHSLGFEQLSLGMGLANLLYAPVLLLLRNVGLLTRSRSERDVLLDEPP
QGLYDAVRLRERPVSGQDGEPRSPPGPFDACEDDYNYYYTRS

Day >35 (enteric neurons)—NOS1

(Isoform 1) NM_000620.5
(SEQ ID NO: 45)
CTTGCCATTAGGAAATCTGTGGACTTCTAGCCTGTGTTTTAAACCAGCCATGTTTCCTTGTATATTTCCC
TACCCGCTGCCCCACATACCCAGCATGCCGCTGTGGCCACCATGTCCTCAAAGCCTTCTGTCTGTATCAG
GAATGTAGTCTGAGACTGCCAGGAAGCAACAAGGAGAGAGAAACACTAACTAGTCTTCCTTTATAACCCA
TTCATACTCTCTGGCTGTCCCCAACCTTCATAGTCTCCTGCATCCAAATGTCCTCTTTGGCTCAAAAAGT
AGGCCAGGCATGGTGGTTCATGCCTGTAATAGCACTTTGGGAGACTGAGGTGGGAGGATCACTTGGGGCC
AGGAGTTTGAGACCAGCTTGGGCAACACAGCGCAATCTCGTCTCTACTAAAAAAAAAAAAAAAAAAAAAT
TAGCTGGGCATGATGGCATGCTCCTGTGGTCCCAGCTACTTGGGAGGCTGAGGCAGGAGGATCACTTGGT
CCCAGGAGTTTGAGGCGACAGTGAGCTAGGATCGCACCACTGCACTCCAGCCTGAGTGACAGAGCAAGAC
CCTGTCTCTAAAAAAAATTAAAATGAAAGACCAGGTGCTGGGATTAAGGAAACACAGGTCTGAGGGTCTG
AGGGAAGGGGCCTGCCTCCCAGGGAGTCAACATAGATGTTCCCCATGAACAGGGATTTGACTTTGGAGGC
CAACCTGGCCTGGCCTCTGCCCTTTATCTCACACTCCCTATCCTTGGCCCACTGCCAGTCCCTGCCTTGT
GGCAAAGGGGCCCCAAAAGAAAAGCTGCCCTTCCCCAAATGTAAGGACCCAGGTACACTTTCACCCGTGG
AAAGCAGTGTCTGTCGAGAGTCTGTTTCCTATTAATACTTATCAAAGCCATGTGCGAGGGAGGTGGTCAG
CTGTCAATATGCCTTAGTATGTTTATATGAGTTTGTTTTGTTCTAAAATACCCAAACAGTTCTGGTCAAG
CGGGGCTATGCCCGTCTGGCCCAAAACACAGTCCGTTATTAACGAGATGGCCCTGGCAGGCGGGAACAAA
TCTGCCTCCATGCACTGCTTCCTGTAGTCTTTTAGAAAGTAACTCCAGGACATCGAAGTGCCCAGATTTG
ACTCCTAAGTTCTAGGAGACTGTAGCGCAGGGTCTGTCAACCTTAGCACTATTGGCATTTGGGGCTGGGT
AATTCTTTCTTGTGGGGGCCGTCTTGGGTACTGTAGGAAGCTGAGCAGCATTCCTGGCCTCCATCCACAA
GATACCTGTAGCAGTGTCCTGCCAACGGTAACAATCAAGTATGTCATCAGACATTGCCCAATGTCCCCAG
GGGGCAACACCCCTCTCTTGGACTTCAGGGTCAAGAGAATCTCTGCTGGCTACCCCAGGACTTCTCATTA
TAGATTTCCTGGAGCACGCAGCAGAAACTTTGCCTAGCCCAGTGGTTGTTTCCATTATCTGCTGCCAAAG
TGGGATTTGAGGGTGTCCGGGGGAGGGGGCATGGGGAGGGCAGTATGCTTTCAAAAACCCCTCCCAGGCC
AGGCGTGGTGGCTCATGCCTGTAATCACAGGACTTTGGGAGGCCGAGGCTGGCAGATCACTTGAGGCTGG
GAGTTAGAGACCAACCTGGCTAACATGGCAAAACCTCGTCTCTACTAAAAATACAAAAATCAGCCCGGCG
TGGTGGCGGGCATCTGTAATCCCATCTACTCGGGAGGCTGAGGCAGGAGAATTACTTGAACCCAGGAGGC
AGAGGCTGCAGTGAGCCGAGATGGCACCACTGCACTCCAGCTTGTTGACAGAATGAGACCCTGTGGAAAA
AAAAAAAAAAGCCCTCCCATGCCAGAACAGAGGATGGCAGTCTGTTTCAATAAGACACTGTGTCCTTGGT
GTTGGTTCTGATTAAGACTCACTGAGATCCAGTGCTCTTGAGCTGGGTCTCAGTCCCCTCCCATGTCCTG
TGCTCTGCCGCCACTGTTTTCATTGTTGTGTTCTCGTTGTGATTGTTAAGACTCACACTCCTGGCTCAGC
AGTGGTTTTCCAGAAGGCCCAAAGAGCGGTGCCGGGCACCCCACGTCGCAGTGTCCGTTCCGGGCTTGGG
AAGCTGGGGAGGTGGGCAGACCTGGTCGCATCTCACCACACACACACACACACACACACACACACACGCT
GTCAGAAACTCGGCCGTCCCCCCTACCTCTGAGCTCTCAATGCTGCTAATCTCTGCCAAGTGTCCCTGTG
CTCCAGCACCTTCCTTGAAGGACTGACGCCCACCCCACGCTCTTTGCGAGGTTGTCCAGGCTGTGTTTGT
CGCATGCTCTTCTTCTGTATAGTTCTCATCTTCCAATTTTATGGGATTCAACAAAAGCCTATTATGCTTG
TTTGCATTATGGTTACAATATTAAAAAGTGGATTCAA
(Isoform 1) NP_00061 1.1
(SEQ ID NO: 46)
MEDHMFGVQQIQPNVISVRLFKRKVGGLGFLVKERVSKPPVIISDLIRGGAAEQSGLIQAGDIILAVNGR
PLVDLSYDSALEVLRGIASETHVVLILRGPEGFTTHLETTFTGDGTPKTIRVTQPLGPPTKAVDLSHQPP
AGKEQPLAVDGASGPGNGPQHAYDDGQEAGSLPHANGLAPRPPGQDPAKKATRVSLQGRGENNELLKEIE
PVLSLLTSGSRGVKGGAPAKAEMKDMGIQVDRDLDGKSHKPLPLGVENDRVFNDLWGKGNVPVVLNNPYS
EKEQPPTSGKQSPTKNGSPSKCPRFLKVKNWETEVVLTDTLHLKSTLETGCTEYICMGSIMHPSQHARRP
EDVRTKGQLFPLAKEFIDQYYSSIKRFGSKAHMERLEEVNKEIDTTSTYQLKDTELIYGAKHAWRNASRC
VGRIQWSKLQVFDARDCTTAHGMFNYICNHVKYATNKGNLRSAITIFPQRTDGKHDFRVWNSQLIRYAGY
KQPDGSTLGDPANVQFTEICIQQGWKPPRGRFDVLPLLLQANGNDPELFQIPPELVLEVPIRHPKFEWFK
DLGLKWYGLPAVSNMLLEIGGLEFSACPFSGWYMGTEIGVRDYCDNSRYNILEEVAKKMNLDMRKTSSLW
KDQALVEINIAVLYSFQSDKVTIVDHHSATESFIKHMENEYRCRGGCPADWVWIVPPMSGSITPVFHQEM
LNYRLTPSFEYQPDPWNTHVWKGTNGTPTKRRAIGFKKLAEAVKFSAKLMGQAMAKRVKATILYATETGK
SQAYAKTLCEIFKHAFDAKVMSMEEYDIVHLEHETLVLVVTSTFGNGDPPENGEKFGCALMEMRHPNSVQ
EERKSYKVRFNSVSSYSDSQKSSGDGPDLRDNFESAGPLANVRFSVFGLGSRAYPHFCAFGHAVDTLLEE
LGGERILKMREGDELCGQEEAFRTWAKKVFKAACDVFCVGDDVNIEKANNSLISNDRSWKRNKFRLTFVA
EAPELTQGLSNVHKKRVSAARLLSRQNLQSPKSSRSTIFVRLHTNGSQELQYQPGDHLGVFPGNHEDLVN
ALIERLEDAPPVNQMVKVELLEERNTALGVISNWTDELRLPPCTIFQAFKYYLDITTPPTPLQLQQFASL
ATSEKEKQRLLVLSKGLQEYEEWKWGKNPTIVEVLEEFPSIQMPATLLLTQLSLLQPRYYSISSSPDMYP
DEVHLTVAIVSYRTRDGEGPIHHGVCSSWLNRIQADELVPCFVRGAPSFHLPRNPQVPCILVGPGTGIAP
FRSFWQQRQFDIQHKGMNPCPMVLVFGCRQSKIDHIYREETLQAKNKGVFRELYTAYSREPDKPKKYVQD
ILQEQLAESVYRALKEQGGHIYVCGDVTMAADVLKAIQRIMTQQGKLSAEDAGVFISRMRDDNRYHEDIF
GVTLRTYEVTNRLRSESIAFIEESKKDTDEVFSS

Isoform/Variant includes: (Isoform 3) NM 001204213.1 and NP_001191142.1; (Isoform 3) NM_001204214.1 and NP_001191143.1; (Isoform 2) NM_001204218.1 and NP_001191147.1
Day >35 (enteric neurons)—NOS1AP

(Isoform 2) NM_001126060.1
(SEQ ID NO: 47)
GAGATTCTGCTTTGGAGGGTAAAGTGGGGAAGAAATCGGATTCCCAGAGGTGAATCAGCTCCTCTCCTAC
TTGTGACTAGAGGGTGGTGGAGGTAAGGCCTTCCAGAGCCCATGGCTTCAGGAGAGGGTCTCTCTCCAGG
ACTGCCAGGCTGCTGGAGGACCTGCCCCTACCTGCTGCATCGTCAGGCTCCCACGCTTTGTCCGTGATGC
CCCCCTACCCCCTCACTCTCCCCGTCTCCATGGTCCCGACCAGGAAGGGAAGCCATCGGTACCTTCTCAG
GTACTTTGTTTCTGGATATCACGATGCTGCGAGTTGCCTAACCCTCCCCCTACCTTTATGAGAGGAATTC
CTTCTCCAGGCCCTTGCTGAGATTGTAGAGATTGAGTGCTCTGGACCGCAAAAGCCAGGCTAGTCCTTGT
AGGGTGAGCATGGAATTGGAATGTGTCACAGTGGATAAGCTTTTAGAGGAACTGAATCCAAACATTTTCT
CCAGCCGGACATTGAATGTTGCTACAAAGGGAGCCTTGAAGCTTTAACATGGTTCAGGCCCTTGGTGTGA
GAGCCCAGGGGGAGGACAGCTTGTCTGCTGCTCCAAATCACTTAGATCTGATTCCTGTTTTGAAAGTCCT
GCCCTGCCTTCCTCCTGCCTGTAGCCCAGCCCATCTAAATGGAAGCTGGGAATTGCCCCTCACCTCCCCT
GTGTCCTGTCCAGCTGAAGCTTTTGCAGCACTTTACCTCTCTGAAAGCCCCAGAGGACCAGAGCCCCCAG
CCTTACCTCTCAACCTGTCCCCTCCACTGGGCAGTGGTGGTCAGTTTTTACTGCAAAAAAAAAAAAAGAA
AAAAGAGAAAGAAAAAAAAGAATGAATGCAAGCTGATAGCTGAGACTGTGAGACTGTTTTTGTCCACTCT
TCTGAATCACTGCCACTTGGGTCAGGGACCACAGCCATTGCCACCCTTGGCCCATCTCTCTGCGTGCGTG
CCTTGAGCACACATATAAAAAGTGCCATGTGCAATTGTCTTATCTTTTATGATCTAGGCTTTGCCTAGGG
ATCACTACTCCTTAACGGGCTGGCTGGGGCAATGAGGAAAAGCTCCTTTGCTCCTGTAAGGCCATAAGTG
GCTGTTAACAGATTTTCAAATGCCTGAAGAGATTGCTGAGACCTGCTAGAGTCATATGTTCGGGGAATTA
AGTCTTTATCCTAGACAACAAGGTACAGATGCAAACTGCAGTGTTATTGGAGGGTCAATCGGCAAGGATA
TGATTATCCCAAAATGGAGTTCATCGACCCTAGCTTTCCTTTAGATTATATATAAATAAAAGTGCAGTCC
TCTTCTAATGGCCACAGTTGGTTTTCTTGTAGCCCAGAAAGTCCAAATTAAAGGAAATAAATTCAGTTTT
ATGTTAGCCTTCCTTGGTGCATCAGGGTGTCAGTGGAAATAGGATCAGGTGGTGTGTGTGTGTGTGTTTT
GTGTGTGTGTGTACACATGTGTTTATATATACATGTGTGAGGGAAAGTGTGTACATATATGTAGGATTGT
AACCAGACGGAAAAGAACGAGGATCTCCAGGGTGTTTGAATCAGCAACAGATTTGTGTTTTCTAACATGC
ATTTAGTTGGAGAGGCATGGTTCTGTTTGTTTTGTTTTGATCTAATTTGCCATTGGAAATAGGTACAGTT
ACACAGAGAAGGAAGAACCAGGAAAGTGAGATCCATGAAACTAAATGAGCAGCTGTCAGAATCCAGTGTG
GCTGAGCCTACCTAGCTTATGAAATCTAACCCAGGGTTCCCTGAGTCCAAGACCACTTAGATTATTAAGA
TTTTGAACGTCCAGAGGAGTGAAAAGTCTGTTTTCTGACGTAAGCCGGAGCTGAGGATAAAGCCAGAGGC
CAGTGGATTAGGTGTATGGAATGTGGATGGAGAGGGCTTGTGTGGGATGTGGCCAGGGAGTGGGTGAGGA
AGGCCGCTTCTAAATGGCCTGTAAAAACTTGAGATTGGATAGACGAAAGGAAATGGAGAAATTAAAGAAT
TGGAGAAACTAGTTATCTGTGTTGCTGACTTTGGGACCCATCCAAGACTCCTGCCCTTGGGGTGTTCCAT
GGTGGTTTCTTCCTGCCTGGGCGCCACCCTTTCCCCAGTTCAGGCCCTCCCTGGAGGACTAGTTTGTGTA
TTGGTATCCTCCCCAGTGGACCCAAACCAGCGCATACTTGGTGTGTGGAGATGGGAGACAAAGGACAGAT
CTAGGAGCCTTGAAGGATCACCAGCCACCGACCCTCCATCAGGGCCAACTGGGCAGGAAAGGGAACATTG
CAGACCTGATTTCCCGACGATGTCACCCTGTCCTCCCTCCTTGCTTCTTGCTCTGCTAACTCAACTCTGC
CTTCCTCTTTTTCATTCTTCTACTCTGCC
(Isoform 2) NP_001119532.2
(SEQ ID NO: 48)
MSLSSLCPVFSAAASSLQVHLLKDQLAAEAAARLEAQARVHQLLLQNKDMLQHISLLVKQVQELELKLSG
QNAMGSQDSLLEITFRSGALPVLCDPTTPKPEDLHSPPLGAGLADFAHPAGSPLGRRDCLVKLECFRFLP
PEDTPPPAQGEALLGGLELIKFRESGIASEYESNTDESEERDSWSQEELPRLLNVLQRQELGDGLDDEIA
V

Isoform/Variant includes: (Isoform 3) NM 001164757.2 and NP_001158229.1; (Isoform 1) NM_014697.3 and NP_055512.1
Day >35 (enteric neurons)—GAD1

(Isoform GAD67) NM_000817.3
(SEQ ID NO: 49)
CTCTTCTCCTGGCGCTCGCGTGCGAGAGGGAACTAGCGAGAACGAGGAAGCAGCTGGAGGTGACGCCGGG
CAGATTACGCCTGTCAGGGCCGAGCCGAGCGGATCGCTGGGCGCTGTGCAGAGGAAAGGCGGGAGTGCCC
GGCTCGCTGTCGCAGAGCCGAGCCTGTTTCTGCGCCGGACCAGTCGAGGACTCTGGACAGTAGAGGCCCC
GGGACGACCGAGCTGATGGCGTCTTCGACCCCATCTTCGTCCGCAACCTCCTCGAACGCGGGAGCGGACC
CCAATACCACTAACCTGCGCCCCACAACGTACGATACCTGGTGCGGCGTGGCCCATGGATGCACCAGAAA
ACTGGGGCTCAAGATCTGCGGCTTCTTGCAAAGGACCAACAGCCTGGAAGAGAAGAGTCGCCTTGTGAGT
GCCTTCAAGGAGAGGCAATCCTCCAAGAACCTGCTTTCCTGTGAAAACAGCGACCGGGATGCCCGCTTCC
GGCGCACAGAGACTGACTTCTCTAATCTGTTTGCTAGAGATCTGCTTCCGGCTAAGAACGGTGAGGAGCA
AACCGTGCAATTCCTCCTGGAAGTGGTGGACATACTCCTCAACTATGTCCGCAAGACATTTGATCGCTCC
ACCAAGGTGCTGGACTTTCATCACCCACACCAGTTGCTGGAAGGCATGGAGGGCTTCAACTTGGAGCTCT
CTGACCACCCCGAGTCCCTGGAGCAGATCCTGGTTGACTGCAGAGACACCTTGAAGTATGGGGTTCGCAC
AGGTCATCCTCGATTTTTCAACCAGCTCTCCACTGGATTGGATATTATTGGCCTAGCTGGAGAATGGCTG
ACATCAACGGCCAATACCAACATGTTTACATATGAAATTGCACCAGTGTTTGTCCTCATGGAACAAATAA
CACTTAAGAAGATGAGAGAGATAGTTGGATGGTCAAGTAAAGATGGTGATGGGATATTTTCTCCTGGGGG
CGCCATATCCAACATGTACAGCATCATGGCTGCTCGCTACAAGTACTTCCCGGAAGTTAAGACAAAGGGC
ATGGCGGCTGTGCCTAAACTGGTCCTCTTCACCTCAGAACAGAGTCACTATTCCATAAAGAAAGCTGGGG
CTGCACTTGGCTTTGGAACTGACAATGTGATTTTGATAAAGTGCAATGAAAGGGGGAAAATAATTCCAGC
TGATTTTGAGGCAAAAATTCTTGAAGCCAAACAGAAGGGATATGTTCCCTTTTATGTCAATGCAACTGCT
GGCACGACTGTTTATGGAGCTTTTGATCCGATACAAGAGATTGCAGATATATGTGAGAAATATAACCTTT
GGTTGCATGTCGATGCTGCCTGGGGAGGTGGGCTGCTCATGTCCAGGAAGCACCGCCATAAACTCAACGG
CATAGAAAGGGCCAACTCAGTCACCTGGAACCCTCACAAGATGATGGGCGTGCTGTTGCAGTGCTCTGCC
ATTCTCGTCAAGGAAAAGGGTATACTCCAAGGATGCAACCAGATGTGTGCAGGATACCTCTTCCAGCCAG
ACAAGCAGTATGATGTCTCCTACGACACCGGGGACAAGGCAATTCAGTGTGGCCGCCACGTGGATATCTT
CAAGTTCTGGCTGATGTGGAAAGCAAAGGGCACAGTGGGATTTGAAAACCAGATCAACAAATGCCTGGAA
CTGGCTGAATACCTCTATGCCAAGATTAAAAACAGAGAAGAATTTGAGATGGTTTTCAATGGCGAGCCTG
AGCACACAAACGTCTGTTTTTGGTATATTCCACAAAGCCTCAGGGGTGTGCCAGACAGCCCTCAACGACG
GGAAAAGCTACACAAGGTGGCTCCAAAAATCAAAGCCCTGATGATGGAGTCAGGTACGACCATGGTTGGC
TACCAGCCCCAAGGGGACAAGGCCAACTTCTTCCGGATGGTCATCTCCAACCCAGCCGCTACCCAGTCTG
ACATTGACTTCCTCATTGAGGAGATAGAAAGACTGGGCCAGGATCTGTAATCATCCTTCGCAGAACATGA
GTTTATGGGAATGCCTTTTCCCTCTGGCACTCCAGAACAAACCTCTATATGTTGCTGAAACACACAGGCC
ATTTCATTGAGGGAAAACATAATATCTTGAAGAATATTGTTAAAACCTTACTTAAAGCTTGTTTGTTCTA
GTTAGCAGGAAATAGTGTTCTTTTTAAAAAGTTGCACATTAGGAACAGAGTATATATGTACAGTTATACA
TACCTCTCTCTATATATACATGTATAGTGAGTGTGGCTTAGTAATAGATCACGGCATGTTTCCCGCTCCA
AGAGAATTCACTTTACCTTCAGCAGTTACCGAGGAGCTAAACATGCTGCCAACCAGCTTGTCCAACAACT
CCAGGAAAACTGTTTTTCAAAACGCCATGTCCTAGGGGCCGAGGGAAATGCTGTTGGTGAGAATCGACCT
CACTGTCAGCGTTTCTCCACCTGAAGTGATGATGGATGAGAAAAAACACCACCAAATGACAAGTCACACC
CTCCCCATTAGTATCCTGTTAGGGGAAAATAGTAGCAGAGTCATTGTTACAGGTGTACTATGGCTGTATT
TTTAGAGATTAATTTGTGTAGATTGTGTAAATTCCTGTTGTCTGACCTTGGTGGTGGGAGGGGGAGACTA
TGTGTCATGATTTCAATGATTGTTTAATTGTAGGTCAATGAAATATTTGCTTATTTATATTCAGAGATGT
ACCATGTTAAAGAGGCGTCTTGTATTTTCTTCCCATTTGTAATGTATCTTATTTATATATGAAGTAAGTT
CTGAAAACTGTTTATGGTATTTTCGTGCATTTGTGAGCCAAAGAGAAAAGATTAAAATTAGTGAGATTTG
TATTTATATTAGAGTGCCCTTAAAATAATGATTTAAGCATTTTACTGTCTGTAAGAGAATTCTAAGATTG
TACATAAAGTCATATATATGGAAATCCTGTTACTTAAATAGCATCTGCTCTTCTCTTACGCTCTCTGTCT
GGCTGTACGTCTGGTGTTCTCAATGCTTTTCTAGCAACTGTTGGATAATAACTAGATCTCCTGTAATTTT
GTAGTAGTTGATGACCAATCTCTGTGACTCGCTTAGCTGAAACCTAAGGCAACATTTCCGAAGACCTTCT
GAAGATCTCAGATAAAGTGACCAGGCTCACAACTGTTTTTGAAGAAGGGAAATTCACACTGTGCGTTTTA
GAGTAT GCAAGAAGAATATAAATAAATAAAAATATTCTCCATGGAGAATTTGAACAAAA
(Isoform GAD67) NP_000808.2
(SEQ ID NO: 50)
MASSTPSSSATSSNAGADPNTTNLRPTTYDTWCGVAHGCTRKLGLKICGFLQRTNSLEEKSRLVSAFKER
QSSKNLLSCENSDRDARFRRTETDFSNLFARDLLPAKNGEEQTVQFLLEVVDILLNYVRKTFDRSTKVLD
FHHPHQLLEGMEGFNLELSDHPESLEQILVDCRDTLKYGVRTGHPRFFNQLSTGLDIIGLAGEWLTSTAN
TNMFTYEIAPVFVLMEQITLKKMREIVGWSSKDGDGIFSPGGAISNMYSIMAARYKYFPEVKTKGMAAVP
KLVLFTSEQSHYSIKKAGAALGFGTDNVILIKCNERGKIIPADFEAKILEAKQKGYVPFYVNATAGTTVY
GAFDPIQEIADICEKYNLWLHVDAAWGGGLLMSRKHRHKLNGIERANSVTWNPHKMMGVLLQCSAILVKE
KGILQGCNQMCAGYLFQPDKQYDVSYDTGDKAIQCGRHVDIFKFWLMWKAKGTVGFENQINKCLELAEYL
YAKIKNREEFEMVFNGEPEHTNVCFWYIPQSLRGVPDSPQRREKLHKVAPKIKALMMESGTTMVGYQPQG
DKANFFRMVISNPAATQSDIDFLIEEIERLGQDL

Isoform/Variant includes: (Isoform GAD25) NM_013445.3 and NP_038473.2
Day >35 (enteric neurons)—GAD2

(Variant 1) NM_000818.2
(SEQ ID NO: 51)
ACGCACGCGCGCGCAGGGCCAAGCCCGAGGCAGCTCGCCCGCAGCTCGCACTCGCAGGCGACCTGCTCCA
GTCTCCAAAGCCGATGGCATCTCCGGGCTCTGGCTTTTGGTCTTTCGGGTCGGAAGATGGCTCTGGGGAT
TCCGAGAATCCCGGCACAGCGCGAGCCTGGTGCCAAGTGGCTCAGAAGTTCACGGGCGGCATCGGAAACA
AACTGTGCGCCCTGCTCTACGGAGACGCCGAGAAGCCGGCGGAGAGCGGCGGGAGCCAACCCCCGCGGGC
CGCCGCCCGGAAGGCCGCCTGCGCCTGCGACCAGAAGCCCTGCAGCTGCTCCAAAGTGGATGTCAACTAC
GCGTTTCTCCATGCAACAGACCTGCTGCCGGCGTGTGATGGAGAAAGGCCCACTTTGGCGTTTCTGCAAG
ATGTTATGAACATTTTACTTCAGTATGTGGTGAAAAGTTTCGATAGATCAACCAAAGTGATTGATTTCCA
TTATCCTAATGAGCTTCTCCAAGAATATAATTGGGAATTGGCAGACCAACCACAAAATTTGGAGGAAATT
TTGATGCATTGCCAAACAACTCTAAAATATGCAATTAAAACAGGGCATCCTAGATACTTCAATCAACTTT
CTACTGGTTTGGATATGGTTGGATTAGCAGCAGACTGGCTGACATCAACAGCAAATACTAACATGTTCAC
CTATGAAATTGCTCCAGTATTTGTGCTTTTGGAATATGTCACACTAAAGAAAATGAGAGAAATCATTGGC
TGGCCAGGGGGCTCTGGCGATGGGATATTTTCTCCCGGTGGCGCCATATCTAACATGTATGCCATGATGA
TCGCACGCTTTAAGATGTTCCCAGAAGTCAAGGAGAAAGGAATGGCTGCTCTTCCCAGGCTCATTGCCTT
CACGTCTGAACATAGTCATTTTTCTCTCAAGAAGGGAGCTGCAGCCTTAGGGATTGGAACAGACAGCGTG
ATTCTGATTAAATGTGATGAGAGAGGGAAAATGATTCCATCTGATCTTGAAAGAAGGATTCTTGAAGCCA
AACAGAAAGGGTTTGTTCCTTTCCTCGTGAGTGCCACAGCTGGAACCACCGTGTACGGAGCATTTGACCC
CCTCTTAGCTGTCGCTGACATTTGCAAAAAGTATAAGATCTGGATGCATGTGGATGCAGCTTGGGGTGGG
GGATTACTGATGTCCCGAAAACACAAGTGGAAACTGAGTGGCGTGGAGAGGGCCAACTCTGTGACGTGGA
ATCCACACAAGATGATGGGAGTCCCTTTGCAGTGCTCTGCTCTCCTGGTTAGAGAAGAGGGATTGATGCA
GAATTGCAACCAAATGCATGCCTCCTACCTCTTTCAGCAAGATAAACATTATGACCTGTCCTATGACACT
GGAGACAAGGCCTTACAGTGCGGACGCCACGTTGATGTTTTTAAACTATGGCTGATGTGGAGGGCAAAGG
GGACTACCGGGTTTGAAGCGCATGTTGATAAATGTTTGGAGTTGGCAGAGTATTTATACAACATCATAAA
AAACCGAGAAGGATATGAGATGGTGTTTGATGGGAAGCCTCAGCACACAAATGTCTGCTTCTGGTACATT
CCTCCAAGCTTGCGTACTCTGGAAGACAATGAAGAGAGAATGAGTCGCCTCTCGAAGGTGGCTCCAGTGA
TTAAAGCCAGAATGATGGAGTATGGAACCACAATGGTCAGCTACCAACCCTTGGGAGACAAGGTCAATTT
CTTCCGCATGGTCATCTCAAACCCAGCGGCAACTCACCAAGACATTGACTTCCTGATTGAAGAAATAGAA
CGCCTTGGACAAGATTTATAATAACCTTGCTCACCAAGCTGTTCCACTTCTCTAGAGAACATGCCCTCAG
CTAAGCCCCCTACTGAGAAACTTCCTTTGAGAATTGTGCGACTTCACAAAATGCAAGGTGAACACCACTT
TGTCTCTGAGAACAGACGTTACCAATTATGGAGTGTCACCAGCTGCCAAAATCGTAGGTGTTGGCTCTGC
TGGTCACTGGAGTAGTTGCTACTCTTCAGAATATGGACAAAGAAGGCACAGGTGTAAATATAGTAGCAGG
ATGAGGAACCTCAAACTGGGTATCATTTTGCACGTGCTCTTCTGTTCTCAAATGCTAAATGCAAACACTG
TGTATTTATTAGTTAGGTGTGCCAAACTACCGTTCCCAAATTGGTGTTTCTGAATGACATCAACATTCCC
CCAACATTACTCCATTACTAAAGACAGAAAAAAATAAAAACATAAAATATACAAACATGTGGCAACCTGT
TCTTCCTACCAAATATAAACTTGTGTATGATCCAAGTATTTTATCTGTGTTGTCTCTCTAAACCCAAATA
AATGTGTAAATGTGGACACATCTC
(Variant 1) NP_000809.1
(SEQ ID NO: 52)
MASPGSGFWSFGSEDGSGDSENPGTARAWCQVAQKFTGGIGNKLCALLYGDAEKPAESGGSQPPRAAARK
AACACDQKPCSCSKVDVNYAFLHATDLLPACDGERPTLAFLQDVMNILLQYVVKSFDRSTKVIDFHYPNE
LLQEYNWELADQPQNLEEILMHCQTTLKYAIKTGHPRYFNQLSTGLDMVGLAADWLTSTANTNMFTYEIA
PVFVLLEYVTLKKMREIIGWPGGSGDGIFSPGGAISNMYAMMIARFKMFPEVKEKGMAALPRLIAFTSEH
SHFSLKKGAAALGIGTDSVILIKCDERGKMIPSDLERRILEAKQKGFVPFLVSATAGTTVYGAFDPLLAV
ADICKKYKIWMHVDAAWGGGLLMSRKHKWKLSGVERANSVTWNPHKMMGVPLQCSALLVREEGLMQNCNQ
MHASYLFQQDKHYDLSYDTGDKALQCGRHVDVFKLWLMWRAKGTTGFEAHVDKCLELAEYLYNIIKNREG
YEMVFDGKPQHTNVCFWYIPPSLRTLEDNEERMSRLSKVAPVIKARMMEYGTTMVSYQPLGDKVNFFRMV
ISNPAATHQDIDFLIEEIERLGQDL

Isoform/Variant includes: (Variant 2) NM_001134366.2 and NP_001127838.1
Day >35 (enteric neurons)—SERT (SLC6A4)

NM_001045.6
(SEQ ID NO: 53)
CTAACTCCATTATCTTCACTCTTTCCTTCTTCCTGCCACCTCATGCCCATTCTCTTTACTGTCTAGCATG
CTGAAAGAAGGAAGTGATCTAAATGCCAGCGTGTTCAGTGGTAAATATTAGTTGGTGCAAAAGAAAAACC
ATGATTACTTTTGCACTAACCTAATAGCTTTGCAAATTTTAAGAACTTGCTTTATGAAGATATTCGGATA
TGGATTCTCCCCACCCCACATACTTAGACATTGTTCAAATATACTACTTTTAAAAAAACACCTTTTCAAA
CAGAATTAGCGTTTTGCCAAGTCTGGTATTAATGGAATTGTACAGGAGCTTTGAAAGTTTTCAAACTTTA
TTAAACTAAAAAAAAAAAATCGAAAATCTCTGTCTGTTCCGCATAGTATGCATTTATTTGACCCCTATTT
ATCAATACTATGATGGGGTTTTTTTTTTTTAAAGAAAATTTAAGAGTAGGTAGGTGGATTTTAAAATAAT
ATTTTAAAGACCTTTTATATCTATATGTAGCATTTATAGAAAAATAAAAACTAAAAATAGAATTGAATTG
TAACATTATTTAAGGACTGAAGTTTTTTTTCTTGTATCAGTAAGAAATACCCAAGAGGCTGGGTGCAGTG
ACTCACACCTGTAATCCCAGCACTTTGGGAGGCTGCAGTGGGAGGATCACATGACATCAGGAGTTTGAGA
CCAGCTTGGCCAACATAGTGAAACGCCGTCTCTATTAAAAATACAGAAAATTAGCTGAATGTGGTGGCAG
GCGCCTATAATTCCTGCTACTTGGGAGGCTGAGGCAGGAGAATTGCTTGAACCCAGGAGGCAGAGGTTGC
AGTGAGCCAAACGTTCCACTGCATTCCAGCCTGGATGACAAGAGCGAAACTCCGTCTCAAAAAAAAAAAA
AAAAATTGTTATGCTTAGTTCCATGGAAAGACTATTCTGAAGCTTTAAGTCTTCTTTTTCTATTTTCCAT
AGTATTGCCCCTTCCCCACTTCATTGCTTAACTGTCTCTAAATTTTAATGATAATAATATTTTAAAGGTC
AGATAATGCCATTAGAGGCAGAGACAAACCTGGGGACCAAGCTAATTTCTCTGTTATTGAGAGCGCTAAA
GACAGGTGAACTGGAGTTTTATTTCCTCTGCTAGAGTGAAATAATACCCTCTCCACCAGGCACAATGGCT
CACACCTGTAATTCCAGCACTTTGGGAGGCCAAGGTGGGCGGATCACTTGAGGTCAGGAGTTCGAGACCA
GCCTGGCCAACATGGTGAAACCCCATCTCTACTAAAAATACAAAAATTAGCCAGCCATGGTGGCATGCTC
CTGTAATCCCAGCTACTTGGGAGACTGAGGTGGGATAATCACTTGAACCTGGGAGGCGGAGGTTGCAGTG
AGCTGAGATTGTGCCACTGCACTCCAGCCTGGGTGACAGAGCAAGACTCCATCTAAAAAACAAAAACAAA
AACAAAAAAACCCTCTCAATTGGTTTAATACCACGATAGAAAAGATAAATATTTTAGGATGGAATCTTAA
ATATGTCTGTCCTTTTGTTTCATATAGTTGAAATCAATTCAGTATTGTTTCTACATTAGGTGTTTCAAAA
ACAGTCACCTTTGCAACAGAAGAGCTCTTTTGTTGAAAATGATTCACAATATATTTCAGTTGGAATGTCA
GGTGGTATTTCTCTTACCAACACCTCACAGAATCTATGCCAAGCTGCTCTACCACCAATGCAAGTATTTT
TTTAAAGCTACTAAAATAAACTTTCTTCACCAGCCAACTCTAATTTGGAGACAGTGTGCATTGGTGAAGG
ACCTCGTCAGAATAAGTTTGAATGTCTACTGAACTAAGAAGAATTTTGTCGTTTGGGGGAGAGAATAGAT
GGCATCAGTCCTTCAATTCTGTAACTGAAGACTCCAATTATAGTAGATAAGAATTGTGTCTAGCAATTTT
TAAACACTGACAGTCCAAACAAAAATATTTGGTGAGGAAGGATGTCCCATATTTTTGCTTAAATATCATA
AACAAATATGAGCATTTACTAATTTTTTAAATGGCATTTTGAAAGATTATTCTTATTTACACTCTAAAAT
TAAAGGTGTACTTTATCTTAAGAAAATGATATATTAAAAATTCATATTTTAAAAGATAAAATTGGGGAAT
TTACAGTTATTTTGTGAATGGCCTTTAAACTATGATTTGATCTATATACAACTTTTCAGAATACTTTTGA
TTGTGTGTTGGACATATCTAAAATTAATTTTATCTGGCAGAATTAAACCTAAATTTATTTGTATAAAAGA
GTCCCCTTTCTAAAATTCATACAGTGCCCTTGTATTCATTTATGCAGTGTTTATAAATATTTCAAGTTAG
ATTGTGAGATATTAATAAATGATTTATGCTGTGAA
NP_001036.1
(SEQ ID NO: 54)
METTPLNSQKQLSACEDGEDCQENGVLQKVVPTPGDKVESGQISNGYSAVPSPGAGDDTRHSIPATTTTL
VAELHQGERETWGKKVDFLLSVIGYAVDLGNVWRFPYICYQNGGGAFLLPYTIMAIFGGIPLFYMELALG
QYHRNGCISIWRKICPIFKGIGYAICIIAFYIASYYNTIMAWALYYLISSFTDQLPWTSCKNSWNTGNCT
NYFSEDNITWTLHSTSPAEEFYTRHVLQIHRSKGLQDLGGISWQLALCIMLIFTVIYFSIWKGVKTSGKV
VWVTATFPYIILSVLLVRGATLPGAWRGVLFYLKPNWQKLLETGVWIDAAAQIFFSLGPGFGVLLAFASY
NKFNNNCYQDALVTSVVNCMTSFVSGFVIFTVLGYMAEMRNEDVSEVAKDAGPSLLFITYAEAIANMPAS
TFFAIIFFLMLITLGLDSTFAGLEGVITAVLDEFPHVWAKRRERFVLAVVITCFFGSLVTLTFGGAYVVK
LLEEYATGPAVLTVALIEAVAVSWFYGITQFCRDVKEMLGFSPGWFWRICWVAISPLFLLFIICSFLMSP
PQLRLFQYNYPYWSIILGYCIGTSSFICIPTYIAYRLIITPGTFKERIIKSITPETPTEIPCGDIRLNAV

Day >35 (enteric neurons)—TPH1

NM_004179.3
(SEQ ID NO: 55)
AAGGTATAAGGTGAGGCCATAAACAGAAAGGTATGCAATGAGAGTAGAGGCTGCAGAAAAGGGGAGGAAA
TAGAATGTCAGAAAGGTCAAGAAGAGTGGTTTAGGACTGAGACTGTAAGTAATGTAAGGATTGCCAAGGA
ATGGTTTTTATGCAGAGTCAGCTGTGTAGCACAGGGAGTAAGCAGTCCTGTTATAAATGGGTGAAGCTGA
ACTCCAAAATACAAACTTATGTCTCTTTAATTTGCCCCTTTCCTCCCTCTAGTCACTAAATGAAGATGTT
CTTCAAGTTTCTGTCTTTGCACTTCTACTCTTTTTGCCATCTCTTCATGGGGAATGTCACCCAGATACAT
GACTTCAGTTCTTATTTTGAATGACTCCTCTATTTGCAGTTATAACCCTGACATCTTTCTTGAATTTTTT
TGCCCCTTAATTCCAACGAACTCCTGGATGTCTATGTAGATGTCTCATAATTTAGGCCAATGCTGTACAA
CTTCATGTGGCATCATCCACATAGAATGAATAGGGGTTGTTCAATATGACAGCCCTGCCAAGCATCTCAA
ATTGAACAGGTCTATAAAGGAACTCAGAATCACTTGAATATCACAGTCCATCAGCTTCCTTTCCCAGTAA
TTTAATTTCCATTAATGGTATATTAAACATCGTATTTTCAATCATTTTTAGGTTTAGTCAGCTACTTAAA
AATAGGCTTTTTCTTCACATAAATAAATTGTATACCATTTAATGTGTTTTATTTCAACAGACTAATAGAT
CTTTTAACACTTCTTAGTCCTCAAAATAAATTATTAAAAACTTTGTATTTCATGTCATATTATATAAGGA
AATAGAATTTTGAAGAATAATTCATGCTGTTTTAAATTTTTACATTGTACATTTTAATAGCTAATCAACT
CTTAAGTATACATTTGATGGTAAATAGATGCTAGCTAATCTAGAATCATTTGAAATATTGATTATCTGAT
TTATAGTTTGGTTGCAAGGAAATCTACTGTAAGCAAATCATAATAGATAGCAAATCTTAGAACATGTAGA
AAAAAAGAAATTAAAAAAAGCAAATCTTAGAACATGTAGAAAAAGGAAATTAAAAAATAGATAGCAAATA
ACACAAATTATGCATACTATATGTGGTTATTTTCATACAACTTTGATAACGGTGAAGAATCATACTTATT
GAATTTTAAAATGATGATGTTTAAAATCACTCTATGACTAAAGCACATGAGAATAAATGTTAAAGGCACA
GCCTTATAATCCACAACTTCAGAACAATGGAATTTTTTTTTTTTTTTTTTTTTTTTTTTTTTTTTTTTTT
TTTTTGAGACGGAGTCTTGCTCTGTCGCCCAGGCTGGAGTGCAGTGGTGCGATCTTGGCTCACTGCAAGC
TCCACCTCCCAGGTTCACGGCATTCTCCTGCCTCTGTCTCCTGAGTAGCTGGGACTACAGGCGCCCGCCA
CCACGTCCGGCTCATTTTTTGTATTTTTAGTAGAGACAAGGTTTCACCGTGTTAGCCAGGATGGTTTCAA
TCTCCTGACATTGTGATCTGCCCACCTCGGCCTCCCAAAGTGCTGGGATTACAGGCGTGAGCCACTGCAC
CCGGCCAAACATTGGAAATTTTTAATGATGGATTTCAGTTCTTCTCACCTCTGGGTATTTGTAATATTTG
GTAAATCAGGAGTCCTGAGAGTCTTAGAATTTTCATGCATTTTAAATATATGTCTTCAAAGCACCTAGGA
ATATGTAAATAGTTACCCAAATGTGAGAACAGAATTGAACTAAAAGCTTCGTTTATAATTTTTTTTTTTT
TTTGAGACAGAGTCTCACTCTGTTGCCCAGGCTGGAGCGCAGTTGCGTGATTTCAGCTCGCTGCAACCTG
GGACTGCAGGCGTGCTTCACCATGCCCCGCTAATTTTTGTTTTTTTAGTAGAGATGGGGTTTCGCCATGT
TGGCCAGGCTGGTCTCGAACTTCTGACCGCAGGTGATCCATCCGCCTCGGCCTCCCAAAGTGCTGGGATT
ACAAGCATGAGCCACCATGCCCAGCCGGTTAATAATTCTTAATCAGTAAATCATTATTCAAAGACATAGT
TAATATCTTCATGACTTCTTAAGGATGCTCAAAAGCTGGTCTTCAGAATACCTACATTTCTTTATCTACA
TATTCTGTCATTTGGAGTTATACAAGTAGTAATGACACACCAAAATGTAAATAATTATTGTTAGCTTTTA
AGTTGGCTTCCAAATTCAAAAAAGAAAAAAATCCTGAATTCTCTAAAGGAACATATGATAGAGTCCATAT
GTTAATTCATAGGAAGTGTTTAAGGTACTATGGTCTATTTTGGTCTAATCTTTGTTTTACTATGTAATAT
ATATTTATGATTTACAAGTTTGTTATTCAGTGGGATAATAAATGAACACAAAATTTATGTTCTCAA
NP_004170.1
(SEQ ID NO: 56)
MIEDNKENKDHSLERGRASLIFSLKNEVGGLIKALKIFQEKHVNLLHIESRKSKRRNSEFEIFVDCDINR
EQLNDIFHLLKSHTNVLSVNLPDNFTLKEDGMETVPWFPKKISDLDHCANRVLMYGSELDADHPGFKDNV
YRKRRKYFADLAMNYKHGDPIPKVEFTEEEIKTWGTVFQELNKLYPTHACREYLKNLPLLSKYCGYREDN
IPQLEDVSNFLKERTGFSIRPVAGYLSPRDFLSGLAFRVFHCTQYVRHSSDPFYTPEPDTCHELLGHVPL
LAEPSFAQFSQEIGLASLGASEEAVQKLATCYFFTVEFGLCKQDGQLRVFGAGLLSSISELKHALSGHAK
VKPFDPKITCKQECLITTFQDVYFVSESFEDAKEKMREFTKTIKRPFGVKYNPYTRSIQILKDTKSITSA
MNELQHDLDVVSDALAKVSRKPSI

Day >35 (enteric neurons)—TPH2

NM_173353.4
(SEQ ID NO: 57)
GCATTGCTCTTCAGCACCAGGGTTCTGGACAGCGCCCCAAGCAGGCAGCT
GATCGCACGCCCCTTCCTCTCAATCTCCGCCAGCGCTGCTACTGCCCCTC
TAGTACCCCCTGCTGCAGAGAAAGAATATTACACCGGGATCCATGCAGCC
AGCAATGATGATGTTTTCCAGTAAATACTGGGCACGGAGAGGGTTTTCCC
TGGATTCAGCAGTGCCCGAAGAGCATCAGCTACTTGGCAGCTCAACACTA
AATAAACCTAACTCTGGCAAAAATGACGACAAAGGCAACAAGGGAAGCAG
CAAACGTGAAGCTGCTACCGAAAGTGGCAAGACAGCAGTTGTTTTCTCCT
TGAAGAATGAAGTTGGTGGATTGGTAAAAGCACTGAGGCTCTTTCAGGAA
AAACGTGTCAACATGGTTCATATTGAATCCAGGAAATCTCGGCGAAGAAG
TTCTGAGGTTGAAATCTTTGTGGACTGTGAGTGTGGGAAAACAGAATTCA
ATGAGCTCATTCAGTTGCTGAAATTTCAAACCACTATTGTGACGCTGAAT
CCTCCAGAGAACATTTGGACAGAGGAAGAAGAGCTAGAGGATGTGCCCTG
GTTCCCTCGGAAGATCTCTGAGTTAGACAAATGCTCTCACAGAGTTCTCA
TGTATGGTTCTGAGCTTGATGCTGACCACCCAGGATTTAAGGACAATGTC
TATCGACAGAGAAGAAAGTATTTTGTGGATGTGGCCATGGGTTATAAATA
TGGTCAGCCCATTCCCAGGGTGGAGTATACTGAAGAAGAAACTAAAACTT
GGGGTGTTGTATTCCGGGAGCTCTCCAAACTCTATCCCACTCATGCTTGC
CGAGAGTATTTGAAAAACTTCCCTCTGCTGACTAAATACTGTGGCTACAG
AGAGGACAATGTGCCTCAACTCGAAGATGTCTCCATGTTTCTGAAAGAAA
GGTCTGGCTTCACGGTGAGGCCGGTGGCTGGATACCTGAGCCCACGAGAC
TTTCTGGCAGGACTGGCCTACAGAGTGTTCCACTGTACCCAGTACATCCG
GCATGGCTCAGATCCCCTCTACACCCCAGAACCAGACACATGCCATGAAC
TCTTGGGACATGTTCCACTACTTGCGGATCCTAAGTTTGCTCAGTTTTCA
CAAGAAATAGGTCTGGCGTCTCTGGGAGCATCAGATGAAGATGTTCAGAA
ACTAGCCACGTGCTATTTCTTCACAATCGAGTTTGGCCTTTGCAAGCAAG
AAGGGCAACTGCGGGCATATGGAGCAGGACTCCTTTCCTCCATTGGAGAA
TTAAAGCACGCCCTTTCTGACAAGGCATGTGTGAAAGCCTTTGACCCAAA
GACAACTTGCTTACAGGAATGCCTTATCACCACCTTCCAGGAAGCCTACT
TTGTTTCAGAAAGTTTTGAAGAAGCCAAAGAAAAGATGAGGGACTTTGCA
AAGTCAATTACCCGTCCCTTCTCAGTATACTTCAATCCCTACACACAGAG
TATTGAAATTCTGAAAGACACCAGAAGTATTGAAAATGTGGTGCAGGACC
TTCGCAGCGACTTGAATACAGTGTGTGATGCTTTAAACAAAATGAACCAA
TATCTGGGGATTTGATGCCTGGAACTATGTTGTTGCCAGCATGATCTTTT
TGGGGCTTAGCAGCAGTTCAGTCAATGTCATATAACGCAAATAACCTTCT
GTGTCATGGCTTGGCTAATAAGCATGCAATTCCATATATCTATACCATCT
TGTAACTCACTGTGTTAGTATATAAAGCACCATAAGAAATCCAATGGCAG
ATAACCACTCATTGTATGAAATAACGTATTATGTTTAAACATCTTAAAAA
GATTTGACATTCCTGCTTAGTGTCCTTAACCAAACTGCATCTAGTTAAAA
TTTGTAACAAATAGCCCTCTTATGAGTCTCATTTATGCCCTTTTCTTTTT
CAGATCTAAGCCTTTCCTCTGTGTTCATTAGATAAAATGAAAAAAAGCAG
TGAAGCTGTTTCCATTTTCAATAGTATCAGTGTTTTCACGCATTATTTGA
GATAAACCCAGAATTGTAGGAAACTTCCCATCACAATAACAAAGGTTCAA
TATTCTATTTCAAAAATTGTTGAGGTAACACAGCAGTTGGAATGATTTTT
AGGTTGAGTATTTACACAATGCAAGAAAACACCTTTTTACAAATGGAATT
ATGTAGGTTGCGTTGACCTTGTAGAACCTGAGTTATGACAAGCTTCCTGA
AGTATTTTGGAAGATAGTACTTCCGGAAAGGACATTAGGAAAGACTAAAC
AGTGGACAATCAATCTTGGGACTATGAATTTTATGCTGGAATAAAGTAAA
TTATCATGTT
NP_775489.2
(SEQ ID NO: 58)
MQPAMMMFSSKYWARRGFSLDSAVPEEHQLLGSSTLNKPNSGKNDDKGNK
GSSKREAATESGKTAVVFSLKNEVGGLVKALRLFQEKRVNMVHIESRKSR
RRSSEVEIFVDCECGKTEFNELIQLLKFQTTIVTLNPPENIWTEEEELED
VPWFPRKISELDKCSHRVLMYGSELDADHPGFKDNVYRQRRKYFVDVAMG
YKYGQPIPRVEYTEEETKTWGVVFRELSKLYPTHACREYLKNFPLLTKYC
GYREDNVPQLEDVSMFLKERSGFTVRPVAGYLSPRDFLAGLAYRVFHCTQ
YIRHGSDPLYTPEPDTCHELLGHVPLLADPKFAQFSQEIGLASLGASDED
VQKLATCYFFTIEFGLCKQEGQLRAYGAGLLSSIGELKHALSDKACVKAF
DPKTTCLQECLITTFQEAYFVSESFEEAKEKMRDFAKSITRPFSVYFNPY
TQSIEILKDTRSIENVVQDLRSDLNTVCDALNKMNQYLGI

The presence of any given molecular identifier to determine the stage of differentiation can be detected using any method known in the art. In some embodiments, the presence of one or a plurality of molecular identifiers is determined by PCR amplification, such as qPCR or qRT-PCR. In some embodiments, the presence of one or a plurality of molecular identifiers is determined by using antibodies that are specific to the one or plurality of molecular identifiers.

The enteric nitrergic neurons produced by the methods of the disclosure can be isolated and/or enriched by using one or a plurality of surface markers. The enteric nitrergic neurons produced by the methods of the disclosure can be isolated and/or enriched by using one or a plurality of surface antigens specific for the NOS-expressing enteric neurons, or surface markers. The term “surface marker” is used herein interchangeably with the term “surface antigens.” Exemplary surface markers that may be used include, but not limited to, the following:

CD47 - (Isoform 1 precursor) NM_001777.3
(SEQ ID NO: 59)
TTCTTCCTTGATTGTGTAGCTGTCCAAAATAATAACATATATAGAGGGAG
CTGTATTCCTTTATACAAATCTGATGGCTCCTGCAGCACTTTTTCCTTCT
GAAAATATTTACATTTTGCTAACCTAGTTTGTTACTTTAAAAATCAGTTT
TGATGAAAGGAGGGAAAAGCAGATGGACTTGAAAAAGATCCAAGCTCCTA
TTAGAAAAGGTATGAAAATCTTTATAGTAAAATTTTTTATAAACTAAAGT
TGTACCTTTTAATATGTAGTAAACTCTCATTTATTTGGGGTTCGCTCTTG
GATCTCATCCATCCATTGTGTTCTCTTTAATGCTGCCTGCCTTTTGAGGC
ATTCACTGCCCTAGACAATGCCACCAGAGATAGTGGGGGAAATGCCAGAT
GAAACCAACTCTTGCTCTCACTAGTTGTCAGCTTCTCTGGATAAGTGACC
ACAGAAGCAGGAGTCCTCCTGCTTGGGCATCATTGGGCCAGTTCCTTCTC
TTTAAATCAGATTTGTAATGGCTCCCAAATTCCATCACATCACATTTAAA
TTGCAGACAGTGTTTTGCACATCATGTATCTGTTTTGTCCCATAATATGC
TTTTTACTCCCTGATCCCAGTTTCTGCTGTTGACTCTTCCATTCAGTTTT
ATTTATTGTGTGTTCTCACAGTGACACCATTTGTCCTTTTCTGCAACAAC
CTTTCCAGCTACTTTTGCCAAATTCTATTTGTCTTCTCCTTCAAAACATT
CTCCTTTGCAGTTCCTCTTCATCTGTGTAGCTGCTCTTTTGTCTCTTAAC
TTACCATTCCTATAGTACTTTATGCATCTCTGCTTAGTTCTATTAGTTTT
TTGGCCTTGCTCTTCTCCTTGATTTTAAAATTCCTTCTATAGCTAGAGCT
TTTCTTTCTTTCATTCTCTCTTCCTGCAGTGTTTTGCATACATCAGAAGC
TAGGTACATAAGTTAAATGATTGAGAGTTGGCTGTATTTAGATTTATCAC
TTTTTAATAGGGTGAGCTTGAGAGTTTTCTTTCTTTCTGTTTTTTTTTTT
TGTTTTTTTTTTTTTTTTTTTTTTTTTTTTTTTTGACTAATTTCACATGC
TCTAAAAACCTTCAAAGGTGATTATTTTTCTCCTGGAAACTCCAGGTCCA
TTCTGTTTAAATCCCTAAGAATGTCAGAATTAAAATAACAGGGCTATCCC
GTAATTGGAAATATTTCTTTTTTCAGGATGCTATAGTCAATTTAGTAAGT
GACCACCAAATTGTTATTTGCACTAACAAAGCTCAAAACACGATAAGTTT
ACTCCTCCATCTCAGTAATAAAAATTAAGCTGTAATCAACCTTCTAGGTT
TCTCTTGTCTTAAAATGGGTATTCAAAAATGGGGATCTGTGGTGTATGTA
TGGAAACACATACTCCTTAATTTACCTGTTGTTGGAAACTGGAGAAATGA
TTGTCGGGCAACCGTTTATTTTTTATTGTATTTTATTTGGTTGAGGGATT
TTTTTATAAACAGTTTTACTTGTGTCATATTTTAAAATTACTAACTGCCA
TCACCTGCTGGGGTCCTTTGTTAGGTCATTTTCAGTGACTAATAGGGATA
ATCCAGGTAACTTTGAAGAGATGAGCAGTGAGTGACCAGGCAGTTTTTCT
GCCTTTAGCTTTGACAGTTCTTAATTAAGATCATTGAAGACCAGCTTTCT
CATAAATTTCTCTTTTTGAAAAAAAGAAAGCATTTGTACTAAGCTCCTCT
GTAAGACAACATCTTAAATCTTAAAAGTGTTGTTATCATGACTGGTGAGA
GAAGAAAACATTTTGTTTTTATTAAATGGAGCATTATTTACAAAAAGCCA
TTGTTGAGAATTAGATCCCACATCGTATAAATATCTATTAACCATTCTAA
ATAAAGAGAACTCCAGTGTTGCTATGTGCAAGATCCTCTCTTGGAGCTTT
TTTGCATAGCAATTAAAGGTGTGCTATTTGTCAGTAGCCATTTTTTTGCA
GTGATTTGAAGACCAAAGTTGTTTTACAGCTGTGTTACCGTTAAAGGTTT
TTTTTTTTATATGTATTAAATCAATTTATCACTGTTTAAAGCTTTGAATA
TCTGCAATCTTTGCCAAGGTACTTTTTTATTTAAAAAAAAACATAACTTT
GTAAATATTACCCTGTAATATTATATATACTTAATAAAACATTTTAAGCT
ATTTTGTTGGGCTATTTCTATTGCTGCTACAGCAGACCACAAGCACATTT
CTGAAAAATTTAATTTATTAATGTATTTTTAAGTTGCTTATATTCTAGGT
AACAATGTAAAGAATGATTTAAAATATTAATTATGAATTTTTTGAGTATA
ATACCCAATAAGCTTTTAATTAGAGCAGAGTTTTAATTAAAAGTTTTAAA
TCAGTC
(Isoform 1 precursor) NP_001768.1
(SEQ ID NO: 60)
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRKAVEEPLNAFKESKGMMNDE

Isoform/Variant includes: (Isoform 2 precursor) NM_198793.2 and NP_942088.1

NCAM1 - (Isoform 1 precursor) NM_000615.7
(SEQ ID NO: 61)
CAGCTTCTGGGGGTAGATCTCAATTTGCAGTATTCAGACTTCTTTTTCTT
TCTTTTACATTCTTTTTTCTTTCTTTCTTTCTGCCAACTTTGTTTTCCAG
TGTTTACAAGGTGACAAATGTTTGACTTTGGTTGTGTTTAAATGTCCGTG
TAAAATAGCTGCCTTTTATTTTTTAAGGTAACAAATACCACCTAGAGGTA
GGTAGGATCATCCCACGCTTGCTTTAGCACAGGACAACTTTACAAAACAT
GATTGTTTACAGCTGCTCTTCCCCTCTTTTCTGATCTGCAGTTTTTGCCT
GGGTCCCACTCAGGTGAAAATCCATCTCATTCTGGAATGGTTTTGCTTTT
GAATTTTTGGTTATTTTTGTGTTTCTTTGGGGGTTAGACCACTTTCTGAT
TAGCCGCCACCTGCCTGCATCTGTGAAAAGGGATCTGCTCCCAGGCGTTC
TCACCCTTCTTTTGAAGGACTCCTTAGGCTTTGTTGAATGAAGCAGAGAA
GATTGTATAGTTGGGGCTGGTCTTGGTGAACACACATTATTACCCCACAC
ATCCCCTTTGTGTAGAAAGCCAAATAAAATCTATACATACCATTTCCTTT
TGAGCCCAGAATCTAGATTTGAGCGGAAGAGCATGTGTGCTTCAGGGAAT
TAGTGTCTTTTTTTGGAAATCTGTTGAAGTAAAGTAACATCGGCCTTCTG
TTCACTTAGGCAGCATTTATAGAAACAAAAGAAGAAAGAAACAACCTACT
GTCTGGAGTCATAACACAACTTTCCTGGATTGGAAACCAAGTGGGGGAAA
AAATACAGAAACTTTAAGGGGGATGGGAGGGGGGGGAGAAGGGAAAAGCC
AGCCCTTTGTATAGAAATTTTGCTTTTTTTTCCCTCATTCTACTTTAGAA
CTGCAAGCTTGTGCACTGTGGATGCGTGAATATTTTAGTGTGAAACGTGT
TTTTGTCATAGTATTGAAATAAAACTTCAACATAGTTTGGTTGTGGAAGG
TATAGCAGATAGTTCAGAAAAAATATTCAGGAAACAAAAATCACTCAAAC
GGAATCGAAGCCTTTTAACAAAGAAAATGAAATACAGATGATGATGATGA
TGATGAAGATGATGCTAAGTAAACAGAAATCAGTACTCCGCATGCGCTCC
TCTCCTAAGGTACAAAGCAGCAAGAGGTTAGGGTGGCAAGGCTGCCTCTG
GGTCCATTCTGTGGGCCACTCTCCCCAACGTTCTGACACTTCTGCAGTCT
GATCAGTGGCGATGCTAGATTATAATTTCAAACTGTGAAGAATAATGGTC
TTGTCATTTGCTCAATGTGGGGTTATGTTGCATTTTCTCAGCTCCTGGGG
ATGGAAATGGAGGATCCCAGAACACACAGCCCTGGCCCCTTTGATTCTAG
GGCCTGCACAGATCTCTGGTTCAAATGCACAGGCCCTCAGAATAGAGGAA
CATGAAGAGAGATCTTAGAGCACACAGTAGAATGTGAGAGCCTGGGTGTC
TGAGACCGGGAGGGCCCAGCAGTGAGGGGCAGGCTCTTCTGGTCACCAGG
CTGTTCAGTGGACTCAGTTCTTCATCTTGTAATGTCGATGGCTTTGCCAC
ACCAGGCCAAGCCCATGCCATACCTTGTCAAGACTGTCAAAGTGGTTGTG
GTTAGGTCAAACTGGTTTTGGTTCTGATGGTTAGGAAGAAACAGGTCAGC
CCTCAGATCACCTGGCCCGGGACAGCTGACCCCCTAGAACCCTGGCTCTG
CCATTAGCTAGGACCTAAGACTCTGCCCACATTTTGGTCTGTTCTCTCCC
ATTACACATAGGTTTGTCTCAGCATGCAAGAGTTTTTCCTTTAAAAAAAA
AAAAAAAAAAAAAAAAAAAGCAATGCTTTCTCTAAAATCAAAGAGGGAGT
CATTTTATTCCAAGATGTTTTATCTTTTATGTTAAGAGATCAAAGCTTAT
AATTTTCTTTTTTAATTTTTGAAGGAGGGATCAACTCCAGTTTCCAATGT
CTATGTGTCTATGTGTGTATGTGCCATACATATGTATTCACATGAAGACC
GGCATGGCCAAGTTCTGCTGGAGGAGCACTCAAGTGTGACGAGCAGGGCC
ACTGGACCCTGCAGGGCTGTGGTGTATATAGTGCAGCTTTGGAGGTGGAA
CTCTATTTTCACACTTTTCTATGGAGCCTTCCGAGTCCCAGGTTTTCACT
TGAGGCTGTCTGTCTGGATGGCGGTTTTCAGACCTCCATTAACATCCCTA
CCCAGCATTCTGTACTTCGGGGGCCTTCTCTCTTGTTATAAAACTTTTTA
CCAAGTGAAACATCGATACCACCTTTGTTTCCATTCTCACTGGTGTAAAT
ACTGAGTACTAACTGAGAATTTTGACTTTGCATTCTGTCGGAATACTTGT
GTTCAATAAAAATTGAAAGAAAAAAGCTA
(Isoform 1 precursor) NP_000606.3
(SEQ ID NO: 62)
MLQTKDLIWTLFFLGTAVSLQVDIVPSQGEISVGESKFFLCQVAGDAKDK
DISWFSPNGEKLTPNQQRISVVWNDDSSSTLTIYNANIDDAGIYKCVVTG
EDGSESEATVNVKIFQKLMFKNAPTPQEFREGEDAVIVCDVVSSLPPTII
WKHKGRDVILKKDVRFIVLSNNYLQIRGIKKTDEGTYRCEGRILARGEIN
FKDIQVIVNVPPTIQARQNIVNATANLGQSVTLVCDAEGFPEPTMSWTKD
GEQIEQEEDDEKYIFSDDSSQLTIKKVDKNDEAEYICIAENKAGEQDATI
HLKVFAKPKITYVENQTAMELEEQVTLTCEASGDPIPSITWRTSTRNISS
EEKTLDGHMVVRSHARVSSLTLKSIQYTDAGEYICTASNTIGQDSQSMYL
EVQYAPKLQGPVAVYTWEGNQVNITCEVFAYPSATISWFRDGQLLPSSNY
SNIKIYNTPSASYLEVTPDSENDFGNYNCTAVNRIGQESLEFILVQADTP
SSPSIDQVEPYSSTAQVQFDEPEATGGVPILKYKAEWRAVGEEVWHSKWY
DAKEASMEGIVTIVGLKPETTYAVRLAALNGKGLGEISAASEFKTQPVQG
EPSAPKLEGQMGEDGNSIKVNLIKQDDGGSPIRHYLVRYRALSSEWKPEI
RLPSGSDHVMLKSLDWNAEYEVYVVAENQQGKSKAAHFVFRTSAQPTAIP
ANGSPTSGLSTGAIVGILIVIFVLLLVVVDITCYFLNKCGLFMCIAVNLC
GKAGPGAKGKDMEEGKAAFSKDESKEPIVEVRTEEERTPNHDGGKHTEPN
ETTPLTEPEKGPVEAKPECQETETKPAPAEVKTVPNDATQTKENESKA

Isoform/Variant includes: (Isoform 3 precursor) NM_001076682.4 and NP_001070150.1; (Isoform 5 precursor) NM_001242607.2 and NP_001229536.1; (Isoform 4 precursor) NM_001242608.2 and NP_001229537.1; (Isoform 2 precursor) NM_181351.5 and NP_851996.2

CD58 - (Isoform 2) NM_001144822.1
(SEQ ID NO: 63)
GGGCCGCCGGCTGCCAGCCCAGGGCGGGGCGGAGCCCTACTTCTGGCCGA
CCGCGTAGGCGGTGCTTGAACTTAGGGCTGCTTGTGGCTGGGCACTCGCG
CAGAGGCCGGCCCGACGAGCCATGGTTGCTGGGAGCGACGCGGGGCGGGC
CCTGGGGGTCCTCAGCGTGGTCTGCCTGCTGCACTGCTTTGGTTTCATCA
GCTGTTTTTCCCAACAAATATATGGTGTTGTGTATGGGAATGTAACTTTC
CATGTACCAAGCAATGTGCCTTTAAAAGAGGTCCTATGGAAAAAACAAAA
GGATAAAGTTGCAGAACTGGAAAATTCTGAATTCAGAGCTTTCTCATCTT
TTAAAAATAGGGTTTATTTAGACACTGTGTCAGGTAGCCTCACTATCTAC
AACTTAACATCATCAGATGAAGATGAGTATGAAATGGAATCGCCAAATAT
TACTGATACCATGAAGTTCTTTCTTTATGTGCTTGAGTCTCTTCCATCTC
CCACACTAACTTGTGCATTGACTAATGGAAGCATTGAAGTCCAATGCATG
ATACCAGAGCATTACAACAGCCATCGAGGACTTATAATGTACTCATGGGA
TTGTCCTATGGAGCAATGTAAACGTAACTCAACCAGTATATATTTTAAGA
TGGAAAATGATCTTCCACAAAAAATACAGTGTACTCTTAGCAATCCATTA
TTTAATACAACATCATCAATCATTTTGACAACCTGTATCCCAAGCAGCGG
TCATTCAAGACACAGATATGCACTTATACCCATACCATTAGCAGTAATTA
CAACATGTATTGTGCTGTATATGAATGGTATTCTGAAATGTGACAGAAAA
CCAGACAGAACCAAGTAAGTACACTGCAGGCTGTGCACTCTGTGCCAGAA
CAACAGCAGCCCTGCTGTAGATGCCCATTACTTGGGAAGCCAGATTGTAC
TCATCTGTTGGTTGCCTTGTACAGTAATAAGAAGTAACATCAGCAGATAT
ACTAACCATAGTGTATTAAGTACCAACTGTGTGCCATGCACTGCACTAGC
CACTCTCTGCATATCGTTTCCTGGAACCCTCTCAATGAGCCTTGCAGGAG
GAGTATCATACCCCTTGCTTAGAGACAGCGCATATGAGATTCATAGAAGT
CAAGTGAGATATCAAAGTCACGTTGCTAGTAAGTGATGGGCTGGGATTTA
AATCTAGGTCTTTGGACTCCAAGAGTTATACTGTTAACCACTACAATACA
CTCCCTCCACCTCCCATCCCAGAAAAAGTTGGATCAGGGATAGGGTTCAC
TTCAGGAGCCAGGGTCAGAGCTGGGCCTGTAGTCACATATAAAAGATAGG
ACACAAACACAATTAGCAGCAGTGTTGGTTTAATAAACCGCCGTA
(Isoform 2) NP_001138294.1
(SEQ ID NO: 64)
MVAGSDAGRALGVLSVVCLLHCFGFISCFSQQIYGVVYGNVTFHVPSNVP
LKEVLWKKQKDKVAELENSEFRAFSSFKNRVYLDTVSGSLTIYNLTSSDE
DEYEMESPNITDTMKFFLYVLESLPSPTLTCALTNGSIEVQCMIPEHYNS
HRGLIMYSWDCPMEQCKRNSTSIYFKMENDLPQKIQCTLSNPLFNTTSSI
ILTTCIPSSGHSRHRYALIPIPLAVITTCIVLYMNGILKCDRKPDRTK

Isoform/Variant includes: (Isoform 1) NM_001779.3 and NP_001770.1

CD59 - (Variant 2) NM_000611.6
(SEQ ID NO: 65)
AGAAGCGGCTCGAGGCTGGAAGAGGATCTTGGGCGCCGCCAGGTTCTGTGGACAATCACAATGGGAATCC
AAGGAGGGTCTGTCCTGTTCGGGCTGCTGCTCGTCCTGGCTGTCTTCTGCCATTCAGGTCATAGCCTGCA
GTGCTACAACTGTCCTAACCCAACTGCTGACTGCAAAACAGCCGTCAATTGTTCATCTGATTTTGATGCG
TGTCTCATTACCAAAGCTGGGTTACAAGTGTATAACAAGTGTTGGAAGTTTGAGCATTGCAATTTCAACG
ACGTCACAACCCGCTTGAGGGAAAATGAGCTAACGTACTACTGCTGCAAGAAGGACCTGTGTAACTTTAA
CGAACAGCTTGAAAATGGTGGGACATCCTTATCAGAGAAAACAGTTCTTCTGCTGGTGACTCCATTTCTG
GCAGCAGCCTGGAGCCTTCATCCCTAAGTCAACACCAGGAGAGCTTCTCCCAAACTCCCCGTTCCTGCGT
AGTCCGCTTTCTCTTGCTGCCACATTCTAAAGGCTTGATATTTTCCAAATGGATCCTGTTGGGAAAGAAT
AAAATTAGCTTGAGCAACCTGGCTAAGATAGAGGGGCTCTGGGAGACTTTGAAGACCAGTCCTGTTTGCA
GGGAAGCCCCACTTGAAGGAAGAAGTCTAAGAGTGAAGTAGGTGTGACTTGAACTAGATTGCATGCTTCC
TCCTTTGCTCTTGGGAAGACCAGCTTTGCAGTGACAGCTTGAGTGGGTTCTCTGCAGCCCTCAGATTATT
TTTCCTCTGGCTCCTTGGATGTAGTCAGTTAGCATCATTAGTACATCTTTGGAGGGTGGGGCAGGAGTAT
ATGAGCATCCTCTCTCACATGGAACGCTTTCATAAACTTCAGGGATCCCGTGTTGCCATGGAGGCATGCC
AAATGTTCCATATGTGGGTGTCAGTCAGGGACAACAAGATCCTTAATGCAGAGCTAGAGGACTTCTGGCA
GGGAAGTGGGGAAGTGTTCCAGATAGCAGGGCATGAAAACTTAGAGAGGTACAAGTGGCTGAAAATCGAG
TTTTTCCTCTGTCTTTAAATTTTATATGGGCTTTGTTATCTTCCACTGGAAAAGTGTAATAGCATACATC
AATGGTGTGTTAAAGCTATTTCCTTGCCTTTTTTTTATTGGAATGGTAGGATATCTTGGCTTTGCCACAC
ACAGTTACAGAGTGAACACTCTACTACATGTGACTGGCAGTATTAAGTGTGCTTATTTTAAATGTTACTG
GTAGAAAGGCAGTTCAGGTATGTGTGTATATAGTATGAATGCAGTGGGGACACCCTTTGTGGTTACAGTT
TGAGACTTCCAAAGGTCATCCTTAATAACAACAGATCTGCAGGGGTATGTTTTACCATCTGCATCCAGCC
TCCTGCTAACTCCTAGCTGACTCAGCATAGATTGTATAAAATACCTTTGTAACGGCTCTTAGCACACTCA
CAGATGTTTGAGGCTTTCAGAAGCTCTTCTAAAAAATGATACACACCTTTCACAAGGGCAAACTTTTTCC
TTTTCCCTGTGTATTCTAGTGAATGAATCTCAAGATTCAGTAGACCTAATGACATTTGTATTTTATGATC
TTGGCTGTATTTAATGGCATAGGCTGACTTTTGCAGATGGAGGAATTTCTTGATTAATGTTGAAAAAAAA
CCCTTGATTATACTCTGTTGGACAAACCGAGTGCAATGAATGATGCTTTTCTGAAAATGAAATATAACAA
GTGGGTGAATGTGGTTATGGCCGAAAAGGATATGCAGTATGCTTAATGGTAGCAACTGAAAGAAGACATC
CTGAGCAGTGCCAGCTTTCTTCTGTTGATGCCGTTCCCTGAACATAGGAAAATAGAAACTTGCTTATCAA
AACTTAGCATTACCTTGGTGCTCTGTGTTCTCTGTTAGCTCAGTGTCTTTCCTTACATCAATAGGTTTTT
TTTTTTTTTTTTGGCCTGAGGAAGTACTGACCATGCCCACAGCCACCGGCTGAGCAAAGAAGCTCATTTC
ATGTGAGTTCTAAGGAATGAGAAACAATTTTGATGAATTTAAGCAGAAAATGAATTTCTGGGAACTTTTT
TGGGGGCGGGGGGGTGGGGAATTCAGCCACACTCCAGAAAGCCAGGAGTCGACAGTTTTGGAAGCCTCTC
TCAGGATTGAGATTCTAGGATGAGATTGGCTTACTGCTATCTTGTGTCATGTACCCACTTTTTGGCCAGA
CTACACTGGGAAGAAGGTAGTCCTCTAAAGCAAAATCTGAGTGCCACTAAATGGGGAGATGGGGCTGTTA
AGCTGTCCAAATCAACAAGGGTCATATAAATGGCCTTAAACTTTGGGGTTGCTTTCTGCAAAAAGTTGCT
GTGACTCATGCCATAGACAAGGTTGAGTGCCTGGACCCAAAGGCAATACTGTAATGTAAAGACATTTATA
GTACTAGGCAAACAGCACCCCAGGTACTCCAGGCCCTCCTGGCTGGAGAGGGCTGTGGCAATAGAAAATT
AGTGCCAACTGCAGTGAGTCAGCCTAGGTTAAATAGAGAGTGTAAGAGTGCTGGACAGGAACCTCCACCC
TCATGTCACATTTCTTCAATGTGACCCTTCTGGCCCCTCTCCTCCTGACAGCGGAACAATGACTGCCCCG
ATAGGTGAGGCTGGAGGAAGAATCAGTCCTGTCCTTGGCAAGCTCTTCACTATGACAGTAAAGGCTCTCT
GCCTGCTGCCAAGGCCTGTGACTTTCTAACCTGGCCTCACGCTGGGTAAGCTTAAGGTAGAGGTGCAGGA
TTAGCAAGCCCACCTGGCTACCAGGCCGACAGCTACATCCTCCAACTGACCCTGATCAACGAAGAGGGAT
TCATGTGTCTGTCTCAGTTGGTTCCAAATGAAACCAGGGAGCAGGGGAGTTAGGAATCGAACACCAGTCA
TGCCTACTGGCTCTCTGCTCGAGAGCCAATACCCTGTGCCCTCCACTCATCTGGATTTACAGGAACTGTC
ATAGTGTTCAGTATTGGGTGGTGATAAGCCCATTGGATTGTCCCCTTGGGGGGATGAGCTAGGGGTGCAA
GGAACACCTGATGAGTAGATAAGTGGAGCTCATGGTATTTCCTGAAAGATGCTAATCTATTTGCCAAACT
TGGTCTTGAATGTACTGGGGGCTTCAAGGTATGGGTATATTTTTCTTGTGTCCTTGCAGTTAGCCCCCAT
GTCTTATGTGTGTCCTGAAAAAATAAGAGCCTGCCCAAGACTTTGGGCCTCTTGACAGAATTAACCACTT
TTATACATCTGAGTTCTCTTGGTAAGTTCTTTAGCAGTGTTCAAAGTCTACTAGCTCGCATTAGTTTCTG
TTGCTGCCAACAGATCTGAACTAATGCTAACAGATCCCCCTGAGGGATTCTTGATGGGCTGAGCAGCTGG
CTGGAGCTAGTACTGACTGACATTCATTGTGATGAGGGCAGCTTTCTGGTACAGGATTCTAAGCTCTATG
TTTTATATACATTTTCATCTGTACTTGCACCTCACTTTACACAAGAGGAAACTATGCAAAGTTAGCTGGA
TCGCTCAAGGTCACTTAGGTAAGTTGGCAAGTCCATGCTTCCCACTCAGCTCCTCAGGTCAGCAAGTCTA
CTTCTCTGCCTATTTTGTATACTCTCTTTAATATGTGCCTAGCTTTGGAAAGTCTAGAATGGGTCCCTGG
TGCCTTTTTACTTTGAAGAAATCAGTTTCTGCCTCTTTTTGGAAAAGAAAACAAAGTGCAATTGTTTTTT
ACTGGAAAGTTACCCAATAGCATGAGGTGAACAGGACGTAGTTAGGCCTTCCTGTAAACAGAAAATCATA
TCAAAACACTATCTTCCCATCTGTTTCTCAATGCCTGCTACTTCTTGTAGATATTTCATTTCAGGAGAGC
AGCAGTTAAACCCGTGGATTTTGTAGTTAGGAACCTGGGTTCAAACCCTCTTCCACTAATTGGCTATGTC
TCTGGACAAGTTTTTTTTTTTTTTTTTTTTTAAACCCTTTCTGAACTTTCACTTTCTATGTCTACCTCAA
AGAATTGTTGTGAGGCTTGAGATAATGCATTTGTAAAGGGTCTGCCAGATAGGAAGATGCTAGTTATGGA
TTTACAAGGTTGTTAAGGCTGTAAGAGTCTAAAACCTACAGTGAATCACAATGCATTTACCCCCACTGAC
TTGGACATAAGTGAAAACTAGCCAGAAGTCTCTTTTTCAAATTACTTACAGGTTATTCAATATAAAATTT
TTGTAATGGATAATCTTATTTATCTAAACTAAAGCTTCCTGTTTATACACACTCCTGTTATTCTGGGATA
AGATAAATGACCACAGTACCTTAATTTCTAGGTGGGTGCCTGTGATGGTTCATTGTAGGTAAGGACATTT
TCTCTTTTTCAGCAGCTGTGTAGGTCCAGAGCCTCTGGGAGAGGAGGGGGGTAGCATGCACCCAGCAGGG
GACTGAACTGGGAAACTCAAGGTTCTTTTTACTGTGGGGTAGTGAGCTGCCTTTCTGTGATCGGTTTCCC
TAGGGATGTTGCTGTTCCCCTCCTTGCTATTCGCAGCTACATACAACGTGGCCAACCCCAGTAGGCTGAT
CCTATATATGATCAGTGCTGGTGCTGACTCTCAATAGCCCCACCCAAGCTGGCTATAGGTTTACAGATAC
ATTAATTAGGCAACCTAAAATATTGATGCTGGTGTTGGTGTGACATAATGCTATGGCCAGAACTGAAACT
TAGAGTTATAATTCATGTATTAGGGTTCTCCAGAGGGACAGAATTAGTAGGATATATGTATATATGAAAG
GGAGGTTATTAGGGAGAACTGGCTCCCACAGTTAGAAGGCGAAGTCGCACAATAGGCCGTCTGCAAGCTG
GGTTAGAGAGAAGCCAGTAGTGGCTCAGCCTGAGTTCAAAAACCTCAAAACTGGGGAAGCTGACAGTGCA
GCCAGCCTTCAGTCTGTGGCCAAAGGCCCAAGAGCCCCTGGCAACCAACCCACTGGTGCAAGTCCTAGAT
TCCAAAGGCTGAAGAACCTGGAGTCTGATGTCCAAGAGCAGGAAGAGTGGAAGAAAGCCAGAAGACTCAG
CAAACAAGGTAGACAGTGTCTACCACCATAGTGGCCATACCAAAGAGGCTACCGATTCCTTCCTGCTACC
TGGATCCCTGAAGTTGCCCTGGTCTCTGCACCTTCTAAACCTAGTTCTTAAGAGCTTTCCATTACATGAG
CTGTCTCAAAGCCCTCCAATAAATTCTCAGTGTAAGCTTCTGTTGCTTGTGGACAGAAAATTCTGACAGA
CCTACCCTATAAGTGTTACTGTCAGGATAACATGAGAACGCACAACAGTAAGTGGTCACTAAGTGTTAGC
TACGGTTATTTTGCCCAAGGTAGCATGGCTAGTTGATGCCGGTTGATGGGGCTTAAACCCAGCTCCCTCA
TCTTCCAGGCCTCTGTACTCCCTATTCCACTAAACTACCTCTCAGGTTTATTTTTTTAAATTCTTACTCT
GCAAGTACATAGGACCACATTTACCTGGGAAAACAAGAATAAAGGCTGCTCTGCATTTTTTAGAAACTTT
TTTGAAAGGGAGATGGGAATGCCTGCACCCCCAAGTCCAGACCAACACAATGGTTAATTGAGATGAATAA
TAAAGGAAAGACTGTTCTGGGCTTCCCAGAATAGCTTGGTCCTTAAATTGTGGCACAAACAACCTCCTGT
CAGAGCCAGCCTCCTGCCAGGAAGAGGGGTAGGAGACTAGAGGCCGTGTGTGCAGCCTTGCCCTGAAGGC
TAGGGTGACAATTTGGAGGCTGTCCAAACACCCTGGCCTCTAGAGCTGGCCTGTCTATTTGAAATGCCGG
CTCTGATGCTAATCGGCGACCCTCAGGCAAGTTACTTAACCTTACATGCCTCAGTTTTCTCATCTGGAAA
ATGAGAACCCTAGGTTTAGGGTTGTTAGAAAAGTTAAATGAGTTAAGACAAGTGCCTGGGACACAGTAGC
CTCTTGTGTGTGTTTATCATTATGTCCTCAGCAGGTCGTAGAAGCAGCTTCTCAGGTGTGAGGCTGGCGC
GATTATCTGGAGTGGGTTGGGTTTTCTAGGATGGACCCCCTGCTGCATTTTCCTCATTCATCCACCAGGG
CTTAATGGGGAATCAAGGAATCCATGTGTAACTGTATAATAACTGTAGCCACACTCCAATGACCACCTAC
TAGTTGTCCCTGGCACTGCTTATACATATGTCCATCAAATCAATCCTATGAAGTAGATACTGTCTTCATT
TTATAGATCAGAGACAATTGGGGTTCAGAGAGCTGATGTGATTTTCCCAGGGTCACAGAGAGTCCCAGAT
TCAGGCACAACTCTTGTATTCCAAGACACAACCACTACATGTCCAAAGGCTGCCCAGAGCCACCGGGCAC
GGCAAATTGTGACATATCCCTAAAGAGGCTGAGCACCTGGTCAGGATCTGATGGCTGACAGTGTGTCCAG
ATGCAGAGCTGGAGTGGGGGAGGGGAAGGGGGGCTCCTTGGGACAGAGAAGGCTTTCTGTGCTTTCTCTG
AAGGGAGCAGTCTGAGGACCAAGGGAACCCGGCAAACAGCACCTCAGGTACTCCAGGCCCTCCTGGCTGG
AGAGGGCTGTGGCAATGGAAAATTAGTGCCAACTGCAATGAGTCAGCCTCGGTTAAATAGAGAGTGAAGA
ATGCTGGACAGGAACCTCCACCCTCATGTCACATTTCTTCAGTGTGACCCTTCTGGCCCCTCTCCTCCTG
ACAGCGGAACAATGACTGCCCCGATAGGTGAGGCTGGAGGAAGAATCAGTCCTGTCCTTGGCAAGCTCTT
CACTATGACAGTAAAGGCTCTCTGCCTGCTGCCAAGGCCTGTGACTTTCTAACCTGGCCTCACGCTGGGT
AAGCTTAAGGTAGAGGTGCAGGATTAGCAAGCCCACCTGGCTACCAGGCCGACAGCTACATCTTTCAACT
GACCCTGATCAACGAAGAGGGACTTGTGTCTCTCAGTTGGTTCCAAATGAAACCAGGGAGCAGGGGCGTT
AGGAAGCTCCAACAGGATGGTACTTAATGGGGCATTTGAGTGGAGAGGTAGGTGACATAGTGCTTTGGAG
CCCAGGGAGGGAAAGGTTCTGCTGAAGTTGAATTCAAGACTGTTCTTTCATCACAAACTTGAGTTTCCTG
GACATTTGTTTGCAGAAACAACCGTAGGGTTTTGCCTTAACCTCGTGGGTTTATTATTACCTCATAGGGA
CTTTGCCTCCTGACAGCAGTTTATGGGTGTTCATTGTGGCACTTGAGTTTTCTTGCATACTTGTTAGAGA
AACCAAGTTTGTCATCAACTTCTTATTTAACCCCCTGGCTATAACTTCATGGATTATGTTATAATTAAGC
CATCCAGAGTAAAATCTGTTTAGATTATCTTGGAGTAAGGGGGAAAAAATCTGTAATTTTTTCTCCTCAA
CTAGATATATACATAAAAAATGATTGTATTGCTTCATTTAAAAAATATAACGCAAAATCTCTTTTCCTTC
TAA
(Variant 2) NP_000602.1
(SEQ ID NO: 66)
MGIQGGSVLFGLLLVLAVFCHSGHSLQCYNCPNPTADCKTAVNCSSDFDACLITKAGLQVYNKCWKFEHC
NFNDVTTRLRENELTYYCCKKDLCNFNEQLENGGTSLSEKTVLLLVTPFLAAAWSLHP

Isoform/Variant includes: (Variant 5) NM_001127223.1 and NP_001120695.1; (Variant 6) NM_001127225.1 and NP_001120697.1; (Variant 7) NM_001127226.1 and NP_001120698.1; (Variant 8) NM_001127227.1 and NP_001120699.1; (Variant 3) NM_203329.2 and NP_976074.1; (Variant 1) NM 203330.2 and NP_976075.1; (Variant 4) NM_203331.2 and NP_976076.1

CD81 - (Isoform 2) NM_001297649.1
(SEQ ID NO: 67)
TTTGGTTCCTTGTGGCCACATTTCCAGTACCCAGTAGTCATCTGTGCCAG
GGGGTTATCCAGGTACAGAACATTCCCATCGTTGCAGAAGGTTCTATCAG
CTAGCACTGGGTTGGACGACACTTGCCAAGACGAGCTGGCTAGAGGATGG
TTCTCCGGACCTGGTCCCACGTGGTTCCCAGCTGGCTGGAGGCGTGATCC
TGGGTGTGGCCCTGTGGCTCCGCCATGACCCGCAGACCACCAACCTCCTG
TATCTGGAGCTGGGAGACAAGCCCGCGCCCAACACCTTCTATGTAGGCAT
CTACATCCTCATCGCTGTGGGCGCTGTCATGATGTTCGTTGGCTTCCTGG
GCTGCTACGGGGCCATCCAGGAATCCCAGTGCCTGCTGGGGACGTTCTTC
ACCTGCCTGGTCATCCTGTTTGCCTGTGAGGTGGCCGCCGGCATCTGGGG
CTTTGTCAACAAGGACCAGATCGCCAAGGATGTGAAGCAGTTCTATGACC
AGGCCCTACAGCAGGCCGTGGTGGATGATGACGCCAACAACGCCAAGGCT
GTGGTGAAGACCTTCCACGAGACGCTTGACTGCTGTGGCTCCAGCACACT
GACTGCTTTGACCACCTCAGTGCTCAAGAACAATTTGTGTCCCTCGGGCA
GCAACATCATCAGCAACCTCTTCAAGGAGGACTGCCACCAGAAGATCGAT
GACCTCTTCTCCGGGAAGCTGTACCTCATCGGCATTGCTGCCATCGTGGT
CGCTGTGATCATGATCTTCGAGATGATCCTGAGCATGGTGCTGTGCTGTG
GCATCCGGAACAGCTCCGTGTACTGAGGCCCCGCAGCTCTGGCCACAGGG
ACCTCTGCAGTGCCCCCTAAGTGACCCGGACACTTCCGAGGGGGCCATCA
CCGCCTGTGTATATAACGTTTCCGGTATTACTCTGCTACACGTAGCCTTT
TTACTTTTGGGGTTTTGTTTTTGTTCTGAACTTTCCTGTTACCTTTTCAG
GGCTGACGTCACATGTAGGTGGCGTGTATGAGTGGAGACGGGCCTGGGTC
TTGGGGACTGGAGGGCAGGGGTCCTTCTGCCCTGGGGTCCCAGGGTGCTC
TGCCTGCTCAGCCAGGCCTCTCCTGGGAGCCACTCGCCCAGAGACTCAGC
TTGGCCAACTTGGGGGGCTGTGTCCACCCAGCCCGCCCGTCCTGTGGGCT
GCACAGCTCACCTTGTTCCCTCCTGCCCCGGTTCGAGAGCCGAGTCTGTG
GGCACTCTCTGCCTTCATGCACCTGTCCTTTCTAACACGTCGCCTTCAAC
TGTAATCACAACATCCTGACTCCGTCATTTAATAAAGAAGGAACATCAGG
CATGCTACCAGGCCTGTGCAGTCCCTCAG
(Isoform 2) NP_001284578.1
(SEQ ID NO: 68)
MMFVGFLGCYGAIQESQCLLGTFFTCLVILFACEVAAGIWGFVNKDQIAK
DVKQFYDQALQQAVVDDDANNAKAVVKTFHETLDCCGSSTLTALTTSVLK
NNLCPSGSNIISNLFKEDCHQKIDDLFSGKLYLIGIAAIVVAVIMIFEMI
LSMVLCCGIRNSSVY

Isoform/Variant includes: (Isoform 1) NM_004356.4 and NP_004347.1

THY1 - (Isoform 1 Preproprotein)
NM_001311160.2
(SEQ ID NO: 69)
GTGAATCTGCGCTAAGCTATGCAGTCTGCTTTTTCTTCTCAGCTCTGGTA
GTTCTTCAGAAATGTACCCTCCAGGCACATCCACTATTGCGAGGGTGAGC
ACGAAGGGTGGGAGATGCCCATGTCCTCAAGGCATCACTTCCTAAATCCA
AAAGCATCGGCAGGAGAAAGGACTGGGGACAAATACTGTCCCTTCGGGAG
TAGGGAGGGAACACTGAGGCCCATCCCTGGCTCCTTCCCTAAAAGTAGAG
TAAAATGGAAGCGAGCATCCTGGGATTGGGGGCAAGAGGGGGACCGCAGG
GTAGCTGTGGGTTCCAACTGCTGTCAGAGTCAGAGAGGCAGCCCCAAGCC
AGCCTCCCTGCTTTGCCAGGGAATTTGGGGGAGGAAGGTGACAGCTGCCC
AGAGGCTGACTCATCTGATATTTAGCACTGGGTAGGATGATTGTTTCTGA
GCATTTTTCTTAAAGGCCTCAGATCTAAATTATGCCACCGGCTCCCACTC
TTGCTACCTCCCGTCAACTTCTCTGCCTTGCCTTCCACCCCTGTAGTTAC
CATACACAGAGGAGGAGGAGCTGTCCTTGTCCCAGGTTGGGAGGCTGACA
ACCCCTTAGCAAGATGCTGCCAGCCCAGAGCTCTCCAAGGGGAGGAACAC
CCCTGAGACTCAGGCCCCTCTCCTTCAGCCCTGCTTGGGCTGCAAGCGCC
GTGCCAAGGAAAGGCATCTTGGTGAGAAGAGCTGCTGTGGGGGAAGGGAG
ATCAAATGCCAGAGAAATGTGGGGTGCCCCACCCTCAGGATAGTAAAAGA
GTATGGAGGTATTTCTGGAAGGAAATGAGCGGCACTGTGTGAAGCCTCGC
ACCTGTGTGACACTTCCTATGGGGTCTTTGTCACACTCTAGTACTATGTC
CCTGAAGAGTTTAGCAGCCACACTCTTAGAAGGGTGCTGGGAGATGGTGT
TGCCCTCTGCAGCCATGTTTAGGGGAGCGGAACCTGAGGCCCACAGTGGG
TGAGATTAGCTCAAGAAGCCACAGAGGCCACCAGAGGGCCACGGACTTCG
GAAAGGAGAAGAGAAGAACAGGGCATCAGGCCTCACAACGCAAACCTACC
CAGAGATGGGCACAGTGGCTCATGCCTGTAATCCCACCACTTTGGGAAGA
GGCGGATCGCTTGAGGTCAGGAGTTCGAGACTAGCCTCGAAACCCTATCT
CTACTAAAAATACAAAAATTAGCCAGGCATGGTGGCCTGCGCCTGTAATC
CCAGCTACTCAGGAGGCTGAGGCAGGAGAATCACTTGAACCCGGGAGGCG
GAGGTTGCAGTGAGCCAAGATTGCACCACTGCACTCCAGCCTGGGAGATA
GAGTGAGACTCCATCTCAAAAAAATAAAAAATAAAATAAACCTACCCGGA
ATGACCATGCTGAGGACTGGGAGCCCGCAGACTTTCAGCCACAGGCCGCG
ACAGCCGTGGGTCCCTCCCTGGTCAAGTCAGCAGGCCTTGTGGAGGCTGT
GGGGTATCTGTGGTGACTCAGGTAATTATAGAGGGCTGGCCCCCAGCCCT
GGTTCCTGTACACATGCCCCAACCCCATCCCCATCCACTCCCTCGCCAGT
CCTAACCTCTTTCCTGGGTCCCCCCCCTTCAGCACCTAAGTCCATACCTA
GGGCCGTGGAATTCCCGCTCAAGAGCAACAGAAGCCCCTCTCTGCACCCC
CATTTCTGGACTGGATTGTCCACTGAGACGCGCAATGTCTGCATCTCTGA
CATCTAGAGGCTTCCTCGGGAAGGGCATGGGGATCTCCGTGAGATGTGGG
GACTTTCACTGGCCAACCAAGAAATCTACACAGCGTCCGGGGACCTGTGA
CACACATCCCTCCCGCCTCCTCAACCTGATGTCCCTCTCTGAATCTGCAG
CTTTCGTGCTGTGAAGGTGTCTTTACATGTGAAACAAACAAACCCAAGTC
AAGAGTAAATCATCTCATTTACTAGTGAGAAAATGTTGGAGCTGGAGTCC
TTCAGAGAGTCCTGGCCAGGCAAGAGGGCCATCAGCTCTCTTCTGCTCAA
CAGGGGCTCTCAGCCTCAGGACACTCTCAGGCCTGGAATGTCCCCAACAC
ACTCAAGGAGAAACATGTCCTGTGCAGACCCACAGGAGGCATCTTTGCCC
GGCACAAGGAAGAGCTGGGGTCAGTGGGACCTGTAGATGTAGACACATCA
TATGGAGGGTGGGTAGGACCAATGTGGCAGCTTCATGGAGGCCAAGTGTG
GCTCTGCACCAGGAAGGGGCTGTGATGGCTGGAGGTGCCCAGCAGTGCAG
GCGGGGAGTGCCTGGCAGTGGCGTGGCCAGGTGGAGGCCACCTGTCAAGT
TTGCAATAAAGCAGTTTCCTGAATTTGGTGAGAA
(Isoform 1 Preproprotein)
NP_001298089.1
(SEQ ID NO: 70)
MNLAISIALLLTVLQVSRGQKVTSLTACLVDQSLRLDCRHENTSSSPIQY
EFSLTRETKKHVLFGTVGVPEHTYRSRTNFTSKYNMKVLYLSAFTSKDEG
TYTCALHHSGHSPPISSQNVTVLRDKLVKCEGISLLAQNTSWLLLLLLSL
SLLQATDFMSL

Isoform/Variant includes: (Isoform 1 Preproprotein) NM_001311162.2 and NP_001298091.1; (Isoform 2 precursor) NM_001372050.1 and NP_001358979.1; (Isoform 1 Preproprotein) NM_006288.5 and NP_006279.2

LAMP1 - NM_005561.4
(SEQ ID NO: 71)
GCAATGTTTATGGTGAAAAATGGCAACGGGACCGCGTGCATAATGGCCAACTTCTCTGCTGCCTTCTCAG
TGAACTACGACACCAAGAGTGGCCCTAAGAACATGACCTTTGACCTGCCATCAGATGCCACAGTGGTGCT
CAACCGCAGCTCCTGTGGAAAAGAGAACACTTCTGACCCCAGTCTCGTGATTGCTTTTGGAAGAGGACAT
ACACTCACTCTCAATTTCACGAGAAATGCAACACGTTACAGCGTCCAGCTCATGAGTTTTGTTTATAACT
TGTCAGACACACACCTTTTCCCCAATGCGAGCTCCAAAGAAATCAAGACTGTGGAATCTATAACTGACAT
CAGGGCAGATATAGATAAAAAATACAGATGTGTTAGTGGCACCCAGGTCCACATGAACAACGTGACCGTA
ACGCTCCATGATGCCACCATCCAGGCGTACCTTTCCAACAGCAGCTTCAGCCGGGGAGAGACACGCTGTG
AACAAGACAGGCCTTCCCCAACCACAGCGCCCCCTGCGCCACCCAGCCCCTCGCCCTCACCCGTGCCCAA
GAGCCCCTCTGTGGACAAGTACAACGTGAGCGGCACCAACGGGACCTGCCTGCTGGCCAGCATGGGGCTG
CAGCTGAACCTCACCTATGAGAGGAAGGACAACACGACGGTGACAAGGCTTCTCAACATCAACCCCAACA
AGACCTCGGCCAGCGGGAGCTGCGGCGCCCACCTGGTGACTCTGGAGCTGCACAGCGAGGGCACCACCGT
CCTGCTCTTCCAGTTCGGGATGAATGCAAGTTCTAGCCGGTTTTTCCTACAAGGAATCCAGTTGAATACA
ATTCTTCCTGACGCCAGAGACCCTGCCTTTAAAGCTGCCAACGGCTCCCTGCGAGCGCTGCAGGCCACAG
TCGGCAATTCCTACAAGTGCAACGCGGAGGAGCACGTCCGTGTCACGAAGGCGTTTTCAGTCAATATATT
CAAAGTGTGGGTCCAGGCTTTCAAGGTGGAAGGTGGCCAGTTTGGCTCTGTGGAGGAGTGTCTGCTGGAC
GAGAACAGCATGCTGATCCCCATCGCTGTGGGTGGTGCCCTGGCGGGGCTGGTCCTCATCGTCCTCATCG
CCTACCTCGTCGGCAGGAAGAGGAGTCACGCAGGCTACCAGACTATCTAGCCTGGTGCACGCAGGCACAG
CAGCTGCAGGGGCCTCTGTTCCTTTCTCTGGGCTTAGGGTCCTGTCGAAGGGGAGGCACACTTTCTGGCA
AACGTTTCTCAAATCTGCTTCATCCAATGTGAAGTTCATCTTGCAGCATTTACTATGCACAACAGAGTAA
CTATCGAAATGACGGTGTTAATTTTGCTAACTGGGTTAAATATTTTGCTAACTGGTTAAACATTAATATT
TACCAAAGTAGGATTTTGAGGGTGGGGGTGCTCTCTCTGAGGGGGTGGGGGTGCCGCTGTCTCTGAGGGG
TGGGGGTGCCGCTGTCTCTGAGGGGTGGGGGTGCCGCTCTCTCTGAGGGGGTGGGGGTGCCGCTTTCTCT
GAGGGGGTGGGGGTGCCGCTCTCTCTGAGGGGGTGGGGGTGCTGCTCTCTCCGAGGGGTGGAATGCCGCT
GTCTCTGAGGGGTGGGGGTGCCGCTCTAAATTGGCTCCATATCATTTGAGTTTAGGGTTCTGGTGTTTGG
TTTCTTCATTCTTTACTGCACTCAGATTTAAGCCTTACAAAGGGAAAGCCTCTGGCCGTCACACGTAGGA
CGCATGAAGGTCACTCGTGGTGAGGCTGACATGCTCACACATTACAACAGTAGAGAGGGAACATCCTAAG
ACAGAGGAACTCCAGAGATGAGTGTCTGGAGCGCTTCAGTTCAGCTTTAAAGGCCAGGACGGGCCACACG
TGGCTGGCGGCCTCGTTCCAGTGGCGGCACGTCCTTGGGCGTCTCTAATGTCTGCAGCTCAAGGGCTGGC
ACTTTTTTAAATATAAAAATGGGTGTTATTTTTATTTTTATTTGTAAAGTGATTTTTGGTCTTCTGTTGA
CATTCGGGGTGATCCTGTTCTGCGCTGTGTACAATGTGAGATCGGTGCGTTCTCCTGATGTTTTGCCGTG
GCTTGGGGATTGTACACGGGACCAGCTCACGTAATGCATTGCCTGTAACAATGTAATAAAAAGCCTCTTT
CTTTTTGGGGTGGGCCTTGTCCTTCTGTCAGCTAAAATGGGAGCTCATGAGAGAAGGACGTCAGGGAAAC
GGGGTTGAGGGTGGTCTCGGTGCAGAGAGAAGGGTGTCAGGGAAACGGGGGGTGAGGGTGGTCTTGGTGC
CAGACGTAGGGAATGGTGTTGGGAGTGGCCCGAGTGCCTGGCACAGTTGTCTGGTTCATTCATGTAACAT
GATAATTTTTAAATCATTAAAAAAATTACCTTTCATACAGA
NP_005552.3
(SEQ ID NO: 72)
MAAPGSARRPLLLLLLLLLLGLMHCASAAMFMVKNGNGTACIMANFSAAFSVNYDTKSGPKNMTFDLPSD
ATVVLNRSSCGKENTSDPSLVIAFGRGHTLTLNFTRNATRYSVQLMSFVYNLSDTHLFPNASSKEIKTVE
SITDIRADIDKKYRCVSGTQVHMNNVTVTLHDATIQAYLSNSSFSRGETRCEQDRPSPTTAPPAPPSPSP
SPVPKSPSVDKYNVSGTNGTCLLASMGLQLNLTYERKDNTTVTRLLNINPNKTSASGSCGAHLVTLELHS
EGTTVLLFQFGMNASSSREELQGIQLNTILPDARDPAFKAANGSLRALQATVGNSYKCNAEEHVRVTKAE
SVNIFKVWVQAFKVEGGQFGSVEECLLDENSMLIPIAVGGALAGLVLIVLIAYLVGRKRSHAGYQTI
LAMP2 - (Isoform C Precursor) NM_001122606.1
(SEQ ID NO: 73)
AAGAAAGAGCCCCGCCCCTAGTCTTATGACTCGCACTGAAGCGCCGATTCCTGGCTTTTGCAAGGCTGTG
GTCGGTGGTCATCAGTGCTCTTGACCCAGGTCCAGCGAGCCTTTTCCCTGGTGTTGCAGCTGTTGTTGTA
CCGCCGCCGTCGCCGCCGTCGCCGCCTGCTCTGCGGGGTCATGGTGTGCTTCCGCCTCTTCCCGGTTCCG
GGCTCAGGGCTCGTTCTGGTCTGCCTAGTCCTGGGAGCTGTGCGGTCTTATGCATTGGAACTTAATTTGA
CAGATTCAGAAAATGCCACTTGCCTTTATGCAAAATGGCAGATGAATTTCACAGTACGCTATGAAACTAC
AAATAAAACTTATAAAACTGTAACCATTTCAGACCATGGCACTGTGACATATAATGGAAGCATTTGTGGG
GATGATCAGAATGGTCCCAAAATAGCAGTGCAGTTCGGACCTGGCTTTTCCTGGATTGCGAATTTTACCA
AGGCAGCATCTACTTATTCAATTGACAGCGTCTCATTTTCCTACAACACTGGTGATAACACAACATTTCC
TGATGCTGAAGATAAAGGAATTCTTACTGTTGATGAACTTTTGGCCATCAGAATTCCATTGAATGACCTT
TTTAGATGCAATAGTTTATCAACTTTGGAAAAGAATGATGTTGTCCAACACTACTGGGATGTTCTTGTAC
AAGCTTTTGTCCAAAATGGCACAGTGAGCACAAATGAGTTCCTGTGTGATAAAGACAAAACTTCAACAGT
GGCACCCACCATACACACCACTGTGCCATCTCCTACTACAACACCTACTCCAAAGGAAAAACCAGAAGCT
GGAACCTATTCAGTTAATAATGGCAATGATACTTGTCTGCTGGCTACCATGGGGCTGCAGCTGAACATCA
CTCAGGATAAGGTTGCTTCAGTTATTAACATCAACCCCAATACAACTCACTCCACAGGCAGCTGCCGTTC
TCACACTGCTCTACTTAGACTCAATAGCAGCACCATTAAGTATCTAGACTTTGTCTTTGCTGTGAAAAAT
GAAAACCGATTTTATCTGAAGGAAGTGAACATCAGCATGTATTTGGTTAATGGCTCCGTTTTCAGCATTG
CAAATAACAATCTCAGCTACTGGGATGCCCCCCTGGGAAGTTCTTATATGTGCAACAAAGAGCAGACTGT
TTCAGTGTCTGGAGCATTTCAGATAAATACCTTTGATCTAAGGGTTCAGCCTTTCAATGTGACACAAGGA
AAGTATTCTACAGCTGAAGAATGTTCTGCTGACTCTGACCTCAACTTTCTTATTCCTGTTGCAGTGGGTG
TGGCCTTGGGCTTCCTTATAATTGTTGTCTTTATCTCTTATATGATTGGAAGAAGGAAAAGTCGTACTGG
TTATCAGTCTGTGTAATCAGTTAAATCTAGTGTTTGTTTGTTTTTTTCAATTAGAAGTTACGTTTCCATT
GGCTAAAAGCCAGGACATGCTGTGCAATAGATTGTTTAAGATATGCAGACTAACTTCAGTGAGTTCCTAG
CTAACTTGGGCATGAGTACACTTATTTAAGACAAAATATATTAGGACCAATTTTTTTCTGTTTTTTTTCT
TCCTTTGTTAAAGTATAATTAAAAGAAAAATTGTGGCTTAGAATTTTTTAAGTAAATAATGATTTTAAGC
CCCTGGATCCAATTATGAAAGCATTTTTGCTGATGTGTAATTTTATATGTTACAGTTACTTATATTTTAC
TACTTTGATGTTATTTGCAAAATCAAAGGTGTTAAAGAATTTAACTTGCTTCAGGAAATAAATTCAAGAA
CATAGTGGATTCATTTTCATTGGTGGCAGACACGAAATTTGGTTCATGATAAGACTTCCTTTCCCCACCT
CCTGATCAGCATTATTTAAATCTGTATTTTTCTGTTAGTTAAGAAAGAAATGGCTTCATGATATTGTATT
TAATAGCAAAAGTTTGGCTGTCTTCTTCATTACTGTTAATAGCTACTATATTTTAACAAGGAGATTTCTT
TTTTTGTTGTTGTTGTTCTAGAGTTTGGAATATACTGATTATCTCAGACTTGACATTTATACTGAAGGAT
GAAGTAAGACCTCCAGCTTTTTTTAAAAAAGGTGTTGATTTGGAACACCTGTATGGGTTATGGTTTATTA
AGGTTATGGTTTAGAAAGTTTTTTTCCCTCAGAGCCTTAACTTGTTAAGAAGGTTCATTTATCCTGCACT
GAAAACAAAAACTCTATATACTTTGTTTGTGTGCCTCCTGCACTCTCCCATTCCCTATGTGAATATGCTC
TAGTTGATATTTTTAATATATTGATTTCTTTTTTCTCACAGCAACAAGTGCTTACTCTAGAGGTTAGTGG
GCCCTGATATGTCATCAGTCAGATGCCTGCCTAGCCAAAGCTGGACTAAGATTATTCTGTACATTTGTTG
ATCTTGATATAGACTTATATCCCTGTAGGGACTGCTAATGGCTCCGGCTTCTGGAGTAAGGTACTGGAGA
CCACTCATCCCTGTGTCTGCTTGATTGGTTCAGCTGTTGAATTGCCCTTTTATTTGGAAGCAGTGTTGAA
GTTGTCTAGGGTTCAAATGGCTGCTTTGTACACCTGTCATTAGTATAAGGCAGATGTTTATTTTATCAAG
CTATTTTATCTCTACATTTAACTAAAAACAAAAGTTCCCAAAGATCTGCCTTCACTTCAGAAATTTTTTT
TGGATTAAAAAAATTAAGCCTGAACCTTAAATAAAGTGAGTTGGTTATTCATTCCAAGGATTAAGTCCCA
ATCTACCTCTCAGCACAATGCAGAAGCTCACCACTGTATTGCTGCCATTAACTCATGCCAGAACCCTTTG
CCAATAACTGGAATTACAAATTTTTGTTAAAGAAAATTTATCAAGATCTTTCTTTACTGCCTTCTCTATA
TGTACATCTCAAAAACATGTACATCTCAAAAACTGGAGTAGAAAGTTAGATTGCTCAACTACAACTCCTC
TAGAACTCTATAGCTCTGACATACAGATTCACACTCTCCTCTATTTGCTAAGTATGTAAAGAATGTTTTC
TTTTAAAATGTTCTCTTTTGAGAACAACTGCTTATTTGTTATAAAAGCATTTGGTTAAAATGATGTCATC
ATAAAAAACAGTGGCTTTGTTTCAATACATATTTTTGAGATGATTATCTAGAAGCCAGATTAATAAAATC
AGCTTGTGACCTTGCTAAGCATATAAACTGGAAATTCAGATACATTCAAAATTATGGGTTCATTTAAAAG
TGTTCTACCTTTTGGGTATGAGACTAATATCACTAATTCCTCAATAGTTATCATGGCTCTATCTTAATTA
ATTAGAAAATATGTGTGTTTAATTCTTTGAGAATTAAAATAGAGAATATTAACAGAGGGTTAAAAACTGC
TTCAACTCCAATAAGATAAAGGAAGCTCAAAATCTATGAGCTGAGTGTTCAATTAGCTTTGCCTACTGAG
TTCAATTTTATGTCAATACAACAGTGGATCAGACAGTACGACTTTGAACTGGTGAATGTAAACAATTGTT
TTTCACCTAAGCTGCTTTGGAAGAACTGATGCTTGCTGCTAACTAAAGTTTTGGATGTATCGATTTAGAG
AACCAATTAATACCTGCAAAATAAAGCATACTGTGGTACTTCTGTTTGATCTAGTATGTGTGATTTTAGA
TTGATGGATTAAAAATTAATAAAGATCATACATTCCATACCAAAAAAAAAAAAAAAA
(Isoform C Precursor) NP_001116078.1
(SEQ ID NO: 74)
MVCFRLFPVPGSGLVLVCLVLGAVRSYALELNLTDSENATCLYAKWQMNFTVRYETTNKTYKTVTISDHG
TVTYNGSICGDDQNGPKIAVQFGPGFSWIANFTKAASTYSIDSVSFSYNTGDNTTFPDAEDKGILTVDEL
LAIRIPLNDLFRCNSLSTLEKNDVVQHYWDVLVQAFVQNGTVSTNEFLCDKDKTSTVAPTIHTTVPSPTT
TPTPKEKPEAGTYSVNNGNDTCLLATMGLQLNITQDKVASVININPNTTHSTGSCRSHTALLRLNSSTIK
YLDFVFAVKNENRFYLKEVNISMYLVNGSVFSIANNNLSYWDAPLGSSYMCNKEQTVSVSGAFQINTFDL
RVQPFNVTQGKYSTAEECSADSDLNFLIPVAVGVALGFLIIVVFISYMIGRRKSRTGYQSV

Isoform/Variant includes: (Isoform A Precursor) NM_002294.3 and NP_002285.1; (Isoform B Precursor) NM_013995.2 and NP_054701.1

CXCR1 - NM_000634.3
(SEQ ID NO: 75)
GCTGAAACTGAAGAGGACATGTCAAATATTACAGATCCACAGATGTGGGATTTTGATGATCTAAATTTCA
CTGGCATGCCACCTGCAGATGAAGATTACAGCCCCTGTATGCTAGAAACTGAGACACTCAACAAGTATGT
TGTGATCATCGCCTATGCCCTAGTGTTCCTGCTGAGCCTGCTGGGAAACTCCCTGGTGATGCTGGTCATC
TTATACAGCAGGGTCGGCCGCTCCGTCACTGATGTCTACCTGCTGAACCTGGCCTTGGCCGACCTACTCT
TTGCCCTGACCTTGCCCATCTGGGCCGCCTCCAAGGTGAATGGCTGGATTTTTGGCACATTCCTGTGCAA
GGTGGTCTCACTCCTGAAGGAAGTCAACTTCTACAGTGGCATCCTGCTGTTGGCCTGCATCAGTGTGGAC
CGTTACCTGGCCATTGTCCATGCCACACGCACACTGACCCAGAAGCGTCACTTGGTCAAGTTTGTTTGTC
TTGGCTGCTGGGGACTGTCTATGAATCTGTCCCTGCCCTTCTTCCTTTTCCGCCAGGCTTACCATCCAAA
CAATTCCAGTCCAGTTTGCTATGAGGTCCTGGGAAATGACACAGCAAAATGGCGGATGGTGTTGCGGATC
CTGCCTCACACCTTTGGCTTCATCGTGCCGCTGTTTGTCATGCTGTTCTGCTATGGATTCACCCTGCGTA
CACTGTTTAAGGCCCACATGGGGCAGAAGCACCGAGCCATGAGGGTCATCTTTGCTGTCGTCCTCATCTT
CCTGCTTTGCTGGCTGCCCTACAACCTGGTCCTGCTGGCAGACACCCTCATGAGGACCCAGGTGATCCAG
GAGAGCTGTGAGCGCCGCAACAACATCGGCCGGGCCCTGGATGCCACTGAGATTCTGGGATTTCTCCATA
GCTGCCTCAACCCCATCATCTACGCCTTCATCGGCCAAAATTTTCGCCATGGATTCCTCAAGATCCTGGC
TATGCATGGCCTGGTCAGCAAGGAGTTCTTGGCACGTCATCGTGTTACCTCCTACACTTCTTCGTCTGTC
AATGTCTCTTCCAACCTCTGAAAACCATCGATGAAGGAATATCTCTTCTCAGAAGGAAAGAATAACCAAC
ACCCTGAGGTTGTGTGTGGAAGGTGATCTGGCTCTGGACAGGCACTATCTGGGTTTTGGGGGGACGCTAT
AGGATGTGGGGAAGTTAGGAACTGGTGTCTTCAGGGGCCACACCAACCTTCTGAGGAGCTGTTGAGGTAC
CTCCAAGGACCGGCCTTTGCACCTCCATGGAAACGAAGCACCATCATTCCCGTTGAACGTCACATCTTTA
ACCCACTAACTGGCTAATTAGCATGGCCACATCTGAGCCCCGAATCTGACATTAGATGAGAGAACAGGGC
TGAAGCTGTGTCCTCATGAGGGCTGGATGCTCTCGTTGACCCTCACAGGAGCATCTCCTCAACTCTGAGT
GTTAAGCGTTGAGCCACCAAGCTGGTGGCTCTGTGTGCTCTGATCCGAGCTCAGGGGGGTGGTTTTCCCA
TCTCAGGTGTGTTGCAGTGTCTGCTGGAGACATTGAGGCAGGCACTGCCAAAACATCAACCTGCCAGCTG
GCCTTGTGAGGAGCTGGAAACACATGTTCCCCTTGGGGGTGGTGGATGAACAAAGAGAAAGAGGGTTTGG
AAGCCAGATCTATGCCACAAGAACCCCCTTTACCCCCATGACCAACATCGCAGACACATGTGCTGGCCAC
CTGCTGAGCCCCAAGTGGAACGAGACAAGCAGCCCTTAGCCCTTCCCCTCTGCAGCTTCCAGGCTGGCGT
GCAGCATCAGCATCCCTAGAAAGCCATGTGCAGCCACCAGTCCATTGGGCAGGCAGATGTTCCTAATAAA
GCTTCTGTTCCGTGCTTGTCCCTGTGGAAGTATCTTGGTTGTGACAGAGTCAAGGGTGTGTGCAGCATTG
TTGGCTGTTCCTGCAGTAGAATGGGGGCAGCACCTCCTAAGAAGGCACCTCTCTGGGTTGAAGGGCAGTG
TTCCCTGGGGCTTTAACTCCTGCTAGAACAGTCTCTTGAGGCACAGAAACTCCTGTTCATGCCCATACCC
CTGGCCAAGGAAGATCCCTTTGTCCACAAGTAAAAGGAAATGCTCCTCCAGGGAGTCTCAGCTTCACCCT
GAGGTGAGCATCATCTTCTGGGTTAGGCCTTGCCTAGGCATAGCCCTGCCTCAAGCTATGTGAGCTCACC
AGTCCCTCCCCAAATGCTTTCCATGAGTTGCAGTTTTTTCCTAGTCTGTTTTCCCTCCTTGGAGACAGGG
CCCTGTCGGTTTATTCACTGTATGTCCTTGGTGCCTGGAGCCTACTAAATGCTCAATAAATAATGATCAC
AGGAATGAA
NP_000625.1
(SEQ ID NO: 76)
MSNITDPQMWDFDDLNFTGMPPADEDYSPCMLETETLNKYVVIIAYALVFLLSLLGNSLVMLVILYSRVG
RSVTDVYLLNLALADLLFALTLPIWAASKVNGWIFGTFLCKVVSLLKEVNFYSGILLLACISVDRYLAIV
HATRTLTQKRHLVKFVCLGCWGLSMNLSLPFFLFRQAYHPNNSSPVCYEVLGNDTAKWRMVLRILPHTFG
FIVPLFVMLFCYGFTLRTLFKAHMGQKHRAMRVIFAVVLIFLLCWLPYNLVLLADTLMRTQVIQESCERR
NNIGRALDATEILGFLHSCLNPIIYAFIGQNFRHGFLKILAMHGLVSKEFLARHRVTSYTSSSVNVSSNL
CCR6 - (Variant 1) NM_004367.5
(SEQ ID NO: 77)
CTCCGATCCAGAACACTACCGCGCAGCAAAATCATCTGCCTTGTTGTGTGGGGGCTGTCAGTCATCATCT
CCAGCTCAACTTTTGTCTTCAACCAAAAATACAACACCCAAGGCAGCGATGTCTGTGAACCCAAGTACCA
GACTGTCTCGGAGCCCATCAGGTGGAAGCTGCTGATGTTGGGGCTTGAGCTACTCTTTGGTTTCTTTATC
CCTTTGATGTTCATGATATTTTGTTACACGTTCATTGTCAAAACCTTGGTGCAAGCTCAGAATTCTAAAA
GGCACAAAGCCATCCGTGTAATCATAGCTGTGGTGCTTGTGTTTCTGGCTTGTCAGATTCCTCATAACAT
GGTCCTGCTTGTGACGGCTGCAAATTTGGGTAAAATGAACCGATCCTGCCAGAGCGAAAAGCTAATTGGC
TATACGAAAACTGTCACAGAAGTCCTGGCTTTCCTGCACTGCTGCCTGAACCCTGTGCTCTACGCTTTTA
TTGGGCAGAAGTTCAGAAACTACTTTCTGAAGATCTTGAAGGACCTGTGGTGTGTGAGAAGGAAGTACAA
GTCCTCAGGCTTCTCCTGTGCCGGGAGGTACTCAGAAAACATTTCTCGGCAGACCAGTGAGACCGCAGAT
AACGACAATGCGTCGTCCTTCACTATGTGATAGAAAGCTGAGTCTCCCTAAGGCATGTGTGAAACATACT
CATAGATGTTATGCAAAAAAAAGTCTATGGCCAGGTATGCATGGAAAATGTGGGAATTAAGCAAAATCAA
GCAAGCCTCTCTCCTGCGGGACTTAACGTGCTCATGGGCTGTGTGATCTCTTCAGGGTGGGGTGGTCTCT
GATAGGTAGCATTTTCCAGCACTTTGCAAGGAATGTTTTGTAGCTCTAGGGTATATATCCGCCTGGCATT
TCACAAAACAGCCTTTGGGAAATGCTGAATTAAAGTGAATTGTTGACAAATGTAAACATTTTCAGAAATA
TTCATGAAGCGGTCACAGATCACAGTGTCTTTTGGTTACAGCACAAAATGATGGCAGTGGTTTGAAAAAC
TAAAACAGAAAAAAAAATGGAAGCCAACACATCACTCATTTTAGGCAAATGTTTAAACATTTTTATCTAT
CAGAATGTTTATTGTTGCTGGTTATAAGCAGCAGGATTGGCCGGCTAGTGTTTCCTCTCATTTCCCTTTG
ATACAGTCAACAAGCCTGACCCTGTAAAATGGAGGTGGAAAGACAAGCTCAAGTGTTCACAACCTGGAAG
TGCTTCGGGAAGAAGGGGACAATGGCAGAACAGGTGTTGGTGACAATTGTCACCAATTGGATAAAGCAGC
TCAGGTTGTAGTGGGCCATTAGGAAACTGTCGGTTTGCTTTGATTTCCCTGGGAGCTGTTCTCTGTCGTG
AGTGTCTCTTGTCTAAACGTCCATTAAGCTGAGAGTGCTATGAAGACAGGATCTAGAATAATCTTGCTCA
CAGCTGTGCTCTGAGTGCCTAGCGGAGTTCCAGCAAACAAAATGGACTCAAGAGAGATTTGATTAATGAA
TCGTAATGAAGTTGGGGTTTATTGTACAGTTTAAAATGTTAGATGTTTTTAATTTTTTAAATAAATGGAA
TACTTTTTTTTTTTTTTTAAAGAAAGCAACTTTACTGAGACAATGTAGAAAGAAGTTTTGTTCCGTTTCT
TTAATGTGGTTGAAGAGCAATGTGTGGCTGAAGACTTTTGTTATGAGGAGCTGCAGATTAGCTAGGGGAC
AGCTGGAATTATGCTGGCTTCTGATAATTATTTTAAAGGGGTCTGAAATTTGTGATGGAATCAGATTTTA
ACAGCTCTCTTCAATGACATAGAAAGTTCATGGAACTCATGTTTTTAAAGGGCTATGTAAATATATGAAC
ATTAGAAAAATAGCAACTTGTGTTACAAAAATACAAACACATGTTAGGAAGGTACTGTCATGGGCTAGGC
ATGGTGGCTCACACCTGTAATCCCAGCATTTTGGGAAGCTAAGATGGGTGGATCACTTGAGGTCAGGAGT
TTGAGACCAGCCTGGCCAACATGGCGAAACCCCTCTCTACTAAAAATACAAAAATTTGCCAGGCGTGGTG
GCGGGTGCCTGTAATCCCAGCTACTTGGGAGGCTGAGGCAAGAGAATCGCTTGAACCCAGGAGGCAGAGG
TTGCAGTGAGCCGAGATCGTGCCATTGCACTCCAGCCTGGGTGACAAAGCGAGACTCCATCTCAAAAAAA
AAAAAAAAAAAAAAGGAAAGAACTGTCATGTAAACATACCAACATGTTTAAACCTGACAATGGTGTTATT
TGAAACTTTATATTGTTCTTGTAAGCTTTAACTATATCTCTCTTTAAAATGCAAAATAATGTCTTAAGAT
TCAAAGTCTGTATTTTTAAAGCATGGCTTTGGCTTTGCAAAATAAAAAATGTGTTTTGTACATGAA
(Variant 1) NP_004358.2
(SEQ ID NO: 78)
MSGESMNFSDVFDSSEDYFVSVNTSYYSVDSEMLLCSLQEVRQFSRLFVPIAYSLICVFGLLGNILVVIT
FAFYKKARSMTDVYLLNMAIADILFVLTLPFWAVSHATGAWVFSNATCKLLKGIYAINFNCGMLLLTCIS
MDRYIAIVQATKSFRLRSRTLPRSKIICLVVWGLSVIISSSTFVFNQKYNTQGSDVCEPKYQTVSEPIRW
KLLMLGLELLFGFFIPLMFMIFCYTFIVKTLVQAQNSKRHKAIRVIIAVVLVFLACQIPHNMVLLVTAAN
LGKMNRSCQSEKLIGYTKTVTEVLAFLHCCLNPVLYAFIGQKFRNYFLKILKDLWCVRRKYKSSGFSCAG
RYSENISRQTSETADNDNASSFTM

Isoform/Variant includes: (Variant 2) NM_031409.3 and NP_113597.2

GYP A - (Isoform 2 precursor)
NM_001308187.2
(SEQ ID NO: 79)
AGGCTAAGGTCAGACACTGACACTTGCAGTTGTCTTTGGTAGTTTTTTTG
CACTAACTTCAGGAACCAGCTCATGATCTCAGGATGTATGGAAAAATAAT
CTTTGTATTACTATTGTCAGAAATTGTGAGCATATCAGCATTAAGTACCA
CTGAGGTGGCAATGCACACTTCAACTTCTTCTTCAGTCACAAAGAGTTAC
ATCTCATCACAGACAAATGATACGCACAAACGGGACACATATGCAGCCAC
TCCTAGAGCTCATGAAGTTTCAGAAATTTCTGTTAGAACTGTTTACCCTC
CAGAAGAGGAAACCGAGATAACACTCATTATTTTTGGGGTGATGGCTGGT
GTTATTGGAACGATCCTCTTAATTTCTTACGGTATTCGCCGACTGATAAA
GAAAAGCCCATCTGATGTAAAACCTCTCCCCTCACCTGACACAGACGTGC
CTTTAAGTTCTGTTGAAATAGAAAATCCAGAGACAAGTGATCAATGAGAA
TCTGTTCACCAAACCAAATGTGGAAAGAACACAAAGAAGACATAAGACTT
CAGTCAAGTGAAAAATTAACATGTGGACTGGACACTCCAATAAATTATAT
ACCTGCCTAAGTTGTACAATTTCAGAATGCAATTTTCATTATAATGAGTT
CCAGTGACTCAATGATGGGGAAAAAAATCTCTGCTCATTAATATTTCAAG
ATAAAGAACAAATGTTTCCTTGAATGCTTGCTTTTGTGTGTTAGCATAAT
TTTTAGAATTGTTTGAGAATTCTGATCCAAAACTTTAGTTGAATTCATCT
ACGTTTGTTTAATATTAACTTAACCTATTCTATTGTATTATAATGATGAT
TCTGTCAAATGAAAGGCTTGAAATACCTAGATGAAGTTTAGATTTTCTTC
CTATTGTAAACTTTTGAGTCTGGTTTCATTGTTTTAAATAAATTAAGGGG
ACACTAAAGTCCTATCATTCATTTCCTTCATTGCTGAACAGGCAAGATAT
AATATTACATGAATGATTACTATATTTTGTTCACACTAATAAAGCTTATG
CTCAGAAATGCCATACACACACACACACACACACACAAACACACACATTT
ATCATTTAATGCATAAATCAACACAAAAGGTTTTCCCATTAATATGAAAT
ATTACATATATATAAGTGCCATATTTAAAATAATTTGTCTAACAGTAGAA
CTGTGTCGGAGCACTCACTGAAGCTTGCATTCCACTGAAAGAGTTATTTG
TGTAAGTAGAGTATCCGGAGAAGGAAAAGAACTTACGACCTTTCTTTATA
ACAGAAACTCAACTCTAAATTCAACAAGATGTGCAAACCGGACATGCAGG
TGAATATTTTAATAGGTTACTATAAGGTTCTCAATTAAATTCTTTAATCT
GTCCAGTCCCAGTTTCTCTTATTAATAAAACTTTGGAAATTGCTTTAAAC
CATTTAAAGGAAATTTCTAGATATAGAAACTAAGGACTGTGACTATACAG
CTGTCACTCATTTGTAGTAAAACTTAAAAAGCAAAAACAAAAAACAAAAA
AGACCTTCCTGTGATACTTTATTTCCGAACTAATAAAAATCTATATGACT
TTTTATTATTGTGTGATAACCAAGTAAATGTTTTCTATTTTGCATATTTT
CAGGCATGGTAACAGAAATTTACCTTTTAATAAATTAAAAAATCTAAATT
TTAACCTACTTGTATGTTCGGAGAGTGTTTTTGTACTATATTGACTACTT
AAAATAGAGAATGAGACTAAGAAGGGAACATTTCTGTTGATACATGTTTT
TTAAAAGTAATTTTAAGAGCATTATTAGGTTAATTAATCCAATTAATGAC
CCAAATGCCAAGGTAATTTTAAATTTACATTTTTAATAAAAGCAACATGT
TGAAACAAGAGAGGGTGAGATTAACCTTTTTGCTAAAGTAATTTACAAGT
CAAAGACAGGAAGAGATCAGAGTGAATGTGCCTTCTTAACCAGAGCTACA
GAATTTAGTGAATAATTAAAGTACAAACTGCTTTGACCTCCTTGAACTTT
TCCAAGCAATTTCTCTGTACTTCTATATATGAATGTCTTAGCCAATTTTC
TGCTACTATAACAGAATACGACAGACTGGGTAATTTAAAAAGAAAAGAAA
TTTATTTTCTTCCTAGTTCTGGAGGCTGGGAAGGCGAAGGGCATGGCACT
GACATCTGCCTTGTAACTGATGAGAACCTTCTTACTGCATGATAACAAAG
CAGCAAGGCAAGCAAAAGCGTAAGATGAAGAGAGAGGAAATGAAGCCAAA
CACATCCTTTCATCAGAAGCCCATTCCCTCTATAAGGCGTTATTACATTT
ATGAGAATGGAGTCCTCATGACCTAATCGTGACCTTAAAGGCCCCTCCCA
ACACTGTTACAATGGCAATTAAATTTCAACAAAGGTTCCAGAGGTGACAT
TCGAATCAGCAATGAAATTTTCATAGTTAAATTTGGTATTCGTGGGGGAA
GAAATGACCATTTCCCTTGTATTTTTATAATTAAATCAGCAAAATATTGT
AATAAAGAAATCTTTCCTGTGAAGATACCATGACCCCA
(Isoform 2 precursor)
NP_001295116.1
(SEQ ID NO: 80)
MYGKIIFVLLLSEIVSISALSTTEVAMHTSTSSSVTKSYISSQTNDTHKR
DTYAATPRAHEVSEISVRTVYPPEEETEITLIIFGVMAGVIGTILLISYG
IRRLIKKSPSDVKPLPSPDTDVPLSSVEIENPETSDQ

Isoform/Variant includes: (Isoform 3) NM 001308190.2 and NP_001295119.1; (Isoform 1 precursor) NM_002099.8 and NP_002090.4

HLA-A - (Variant 2 A*01:01:01:01 Allele)
NM_00124275 8.1
(SEQ ID NO: 81)
GAGAAGCCAATCAGTGTCGTCGCGGTCGCTGTTCTAAAGTCCGCACGCAC
CCACCGGGACTCAGATTCTCCCCAGACGCCGAGGATGGCCGTCATGGCGC
CCCGAACCCTCCTCCTGCTACTCTCGGGGGCCCTGGCCCTGACCCAGACC
TGGGCGGGCTCCCACTCCATGAGGTATTTCTTCACATCCGTGTCCCGGCC
CGGCCGCGGGGAGCCCCGCTTCATCGCCGTGGGCTACGTGGACGACACGC
AGTTCGTGCGGTTCGACAGCGACGCCGCGAGCCAGAAGATGGAGCCGCGG
GCGCCGTGGATAGAGCAGGAGGGGCCGGAGTATTGGGACCAGGAGACACG
GAATATGAAGGCCCACTCACAGACTGACCGAGCGAACCTGGGGACCCTGC
GCGGCTACTACAACCAGAGCGAGGACGGTTCTCACACCATCCAGATAATG
TATGGCTGCGACGTGGGGCCGGACGGGCGCTTCCTCCGCGGGTACCGGCA
GGACGCCTACGACGGCAAGGATTACATCGCCCTGAACGAGGACCTGCGCT
CTTGGACCGCGGCGGACATGGCAGCTCAGATCACCAAGCGCAAGTGGGAG
GCGGTCCATGCGGCGGAGCAGCGGAGAGTCTACCTGGAGGGCCGGTGCGT
GGACGGGCTCCGCAGATACCTGGAGAACGGGAAGGAGACGCTGCAGCGCA
CGGACCCCCCCAAGACACATATGACCCACCACCCCATCTCTGACCATGAG
GCCACCCTGAGGTGCTGGGCCCTGGGCTTCTACCCTGCGGAGATCACACT
GACCTGGCAGCGGGATGGGGAGGACCAGACCCAGGACACGGAGCTCGTGG
AGACCAGGCCTGCAGGGGATGGAACCTTCCAGAAGTGGGCGGCTGTGGTG
GTGCCTTCTGGAGAGGAGCAGAGATACACCTGCCATGTGCAGCATGAGGG
TCTGCCCAAGCCCCTCACCCTGAGATGGGAGCTGTCTTCCCAGCCCACCA
TCCCCATCGTGGGCATCATTGCTGGCCTGGTTCTCCTTGGAGCTGTGATC
ACTGGAGCTGTGGTCGCTGCCGTGATGTGGAGGAGGAAGAGCTCAGATAG
AAAAGGAGGGAGTTACACTCAGGCTGCAAGCAGTGACAGTGCCCAGGGCT
CTGATGTGTCTCTCACAGCTTGTAAAGTGTGAGACAGCTGCCTTGTGTGG
GACTGAGAGGCAAGAGTTGTTCCTGCCCTTCCCTTTGTGACTTGAAGAAC
CCTGACTTTGTTTCTGCAAAGGCACCTGCATGTGTCTGTGTTCGTGTAGG
CATAATGTGAGGAGGTGGGGAGAGCACCCCACCCCCATGTCCACCATGAC
CCTCTTCCCACGCTGACCTGTGCTCCCTCTCCAATCATCTTTCCTGTTCC
AGAGAGGTGGGGCTGAGGTGTCTCCATCTCTGTCTCAACTTCATGGTGCA
CTGAGCTGTAACTTCTTCCTTCCCTATTAAAATTAGAACCTGAGTATAAA
TTTACTTTCTCAAATTCTTGCCATGAGAGGTTGATGAGTTAATTAAAGGA
GAAGATTCCTAAAATTTGAGAGACAAAATTAATGGAACGCATGAGAACCT
TCCAGAGTCCA
(Variant 2 A*01:01:01:01 Allele)
NP_001229687.1
(SEQ ID NO: 82)
MAVMAPRTLLLLLSGALALTQTWAGSHSMRYFFTSVSRPGRGEPRFIAVG
YVDDTQFVRFDSDAASQKMEPRAPWIEQEGPEYWDQETRNMKAHSQTDRA
NLGTLRGYYNQSEDGSHTIQIMYGCDVGPDGRFLRGYRQDAYDGKDYIAL
NEDLRSWTAADMAAQITKRKWEAVHAAEQRRVYLEGRCVDGLRRYLENGK
ETLQRTDPPKTHMTHHPISDHEATLRCWALGFYPAEITLTWQRDGEDQTQ
DTELVETRPAGDGTFQKWAAVVVPSGEEQRYTCHVQHEGLPKPLTLRWEL
SSQPTIPIVGIIAGLVLLGAVITGAVVAAVMWRRKSSDRKGGSYTQAASS
DSAQGSDVSLTACKV

Isoform/Variant includes: (Variant 1 A*03:01:01:01 Allele) NM_002116.8 and NP_002107.3

In some embodiments therefore, the disclosed methods further comprise isolating one or a plurality of enteric neurons thus generated by exposing the neurons to antibodies specific to one or a plurality of surface antigens specific for the NOS-expressing enteric neurons. In some embodiments, the antibodies specific to the one or plurality of surface antigens specific for the NOS-expressing enteric neurons are immobilized onto a solid support. In some embodiments, the disclosed methods comprise isolating one or a plurality of enteric neurons by exposing the neurons to an antibody specific for CD47. In some embodiments, the disclosed methods comprise isolating one or a plurality of enteric neurons by exposing the neurons to an antibody specific for CD58. In some embodiments, the disclosed methods comprise isolating one or a plurality of enteric neurons by exposing the neurons to an antibody specific for CD59. In some embodiments, the disclosed methods comprise isolating one or a plurality of enteric neurons by exposing the neurons to an antibody specific for CD90. In some embodiments, the disclosed methods comprise isolating one or a plurality of enteric neurons by exposing the neurons to an antibody specific for CD181. In some embodiments, the disclosed methods comprise isolating one or a plurality of enteric neurons by exposing the neurons to an antibody specific for CD235a. An antibody specific for NOS1 can also be included in this isolation procedure. In some embodiments therefore, the disclosed methods comprise isolating one or a plurality of enteric neurons by exposing the neurons to an antibody specific for one or a combination of: CD47, CD58, CD59, CD90, CD181, CD235a and/or NOS1.

Commercially available antibodies to any of the aforementioned surface antigens specific for the NOS-expressing enteric neurons can be used. Exemplary antibodies useful for the disclosed methods include, but not limited to, the following:

| Surface | |
|---|---|
| Marker or | |
| Antigen | Antibody |
| CD47 | Anti-CD47 antibody [SP279] (ab226837) by Abcam |
| | Anti-CD47 antibody [EPR21794] (ab218810) by Abcam |
| | Anti-CD47 antibody [CD47/3019] (ab260419) by Abcam |
| | CD47 Monoclonal Antibody (B6H12) by Invitrogen |
| | CD47 Monoclonal Antibody (miap301) by Invitrogen |
| | CD47 Monoclonal Antibody (2D3) by Invitrogen |
| | Human CD47 Antibody MAB4670 by R&D Systems |
| CD58 | Anti-CD58 antibody [TS2/9] (ab171087) by Abcam |
| | Anti-CD58 antibody [EP15041] (ab196648) by Abcam |
| | Anti-CD58 antibody [EPR24012-147] (ab275392) by Abcam |
| | CD58 Monoclonal Antibody (TS2/9) by Invitrogen |
| | CD58 Recombinant Rabbit Monoclonal Antibody (85) |
| | by Invitrogen |
| CD59 | Anti-CD59 antibody [EPR6425(2)] (ab133707) by Abcam |
| | Anti-CD59 antibody [EPR22394-242] (ab253239) by Abcam |
| | Anti-CD59 antibody [MEM-43/5] (ab9183) by Abcam |
| | CD59 Monoclonal Antibody (MEM-43) by Invitrogen |
| | CD59 Monoclonal Antibody (2E11B6) by Invitrogen |
| CD90 | Recombinant Anti-CD90/Thy1 antibody [EPR3133] |
| | (ab133350) by Abcam |
| | CD90/Thy1 Antibody (OX-7) by Novus Biologicals |
| | CD90.1 (Thy-1.1) Monoclonal Antibody (HIS51) by |
| | Invitrogen |
| | CD90.2 (Thy-1.2) Monoclonal Antibody (53-2.1) by |
| | Invitrogen |
| CD181 | CD181 (CXCR1) Monoclonal Antibody (eBio8F1-1-4 |
| | (8F1-1-4)) by Invitrogen |
| | Monoclonal Mouse anti-Human CXCR1 Antibody (clone |
| | 2B8A1, WB) by LSBio |
| | APC anti-human CD181 (CXCR1) Antibody (clone |
| | 8F1/CXCR1) by BioLegend |
| CD235a | Anti-Glycophorin A antibody [EPR8200] (ab129024) |
| | by Abcam |
| | Anti-Glycophorin A antibody [EPR8199] (ab134111) |
| | by Abcam |
| | CD235a (Glycophorin A) Monoclonal Antibody (HIR2 |
| | (GA-R2)) by Invitrogen |
| | FITC anti-human CD235a (Glycophorin A) Antibody (clone |
| | HI264) by BioLegend |
| NOS1 | Anti-nNOS (neuronal) antibody [EP1855Y] (ab76067) |
| | by Abcam |
| | Anti-nNOS (neuronal) antibody (ab5586) by Abcam |
| | nNOS Monoclonal Antibody (3G6B10) by Invitrogen |

The one or a plurality of enteric neurons produced by the methods of the disclosure may be separated by any method known in the art such as FACS (fluorescent activated cell sorting) and MACS (magnetic activated cell sorting). Methods other than FACS and MACS may also be used. In some embodiments, the enteric neurons are separated using FACS. In other embodiments, the enteric neurons are separated using MACS.

Fluorescence-activated cell sorting (FACS) is a specialized type of flow cytometry. It provides a method for sorting a heterogeneous mixture of biological cells into two or more containers, one cell at a time, based upon the specific light scattering and fluorescent characteristics of each cell. It provides a method for sorting a heterogeneous mixture of biological cells into two or more containers, one cell at a time, based upon the specific light scattering and fluorescent characteristics of each cell. It is a useful scientific instrument, as it provides fast, objective and quantitative recording of fluorescent signals from individual cells as well as physical separation of cells of particular interest.

In FACS, the cell suspension is entrained in the center of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell per droplet. Just before the stream breaks into droplets, the flow passes through a fluorescence measuring station where the fluorescent character of interest of each cell is measured. An electrical charging ring is placed just at the point where the stream breaks into droplets. A charge is placed on the ring based on the immediately prior fluorescence intensity measurement, and the opposite charge is trapped on the droplet as it breaks from the stream. The charged droplets then fall through an electrostatic deflection system that diverts droplets into containers based upon their charge. In some systems, the charge is applied directly to the stream, and the droplet breaking off retains charge of the same sign as the stream. The stream is then returned to neutral after the droplet breaks off.

A wide range of fluorophores can be used as labels in flow cytometry. Fluorophores, or simply “fluors,” are typically attached to an antibody that recognizes a target feature on or in the cell; they may also be attached to a chemical entity with affinity for the cell membrane or another cellular structure. Each fluorophore has a characteristic peak excitation and emission wavelength, and the emission spectra often overlap. Consequently, the combination of labels which can be used depends on the wavelength of the lamp(s) or laser(s) used to excite the fluorochromes and on the detectors available.

The disclosure further relates to a method of evaluating a neuromodulatory effect of an agent, the method comprising: a) culturing the enteric neurons produced by the methods disclosed herein in the presence or absence of the agent; and b) detecting and/or measuring nitric oxide released by the agent, wherein a detectable level of nitric oxide in the presence of the agent is indicative of a neuromodulatory effect, and no detectable level of nitric oxide in the presence of the agent is indicative of the agent not conferring a neuromodulatory effect. The disclosure also relates to a method for screening an agent capable of modulating calcium influx, the method comprising: a) culturing the enteric neurons produced by the methods disclosed herein in the presence or absence of the agent; and b) detecting and/or measuring nitric oxide released by the agent; wherein a detectable level of nitric oxide in the presence of the agent is indicative of the agent capable of modulating calcium influx, and no detectable level of nitric oxide in the presence of the agent is indicative of the agent not conferring ability to modulate calcium influx. The disclosure additionally relates to a method of measuring or quantifying a neuromodulatory effect of an agent, the method comprising: a) culturing the enteric neurons produced by the methods disclosed herein in the presence or absence of the agent; and b) detecting and/or measuring nitric oxide released by the agent, wherein a detectable level of nitric oxide in the presence of the agent is indicative of a neuromodulatory effect, and no detectable level of nitric oxide in the presence of the agent is indicative of the agent not conferring a neuromodulatory effect. In some embodiments, at least about 30% of the enteric neurons used in such methods express nitric oxide synthase. In some embodiments, at least about 40% of the enteric neurons used in such methods express nitric oxide synthase. In some embodiments, at least about 50% of the enteric neurons used in such methods express nitric oxide synthase. In some embodiments, at least about 60% of the enteric neurons used in such methods express nitric oxide synthase. In some embodiments, at least about 70% of the enteric neurons used in such methods express nitric oxide synthase. In some embodiments, at least about 80% of the enteric neurons used in such methods express nitric oxide synthase. In some embodiments, at least about 90% of the enteric neurons used in such methods express nitric oxide synthase.

The disclosure also relates to a method of treating a gastrointestinal motility disorder in a subject in need thereof comprising administering to the subject a pharmaceutical composition comprising a therapeutically effective amount of any of the enteric neurons disclosed herein and a pharmaceutically acceptable carrier. In some embodiments, the gastrointestinal motility disorder is chosen from one or a combination of: gastroparesis, esophageal achalasia, chronic intestinal pseudo-obstruction, and hypertrophic pyloric stenosis. In some embodiments, the method comprises transplanting one or a plurality of the enteric neurons into one or more affected organs of the subject. In some embodiments, the method comprises engrafted the enteric neurons of the pharmaceutical composition in the intestine, stomach or esophagus of the subject.

In some embodiments, the composition comprising any of the enteric neurons disclosed herein is administered at or within a tolerated difference of a desired dose of total cells, such as a desired dose of enteric neurons (or cells). In some embodiments, the desired dose is a desired number of cells, a desired number of cells per unit of body surface area or a desired number of cells per unit of body weight of the subject to whom the cells are administered, e.g., cells/m² or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m² or cells/kg. In some aspects, the desired dose is at or above a minimum number of cells of the population, or minimum number of cells of the population per unit of body surface area or body weight.

In certain embodiments, the disclosed composition is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells is within a range of between at or about 10⁴ and at or about 10⁹ cells/meter² (m²) body surface area, such as between 10⁵ and 10⁶ cells/m² body surface area, for example, at or about 1×10⁵ cells/m², 1.5×10⁵ cells/m², 2×10⁵ cells/m², or 1×10⁶ cells/m² body surface area. For example, in some embodiments, the cells are administered at, or within a certain range of error of, between at or about 10⁴ and at or about 10⁹ cells/meter² (m²) body surface area, such as between 10⁵ and 10⁶ cells/m² body surface area, for example, at or about 1×10⁵ cells/m², 1.5×10⁵ cells/m², 2×10⁵ cells/m², or 1×10⁶ cells/m² body surface area.

In some embodiments, the cells are administered at or within a certain range of error of between at or about 10⁴ and at or about 10⁹ cells/meter² (m²) body weight, such as between 10⁵ and 10⁶ cells/m² body weight, for example, at or about 1×10⁵ cells/m², 1.5×10⁵ cells/m², 2×10⁵ cells/kg, or 1×10⁶ cells/m² body surface area.

Compositions and Systems

Also provided in the disclosure are compositions and systems comprising any of the enteric neurons disclosed herein. In some embodiments, the enteric neurons are produced by any of the disclosed methods. In some embodiments, at least about 20% of the enteric neurons comprised in the disclosed compositions and systems express nitric oxide synthase. In some embodiments, at least about 30% of the enteric neurons comprised in the disclosed compositions and systems express nitric oxide synthase. In some embodiments, at least about 40% of the enteric neurons comprised in the disclosed compositions and systems express nitric oxide synthase. In some embodiments, at least about 50% of the enteric neurons comprised in the disclosed compositions and systems express nitric oxide synthase. In some embodiments, at least about 60% of the enteric neurons comprised in the disclosed compositions and systems express nitric oxide synthase. In some embodiments, at least about 70% of the enteric neurons comprised in the disclosed compositions and systems express nitric oxide synthase. In some embodiments, at least about 80% of the enteric neurons comprised in the disclosed compositions and systems express nitric oxide synthase. In some embodiments, at least about 90% of the enteric neurons comprised in the disclosed compositions and systems express nitric oxide synthase. In some embodiments, more than about 90% of the enteric neurons comprised in the disclosed compositions and systems express nitric oxide synthase.

In some embodiments, the nitric oxide synthase expressed in the enteric neurons comprised in the disclosed compositions and systems comprises at least about 70%, 75%, 80%, 85%, 90%, 95%, 96%, 97%, 98% or 99% sequence identity to SEQ ID NO: 10 or a functional fragment thereof. In some embodiments, the nitric oxide synthase comprises at least about 70%, 75%, 80%, 85%, 90%, 95%, 96%, 97%, 98% or 99% sequence identity to SEQ ID NO: 11 or a functional fragment thereof In some embodiments, the nitric oxide synthase comprises at least about 70%, 75%, 80%, 85%, 90%, 95%, 96%, 97%, 98% or 99% sequence identity to SEQ ID NO: 12 or a functional fragment thereof. In some embodiments, the nitric oxide synthase comprises SEQ ID NO: 10 or a functional fragment thereof. In some embodiments, the nitric oxide synthase comprises SEQ ID NO: 11 or a functional fragment thereof In some embodiments, the nitric oxide synthase comprises SEQ ID NO: 12 or a functional fragment thereof.

In some embodiments, the disclosed compositions and systems further comprise enteric neurons that express CHAT. In some embodiments, at least about 20% of the enteric neurons express CHAT. In some embodiments, at least about 30% of the enteric neurons express CHAT. In some embodiments, at least about 40% of the enteric neurons express CHAT. In some embodiments, at least about 50% of the enteric neurons express CHAT.

In some embodiments, the disclosed compositions and systems further comprise enteric neurons that express GABA. In some embodiments, at least about 5% of the enteric neurons express GABA. In some embodiments, at least about 7% of the enteric neurons express GABA. In some embodiments, at least about 9% of the enteric neurons express GABA. In some embodiments, at least about 11% of the enteric neurons express GABA. In some embodiments, at least about 13% of the enteric neurons express GABA. In some embodiments, at least about 15% of the enteric neurons express GABA.

In some embodiments, the disclosed compositions and systems further comprise enteric neurons that express 5-HT. In some embodiments, at least about 3% of the enteric neurons express 5-HT. In some embodiments, at least about 4% of the enteric neurons express 5-HT. In some embodiments, at least about 5% of the enteric neurons express 5-HT. In some embodiments, at least about 6% of the enteric neurons express 5-HT. In some embodiments, at least about 7% of the enteric neurons express 5-HT. In some embodiments, at least about 8% of the enteric neurons express 5-HT. In some embodiments, at least about 9% of the enteric neurons express 5-HT. In some embodiments, at least about 10% of the enteric neurons express 5-HT.

In some embodiments, the NOS1-expressing enteric neurons comprised in the disclosed compositions and systems are deficient in expression of CHAT. In some embodiments, the NOS1-expressing enteric neurons comprised in the disclosed compositions and systems are deficient in expression of GABA. In some embodiments, the NOS1-expressing enteric neurons comprised in the disclosed compositions and systems are deficient in expression of 5-HT. In some embodiments, the NOS1-expressing enteric neurons comprised in the disclosed compositions and systems are deficient in expression of CHAT and GABA. In some embodiments, the NOS1-expressing enteric neurons comprised in the disclosed compositions and systems are deficient in expression of CHAT and 5-HT. In some embodiments, the NOS1-expressing enteric neurons comprised in the disclosed compositions and systems are deficient in expression of GABA and 5-HT. In some embodiments, the NOS1-expressing enteric neurons comprised in the disclosed compositions and systems are deficient in expression of CHAT, GABA and 5-HT.

In some embodiments, the NOS1-expressing enteric neurons comprised in the disclosed compositions and systems are at least about 40 days in culture. In some embodiments, the NOS1-expressing enteric neurons comprised in the disclosed compositions and systems are at least about 45 days in culture. In some embodiments, the NOS1-expressing enteric neurons comprised in the disclosed compositions and systems are at least about 50 days in culture.

In some embodiments, the NOS1-expressing enteric neurons comprised in the disclosed compositions and systems are derived from cells that express CD49 in culture from about 12 to about 15 days. In some embodiments, the NOS1-expressing enteric neurons comprised in the disclosed compositions and systems are derived from cells that express SOX10 in culture from about 12 to about 15 days. In some embodiments, the NOS1-expressing enteric neurons comprised in the disclosed compositions and systems are derived from cells that express CD49 and SOX10 in culture from about 12 to about 15 days.

In some embodiments, the NOS1-expressing enteric neurons comprised in the disclosed compositions and systems are derived from cells that express TRKC in culture from about 15 to about 30 days. In some embodiments, the NOS1-expressing enteric neurons comprised in the disclosed compositions and systems are derived from cells that express PHOX2B in culture from about 15 to about 30 days. In some embodiments, the NOS1-expressing enteric neurons comprised in the disclosed compositions and systems are derived from cells that express EDNRB in culture from about 15 to about 30 days. In some embodiments, the NOS1-expressing enteric neurons comprised in the disclosed compositions and systems are derived from cells that express TRKC and PHOX2B in culture from about 15 to about 30 days. In some embodiments, the NOS1-expressing enteric neurons comprised in the disclosed compositions and systems are derived from cells that express TRKC and EDNRB in culture from about 15 to about 30 days. In some embodiments, the NOS1-expressing enteric neurons comprised in the disclosed compositions and systems are derived from cells that express PHOX2B and EDNRB in culture from about 15 to about 30 days. In some embodiments, the NOS1-expressing enteric neurons comprised in the disclosed compositions and systems are derived from cells that express TRKC, PHOX2B and EDNRB in culture from about 15 to about 30 days.

In some embodiments, the NOS1-expressing enteric neurons comprised in the disclosed compositions and systems are derived from cells that express TRKC in culture from about 30 days to about 45 days. In some embodiments, the NOS1-expressing enteric neurons comprised in the disclosed compositions and systems are derived from cells that express TUJ1 in culture from about 30 days to about 45 days. In some embodiments, the NOS1-expressing enteric neurons comprised in the disclosed compositions and systems are derived from cells that express TRKC and TUJ1 in culture from about 30 days to about 45 days.

In some embodiments, the NOS1-expressing enteric neurons comprised in the disclosed compositions and systems are derived from a combination of two or more types of inducible pluripotent stem cells. In some embodiments, the NOS1-expressing enteric neurons are derived from cells that are in culture for at least about 12 days and cells that express CD49 and SOX10 in culture from about 12 to about 15 days. In some embodiments, the NOS1-expressing enteric neurons are derived from cells that are in culture for at least about 12 days and cells that express TRKC, PHOX2B and EDNRB in culture from about 15 to about 30 days. In some embodiments, the NOS1-expressing enteric neurons are derived from cells that are in culture for at least about 12 days and cells that express TRKC and/or TUJ1 in culture from about 30 days to about 45 days. In some embodiments, the NOS1-expressing enteric neurons are derived from cells that express CD49 and SOX10 in culture from about 12 to about 15 days and cells that express TRKC, PHOX2B and EDNRB in culture from about 15 to about 30 days. In some embodiments, the NOS1-expressing enteric neurons are derived from cells that express CD49 and SOX10 in culture from about 12 to about 15 days and cells that express TRKC and/or TUJ1 in culture from about 30 days to about 45 days. In some embodiments, the NOS1-expressing enteric neurons are derived from cells that express TRKC, PHOX2B and EDNRB in culture from about 15 to about 30 days and cells that express TRKC and/or TUJ1 in culture from about 30 days to about 45 days. In some embodiments, the NOS1-expressing enteric neurons are derived from cells that are in culture for at least about 12 days, cells that express CD49 and SOX10 in culture from about 12 to about 15 days, and cells that express TRKC, PHOX2B and EDNRB in culture from about 15 to about 30 days. In some embodiments, the NOS1-expressing enteric neurons are derived from cells that are in culture for at least about 12 days, cells that express CD49 and SOX10 in culture from about 12 to about 15 days, and cells that express TRKC and/or TUJ1 in culture from about 30 days to about 45 days. In some embodiments, the NOS1-expressing enteric neurons are derived from cells that are in culture for at least about 12 days, cells that express TRKC, PHOX2B and EDNRB in culture from about 15 to about 30 days, and cells that express TRKC and/or TUJ1 in culture from about 30 days to about 45 days. In some embodiments, the NOS1-expressing enteric neurons are derived from cells that express CD49 and SOX10 in culture from about 12 to about 15 days, cells that express TRKC, PHOX2B and EDNRB in culture from about 15 to about 30 days, and cells that express TRKC and/or TUJ1 in culture from about 30 days to about 45 days. In some embodiments, the NOS1-expressing enteric neurons are derived from cells that are in culture for at least about 12 days, cells that express CD49 and SOX10 in culture from about 12 to about 15 days, cells that express TRKC, PHOX2B and EDNRB in culture from about 15 to about 30 days, and cells that express TRKC and/or TUJ1 in culture from about 30 days to about 45 days.

The enteric neurons comprised in the disclosed compositions and systems can be in form of a spheroid in any suitable width, length, thickness, and/or diameter. In some embodiments, a spheroid may have a width, length, thickness, and/or diameter in a range from about 10 μm to about 50,000 μm, or any range therein, such as, but not limited to, from about 10 μm to about 900 μm, about 100 μm to about 700 μm, about 300 μm to about 600 μm, about 400 μm to about 500 μm, about 500 μm to about 1,000 μm, about 600 μm to about 1,000 μm, about 700 μm to about 1,000 μm, about 800 μm to about 1,000 μm, about 900 μm to about 1,000 μm, about 750 m to about 1,500 μm, about 1,000 μm to about 5,000 μm, about 1,000 μm to about 10,000 μm, about 2,000 to about 50,000 μm, about 25,000 μm to about 40,000 μm, or about 3,000 μm to about 15,000 μm. In some embodiments, a spheroid may have a width, length, thickness, and/or diameter of about 50 μm, 100 μm, 200 μm, 300 μm, 400 μm, 500 μm, 600 μm, 700 μm, 800 μm, 900 μm, 1,000 μm, 5,000 μm, 10,000 μm, 20,000 μm, 30,000 μm, 40,000 μm, or 50,000 μm. In some embodiments, a plurality of spheroids are generated, and each of the spheroids of the plurality may have a width, length, thickness, and/or diameter that varies by less than about 20%, such as, for example, less than about 15%, 10%, or 5%. In some embodiments, each of the spheroids of the plurality may have a different width, length, thickness, and/or diameter within any of the ranges set forth above.

The cells in a spheroid may have a particular orientation. In some embodiments, the spheroid may comprise an interior core and an exterior surface. In some embodiments, the spheroid may be hollow (i.e., may not comprise cells in the interior). In some embodiments, the interior core cells and the exterior surface cells are different types of cell.

In some embodiments, spheroids may be made up of one, two, three or more different cell types, including one or a plurality of neuronal cell types and/or one or a plurality of stem cell types. In some embodiments, the interior core cells may be made up of one, two, three, or more different cell types. In some embodiments, the exterior surface cells may be made up of one, two, three, or more different cell types.

In some embodiments, the spheroids comprise at least two types of cells. In some embodiments the spheroids comprise neuronal cells and non-neuronal cells. In some embodiments, the spheroids comprise neuronal cells and astrocytes at a ratio of about 5:1, 4:1, 3:1, 2:1 or 1:1 of neuronal cells to astrocytes. In some embodiments, the spheroids comprise neuronal cells and non-neuronal cells at a ratio of about 5:1, 4:1, 3:1, 2:1 or 1:1. In some embodiments, the spheroids comprise neuronal cells and non-neuronal cells at a ratio of about 1:5: 1:4, 1:3, or 1:2. Any combination of cell types disclosed herein may be used in the above-identified ratios within the spheroids of the disclosure.

Depending on the particular embodiments, groups of cells may be placed according to any suitable shape, geometry, and/or pattern. For example, independent groups of cells may be deposited as spheroids, and the spheroids may be arranged within a three dimensional grid, or any other suitable three dimensional pattern. The independent spheroids may all comprise approximately the same number of cells and be approximately the same size, or alternatively, different spheroids may have different numbers of cells and different sizes. In some embodiments, multiple spheroids may be arranged in shapes such as an L or T shape, radially from a single point or multiple points, sequential spheroids in a single line or parallel lines, tubes, cylinders, toroids, hierarchically branched vessel networks, high aspect ratio objects, thin closed shells, organoids, or other complex shapes which may correspond to geometries of tissues, vessels or other biological structures.

The systems of the disclosure comprise a cell culture vessel comprising one or a plurality of the enteric neurons disclosed herein supported in a culture medium. In some embodiments, the cell culture vessel comprised in the disclosed system further comprises smooth muscle cells proximate to or adjacent to the plurality of enteric neurons. The term “culture vessel” as used herein is defined as any vessel suitable for growing, culturing, cultivating, proliferating, propagating, or otherwise similarly manipulating cells. A culture vessel may also be referred to herein as a “culture insert.” In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. In some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated stem cells and/or neural crest cells. In some embodiments, the culture vessel comprises enteric neurons. In some embodiments, the culture vessel comprises enteric neurons differentiated in culture form about 12 to about 20 days. In some embodiments, the culture vessel comprises a hydrogel and one or a plurality of isolated pluripotent stem cells.

In some embodiments, the system comprises a solid substrate comprises one or a plurality of vessels or wells within which a flat or round bottom surface is surrounded by plastic walls. In some embodiments, the bottom surface is coasted with one or more layers of hydrogel. In some embodiments, the bottom surface is coated with one or more layers of vitronection and/or fibronectin. In some embodiments, the bottom surface is coated with one or more layers of vitronection and/or fibronectin at concentrations at or about the concentrations as disclosed in the Examples.

In some embodiments, the hydrogel or hydrogel matrixes can have various thicknesses. In some embodiments, the thickness of the hydrogel or hydrogel matrix is from about 100 μm to about 800 μm. In some embodiments, the thickness of the hydrogel or hydrogel matrix is from about 150 μm to about 800 μm. In some embodiments, the thickness of the hydrogel or hydrogel matrix is from about 200 μm to about 800 μm. In some embodiments, the thickness of the hydrogel or hydrogel matrix is from about 250 μm to about 800 μm. In some embodiments, the thickness of the hydrogel or hydrogel matrix is from about 300 μm to about 800 μm. In some embodiments, the thickness of the hydrogel or hydrogel matrix is from about 350 μm to about 800 μm. In some embodiments, the thickness of the hydrogel or hydrogel matrix is from about 400 μm to about 800 μm. In some embodiments, the thickness of the hydrogel or hydrogel matrix is from about 450 μm to about 800 μm. In some embodiments, the thickness of the hydrogel or hydrogel matrix is from about 500 μm to about 800 μm. In some embodiments, the thickness of the hydrogel or hydrogel matrix is from about 550 μm to about 800 μm. In some embodiments, the thickness of the hydrogel or hydrogel matrix is from about 600 μm to about 800 μm. In some embodiments, the thickness of the hydrogel or hydrogel matrix is from about 650 μm to about 800 μm. In some embodiments, the thickness of the hydrogel or hydrogel matrix is from about 700 μm to about 800 μm. In some embodiments, the thickness of the hydrogel or hydrogel matrix is from about 750 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrixes can have various thicknesses. In some embodiments, the thickness of the hydrogel or hydrogel matrix is from about 10 μm to about 3000 μm. In some embodiments, the thickness of the hydrogel or hydrogel matrix is from about 150 μm to about 3000 μm. In some embodiments, the thickness of the hydrogel or hydrogel matrix is from about 200 μm to about 3000 μm. In some embodiments, the thickness of the hydrogel or hydrogel matrix is from about 250 μm to about 3000 μm. In some embodiments, the thickness of the hydrogel or hydrogel matrix is from about 300 μm to about 3000 μm. In some embodiments, the thickness of the hydrogel or hydrogel matrix is from about 350 μm to about 3000 μm. In some embodiments, the thickness of the hydrogel or hydrogel matrix is from about 400 μm to about 3000 μm. In some embodiments, the thickness of the hydrogel or hydrogel matrix is from about 450 μm to about 3000 μm. In some embodiments, the thickness of the hydrogel or hydrogel matrix is from about 500 μm to about 3000 μm. In some embodiments, the thickness of the hydrogel or hydrogel matrix is from about 550 μm to about 3000 μm. In some embodiments, the thickness of the hydrogel or hydrogel matrix is from about 600 μm to about 3000 μm. In some embodiments, the thickness of the hydrogel or hydrogel matrix is from about 650 μm to about 3000 μm. In some embodiments, the thickness of the hydrogel or hydrogel matrix is from about 700 μm to about 3000 μm. In some embodiments, the thickness of the hydrogel or hydrogel matrix is from about 750 μm to about 3000 μm. In some embodiments, the thickness of the hydrogel or hydrogel matrix is from about 800 μm to about 3000 μm. In some embodiments, the thickness of the hydrogel or hydrogel matrix is from about 850 μm to about 3000 μm. In some embodiments, the thickness of the hydrogel or hydrogel matrix is from about 900 μm to about 3000 μm. In some embodiments, the thickness of the hydrogel or hydrogel matrix is from about 950 μm to about 3000 μm. In some embodiments, the thickness of the hydrogel or hydrogel matrix is from about 1000 μm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 μm. In some embodiments, the thickness of the hydrogel or hydrogel matrix is from about 2500 μm to about 3000 μm. In some embodiments, the thickness of the hydrogel or hydrogel matrix is from about 100 μm to about 2500 μm. In some embodiments, the thickness of the hydrogel or hydrogel matrix is from about 100 μm to about 2000 μm. In some embodiments, the thickness of the hydrogel or hydrogel matrix is from about 100 μm to about 1500 μm. In some embodiments, the thickness of the hydrogel or hydrogel matrix is from about 100 μm to about 1000 μm. In some embodiments, the thickness of the hydrogel or hydrogel matrix is from about 100 μm to about 950 μm. In some embodiments, the thickness of the hydrogel or hydrogel matrix is from about 100 μm to about 900 μm. In some embodiments, the thickness of the hydrogel or hydrogel matrix is from about 100 μm to about 850 μm. In some embodiments, the thickness of the hydrogel or hydrogel matrix is from about 100 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof.

In some embodiments the composition or culture vessel is free of a hydrogel.

The present disclosure also relates to a system comprising: (i) a cell culture vessel optionally comprising a hydrogel; (ii) one or a plurality of stem cells or neural crest cells either in suspension or as a component of a spheroid. The present disclosure also relates to a system comprising: (i) a cell culture vessel optionally comprising a hydrogel; (ii) one or a plurality of stem cells or neural crest cells either in suspension or as a component of a spheroid; and (iii) one or plurality of nitrergic agents. In some embodiments, the system further comprises one or combination of culture mediums disclosed herein. The disclosure also relates to a method of culturing enteric neurons in a system, the system comprising: (i) a cell culture vessel optionally comprising a hydrogel; (ii) one or a plurality of stem cells or neural crest cells either in suspension or as a component of a spheroid; and (iii) on or plurality of nitrergic agents. In some embodiments, the system further comprises one or combination of culture mediums disclosed herein. In some embodiments, the methods relate to replacing medium during a culture time of from about 12 to about 21 days at least one time to (i) expose one or a plurality of stem cells to a first cell medium for a time period sufficient to differentiate the one or plurality of stem cells into neural crest cells and the sequentially replacing the medium to (ii) expose one or plurality of neural crest cells to a second cell medium for a time period sufficient to differentiate the one or plurality of neural crest cells into enteric nitrergic neurons.

In some embodiments, the system comprises a solid substrate. The term “solid substrate” as used herein refers to any substance that is a solid support that is free of or substantially free of cellular toxins. In some embodiments, the solid substrate comprise one or a combination of silica, plastic, and metal. In some embodiments, the solid substrate comprises pores of a size and shape sufficient to allow diffusion or non-active transport of proteins, nutrients, and gas through the solid substrate in the presence of a cell culture medium. In some embodiments, the pore size is no more than about 10, 9, 8, 7, 6, 5, 4, 3, 2, 1 micron microns in diameter. One of ordinary skill could determine how big of a pore size is necessary based upon the contents of the cell culture medium and exposure of cells growing on the solid substrate in a particular microenvironment. For instance, one of ordinary skill in the art can observe whether any cultured cells in the system or device are viable under conditions with a solid substrate comprises pores of various diameters. In some embodiments, the solid substrate comprises a base with a predetermined shape that defines the shape of the exterior and interior surface. In some embodiments, the base comprises one or a combination of silica, plastic, ceramic, or metal and wherein the base is in a shape of a cylinder or in a shape substantially similar to a cylinder, such that the first cell-impenetrable polymer and a first cell-penetrable polymer coat the interior surface of the base and define a cylindrical or substantially cylindrical interior chamber; and wherein the opening is positioned at one end of the cylinder. In some embodiments, the base comprises one or a plurality of pores of a size and shape sufficient to allow diffusion of protein, nutrients, and oxygen through the solid substrate in the presence of the cell culture medium. In some embodiments, the solid substrate comprises a plastic base with a pore size of no more than 1 micron in diameter and comprises at least one layer of hydrogel matrix wherein the solid substrate comprises at least one compartment defined at least in part by the shape of an interior surface of the solid substrate and accessible from a point outside of the solid substrate by an opening, optionally positioned at one end of the solid substrate. In embodiments, where the solid substrate comprises a hollow interior portion defined by at least one interior surface, the cells in suspension or tissue explants may be seeded by placement of cells at or proximate to the opening such that the cells may adhere to at least a portion the interior surface of the solid substrate for prior to growth. The at least one compartment or hollow interior of the solid substrate allows a containment of the cells in a particular three-dimensional shape defined by the shape of the interior surface. In some embodiments, the solid substrate and encourages directional growth of the cells away from the opening. In the case of neuronal cells, the degree of containment and shape of the at least one compartment are conducive to axon growth from soma positioned within the at least one compartment and at or proximate to the opening.

The present disclosure provides devices, methods, and systems involving production, maintenance, and physiological interrogation of neural cells in microengineered configurations designed to mimic native nerve tissue anatomy. It is another object of the disclosure to provide a medium to high-throughput assay of neurological function for the screening of pharmacological and/or toxicological properties of chemical and biological agents. In some embodiments, the agents are cells, such as any type of cell disclosed herein, or antibodies, such as antibodies that are used to treat clinical disease. In some embodiments, the agents are any drugs or agents that are used to treat human disease such that toxicities, effects or neuromodulation can be compared among a new agent which is a proposed mammalian treatment and existing treatments from human disease. In some embodiments, new agents for treatment of human disease are treatments for neurodegenerative disease and are compared to existing treatments for neurodegenerative disease.

Similarly, information gathered from imaging can determine quantitative metrics for the degree of cell toxicology and lends further insight into toxic and neuroprotective mechanisms of various agents or compounds of interest. In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

In some embodiments, the at least one agent comprises one or a combination of chemotherapeutics chosen from: Actinomycin, Alitretinoin, All-trans retinoic acid, Azacitidine, Azathioprine, Bexarotene, Bleomycin, Bortezomib, Capecitabine, Carboplatin, Chlorambucil, Cisplatin, Cyclophosphamide, Cytarabine, Dacarbazine (DTIC), Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Irinotecan, Mechlorethamine, Melphalan, Mercaptopurine, Methotrexate, Mitoxantrone, Nitrosoureas, Oxaliplatin, Paclitaxel, Pemetrexed, Romidepsin, Tafluposide, Temozolomide (Oral dacarbazine), Teniposide, Tioguanine (formerly Thioguanine), Topotecan, Tretinoin, Valrubicin, Vemurafenib, Vinblastine, Vincristine, Vindesine, Vinorelbine, Vismodegib, and Vorinostat. In some embodiments, the at least one agent comprises one or a combination of analgesics chosen from: Paracetoamol, Non-steroidal anti-inflammatory drugs (NSAIDs), COX-2 inhibitors, opioids, flupirtine, tricyclic antidepressants, carbamaxepine, gabapentin, and pregabalin.

In some embodiments, the at least one agent comprises one or a combination of cardiovascular modulators chosen from: nepicastat, cholesterol, niacin, scutellaria, prenylamine, dehydroepiandrosterone, monatepil, esketamine, niguldipine, asenapine, atomoxetine, flunarizine, milnacipran, mexiletine, amphetamine, sodium thiopental, flavonoid, bretylium, oxazepam, and honokiol.

In some embodiments, the at least one agent comprises one or a combination of neuroprotectants and/or neuromodulators chosen from: tryptamine, galanin receptor 2, phenylalanine, phenethylamine, N-methylphenethylamine, adenosine, kyptorphin, substance P, 3-methoxytyramine, catecholamine, dopamine, GABA, calcium, acetylcholine, epinephrine, norepinephrine, and serotonin. In some embodiments, the at least one agent comprises one or a combination of immunomodulators chosen from: clenolizimab, enoticumab, ligelizumab, simtuzumab, vatelizumab, parsatuzumab, Imgatuzumab, tregalizaumb, pateclizumab, namulumab, perakizumab, faralimomab, patritumab, atinumab, ublituximab, futuximab, and duligotumab.

In some embodiments, the at least one agent comprises one or a combination of anti-inflammatories chosen from: ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, and salsalate. In some embodiments, the at least one agent comprises one or a combination of anti-microbials chosen from: antibacterials, antifungals, antivirals, antiparasitics, heat, radiation, and ozone.

The disclosure furthers relates to a pharmaceutical composition comprising a therapeutically effective amount of one or a plurality of any of the enteric neurons disclosed herein and a pharmaceutically acceptable carrier. The pharmaceutical compositions of the disclosure can further include one or more compatible active ingredients which are aimed at providing the composition with another pharmaceutical effect in addition to that provided by the disclosed enteric neurons. “Compatible” as used herein means that the active ingredients of such a composition are capable of being combined with each other in such a manner so that there is no interaction that would substantially reduce the efficacy of each active ingredient or the composition under ordinary use conditions. Such one or more compatible active ingredients may include, but not limited to, any of the chemotherapeutic agents, neuroprotectants and/or neuromodulators, or immunosuppressive agents disclosed elsewhere herein. In some embodiments, the agents are administered in the form of a salt, e.g., a pharmaceutically acceptable salt. “Pharmaceutically acceptable salts” means physiologically and pharmaceutically acceptable salts of compounds, such as oligomeric compounds, i.e., salts that retain the desired biological activity of the parent compound and do not impart undesired toxicological effects thereto. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The choice of carrier in the pharmaceutical composition may be determined in part by the particular method used to administer the composition. Accordingly, there is a variety of suitable formulations. For example, the pharmaceutical composition can contain preservatives. Suitable preservatives may include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21^{st} ed. (May 1, 2005) or Remington's Pharmaceutical Sciences, 18^{th} or 19^{th} ed. published by the Mack Publishing Company of Easton, Pa., both are incorporated herein by reference.

In some embodiments, the pharmaceutical composition comprises the disclosed composition in an amount that is effective to treat or prevent the disease or condition, such as a therapeutically effective or prophylactically effective amount. Thus, in some embodiments, the methods of administration include administration of the disclosed composition at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Although the descriptions of pharmaceutical compositions provided herein are principally directed to pharmaceutical compositions, which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the disclosure may be prepared/formulated, packaged, or sold in formulations suitable for oral, rectal, vaginal, parenteral, topical, pulmonary, intranasal, intra-lesional, buccal, ophthalmic, intravenous, intra-organ or another route of administration.

According to some embodiments, the pharmaceutical compositions of the present disclosure may be administered initially, and thereafter maintained by further administrations. For example, according to some embodiments, the pharmaceutical compositions of the described invention may be administered by one method of injection, and thereafter further administered by the same or by different method.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations may include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. For parenteral application, suitable vehicles consist of solutions, e.g., oily or aqueous solutions, as well as suspensions, emulsions, or implants. Aqueous suspensions may contain substances, which increase the viscosity of the suspension and include, for example, sodium carboxymethyl cellulose, sorbitol and/or dextran.

All references and patent applications disclosed herein are incorporated by reference in their entireties.

### EXAMPLES

### Example 1. Defined Enteric Neuron Model System

Materials—Reagents and Equipment

E8-C, hPSC Medium for Maintenance

Combine Essential 8-Flex supplement (20 μl ml⁻¹) with Essential 8™ Flex Medium. Store at 4° C. (use within 2 weeks).

Cocktail A, first ENC Differentiation Medium

Combine BMP4 (1 ng ml⁻¹), SB431542 (10 μM), CHIR 99021 (600 nM), with Essential 6™ Medium. Store at 4° C. (use within 2 weeks).

Cocktail B, Second ENC Differentiation Medium

Combine SB431542 (10 μM), CHIR 99021 (1.5 μM), with Essential 6™ medium. Store at 4° C. (use within 2 weeks).

Cocktail C, Third ENC Differentiation Medium

Combine SB431542 (10 μM), CHIR 99021 (1.5 μM), Retinoic Acid (1 μM), with Essential 6™ medium. Store at 4° C. (use within 2 weeks).

NC-C, ENC Medium for Spheroid Maintenance

Combine FGF2 (10 ng ml⁻¹), CHIR 99021 (3 μM), N2 Supplement (10 μl ml⁻¹), B27 Supplement (20 μl ml⁻¹), Glutagro (10 μl ml⁻¹), MEM Nonessential Amino Acids (10 μl ml⁻¹), with Neurobasal® Medium. Store at 4° C. (use within 2 weeks).

EN-C, EN Medium for Differentiation and Maintenance

Combine GDNF (10 ng ml⁻¹), Ascorbic Acid (100 μM), N2 Supplement (10 μl ml⁻¹), B27 Supplement (20 μl ml⁻¹), Glutagro (10 μl ml⁻¹), MEM Nonessential Amino Acids (10 μl ml⁻¹), with Neurobasal® Medium. Store at 4° C. (use within 2 weeks).

EDTA 1× for Passaging hESCs

Combine EDTA (500 μM) with PBS.

Matrigel®

Thaw frozen vial of Matrigel® overnight at 4° C. Prepare 500 μl aliquots in pre-chilled 50 ml conical tubes using chilled pipette tips and keep frozen at −20° C.

Matrigel®-Coated Plates

Dilute a 500 μl frozen aliquot of Matrigel® in 50 ml of cold DMEM:F12. Pipette up and down vigorously with a 25 ml or 50 ml serological pipette to break frozen Matrigel® pellet. Coat wells with the diluted Matrigel® solution (100 μl/cm² well surface area) and let stand in a 37° C. incubator overnight. Aspirate the Matrigel® solution before plating hPSCs.

Vitronectin-Coated Plates

Dilute vitronectin (10 μl ml⁻¹) with PBS and mix thoroughly. Coat wells with diluted vitronectin solution (100 μl/cm² well surface area) and let plates stand in a 37° C. incubator overnight. Aspirate the vitronectin solution before plating hPSCs. It should be appreciated that Matrigel®-coated plates yield a fully defined system, whereas vitronectin-coated plates yield a partially defined system.

PO/Lam/FN-Coated Plates

Combine PO (15 μg ml⁻¹) with PBS. Coat wells with PO/PBS solution (100 μl/cm² well surface area) and let stand in 37° C. incubator overnight. The following day, combine FN (2 μg ml⁻¹) and Laminin (2 μg ml⁻¹) with PBS. Aspirate PO/PBS and coat well with FN/LM/PBS solution (100 μl/cm² well surface area). Let plates stand in 37° C. incubator for a minimum of 2 hours. Aspirate FN/LM/PBS solution before plating cells.

Methods

Thawing Frozen hPSCs

Store frozen stocks of hPSCs in a liquid nitrogen cryogenic storage system at −156° C. For hPSCs lines that were previously maintained in mTESR1, first establish the line in mTESR1 for the initial passage, before transitioning the cultures to E8 medium. The cultures should be passaged at least twice in new medium before continuing the protocol.
1. Remove vial of hPSCs from liquid nitrogen and transfer vial to a 37° C. water bath.
2. Keep hold of the top of the sealed vial, and gently swirl around the water bath to ensure even thawing of frozen cells. Once only a small pellet of ice remains, remove the vial from water bath, spray the sealed vial with 70% ethanol, and transfer to laminar flow hood. Thawed cells should be plated immediately.
3. Add 0.5-1 ml of E8-C directly into vial and gently mix by pipetting up and down 1-2 times. Transfer cell suspension to a conical tube.
4. Centrifuge the conical tube at 1200 rpm (290×g) for 1 minute.
5. Carefully aspirate supernatant with a sterile pipette tip while avoiding contact with the pellet. Resuspend the pellet with 2 ml of E8-C and plate suspension into a single well of a 6-well or Matrigel®-coated or vitronectin-coated plate.
6. Proceed by expanding colonies as described in Step 1 of the protocol.
Note: A ROCK (Rho kinase) inhibitor such as Y-27632 dihydrochloride may be included in the initial E8-C medium conditions to enhance recovery and prevent excess cell death (27). Combine Y-27632 dihydrochloride (10 μM) with E8-C in a separate conical tube. Use this medium to break cell pellet after centrifugation and initial plating. Aspirate Y-27632 dihydrochloride supplemented medium from wells 3-5 hours after plating, and replace with fresh E8-C. Prolonged ROCK inhibition may adversely affect pluripotency and differentiation (28).

Step 1—Maintaining hPSC Cultures
7. Aspirate old E8-C medium from the corner of well using a sterile pipette tip. Add fresh E8-C (200 μl/cm² well surface area). Replace medium with fresh E8-C every other day.
8. When colonies are ˜₈₀% confluent, begin passage by aspirating E8-C from the corner of a single well.
9. Add PBS (100 μl/cm² well surface area) and gently rock plate to wash off loose debris. Aspirate PBS using a sterile pipette tip.
10. Add EDTA 1× (100 μl/cm² well surface area). Replace lid of plate and watch for detachment of edges of colonies from well surface through an inverted microscope (2-4 minutes).
11. Use a P1000 micropipette or a 5 ml serological pipette to mechanically harvest colonies from the well. Transfer EDTA 1× cell suspension to a 15 ml conical tube.
Note: Pipetting too vigorously may lead to excessive colony dissociation and adversely affect cell viability. Total time in EDTA 1× and pipetting technique should be adjusted to maintain cell viability.
12. Centrifuge the conical tube at 1200 rpm (290×g) for 1 minute.
13. Carefully aspirate supernatant with a sterile pipette tip while avoiding contact with the pellet. Resuspend the pellet with E8-C and plate suspension in new Matrigel-coated or vitronectin-coated 6-well plate.
14. Label plate with cell line, date, and new passage number. Incubate at 5% CO₂ and 37° C.
Note: Passage hPSC cultures once every 5 days when they reach ˜80% confluency. For continued maintenance, passaging ratios generally vary between 1:12 and 1:18 (i.e., resuspend the pellet of cells collected from 1 well at ˜80% confluency with 2-3 ml of E8-C and transfer 1 ml of this suspension to a new 15 ml conical tube. Add fresh E8-C to the new tube to bring the total volume to 12 ml. Add 2 ml of this suspension to each well of a new 6 well plate).

Step 2-ENC Induction (Days 0-12)

Day −2: Replating hPSCs for Differentiation
15. i. Two days before ENC induction, aspirate E8-C from hPSC cultures and use the same passage technique as described above, but use a 5:6 passaging ratio (i.e., all cells from 5 wells to a new 6-well plate) and leave in EDTA for 3-5 minutes for increased cell separation.
   ii. Feed cells with E8-C. Cells will continue to propagate and after 2 days the culture should become nearly confluent as a monolayer while maintaining typical hPSC morphology.

Day 0: ENC Induction Begins
16. Aspirate old E8-C medium from corner of well using a sterile pipette tip. Add Cocktail A (200 μl/cm² well surface area). Record date of day 0 of ENC differentiation. Incubate at 5% CO₂ and 37° C.

Day 2
17. Aspirate Cocktail A from corner of well using a sterile pipette tip. Add Cocktail B (200 μl/cm² well surface area). Incubate at 5% CO₂ and 37° C.

Day 4
18. On day 4, aspirate old Cocktail B using a sterile pipette tip and add fresh Cocktail B (200 μl/cm² well surface area). Incubate at 5% CO₂ and 37° C.

Day 6
19. On day 6, aspirate Cocktail B using a sterile pipette tip. Add Cocktail C (400 μl/cm² well surface area). Incubate at 5% CO₂ and 37° C. At ˜day 6, SOX10::GFP⁺ cells begin to cluster within the monolayer, indicating SOX10⁺ ENC lineage identity. GFP⁺ cluster size and prevalence continue to increase over the remaining ENC differentiation.

Day 8
20. On day 8, aspirate old Cocktail C using a sterile pipette tip and add fresh Cocktail C (400 μl/cm² well surface area). Incubate at 5% CO₂ and 37° C.
Note: As confluency continues to increase over the course of NC induction, cells may detach from the underlying monolayer. Avoid excess loss of cells by tipping the plate and gently adding fresh media to corner and side of well.

Day 10
21. On day 10, aspirate old Cocktail C using a sterile pipette tip and add fresh Cocktail C, increasing volume to 600 μl/cm² of well surface area. Incubate at 5% CO₂ and 37° C.

Day 11/12
22. ENC cells are ready to be removed for further differentiation. ENC cells are characterized by co-expression of SOX10::GFP and CD49D. ENC lineages are confirmed by the expression of HoxB2, HoxB5, and PAX3. Optional purification of ENC populations can be prepared by FACS using CD49D surface marker staining.
Note: Transfer ENC differentiations on day 11 if SOX10::GFP+ clusters are detaching from monolayer. Otherwise, day 12 will mark a complete ENC induction period.

Step 3—ENC Spheroid (Day 12-15)

ENC monolayers are detached from the well surface and transferred to ultra-low attachment plates to form free floating 3D spheroids. Spheroids are maintained in NC-C medium for 3-4 days as part of a NC maintenance process.
23. On day 11- to 12, aspirate Cocktail C from ENC induction phase plate using a sterile pipette tip. Add Accutase (100 μl/cm² well surface area). Incubate for 30 minutes at 37° C. and 5% CO₂.
24. Without aspirating Accutase, add NC-C (100 μl/cm² well surface area). Use a serological pipette to mechanically harvest cells from the surface of well. Add the cell suspension to a 15 ml conical tube.
25. Centrifuge the conical tube at 1200 rpm (290×g) for 1 minute.
26. With a sterile pipette tip, carefully aspirate as much supernatant as possible while avoiding the cell pellet.
27. Resuspend the pellet with the appropriate volume of NC-C and transfer the cell suspension to an ultra-low attachment 6-well plate (2 ml/well). 10 cm² of ENC monolayer will be transferred to 1 well of an ultra-low attachment 6 well plate (i.e. A 6-well ENC induction plate corresponds to a 6 well ultra-low attachment plate). Incubate at 37° C. and 5% CO₂.
28. On day 14, gently swirl ultra-low attachment plates to group the free-floating spheroids into the center of each well. Using a P1000 micropipette, slowly aspirate the old NC-C by moving around the circumference of well, actively avoiding any removal of spheroids.
29. Add 2 ml of fresh NC-C to each ultra-low attachment plate well. Incubate at 37° C. and 5% CO₂. 3D spheroids should form by day 14.

Step 4—Enteric No Neurons Induction Phase (Day 15→)

The enteric NO neurons induction phase starts after the ENC spheroid phase (Step 3) and 15 total days from the start of ENC differentiation. Depending on the culture system, there are two options for the induction.

Option 1 (for 3-D Culture):
30. On day 15, ENC spheroids are gathered in the center of the wells using a swirling motion and neural crest medium is removed using a P1000 micropipette in slow circular motion, avoiding the free-floating spheroids.
31. Spheroids are fed with the same volume of ENC medium containing PP121 (1-5 μM), GDNF (10 ng/ml), ascorbic acid (100 μM), N2 supplement (10 μl/ml), B27 supplement (20 μl/ml), glutagro (10 μl/ml), and MEM NEAAs (10 μl/ml) in neurobasal medium.
32. Cells are fed with ENC every other day until day 30-40, after which, feeding frequency can be reduced to once or twice a week but with a higher volume of medium.

Option 2 (for 2-D Culture):
30. On day 15, ENC spheroids are gathered in the center of the wells using a swirling motion and neural crest medium is removed using a P1000 micropipette in slow circular motion, avoiding the free-floating spheroids.
31. After washing the spheroids with PBS, accutase is added and plates are incubated for 30 minutes at 37° C. to dissociate the spheroids.
32. Then, remaining spheroids are broken by pipetting ENC medium.
33. ENC contains PP121 (1-5 μM), GDNF (10 ng/ml), ascorbic acid (100 μM), N2 supplement (10 μl/ml), B27 supplement (20 μl/ml), glutagro (10 μl/ml), and MEM NEAAs (10 μl/ml) in neurobasal medium.
34. Cells are spun (2 minutes, 290×g, 20-25° C.) and supernatant is removed.
35. Pellet is resuspended in ENC medium and cells are plated on PO/laminin/FN plates at 100,000 viable cells per cm².
36. Feeding continues every other day with ENC medium until day 30-40, after which, feeding frequency can be reduced to once or twice a week but with a higher volume of medium.

Results

The disclosed methods and systems reliably produce populations of hPSC-derived enteric NO neurons under chemically defined conditions. Proportions of cells positive for NO neurons identities may vary between cell lines, as well as between differentiations of a given cell line.

### Example 2. Fluorescence Activated Cell Sorting (FACS)

After 12 days of ENC induction under (Step 3), fluorescence activated cell sorting (FACS) can be used to prepare purified populations of NC cells. Previous NC induction protocols have suggested using p75/HNK1 marker staining for FACS analysis^{11,13}. However, p75 expression is found outside of the ENC and a portion of p75/HNK1 double positive cells have been shown to be SOX10::GFP− (12). We have demonstrated that CD49D (α4 integrin) is a specific marker for SOX10+ hPSC-derived NC lineages¹⁶. Here we present a procedure for the purification of ENC cells by FACS using CD49D. FACS purification is particularly recommended for experiments and assays that involve early ENC progenitors (day 11). Further differentiation under the 3D sphere culture condition is generally sufficient to enhance the purity of NC cells and neurons in the later stages of differentiation without FACS purification.

Reagents
DMEM/F-12, no glutamine (Life Technologies Corporation, 21331020)
BSA, Bovine Serum Albumin (Sigma, A4503)
Anti-human CD49D antibody (Biolegend, 304314)
DAPI (Sigma, D9542)
Normocin, Antimicrobial Reagent (InvivoGen, ant-nr-1)

Equipment
5 ml Round Bottom Polystyrene Test Tube, w/Cell Strainer Cap (Falcon 352235)
5 ml Round Bottom Polystyrene Test Tube, w/Snap Cap (Falcon 352003)
FACS Analyzer (i.e BD LSRFortessa)

Reagent Setup

Staining Medium
Dissolve BSA (0.02 mg ml⁻¹) with DMEM/F-12, no glutamine. Add Pe/Cy7 anti-human CD49D antibody (1.25 μl ml⁻¹). Prepare 2.4 ml per 6-well plate of ENC differentiations (400 μl per well).

Sorting Medium
Dissolve BSA (0.02 mg ml⁻¹) with DMEM/F-12, no glutamine.

Procedure
i. On day 12 of ENC induction, aspirate Cocktail C from ENC induction plate using a sterile pipette tip. Add Accutase (100 μl/cm² well surface area). Incubate at 5% CO₂ and 37° C. for 30 minutes.
ii. DO NOT ASPIRATE Accutase. Use a serological pipet to mechanically harvest cells from the surface of well. Add cell suspension to a 15 ml conical tube.
iii. Centrifuge the conical tube at 1200 rpm (290×g) for 1 minute. With a sterile pipet tip, carefully aspirate as much supernatant as possible while avoiding contact with the cell pellet.
iv. Resuspend the pellet with freshly prepared staining medium (400 μl for every well of a 6-well plate harvested).
v. Place the conical tube of cell suspension in ice for 20 minutes.
vi. After 20 minutes, centrifuge the conical tube at 1200 rpm (290×g) for 1 minute. With a sterile pipet tip, carefully aspirate as much supernatant as possible while avoiding contact with the cell pellet.
vii. Resuspend the pellet with freshly prepared sorting medium (˜1 ml total). Add DAPI (1 μl ml⁻¹).
viii. Transfer the stained cell suspension through the cell strainer cap to a 5 ml round bottom test tube for FACS.
ix. FACS settings may vary per user. Collect CD49D+ population in a sterile 5 ml round bottom test tube and cap.
x. Centrifuge the test tube at 1200 rpm (290×g) for 1 minute. With a sterile pipet tip, carefully aspirate as much supernatant as possible while avoiding contact with the cell pellet.
xi. Resuspend the pellet with NC-C (1 ml/10⁶ cells) and transfer suspension to an ultra-low attachment 6-well plate (2 ml/well). Incubate at 37° C. and 5% CO₂.
xii. Resume protocol Step 4-vi.
   Note: Sorted cells may be fed with NC-C supplemented with Normocin (1 μl/ml). Antimicrobial supplemented medium should be used for a minimum of two days.

### Example 3. NO Release Assay for High Throughput Drug Screening

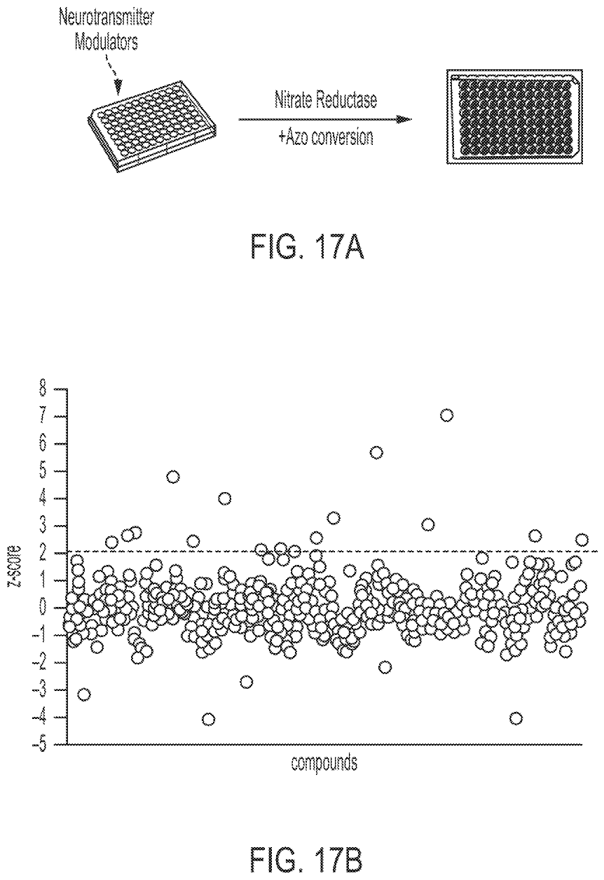

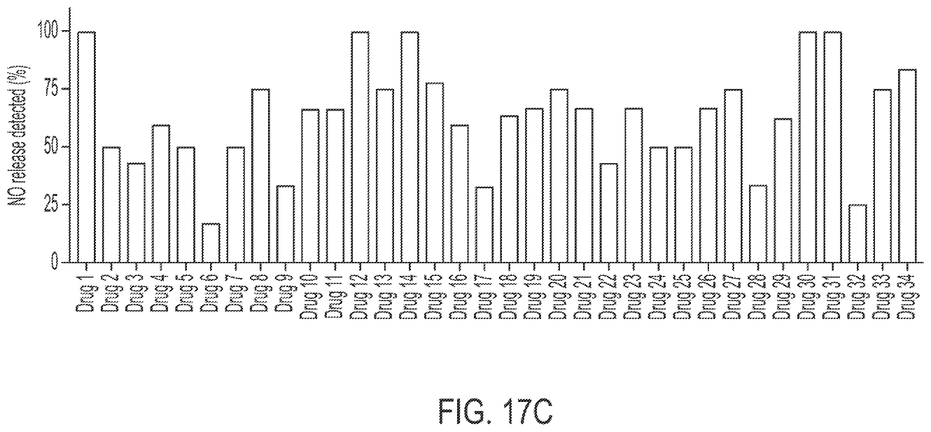

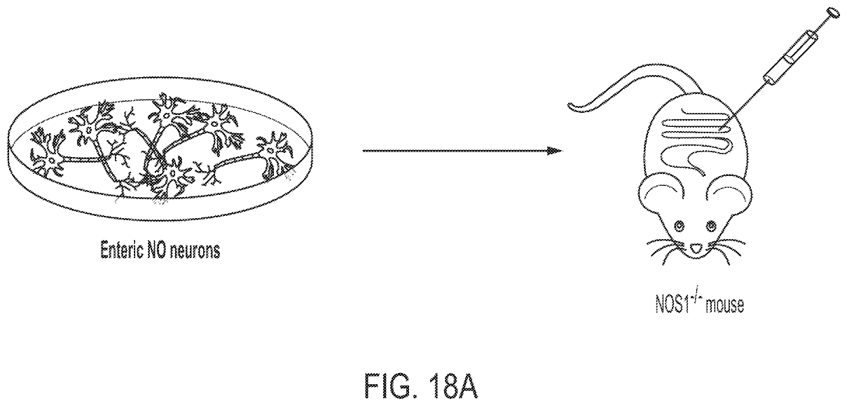

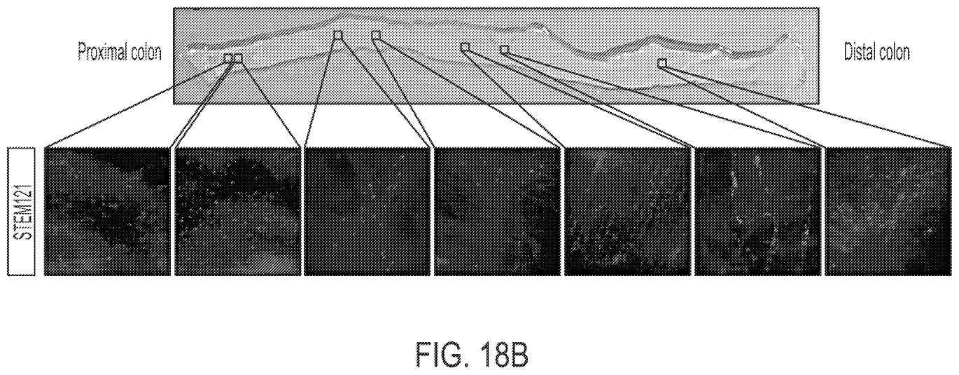

For high-throughput measuring of nitric oxide (NO) release, 2-D cultures of mature neurons (96-well plates) are used (FIG. 17A). After washing cells with Tyrode's solution containing NaCl (129 mM), KCl (5 mM), CaCl₂ (2 mM), MgCl (1 mM), glucose (30 mM) and HEPES (25 mM) at pH 7.4, 70 μl/well of Tyrode's solution is added to each well. HTS library compounds are then added (1 μM) using a 98-head pintool. After 45 minutes incubation at 37° C., supernatants are used to determine NO release following NO assay kit instructions (Invitrogen, EMSNO).

Briefly, the kit uses the enzyme nitrate reductase which converts nitrate to nitrite which is then detected as a colored azo dye that absorbs light at 540 nm (FIG. 17A). NO release for each compound is presented as the A540 nm relative to the negative controls (vehicle DMSO).

After removing Tyrod's solution supernatant from cells, neurons are washed twice with Tyrode's solution and then fed with fresh ENC medium.

### Example 4. Transplantation of hPSC-Derived NO Neurons into NOS1−/− Mouse Colon

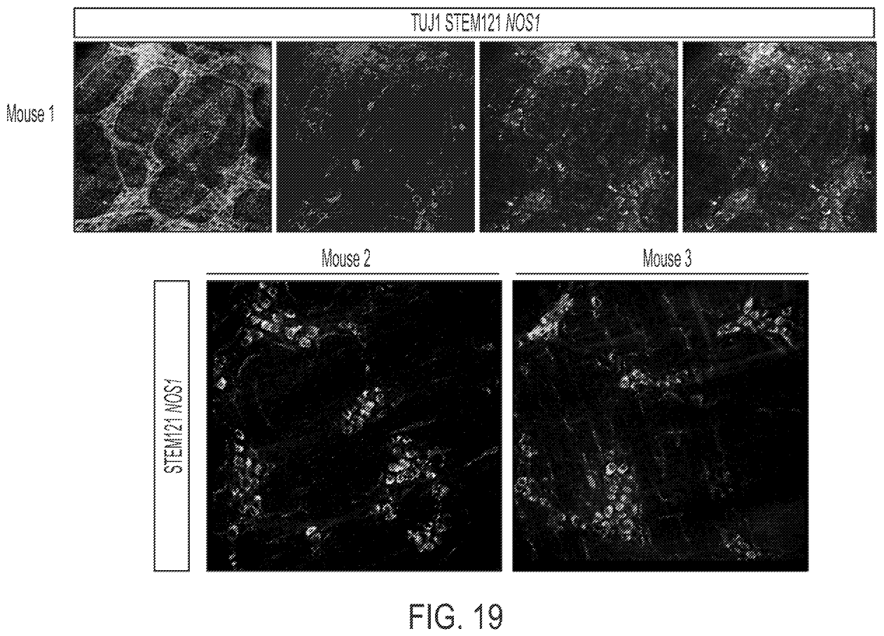

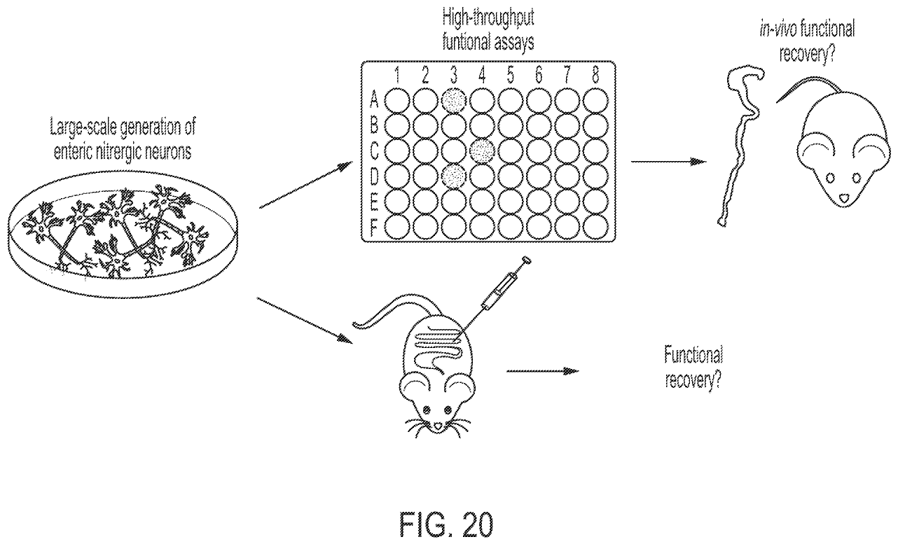

NO neurons were transplanted in a mouse model of GI motility disorder and showed great capacity to engraft and survive (FIGS. 19A and 19B). STEM121 is a marker for a human-specific cytoplasmic protein. Immunofluorescence staining of STEM121 shows a large number of human cells in different segments of mouse colon tissue 8 weeks after transplantation (FIG. 19B).

Immunofluorescence staining of STEM121, TUJ1 and NOS1 shows a large number of human NO neurons in mouse colon tissue 8 weeks after transplantation (FIG. 19). Given the remarkable potential of cells to engraft and survive in intestinal tissue, they show promise for cell therapy in GI motility disorders such as achalasia and gastroparesis.

Materials

Reagents—Cell Culture

Human Embryonic or Induced Pluripotent Stem Cell Lines

The quality of hPSC lines used in the differentiations should be verified by standard characterization of pluripotency including expression of markers such as NANOG and OCT4 and their ability to differentiate into endodermal, mesodermal and ectodermal lineages. The cell lines used in this example are human ES cell line H9 (WA-09) derivative SOX10::GFP (WiCell Research Institute, Memorial Sloan Kettering Cancer Center), human ES cell line UCSF4 (UCSF) and human iPS cell line WTC11 (Coriell Institute, UCSF).

Appropriate consent procedures and administrative regulations must be followed for work involving hESCs and hiPSCs. It is important to assure adherence with national and institutional guidelines and regulations.

The hPSC lines should be STR profiled to confirm their identity and ensure they are not cross contaminated. Regular karyotyping and frequent mycoplasma testing are necessary to monitor genomic stability and to avoid latent contamination.
DMEM/F-12, no glutamine (Life Technologies, 21331020)
Essential 8™ Flex Medium Kit (Life Technologies, A2858501)
Essential 6™ Medium (Life Technologies, A1516401)
Neurobasal™ Medium (Life Technologies, 21103049)
N-2 Supplement (CTS™, A1370701)
B-27™ Supplement, serum free (Life Technologies, 17504044)
MEM Nonessential Amino Acids (Corning, 25-025-CI)
Glutagro™ (Corning, 25-015-CI)
BSA, Bovine Serum Albumin (Sigma, A4503)
PBS, Phosphate-Buffered Saline, Ca²⁺- and Mg²⁺-free (Life Technologies, 10010023)
EDTA (Corning, MT-46034CI)
Accutase™ (Stemcell Technologies, 07920)
STEM-CELLBANKER® DMSO Free (Amsbio, 11897F)
BMP-4, Recombinant Human BMP-4 Protein (R&D Systems, 314-BP) Stock aliquots should be at stored −80° C. One aliquot should be kept at 4° C. to avoid multiple freeze/thaw cycles and used within 4 weeks.
CHIR 99021 (Tocris, 4423) Stock aliquots should be stored at −20° C. One aliquot should be kept at 4° C. and used within 4 weeks.
FGF2, Recombinant Human FGF Basic (R&D Systems #233-FB) Stock aliquots should be stored at −80° C. One aliquot should be kept at 4° C. to avoid multiple freeze/thaw cycles and used within 4 weeks.
GDNF, Recombinant Human Glial Derived Neurotrophic Factor (Peprotech, 450-10) Stock aliquots should be stored at −80° C. One aliquot should be kept at 4° C. to avoid multiple freeze/thaw cycles and used within 4 weeks.
RA, Retinoic Acid (Sigma, R2625) Stock aliquots should be stored at −80° C. One aliquot should be kept at 4° C. to avoid multiple freeze/thaw cycles and used within 4 weeks.
SB431542 (R&D Systems, 1614) Stock aliquots should be stored at 4° C.
Y-27632 dihydrochloride ((Tocris Bioscience, 1254) Stock aliquots should be stored at −20° C. One aliquot should be kept at 4° C. and used within 4 weeks.
Matrigel® hESC-Qualified Matrix, *LDEV-Free, (Corning, 354277)
Vitronectin XF (Stemcell Technologies, 07180)
FN, Fibronectin, Human (Corning, 356008) Stock aliquots should be stored at −80° C. One aliquot should be kept at 4° C. and used within 4 weeks.
LM, Laminin I, Mouse (Cultrex, 3400-010) Stocks should be stored at −80° C.
PO, Poly-L-Ornithine Hydrobromide (Sigma, P3655) Stock aliquots should be stored at −80° C. One aliquot should be kept at 4° C. and used within 4 weeks.
Trypan Blue Solution, 0.4% (Life Technologies, 15250061) Caution: Trypan Blue is a suspected carcinogen and should be handled with care. Collect all materials exposed to Trypan Blue for disposal according to institutional guidelines.
Gelatin, powder (Sigma, G9391)
MEF CF-1 mitomycin C-treated mouse embryonic fibroblasts (Applied StemCell, Inc., ASF-1223)
FBS, fetal bovine serum (Sciencell, 0025)
DMEM, Dulbecco's modified Eagle medium (Life Technologies, 11965-118).
Collagenase IV (Life Technologies, 17104-019)
KSR, Knockout Serum Replacement (Life Technologies, 10828-028)
L-glutamine (Life Technologies, 25030-081)
Knockout DMEM (Life Technologies, 10829-018)
KSR, Knockout Serum Replacement (Life Technologies, 10828-028)
2-mercaptoethanol (Life Technologies, 21985-023)
DMEM/F12 powder (Life Technologies, 12500-062)
Glucose (Sigma, G7021)
Sodium bicarbonate (Sigma, S5761)
Putrescine (Sigma, cat. no. P5780)
Progesterone (Sigma, cat. no. P8783)
Sodium selenite (Bioshop Canada, SEL888)
Transferrin (Celliance/Millipore, 4452-01)
Insulin (Sigma, 16634)

Reagents—QRT-PCR
RNeasy RNA purification kit (Qiagen, 74106)
SYBR™ Green PCR Master Mix (Applied Biosystems, 4309155)
Superscript IV Reverse Transcriptase Kit (Invitrogen, 18090010)
RNaseOUT™ Recombinant Ribonuclease Inhibitor (Invitrogen, 10777019)
Random Primers (Invitrogen, 48190011)
dNTPs for cDNA Probe Synthesis (10 mM) (Invitrogen, AM8200)
Hs_SOX10_1_SG QuantiTect Primer Assay (Qiagen, QT0005540)
Hs_EDNRB_1_SG QuantiTect Primer Assay (Qiagen, QT00014343)
Hs_PHOX2A_1_SG QuantiTect Primer Assay (Qiagen, QT00215467)
Hs_PHOX2B_1_SG QuantiTect Primer Assay (Qiagen, QT00015078)
Hs_HAND2_2_SG QuantiTect Primer Assay (Qiagen, QT01012907)
Hs_ASCL1_1_SG QuantiTect Primer Assay (Qiagen, QT00237755)
Hs_NTRK3_1_SG QuantiTect Primer Assay (Qiagen, QT00052906)
Hs_ASLC6A4_1_SG QuantiTect Primer Assay (Qiagen, QT00058380)
Hs_CHAT_1_SG QuantiTect Primer Assay (Qiagen, QT00029624)
Hs_SERT_1_SG QuantiTect Primer Assay (Qiagen, QT0058380)
Hs_NOS1_1_SG QuantiTect Primer Assay (Qiagen, QT00043372)
Hs_TUBB_1_SG QuantiTect Primer Assay (Qiagen, QT00089775)
Hs_GFAP_1_SG QuantiTect Primer Assay (Qiagen, QT00081151)
Hs_GAPDH_1_SG QuantiTect Primer Assay (Qiagen, QT00079247)

Reagents—Immunocytochemistry and Flow Cytometry
PFA, Paraformaldehyde Solution 4% in PBS (Alfa Aesar, J19943K2)
Caution: PFA is a known mutagen and irritant and should be handled with care. Collect all PFA containing solutions for disposal according to institutional guidelines.
Fixation/Permeabilization Solution Kit (BD Biosciences, 554714)
Perm/Wash Buffer (BD Perm/Wash™, 554723)
Pe/Cy7 CD49D antibody (BioLegend, 304314)
Anti-TUJ1 Antibody (Mouse) (BioLegend, 801202)
Anti-Serotonin-5-HT Antibody (Rabbit) (Sigma, S5545)
Anti-GABA Antibody (Rabbit) ((Sigma, S5545)
Anti-NOS1 Antibody (Rabbit) (Santa Cruz Biotechnology, sc648)
Alexa Fluor 488 donkey anti-mouse IgG (Life Technologies, A21202)
Alexa Fluor 647 donkey anti-rabbit IgG (Life Technologies, A31573)
DAPI (Sigma, D9542)
Caution: DAPI is a known mutagen and should be handled with care. Collect all DAPI containing solutions for disposal according to institutional guidelines.

Equipment
Horizontal Laminar Flow Hood
Cell culture centrifuge (i.e. Eppendorf 5810R)
Inverted microscope (i.e. Evos FL) with fluorescence equipment and digital imaging capture system.
CO2 incubator with controlling and monitoring system for CO2, humidity and temperature
Refrigerator 4° C., freezer −20° C., freezer −80° C.
Cell culture disposables: Petri dishes, multiwell plates, conical tubes, pipettes, pipette tips, cell scrapers, etc.
Hemocytometer (i.e. Hausser Scientific)
qPCR System (i.e. 7900HT Fast Real-Time PCR System)
FACS Analyzer (i.e. BD LSRFortessa)

### REFERENCES

1. Shyamala, K., et al., Neural crest: The fourth germ layer. J. Oral Maxillofac. Pathol. 19, 221 (2015).
2. Crane, J. F., et al., Neural Crest Stem and Progenitor Cells. Annu. Rev. Cell Dev. Biol. 22, 267-286 (2006).
3. Thomas, S., et al. Human neural crest cells display molecular and phenotypic hallmarks of stem cells. Hum. Mol. Genet. 17, 3411-3425 (2008).
4. Chan, K. K., et al., Hoxb3 vagal neural crest-specific enhancer element for controlling enteric nervous system development. Dev. Dyn. 233, 473-483 (2005).
5. Fu, M., et al., HOXB5 expression is spatially and temporarily regulated in human embryonic gut during neural crest cell colonization and differentiation of enteric neuroblasts. Dev. Dyn. 228, 1-10 (2003).
6. Heanue, T. A. & Pachnis, V. Enteric nervous system development and Hirschsprung's disease: advances in genetic and stem cell studies. Nat. Rev. Neurosci. 8, 466 (2007).
7. Cheeseman, B. L., et al., A. Cell lineage tracing in the developing enteric nervous system: superstars revealed by experiment and simulation. J. R. Soc. Interface 11, 20130815 (2014).
8. Wallace, A. S. & Burns, A. J. Development of the enteric nervous system, smooth muscle and interstitial cells of Cajal in the human gastrointestinal tract. Cell Tissue Res. 319, 367-382 (2005).
9. Kim, J., et al., SOX10 maintains multipotency and inhibits neuronal differentiation of neural crest stem cells. Neuron 38, 17-31 (2003).
10. Lasrado, R. et al. Lineage-dependent spatial and functional organization of the mammalian enteric nervous system. Science 356, 722-726 (2017).
11. Lee, G., et al., Derivation of neural crest cells from human pluripotent stem cells. Nat. Protoc. 5, 688-701 (2010).
12. Mica, Y., et al., Modeling neural crest induction, melanocyte specification, and disease-related pigmentation defects in hESCs and patient-specific iPSCs. Cell Rep. 3, 1140-1152 (2013).
13. Fattahi, F., et al., Neural Crest Cells from Dual SMAD Inhibition. Curr. Protoc. Stem Cell Biol. 33, 1H.9.1-9 (2015).
14. Fattahi, F., et al., Deriving human ENS lineages for cell therapy and drug discovery in Hirschsprung disease. Nature 531, 105-109 (2016).
15. Tchieu, J., et al., A Modular Platform for Differentiation of Human PSCs into All Major Ectodermal Lineages. Cell Stem Cell 21, 399-410.e7 (2017).
16. Hackland, J. O. S. et al., Top-Down Inhibition of BMP Signaling Enables Robust Induction of hPSCs Into Neural Crest in Fully Defined, Xeno-free Conditions. Stem Cell Rep. 9, 1043-1052 (2017).
17. Chalazonitis, A., et al., Neurotrophin-3 induces neural crest-derived cells from fetal rat gut to develop in vitro as neurons or glia. J. Neurosci. Off J. Soc. Neurosci. 14, 6571-6584 (1994).
18. Chalazonitis, A., et al., Age-dependent differences in the effects of GDNF and NT-3 on the development of neurons and glia from neural crest-derived precursors immunoselected from the fetal rat gut: expression of GFRalpha-1 in vitro and in vivo. Dev. Biol. 204, 385-406 (1998).
19. Memic, F., et al., Transcription and Signaling Regulators in Developing Neuronal Subtypes of Mouse and Human Enteric Nervous System. Gastroenterology (2017).
20. Chambers, S. M., et al., Combined small-molecule inhibition accelerates developmental timing and converts human pluripotent stem cells into nociceptors. Nat Biotechnol 30, 715-720, (2012).
21. Menendez, L., et al., Wnt signaling and a Smad pathway blockade direct the differentiation of human pluripotent stem cells to multipotent neural crest cells. Proceedings of the National Academy of Sciences of the United States of America 108, 19240-19245, (2011).
22. Lee, G., et al., Isolation and directed differentiation of neural crest stem cells derived from human embryonic stem cells. Nat Biotechnol 25, 1468-1475, (2007).
20. Lai, F. P.-L., et al., Correction of Hirschsprung-Associated Mutations in Human Induced Pluripotent Stem Cells Via Clustered Regularly Interspaced Short Palindromic Repeats/Cas9, Restores Neural Crest Cell Function. Gastroenterology 153, 139-153.e8 (2017).
21. Burns, A. J., et al., White paper on guidelines concerning enteric nervous system stem cell therapy for enteric neuropathies. Dev. Biol. 417, 229-251 (2016).
22. Stamp, L. A., Cell therapy for GI motility disorders: comparison of cell sources and proposed steps for treating Hirschsprung disease. Am. J. Physiol.-Gastrointest. Liver Physiol. 312, G348-G354 (2017).
23. Li, W., et al., Characterization and transplantation of enteric neural crest cells from human induced pluripotent stem cells. Mol. Psychiatry 23, 499-508 (2018).
24. Workman, M. J., et al., Engineered human pluripotent-stem-cell-derived intestinal tissues with a functional enteric nervous system. Nat. Med. 23, 49-59 (2017).
25. Gershon, M. D. & Ratcliffe, E. M. Developmental biology of the enteric nervous system: Pathogenesis of Hirschsprung's disease and other congenital dysmotilities. Semin. Pediatr. Surg. 13, 224-235 (2004).
26. Chen, G., et al., Chemically defined conditions for human iPSC derivation and culture. Nat. Methods 8, 424-429 (2011).
27. Claassen, D. A., et al., ROCK inhibition enhances the recovery and growth of cryopreserved human embryonic stem cells and human induced pluripotent stem cells. Mol. Reprod. Dev. 76, 722-732 (2009).
28. Maldonado, M., et al., ROCK inhibitor primes human induced pluripotent stem cells to selectively differentiate towards mesendodermal lineage via epithelial-mesenchymal transition-like modulation. Stem Cell Res. 17, 222-227 (2016).
29. Sasselli, V., et al., The enteric nervous system. Dev. Biol. 366, 64-73 (2012).

1. A composition comprising a plurality of enteric neurons, wherein at least about 30% of the enteric neurons express nitric oxide synthase (NOS1).

2.-4. (canceled)

5. The composition of claim 1, wherein the NOS1 comprises at least about 70% sequence identity to SEQ ID NO: 10, SEQ ID NO: 11 or SEQ ID NO: 12, or a functional fragment thereof.

6. (canceled)

7. The composition of claim 1, wherein the enteric neurons are derived from one or a plurality of pluripotent stem cells.

8.-14. (canceled)

15. The composition of claim 1, wherein the plurality of cells expressing NOS1 are deficient in expression of any one or combination of: CHAT, 5HT, and GABA.

16.-19. (canceled)

20. The composition of claim 1, wherein the plurality of cells expressing NOS1 are derived from a combination of two or more of: human inducible pluripotent stem cells that are in culture at least about 12 days; cells that express human CD49 and SOX10 in culture from about 12 to about 15 days; cells that express human TRKC, PHOX2B and EDNRB in culture from about 15 to about 30 days; cells that express human TRKC and TUJ1 in culture from about 30 days to about 45 days.

21. A system comprising a cell culture vessel comprising a plurality of enteric neurons supported in a culture medium, wherein at least about 20% of the enteric neurons express nitric oxide synthase.

22. The system of claim 21, wherein from about 20 to about 60% of the enteric neurons express nitric oxide synthase.

23. The system of claim 22, wherein the cell culture comprises the composition of claim 1

24. (canceled)

25. The system of claim 21, wherein the cell culture vessel further comprises: (i) smooth muscle cells proximate to or adjacent to the plurality of enteric neurons; and (ii) a hydrogel.

26. A pharmaceutical composition comprising:
a) a therapeutically effective amount of one or a plurality of enteric neurons; and
b) a pharmaceutically acceptable carrier.

27. The pharmaceutical composition of claim 26, wherein from about 20% to about 100% of the enteric neurons express NOS1.

28. (canceled)

29. The pharmaceutical composition of claim 26, wherein the NOS1 comprises at least about 70% sequence identity to SEQ ID NO: 10, SEQ ID NO: 11 or SEQ ID NO: 12, or a functional fragment thereof.

30.-31. (canceled)

32. The pharmaceutical composition of claim 26, wherein the one or plurality enteric neurons are derived from human inducible pluripotent stem cells.

33. The pharmaceutical composition of claim 26, wherein the enteric neurons are derived from a combination of two or more of: human inducible pluripotent stem cells that are in culture at least about 12 days; cells that express human CD49 and SOX10 in culture from about 12 to about 15 days; cells that express human TRKC, PHOX2B and EDNRB in culture from about 15 to about 30 days; cells that express human TRKC and TUJ1 in culture from about 30 days to about 45 days.

34. The pharmaceutical composition of claim 26, wherein the plurality of cells expressing NOS1 are deficient in expression of any one or combination of: ChAT, 5HT, and GABA.

35. A method of claim 1 wherein producing a plurality of enteric neurons, the method comprising exposing one or more nitrergic agents to a plurality of enteric neural crest cells, wherein at least about 30% of the enteric neurons express NOS1.

36.-41. (canceled)

42. The method of claim 35, wherein the one or more nitrergic agents are receptor tyrosine kinase (RTK) inhibitors.

43-44. (canceled)

45. A method of claim 1 wherein producing nitric oxide synthase (NOS)-expressing enteric neurons, the method comprising exposing one or more nitrergic agents to one or a plurality of enteric neural crest cells.

46. The method of claim 45, wherein the enteric neurons express NOS1 at least about 70% sequence identity to SEQ ID NO: 10, SEQ ID NO: 11 or SEQ ID NO: 12, or a functional fragment thereof.

47.-49. (canceled)

50. The method of claim 45, wherein the one or more nitrergic agents are receptor tyrosine kinase (RTK) inhibitors.

51. The method of claim 45, wherein the nitrergic agents are selected from one or a combination of: PP121, afatinib, ibrutinib, mizoribine, donepezil, cilostazol, RG108, prucalopride, PluriSIn #1, L-Arginine, AMG-458, OG-L002, GSK2801, GSK J4, GSK591, and sodium orthovanadate, or a salt of any of the foregoing.

52. (canceled)

53. The method of claim 45, wherein the nitrergic agent has a structure represented by a formula: 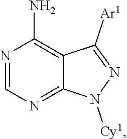
wherein Cy¹ is selected from C3-C8 cycloalkyl and C2-C9 heterocycloalkyl, and is substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, —C(O)R¹, and —CO₂R¹;
wherein R¹, when present, is selected from hydrogen, C1-C4 alkyl, C2-C4 alkenyl, and C1-C4 haloalkyl;
wherein Ar¹ is selected from C6-C10 aryl and C2-C9 heteroaryl, and is substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, (C1-C4)(C1-C4) dialkylamino, Ar², and —OAr²; and
wherein Ar², when present, is selected from C6-C10 aryl and C2-C9 heteroaryl, and is substituted with 0, 1, 2, or 3 groups independently selected from halogen, —NH₂, —NO₂, —CN, —OH, C1-C4 alkyl, C2-C4 alkenyl, C1-C4 haloalkyl, C1-C4 hydroxyalkyl, C1-C4 alkoxy, C1-C4 alkylamino, and (C1-C4)(C1-C4) dialkylamino,
or a pharmaceutically acceptable salt thereof.

54.-76. (canceled)

77. The method of claim 53, wherein the nitrergic agent is selected from: 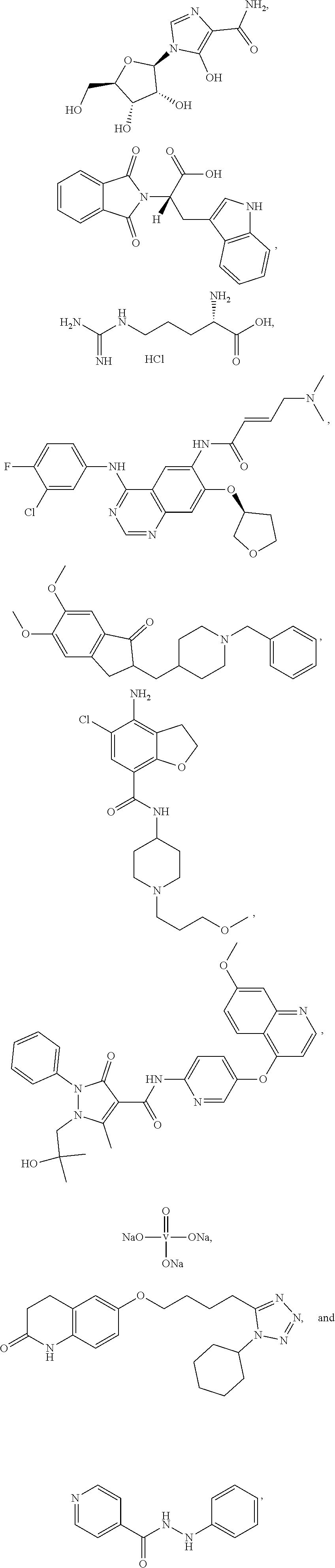
or a pharmaceutically acceptable salt thereof.

78. (canceled)

79. A method of evaluating a neuromodulatory effect of an agent, the method comprising:
a) culturing a composition of claim 1 in the presence or absence of the agent, wherein at least about 30% of the enteric neurons express nitric oxide synthase; and
b) detecting and/or measuring nitric oxide released by the agent;
wherein a detectable level of nitric oxide in the presence of the agent is indicative of a neuromodulatory effect, and no detectable level of nitric oxide in the presence of the agent is indicative of the agent not conferring a neuromodulatory effect.

80.-84. (canceled)

85. A method for screening an agent capable of modulating calcium influx, the method comprising:
a) culturing a composition of claim 1 in the presence or absence of the agent, wherein at least about 30% of the enteric neurons express nitric oxide synthase; and
b) detecting and/or measuring nitric oxide released by the agent;
wherein a detectable level of nitric oxide in the presence of the agent is indicative of the agent capable of modulating calcium influx, and no detectable level of nitric oxide in the presence of the agent is indicative of the agent not conferring ability to modulate calcium influx.

86.-94. (canceled)


SEQUENCE LISTING (82 sequences)

SEQ ID NO: 1 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 2 - protein, 478 aa, Rattus norvegicus
MASLRPFFILALLALVSLADQESCKGRCTQGFMASKKCQCDELCTYYQSCCVDYMEQCKPQVTRGDVFTMPEDEYWSYDYPEETKNSTSTGVQSENTSLHFNLKPRAEETIKPTTPDPQEQSNTQEPEVGQQGVAPRPDTTDEGTSEFPEEELCSGKPFDAFTDLKNGSLFAFRGEYCYELDETAVRPGYPKLIQDVWGIEGPIDAAFTRINCQGKTYLFKGSQYWRFEDGVLDPDYPRNISEGFSGIPDNVDAALALPAHSYSGRERVYFFKGKQYWEYEFQQQPSQEECEGSSLSAVFEHFALLQRDSWENIFELLFWGRSSDGAKGPQFISRDWHGVPGKVDAAMAGRIYITGSTFRSVQAKKQKSGRRSRKRYRSRRGRGHSRSRSRSMSSRRPSRSVWFSLLSSEESGLGTYNYDYDMNWRIPATCEPIQSVYFFSGDKYYRVNLRTRRVDSVNPPYPRSIAQYWLGCPTSEK

SEQ ID NO: 3 - protein, 478 aa, Mus musculus
MAPLRPFFILALVAWVSLADQESCKGRCTQGFMASKKCQCDELCTYYQSCCADYMEQCKPQVTRGDVFTMPEDDYWSYDYVEEPKNNTNTGVQPENTSPPGDLNPRTDGTLKPTAFLDPEEQPSTPAPKVEQQEEILRPDTTDQGTPEFPEEELCSGKPFDAFTDLKNGSLFAFRGQYCYELDETAVRPGYPKLIQDVWGIEGPIDAAFTRINCQGKTYLFKGSQYWRFEDGVLDPGYPRNISEGFSGIPDNVDAAFALPAHRYSGRERVYFFKGKQYWEYEFQQQPSQEECEGSSLSAVFEHFALLQRDSWENIFELLFWGRSSDGAREPQFISRNWHGVPGKVDAAMAGRIYVTGSLSHSAQAKKQKSKRRSRKRYRSRRGRGHRRSQSSNSRRSSRSIWFSLFSSEESGLGTYNNYDYDMDWLVPATCEPIQSVYFFSGDKYYRVNLRTRRVDSVNPPYPRSIAQYWLGCPTSEK

SEQ ID NO: 4 - protein, 748 aa, Homo sapiens
MGLRTAKKRGLGGGGKWKREEGGGTRGRREVRPACFLQSGGRGDPGDVGGPAGNPGCSPHPRAATRPPPLPAHTPAHTPEWCGAASAEAAEPRRAGPHLCIPAPGLTKTPILEKVPRKMAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKGQLSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDAPGGVELSDTHRALQLLHGGGYSKNGANRWYDKSLQFVVGRDGTCGVVCEHSPFDGIVLVQCTEHLLKHVTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQP

SEQ ID NO: 5 - protein, 640 aa, Rattus norvegicus
MPILEKAPQKMPVKASSWEELDLPKLPVPPLQQTLATYLQCMQHLVPEEQFRKSQAIVKRFGAPGGLGETLQEKLLERQEKTANWVSEYWLNDMYLNNRLALPVNSSPAVIFARQHFQDTNDQLRFAACLISGVLSYKTLLDSHSLPTDWAKGQLSGQPLCMKQYYRLFSSYRLPGHTQDTLVAQKSSIMPEPEHVIVACCNQFFVLDVVINFRRLSEGDLFTQLRKIVKMASNEDERLPPIGLLTSDGRSEWAKARTVLLKDSTNRDSLDMIERCICLVCLDGPGTGELSDTHRALQLLHGGGCSLNGANRWYDKSLQFVVGRDGTCGVVCEHSPFDGIVLVQCTEHLLKHMMTSNKKLVRADSVSELPAPRRLRLKCSPETQGHLASSAEKLQRIVKNLDFIVYKFDNYGKTFIKKQKYSPDGFIQVALQLAYYRLYQRLVPTYESASIRRFQEGRVDNIRSATPEALAFVQAMTDHKAAMPASEKLQLLQTAMQAHKQYTVMAITGMAIDNHLLALRELARDLCKEPPEMFMDETYLMSNRFVLSTSQVPTTMEMFCCYGPVVPNGNGACYNPQPEAITFCISSFHSCKETSSVEFAEAVGASLVDMRDLCSSRQPADSKPPAPKEKARGPSQAKQS

SEQ ID NO: 6 - protein, 641 aa, Mus musculus
MPILEKVPPKMPVQASSCEEVLDLPKLPVPPLQQTLATYLQCMQHLVPEEQFRKSQAIVKRFGAPGGLGETLQEKLLERQEKTANWVSEYWLNDMYLNNRLALPVNSSPAVIFARQHFQDTNDQLRFAASLISGVLSYKALLDSQSIPTDWAKGQLSGQPLCMKQYYRLFSSYRLPGHTQDTLVAQKSSIMPEPEHVIVACCNQFFVLDVVINFRRLSEGDLFTQLRKIVKMASNEDERLPPIGLLTSDGRSEWAKARTVLLKDSTNRDSLDMIERCICLVCLDGPGTGDLSDTHRALQLLHGGGCSLNGANRWYDKSLQFVVGRDGTCGVVCEHSPFDGIVLVQCTEHLLKHMMTGNKKLVRVDSVSELPAPRRLRWKCSPETQGHLASSAEKLQRIVKNLDFIVYKFDNYGKTFIKKQKCSPDGFIQVALQLAYYRLYQRLVPTYESASIRRFQEGRVDNIRSATPEALAFVQAMTDHKAAVLASEKLQLLQRAIQAQTEYTVMAITGMAIDNHLLALRELARDLCKEPPEMFMDETYLMSNRFILSTSQVPTTMEMFCCYGPVVPNGYGACYNPHAEAITFCISSFHGCKETSSVEFAEAVGASLVDMRDLCSSRQPADSKPPTAKERARGPSQAKQS

SEQ ID NO: 7 - protein, 422 aa, Homo sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCKFCRQ

SEQ ID NO: 8 - protein, 422 aa, Rattus norvegicus
MDVFSFGQGNNTTASQEPFGTGGNVTSISDVTFSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVRKVEKKGAGTSLGTSSAPPPKKSLNGQPGSGDWRRCAENRAVGTPCTNGAVRQGDDEATLEVIEVHRVGNSKEHLPLPSESGSNSYAPACLERKNERNAEAKRKMALARERKTVKTLGIIMGTFILCWLPFFIVALVLPFCESSCHMPALLGAIINWLGYSNSLLNPVIYAYFNKDFQNAFKKIIKCKFCRR

SEQ ID NO: 9 - protein, 421 aa, Mus musculus
MDMFSLGQGNNTTTSLEPFGTGGNDTGLSNVTFSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSNPNECTISKDHGYTIYSTFGAFYIPLLLMLVLYGRIFRAARFRIRKTVKKVEKKGAGTSFGTSSAPPPKKSLNGQPGSGDCRRSAENRAVGTPCANGAVRQGEDDATLEVIEVHRVGNSKGHLPLPSESGATSYVPACLERKNERTAEAKRKMALARERKTVKTLGIIMGTFILCWLPFFIVALVLPFCESSCHMPELLGAIINWLGYSNSLLNPVIYAYFNKDFQNAFKKIIKCKFCR

SEQ ID NO: 10 - protein, 1434 aa, Homo sapiens
MEDHMFGVQQIQPNVISVRLFKRKVGGLGFLVKERVSKPPVIISDLIRGGAAEQSGLIQAGDIILAVNGRPLVDLSYDSALEVLRGIASETHVVLILRGPEGFTTHLETTFTGDGTPKTIRVTQPLGPPTKAVDLSHQPPAGKEQPLAVDGASGPGNGPQHAYDDGQEAGSLPHANGLAPRPPGQDPAKKATRVSLQGRGENNELLKEIEPVLSLLTSGSRGVKGGAPAKAEMKDMGIQVDRDLDGKSHKPLPLGVENDRVFNDLWGKGNVPVVLNNPYSEKEQPPTSGKQSPTKNGSPSKCPRFLKVKNWETEVVLTDTLHLKSTLETGCTEYICMGSIMHPSQHARRPEDVRTKGQLFPLAKEFIDQYYSSIKRFGSKAHMERLEEVNKEIDTTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKPPRGRFDVLPLLLQANGNDPELFQIPPELVLEVPIRHPKFEWFKDLGLKWYGLPAVSNMLLEIGGLEFSACPFSGWYMGTEIGVRDYCDNSRYNILEEVAKKMNLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKVMSMEEYDIVHLEHETLVLVVTSTFGNGDPPENGEKFGCALMEMRHPNSVQEERKSYKVRFNSVSSYSDSQKSSGDGPDLRDNFESAGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTFVAEAPELTQGLSNVHKKRVSAARLLSRQNLQSPKSSRSTIFVRLHTNGSQELQYQPGDHLGVFPGNHEDLVNALIERLEDAPPVNQMVKVELLEERNTALGVISNWTDELRLPPCTIFQAFKYYLDITTPPTPLQLQQFASLATSEKEKQRLLVLSKGLQEYEEWKWGKNPTIVEVLEEFPSIQMPATLLLTQLSLLQPRYYSISSSPDMYPDEVHLTVAIVSYRTRDGEGPIHHGVCSSWLNRIQADELVPCFVRGAPSFHLPRNPQVPCILVGPGTGIAPFRSFWQQRQFDIQHKGMNPCPMVLVFGCRQSKIDHIYREETLQAKNKGVFRELYTAYSREPDKPKKYVQDILQEQLAESVYRALKEQGGHIYVCGDVTMAADVLKAIQRIMTQQGKLSAEDAGVFISRMRDDNRYHEDIFGVTLRTYEVTNRLRSESIAFIEESKKDTDEVFSS

SEQ ID NO: 11 - protein, 1429 aa, Rattus norvegicus
MEENTFGVQQIQPNVISVRLFKRKVGGLGFLVKERVSKPPVIISDLIRGGAAEQSGLIQAGDIILAVNDRPLVDLSYDSALEVLRGIASETHVVLILRGPEGFTTHLETTFTGDGTPKTIRVTQPLGPPTKAVDLSHQPSASKDQSLAVDRVTGLGNGPQHAQGHGQGAGSVSQANGVAIDPTMKSTKANLQDIGEHDELLKEIEPVLSILNSGSKATNRGGPAKAEMKDTGIQVDRDLDGKSHKAPPLGGDNDRVFNDLWGKDNVPVILNNPYSEKEQSPTSGKQSPTKNGSPSRCPRFLKVKNWETDVVLTDTLHLKSTLETGCTEHICMGSIMLPSQHTRKPEDVRTKDQLFPLAKEFLDQYYSSIKRFGSKAHMDRLEEVNKEIESTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVQFTEICIQQGWKAPRGRFDVLPLLLQANGNDPELFQIPPELVLEVPIRHPKFDWFKDLGLKWYGLPAVSNMLLEIGGLEFSACPFSGWYMGTEIGVRDYCDNSRYNILEEVAKKMDLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKAMSMEEYDIVHLEHEALVLVVTSTFGNGDPPENGEKFGCALMEMRHPNSVQEERKSYKVRFNSVSSYSDSRKSSGDGPDLRDNFESTGPLANVRFSVFGLGSRAYPHFCAFGHAVDTLLEELGGERILKMREGDELCGQEEAFRTWAKKVFKAACDVFCVGDDVNIEKPNNSLISNDRSWKRNKFRLTYVAEAPDLTQGLSNVHKKRVSAARLLSRQNLQSPKFSRSTIFVRLHTNGNQELQYQPGDHLGVFPGNHEDLVNALIERLEDAPPANHVVKVEMLEERNTALGVISNWKDESRLPPCTIFQAFKYYLDITTPPTPLQLQQFASLATNEKEKQRLLVLSKGLQEYEEWKWGKNPTMVEVLEEFPSIQMPATLLLTQLSLLQPRYYSISSSPDMYPDEVHLTVAIVSYHTRDGEGPVHHGVCSSWLNRIQADDVVPCFVRGAPSFHLPRNPQVPCILVGPGTGIAPFRSFWQQRQFDIQHKGMNPCPMVLVFGCRQSKIDHIYREETLQAKNKGVFRELYTAYSREPDRPKKYVQDVLQEQLAESVYRALKEQGGHIYVCGDVTMAADVLKAIQRIMTQQGKLSEEDAGVFISRLRDDNRYHEDIFGVTLRTYEVTNRLRSESIAFIEESKKDADEVFSS

SEQ ID NO: 12 - protein, 1429 aa, Mus musculus
MEEHTFGVQQIQPNVISVRLFKRKVGGLGFLVKERVSKPPVIISDLIRGGAAEQSGLIQAGDIILAVNDRPLVDLSYDSALEVLRGIASETHVVLILRGPEGFTTHLETTFTGDGTPKTIRVTQPLGTPTKAVDLSRQPSASKDQPLAVDRVPGPSNGPQHAQGRGQGAGSVSQANGVAIDPTMKNTKANLQDSGEQDELLKEIEPVLSILTGGGKAVNRGGPAKAEMKDTGIQVDRDLDGKLHKAPPLGGENDRVFNDLWGKGNVPVVLNNPYSENEQSPASGKQSPTKNGSPSRCPRFLKVKNWETDVVLTDTLHLKSTLETGCTEQICMGSIMLPSHHIRKSEDVRTKDQLFPLAKEFLDQYYSSIKRFGSKAHMDRLEEVNKEIESTSTYQLKDTELIYGAKHAWRNASRCVGRIQWSKLQVFDARDCTTAHGMFNYICNHVKYATNKGNLRSAITIFPQRTDGKHDFRVWNSQLIRYAGYKQPDGSTLGDPANVEFTEICIQQGWKPPRGRFDVLPLLLQANGNDPELFQIPPELVLEVPIRHPKFDWFKDLGLKWYGLPAVSNMLLEIGGLEFSACPFSGWYMGTEIGVRDYCDNSRYNILEEVAKKMDLDMRKTSSLWKDQALVEINIAVLYSFQSDKVTIVDHHSATESFIKHMENEYRCRGGCPADWVWIVPPMSGSITPVFHQEMLNYRLTPSFEYQPDPWNTHVWKGTNGTPTKRRAIGFKKLAEAVKFSAKLMGQAMAKRVKATILYATETGKSQAYAKTLCEIFKHAFDAKAMSMEEYDIVHLEHEALVLVVTSTFGNGDPPENGEKFGCALMEMRHPNSVQEERKSYKVRFNSVSSYSDSRKSSGDGPDLRDNFESTGPLANVRFSVFGLGSRAYPHFCAFGHAVDTLLEELGGERILKMREGDELCGQEEAFRTWAKKVFKAACDVFCVGDDVNIEKANNSLISNDRSWKRNKFRLTYVAEAPELTQGLSNVHKKRVSAARLLSRQNLQSPKSSRSTIFVRLHTNGNQELQYQPGDHLGVFPGNHEDLVNALIERLEDAPPANHVVKVEMLEERNTALGVISNWKDESRLPPCTIFQAFKYYLDITTPPTPLQLQQFASLATNEKEKQRLLVLSKGLQEYEEWKWGKNPTMVEVLEEFPSIQMPATLLLTQLSLLQPRYYSISSSPDMYPDEVHLTVAIVSYHTRDGEGPVHHGVCSSWLNRIQADDVVPCFVRGAPSFHLPRNPQVPCILVGPGTGIAPFRSFWQQRQFDIQHKGMNPCPMVLVFGCRQSKIDHIYREETLQAKNKGVFRELYTAYSREPDRPKKYVQDVLQEQLAESVYRALKEQGGHIYVCGDVTMAADVLKAIQRIMTQQGKLSEEDAGVFISRLRDDNRYHEDIFGVTLRTYEVTNRLRSESIAFIEESKKDTDEVFSS

SEQ ID NO: 13 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 14 - protein, 430 aa, Rattus norvegicus
MERRRITSARRSYASSETMVRGHGPTRHLGTIPRLSLSRMTPPLPARVDFSLAGALNAGFKETRASERAEMMELNDRFASYIEKVRFLEQQNKALAAELNQLRAKEPTKLADVYQAELRELRLRLDQLTTNSARLEVERDNLTQDLGTLRQKLQDETNLRLEAENNLAVYRQEADEATLARVDLERKVESLEEEIQFLRKIHEEEVRELQEQLAQQQVHVEMDVAKPDLTAALREIRTQYEAVATSNMQETEEWYRSKFADLTDVASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 15 - protein, 430 aa, Mus musculus
MERRRITSARRSYASETVVRGLGPSRQLGTMPRFSLSRMTPPLPARVDFSLAGALNAGFKETRASERAEMMELNDRFASYIEKVRFLEQQNKALAAELNQLRAKEPTKLADVYQAELRELRLRLDQLTANSARLEVERDNFAQDLGTLRQKLQDETNLRLEAENNLAAYRQEADEATLARVDLERKVESLEEEIQFLRKIYEEEVRELREQLAQQQVHVEMDVAKPDLTAALREIRTQYEAVATSNMQETEEWYRSKFADLTDAASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKDSKQEHKDVVM

SEQ ID NO: 16 - protein, 466 aa, Homo sapiens
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGTAAIQAHYKSAHLDHRHPGEGSPMSDGNPEHPSGQSHGPPTPPTTPKTELQSGKADPKRDGRSMGEGGKPHIDFGNVDIGEISHEVMSNMETFDVAELDQYLPPNGHPGHVSSYSAAGYGLGSALAVASGHSAWISKPPGVALPTVSPPGVDAKAQVKTETAGPQGPPHYTDQPSTSQIAYTSLSLPHYGSAFPSISRPQFDYSDHQPSGPYYGHSGQASGLYSAFSYMGPSQRPLYTAISDPSPSGPQSHSPTHWEQPVYTTLSRP

SEQ ID NO: 17 - protein, 466 aa, Rattus norvegicus
MAEEQDLSEVELSPVGSEEPRCLSPSSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGETDQGGAAAIQAHYKSAHLDHRHPEEGSPMSDGNPEHPSGQSHGPPTPPTTPKTELQSGKADPKRDGRSLGEGGKPHIDFGNVDIGEISHEVMSNMETFDVTELDQYLPPNGHPGHVGSYSAAGYGLSSALAVASGHSAWISKPPGVALPTVSPPAVDAKAQVKTETTGPQGPPHYTDQPSTSQIAYTSLSLPHYGSAFPSISRPQFDYSDHQPSGPYYGHAGQASGLYSAFSYMGPSQRPLYTAISDPSPSGPQSHSPTHWEQPVYTTLSRP

SEQ ID NO: 18 - protein, 466 aa, Mus musculus
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGAAAIQAHYKSAHLDHRHPEEGSPMSDGNPEHPSGQSHGPPTPPTTPKTELQSGKADPKRDGRSLGEGGKPHIDFGNVDIGEISHEVMSNMETFDVTELDQYLPPNGHPGHVGSYSAAGYGLGSALAVASGHSAWISKPPGVALPTVSPPGVDAKAQVKTETTGPQGPPHYTDQPSTSQIAYTSLSLPHYGSAFPSISRPQFDYSDHQPSGPYYGHAGQASGLYSAFSYMGPSQRPLYTAISDPSPSGPQSHSPTHWEQPVYTTLSRP

SEQ ID NO: 19 - DNA, 1589 bp, Homo sapiens
ggaaaaaaggaaagtgcacttggaagagatccaagtgggcaacttgaagaacaagtgccaaatagcacttctgtcatgctggatgtcagggctctttgtccactttgtatagccgctggcttatagaaggtgctcgataaatctcttgaatttaaaaatcaattaggatgcctctatagtgaaaaagatacagtaaagatgagggataatcaatttaaaaaatgagtaagtacacacaaagcactttatccattcttatgacacctgttacttttttgctgtgtttgtgtgtatgcatgccatgttatagtttgtgggaccctcaaagcaagctggggagagtatatactgaatttagcttctgagacatgatgctcttcctttttaattaacccagaacttagcagcttatctatttctctaatctcaaaacatccttaaactgggggtgatacttgagtgagagaattttgcaggtattaaatgaactatcttcttttttttttttctttgagacagagtcttgctctgtcacccaggctggagtgcagtggcgtgatctcagctcactgcaacctccgcctcccgggttcaagtgattctcctgcctcagcctcctgagtagctgggattacagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 20 - protein, 190 aa, Homo sapiens
MGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 21 - DNA, 2049 bp, Homo sapiens
ataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacataa

SEQ ID NO: 22 - protein, 289 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 23 - DNA, 2441 bp, Homo sapiens
gactatcacacaactatttccttggatgtaattctttgttaccctttacaagtataagtgttaccttacatggaaacgaagaaacaaaattcataaatttaaattcataaatttagctgaaagatactgattcaatttgtatacagtgaatataaatgagacgacagcaaaattttcatgaaatgtaaaatatttttatagtttgttcatactatatgaggttctattttaaatgactttctggattttaaaaaatttctttaaatacaatcatttttgtaatatttattttatgcttatgatctagataattgcagaatatcattttatctgactctgccttcataagagagctgtggccgaattttgaacatctgttatagggagtgatcaaattagaaggcaatgtggaaaaacaattctgggaaagatttctttatatgaagtccctgccactagccagccatcctaattgatgaaagttatctgttcacaggcctgcagtgatggtgaggaatgttctgagatttgcgaaggcatttgagtagtgaaatgtaagcacaaaacctcctgaacccagagtgtgtatacacaggaataaactttatgacatttatgtatttttaaaaaactttgtatcgttataaaaaggctagtcattctttcaggagaacatctaggatcatagatgaaaaatcaagccccgatttagaactgtcttctccaggatggtctctaaggaaatttacatttggttctttcctactcagaactactcagaaacaactatatatttcaggttatctgagcacagtgaaagcagagtactatggttgtccaacacaggcctctcagatacaaggggaacacaattacatattgggctagattttgcccagttcaaaatagtatttgttatcaacttactttgttacttgtatcatgaattttaaaaccctaccactttaagaagacagggatgggttattcttttttggcaggtaggctatataactatgtgattttgaaatttaactgctctggattagggagcagtgaatcaaggcagacttatgaaatctgtattatatttgtaacagaatataggaaatttaacataattgatgagctcaaatcctgaaaaatgaaagaatccaaattatttcagaattatctaggttaaatattgatgtattatgatggttgcaaagtttttttgtgtgtccaataaacacattgtaaaaaaaagaatttgaattgatatctaaaaacagaatttgaattgatatttcatcttgacttttaaagccctagaggctaattgttagtaacatcaatttctattaggatatccgtttggccacacagcaggaggttagagcaatggagcattactgagttcctccccctgtcagatcagcagcagcattagattctcatagaagtgcgaaccatatggtgaactggtatgtgagggatctagagtgccatgttcctcaagagaatctaatgcctgatgatctgaggtggaacagttcatcctgaaaccattcccccatccacggaaaaattgtcttccatgaaactggtcccaaaaagggtggggaccacaggtttaaagcatggccacatttctttatattaaaattctagtttgtacatttcttttagaaacaattacatgttactttggaatcatttcttccatgcttcctccataaagactgataagtcttggatgcaatctgtaaagaaaatacattatttcatcaacttattttgttgtttttcacatacacctaataagtatggtacacaatgccaatgccaaatacaaattgataacaaacacagcattcccaacagagctgtaatctagaaaactgagaaggtctgattgataaatcatcaacaacaataattgctctaaaacctccttaactgacttccttgattgtccaatgctctccattacctctgtaaaacagtcagttatgcctctagaacacccatgtctagtgggcacccctgcatgcttcttctaaccactgagtgtcacaatgcctaccaagaatgcgtttgcaggttcctaaacctgtttataccagttgctatgtaaaattgttcccaagggaagttgaatgctctgtaaaggcctaataaaagcaaattactgaacaaaacatgttacagtaattatgagtgagaggaaactaagatggaaggataaaaatctaacactttactattcagatggctccactaaaagatttaagatcttgatccatttttaaaaatccaaaatggaagttgtagacattatctgtagtttatgcacaacaataaattagaaagccaatgtagacacgcataaccaaagaaaatgccttgggtctacataacagttgaataaatgtaaagttgctttttatttattgaaa

SEQ ID NO: 24 - protein, 1032 aa, Homo sapiens [sequence omitted]

SEQ ID NO: 25 - DNA, 2395 bp, Homo sapiens
gacgatgacaagttccccgtgtgcatccgcgaggccgtcagccaggtgctcagcggctacgactggacgctggtgcccatgcccgtgcgcgtcaacggcgccagcaaaagcaagccgcacgtcaagcggcccatgaacgccttcatggtgtgggctcaggcagcgcgcaggaagctcgcggaccagtacccgcacctgcacaacgctgagctcagcaagacgctgggcaagctctggaggctgctgaacgaaagtgacaagcgccccttcatcgaggaggctgagcggctccgtatgcagcacaagaaagaccacccggactacaagtaccagcccaggcggcggaagaacgggaaggccgcccagggcgaggcggagtgccccggtggggaggccgagcaaggtgggaccgccgccatccaggcccactacaagagcgcccacttggaccaccggcacccaggagagggctcccccatgtcagatgggaaccccgagcacccctcaggccagagccatggcccacccacccctccaaccaccccgaagacagagctgcagtcgggcaaggcagacccgaagcgggacgggcgctccatgggggagggcgggaagcctcacatcgacttcggcaacgtggacattggtgagatcagccacgaggtaatgtccaacatggagacctttgatgtggctgagttggaccagtacctgccgcccaatgggcacccaggccatgtgagcagctactcagcagccggctatgggctgggcagtgccctggccgtggccagtggacactccgcctggatctccaagccaccaggcgtggctctgcccacggtctcaccacctggtgtggatgccaaagcccaggtgaagacagagaccgcggggccccaggggcccccacactacaccgaccagccatccacctcacagatcgcctacacctccctcagcctgccccactatggctcagccttcccctccatctcccgcccccagtttgactactctgaccatcagccctcaggaccctattatggccactcgggccaggcctctggcctctactcggccttctcctatatggggccctcgcagcggcccctctacacggccatctctgaccccagcccctcagggccccagtcccacagccccacacactgggagcagccagtatatacgacactgtcccggccctaaagggggccctgtcgccaccaccccccgcccagcccctgcccccagcctgtgtgccctgttccttgcccacctcaggcctggtggtggcagtggaggaggctgaggaggctgaagaggctgacaggtcggggggctttctgtctggctcactgccctgatgacccacccgccccatccaggctccagcagcaaagccccaggagaacaggctggacagaggagaaggaggttgactgttgcacccacactgaaagatgaggggctgcaccttcccccaggaatgaccctctatcccaggacctgagaagggcctgctcaccctcctcggggaggggaagcaccagggttggtggcatcggaggccttaccactcctatgactcctgttttctctctcacagatagtgagggtctgacatgcccatgccacctatgccacagtgcctaagggctaggccacccagagactgtgcccggagctggccgtgtctcccactcaggggctgagagtagctttgaggagcctcattggggagtggggggttcgagggacttagtggagttctcatcccttcaatgccccctccctttctgaaggcaggaaggagttggcacagaggccccctgatccaattctgtgccaataacctcattctttgtctgagaaacagcccccagtcctcctccactacaacctccatgaccttgagacgcatcccaggaggtgacgaggcaggggctccaggaaaggaatcagagacaattcacagagcctccctccctgggctccttgccagctccctcttcccttactaggctctatggcccctgctcagtcagccccactccctgggcttcccagagagtgacagctgctcaggccctaacccttggctccaggagacacagggcccagcacccaggttgctgtcggcaggctgaagacactagaatcctgacctgtacattctgcccttgcctcttaccccttgcctcccagtggtatttgaataaagtatgtagctatatctgcccctattttcctgttctgcagccccccaaatccacatgtaactcattactgtctcctgttatttatctcagtagtcccctctcctagccactctagcccctattaactctgcattaagcattccacataataaaattaaaggttccggtta

SEQ ID NO: 26 - protein, 466 aa, Homo sapiens; identical to SEQ ID NO: 16

SEQ ID NO: 27 - DNA, 2422 bp, Homo sapiens
cccacgccagggccagaggccgaggaaggcgggctaagtgagggggcgcggcgtggagaaccgccggggccgggagcggtagcgagcgcctagtaccgagcgccagggacggcaggagttcgcggagcgcggccgctgggggcggacggcagagcccgcgccacgcgatgcggggccgccgagtgtgagctgagcccagcgggccccaagccacctgcggccccctcccctctccctgccccccatctttcgggggcactcaaaccctcttcccctgagctccgtggcagcccccgaacaccctcatcgcccgctgccccctccccgccgccgctaccaaccccgaggagggatgaccctctccggcggcggcagcgccagcgacatgtccggccagacggtgctgacggccgaggacgtggacatcgatgtggtgggcgagggcgacgacgggctggaagagaaggacagcgacgcaggttgcgatagccccgcggggccgccggagctgcgcctggacgaggcggacgaggtgcccccggcggcaccccatcacggacagcctcagccgccccaccagcagcccctgacattgcccaaggaggcggccggagccggggccggaccggggggcgacgtgggcgcgccggaggcggacggctgcaagggcggtgttggcggcgaggagggcggcgcgagcggcggcgggcctggcgcgggcagcggttcggcgggaggcctggccccgagcaagcccaagaacagcctagtgaagccgccttactcgtacatcgcgctcatcaccatggccatcctgcagagcccgcagaagaagctgaccctgagcggcatctgcgagttcatcagcaaccgcttcccctactacagggagaagttccccgcctggcagaacagcatccgccacaacctctcactcaacgactgcttcgtcaagatcccccgcgagccgggcaacccgggcaagggcaactactggaccctggacccgcagtccgaggacatgttcgacaacggcagcttcctgcggcgccggaaacgcttcaagcgccaccagcaggagcacctgcgcgagcagacggcgctcatgatgcagagcttcggcgcttacagcctggcggcggcggccggcgccgcgggaccctacggccgcccctacggcctgcaccctgcggcggcggccggtgcctattcgcacccggcagcggcggcggccgcggctgctgcggcggcgctccagtacccgtacgcgctgccgccggtggcaccggtgctgcctcccgctgtgccgctgctgccctcgggcgagctgggccgcaaagcggccgccttcggctcacagctcggcccgggcctgcagctgcagctcaatagcctgggcgccgccgcggccgctgcgggcacagcgggcgccgcgggcaccaccgcgtcgctcatcaagtccgagccaagcgcgcggccgtcgttcagcatcgagaacatcataggtgggggccccgcggctcctgggggctcggcggtgggcgctggggtcgccggcggcactgggggttcagggggcggcagcacggcgcagtcgtttctgcggccacccgggaccgtgcagtcggcagcgctcatggccacccaccaaccgctgtcgctgagccggacgactgccaccatcgcgcccattcttagcgtgccactctccggacagtttctgcagcccgcagcctcggccgccgccgctgctgcggccgccgctcaagccaaatggccggcgcaatagggacgcgccaatggccgggacccagggtccggcggcggcctcgagcaacaaatgcacctccaggctgcgcgccctgtcccaagcccggtcccggtcccgctgcccaatcctggactctgcctctccccaatttcctttcccctgagcccccaacgcctaccttccgcggcctccatcccctcgcgcacacctaagctggtcgagcaaactcaccgcgcgcccgccggggatagctttccatacaggtaaaaccgaaaaccgaattttccaaaaatgcaccccgacggcgcctgctcttagtaccgtggggatgggagggaaattctttgtatatatttgtaaaaaaattattgactttccttttggggtttttatttttttaagaaaaaacaaattccgtagatttagagctctgaactttcattttttttgaaggttcactctccgaagttttatctgagaaaagaatgtatagagacgttgggagattttaaatataaaaaattttcaaaaaggcaaaaagtgtcattctattataaaagtctgtttatatatgaatgaatatatatggtattctaaatgttattccatcgtgttgtacacaactttgtaaataaatttttaaaatgccca

SEQ ID NO: 28 - protein, 478 aa, Homo sapiens
MTLSGGGSASDMSGQTVLTAEDVDIDVVGEGDDGLEEKDSDAGCDSPAGPPELRLDEADEVPPAAPHHGQPQPPHQQPLTLPKEAAGAGAGPGGDVGAPEADGCKGGVGGEEGGASGGGPGAGSGSAGGLAPSKPKNSLVKPPYSYIALITMAILQSPQKKLTLSGICEFISNRFPYYREKFPAWQNSIRHNLSLNDCFVKIPREPGNPGKGNYWTLDPQSEDMFDNGSFLRRRKRFKRHQQEHLREQTALMMQSFGAYSLAAAAGAAGPYGRPYGLHPAAAAGAYSHPAAAAAAAAAAALQYPYALPPVAPVLPPAVPLLPSGELGRKAAAFGSQLGPGLQLQLNSLGAAAAAAGTAGAAGTTASLIKSEPSARPSFSIENIIGGGPAAPGGSAVGAGVAGGTGGSGGGSTAQSFLRPPGTVQSAALMATHQPLSLSRTTATIAPILSVPLSGQFLQPAASAAAAAAAAAQAKWPAQ

SEQ ID NO: 29 - DNA, 2391 bp, Homo sapiens
accacatcaaccacggcatcaccacgccctcgtcactggatgccgggcccgacactgtggtcattggcatgactcgcatccctgtcattgagaacccccagtacttccgtcagggacacaactgccacaagccggacacgtgggtcttttcaaacatagacaatcatgggatattaaacttgaaggacaatagagatcatctagtcccatcaactcactatatatatgaggaacctgaggtccagagtggggaagtgtcttacccaaggtcacatggtttcagagaaattatgttgaatccaataagccttcccggacattccaagcctcttaaccatggcatctatgttgaggatgtcaatgtttatttcagcaaaggacgtcatggcttttaaaaactccttttaagcctccttgttttgatgtcaccttggtaggctgggccctctgagaggttggaagctctaggcattgttctctttggatccagggatgctaagtagaaactgcatgagccaccagtgccccggcaccctttaacaccaccagatgggtgttttcccccatccaccactggcagggttgccccttccctccaatcatcactgtgctccttttttcccggcctacgaggcagctcctgccactatctttagagccaataaagagaattaaaaacctgtgcaccaggagcatcttttaaatacactagccattctcttgctttacaaaaacaacctaaccatcacaagaaagcctgatgaagtccagccgtgctccagcctcactttccctgcttggaagcgtggggtctccctggctctcccaggataccatgctgtcctcttagtgacctcgtcgccctgcaacctccagtggggaagagtcacagagagcacctaagcagaggtggagacggcgcggtaagaggagggggagccaggctcaagtattggcaccaagttaggtctcagaggaaagaatggaaaccaatcactttacatttttatttttattttcggtggaaaaatcatccttttttgggacatacttgccccctacttcctcttctctctggaacggctcacaatgagtgtgacattagaaaactccttgcagaggagagtttctccaggctcttcctgggcccttagatctgcagttccgacaagctttggctgcaggaggttttacccatgaactggccatcctactaggaccacaagggaccaagggaatcagggacaaaggcccttcctgccagcccatgatcccgggattggctctcttcccctacttccacttattcttgactctgagaacttttggaacccaatggaatcagcatttcaaggtcaagatgaactgaaggggaagagaagtaaaacttggcctcctccagcccctctcatggcaccaatggaagtgtcctcctgttctctggtcaatatgtgtgtttactttgcttgctttgactcatgccttactccatggccaccctctcccaaagagggggctcgcttcccccattttcaacttgatccactgaggagagggaagggggtgactttcccttcttcagtaggaaaggcacatttgtagggcctgaaactctcccgtatttgctgactcattggtggagtagacttctggctcccagctccactggcccatggggcctccattgtatgaagtcagcataggctgcccacctaatggtggagagcatgaaactgggagcatcctgtggggggcttgtgggggaaaaaggtggttgttttaacccaccgttgttttggggtggtgttgcacactagtagagaatagagtctatgcctttggcaaatttaactgggagtttggattcccacttaagggttttacttcttgggtcctgtggatggtggttcttcgtgtcaggatcccagcccgattctgcaaatgcctccatggggtttaaaaacatgaggctttccaagttcttgcccagtatctggggcagcctccagagtatcacctgggagttcaggttctctccagggctccaggtgtgtgtttatctcgccccctccagctctcctcatcctgctccccattgctccatgtcaggctgttccccattgtgccctgctgatgctttgggtccagggcctcctcccaagtgtggctttaaggagtaagcttgaggatgatgttttttaattattgtaaatcattacctcatttccagcctcccaggctccatccatcccagcatcttttattctgccattttcctcaccttgtgctatgacaatggggcgttgtgtttccacagagacttataggagtgttcagtgtatagtttcttaataaacactttattttctaatgaaaaaaaaaaaaaaaaaa

SEQ ID NO: 30 - protein, 612 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTWVFSNIDNHGILNLKDNRDHLVPSTHYIYEEPEVQSGEVSYPRSHGFREIMLNPISLPGHSKPLNHGIYVEDVNVYFSKGRHGF

SEQ ID NO: 31 - DNA, 2435 bp, Homo sapiens
ccgcagttccttacaaactcttcacggaccacggcggcctcaacgagaagcgcaagcagcggcgcatccgcaccactttcaccagtgcccagctcaaagagctggaaagggtcttcgcggagactcactaccccgacatctacactcgggaggagctggccctgaagatcgacctcacagaggcgcgagtccaggtgtggttccagaaccgccgcgccaagtttcgcaagcaggagcgcgcagcggcagccgcagcggccgcggccaagaacggctcctcgggcaaaaagtctgactcttccagggacgacgagagcaaagaggccaagagcactgacccggacagcactgggggcccaggtcccaatcccaaccccacccccagctgcggggcgaatggaggcggcggcggcgggcccagcccggctggagctccgggggcggcggggcccgggggcccgggaggcgaacccggcaagggcggcgcagcagcagcggcggcggccgcggcagcggcggcggcggcagcggcagcggcggcagctggaggcctggctgcggctgggggccctggacaaggctgggctcccggccccggccccatcacctccatcccggattcgcttgggggtcccttcgccagcgtcctatcttcgctccaaagacccaacggtgccaaagccgccttagtgaagagcagtatgttctgatctggaatcctgcggcggcggcggcggcggcgacagcgggcgagccagggcccgggcgggcgagtgggcgagcgggtaggcccaaggctattgtcgtcgctgctgccatggctttttcattgagggcctaaagtaatcgcgctaagaataaagggaaaacggcgtcgccctcatttcaaccccactcctacccccttcctcaacccccaaacaaaacaaacaaacttccctggcttcgcacctgcctggggcctcgcagcggggccagggctccgcctgctgatcgggggttgtgagcagcgcggcctggacgcggggcactctcagggggctgtgtctgcgtgtcagtttgtgtctgtctcggggaatgtgtgtctgtggcccaagcaggtgacaggaagagatggggggcctcaaccaacttagtgacttgtttagaaaaaaaagacaaaaaagtaaaaataaaaacaaaaaagttggaaggcagaaaccattaaaaaacaaaaagccaacaacccagaaaggtttaaaaaacataaggaaaaaaaagacaaattaaaggaggggctaggggagaagctgcagctggagctgaaggctcgatcttgtgaacccctaaatccgctccctcctaacagcacggattctcttggggctcttcttcagggaagagtagggacgccgttccagccccccttcctatcgtgtccttgggttcgggtcactgcggcgacgacttgctcagactgtcccggcggccggagtgactttctcgcacccccttgcctgtcccacctcgctgaacaccatcccgccattagcgcatcggaaccccacacagttgcaactcccaaccccgaatctttgcagccgttcggccctgaaagatgccctatccatgagatgccttttcatctgcaaactctgcaaaatgtgtctcatgtttcgcaactctttttttccccctcgctcccgcctaccccgtcggcattttcttcttccaccagcttttactgaactttttggcactgctttggattggggtcaattgcagtccacgtaactggctgcagagaaatctaccgagcaaggaaaaggcacacacacacgtttgcaggggtgtctcggtttgcatttctgttggaatgatccgaactggactcacatcctgtatggtggatggactgtatattgagggttccattcttcgcgcagtttagacatctctgttttgattctttgttgttgtttttattttaaaaggcacaaactctagatattagttgaatgttgaggctttaactttttcggtgtctttctacaactgtgttctgtgactcaattgtatcgtgttaatatcagtgcagactgtctcctctacgtgaccgtataatgtttttctcttcttgtagtctctatggcgtgtctttatggtgtaataaggttctcacgggttcaatcttttgtgtttagagaggccacggttcagacaatggtatatatttttgttatcaggtgcatgtctgtctgatttctttttttttcctgttggactatgtttgtgaacataattgtcataagttatgtttcagatttttgaatttatttatatgtgttataatgaatgcttctatttaaaagggaaatatttctacatgtgcatatagttttccaagagtgtaccattaacttgattgttgataataaaaacaaaaagcaagtcta

SEQ ID NO: 32 - protein, 314 aa, Homo sapiens
MYKMEYSYLNSSAYESCMAGMDTSSLASAYADFSSCSQASGFQYNPIRTTFGATSGCPSLTPGSCSLGTLRDHQSSPYAAVPYKLFTDHGGLNEKRKQRRIRTTFTSAQLKELERVFAETHYPDIYTREELALKIDLTEARVQVWFQNRRAKFRKQERAAAAAAAAAKNGSSGKKSDSSRDDESKEAKSTDPDSTGGPGPNPNPTPSCGANGGGGGGPSPAGAPGAAGPGGPGGEPGKGGAAAAAAAAAAAAAAAAAAAAGGLAAAGGPGQGWAPGPGPITSIPDSLGGPFASVLSSLQRPNGAKAALVKSSMF

SEQ ID NO: 33 - DNA, 2411 bp, Homo sapiens
gaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctcctcgccctttcttaaagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgcctttttttttcttactgttttattacaaacttacaaaaatatgtataaccctgttttatacaaactagtttcgtaataaaactttttcctttttttaaaatgaaaa

SEQ ID NO: 34 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 35 - DNA, 2384 bp, Homo sapiens
ctttctaaaaagagatttatttttaaatcaatgggactctgatataaaggaagaataagtcactgtaaaacagaacttttaaatgaagcttaaattactcaatttaaaattttaaaatcctttaaaacaacttttcaattaatattatcacactattatcagattgtaattagatgcaaatgagagagcagtttagttgttgcatttttcggacactggaaacatttaaatgatcaggagggagtaacagaaagagcaaggctgtttttgaaaatcattacactttcactagaagcccaaacctcagcattctgcaatatgtaaccaacatgtcacaaacaagcagcatgtaacagactggcacatgtgccagctgaatttaaaatataatacttttaaaaagaaaattattacatcctttacattcagttaagatcaaacctcacaaagagaaatagaatgtttgaaaggctatcccaaaagacttttttgaatctgtcattcacataccctgtgaagacaatactatctacaattttttcaggattattaaaatcttcttctttcactatcgtagcttaaactctgtttggttttgtcatctgtaaatacttacctacatacactgcatgtagatgattaaatgagggcaggccctgtgctcatagctttacgatggagagatgccagtgacctcataataaagactgtgaactgcctggtgcagtgtccacatgacaaaggggcaggtagcaccctctctcacccatgctgtggttaaaatggtttctagcatatgtataatgctatagttaaaatactatttttcaaaatcatacagattagtacatttaacagctacctgtaaagcttattactaatttttgtattatttttgtaaatagccaatagaaaagtttgcttgacatggtgcttttctttcatctagaggcaaaactgctttttgagaccgtaagaacctcttagctttgtgcgttcctgcctaatttttatatcttctaagcaaagtgccttaggatagcttgggatgagatgtgtgtgaaagtatgtacaagagaaaacggaagagagaggaaatgaggtggggttggaggaaacccatggggacagattcccattcttagcctaacgttcgtcattgcctcgtcacatcaatgcaaaaggtcctgattttgttccagcaaaacacagtgcaatgttctcagagtgactttcgaaataaattgggcccaagagctttaactcggtcttaaaatatgcccaaatttttactttgtttttcttttaataggctgggccacatgttggaaataagctagtaatgttgttttctgtcaatattgaatgtgatggtacagtaaaccaaaacccaacaatgtggccagaaagaaagagcaataataattaattcacacaccatatggattctatttataaatcacccacaaacttgttctttaatttcatcccaatcactttttcagaggcctgttatcatagaagtcattttagactctcaattttaaattaattttgaatcactaatattttcacagtttattaatatatttaatttctatttaaattttagattatttttattaccatgtactgaatttttacatcctgataccttttccttctccatgtcagtatcatgttctctaattatcttgccaaattttgaaactacacacaaaaagcatacttgcattatttataataaaattgcattcagtggctttttaaaaaaatgtttgattcaaaactttaacatactgataagtaagaaacaattataatttctttacatactcaaaaccaagatagaaaaaggtgctatcgttcaacttcaaaacatgtttcctagtattaaggactttaatatagcaacagacaaaattattgttaacatggatgttacagctcaaaagatttataaaagattttaacctattttctcccttattatccactgctaatgtggatgtatgttcaaacaccttttagtattgatagcttacatatggccaaaggaatacagtttatagcaaaacatgggtatgctgtagctaactttataaaagtgtaatataacaatgtaaaaaattatatatctgggaggattttttggttgcctaaagtggctatagttactgattttttattatgtaagcaaaaccaataaaaatttaagtttttttaacaactaccttatttttcactgtacagacactaattcattaaatactaattgattgtttaaaagaaatataaatgtgacaagtggacattatttatgttaaatatacaattatcaagcaagtatgaagttattcaattaaaatgccacatttctggtc

SEQ ID NO: 36 - protein, 442 aa, Homo sapiens
MQPPPSLCGRALVALVLACGLSRIWGEERGFPPDRATPLLQTAEIMTPPTKTLWPKGSNASLARSLAPAEVPKGDRTAGSPPRTISPPPCQGPIEIKETFKYINTVVSCLVFVLGIIGNSTLLRIIYKNKCMRNGPNILIASLALGDLLHIVIDIPINVYKLLAEDWPFGAEMCKLVPFIQKASVGITVLSLCALSIDRYRAVASWSRIKGIGVPKWTAVEIVLIWVVSVVLAVPEAIGFDIITMDYKGSYLRICLLHPVQKTAFMQFYKTAKDWWLFSFYFCLPLAITAFFYTLMTCEMLRKKSGMQIALNDHLKQRREVAKTVFCLVLVFALCWLPLHLSRILKLTLYNQNDPNRCELLSFLLVLDYIGINMASLNSCINPIALYLVSKRFKNCFKSCLCCWCQSFEEKQSLEEKQSCLKFKANDHGYDNFRSSNKYSSS

SEQ ID NO: 37 - DNA, 2414 bp, Homo sapiens
tccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagctgcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctttgcactatccttcctctctgtgatgctttttaaaaatgtttctggtctgaacaaaaccaaagtctgctctgaacctttttatttgtaaatgtctgactttgcatccagtttacatttaggcattattgcaactatgtttttctaaaaggatgtgaaaataagtgtaattaccacattgcccagcaacttaggatggtagaggaaaaaacagatcagggcggaactctcaggggagaccaagaacaggttgaataaggcgcttctggggtgggaatcaagtcatagtacttctactttaactaagtggataaatatacaaatctggggaggtattcagttgagaaaggagccaccagcaccactcagcctgcactgggagcacagccaggttcccccagacccctcctgggcaggcaggtgcctctcagaggccacccggcactggcgagcagccactggccaagcctcagccccagtcccagccacatgtcctccatcaggggtagcgaggttgcaggagctggctggccctgggaggacgcacccccactgctgttttcacatcctttcccttacccaccttcaggacggttgtcacttatgaagtcagtgctaaagctggagcagttgctttttgaaagaacatggtctgtggtgctgtggtcttacaatggacagtaaatatggttcttgccaaaactccttcttttgtctttgattaaatactagaaattttttctgtttcctaacttcatcattgattgtttgaaatcttggagtttcaagcatttttttcaagctgagacggttccttttgcatgcctcctgactcactgactcctcac

SEQ ID NO: 38 - protein, 818 aa, Homo sapiens
MVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 39 - DNA, 1910 bp, Homo sapiens
accagacccctctgaggatggagcaggagctggctgccctgaggctgcaaaacttcttccctcgtggagacagggaggcacctcagacactcaccccggactcccttgaacagggacagggaggaaccccaggcagctagaccccagcagcagccacacgagcacactgtggggcagggaggggcatctcttgagaacaaaagatccatttctcgactttccaaactggagagcttcttgagagaaaagagagagacaggtacaggtccacgccacccacacacagccctgtgcacacagaccggacacaggcgtccacagttctgggaagtcatcagtgatgagcatggcatcgaccccagcggcaactacgtgggcgactcggacttgcagctggagcggatcagcgtctactacaacgaggcctcttctcacaagtacgtgcctcgagccattctggtggacctggaacccggaaccatggacagtgtccgctcaggggcctttggacatctcttcaggcctgacaatttcatctttggtcagagtggggccggcaacaactgggccaagggtcactacacggagggggcggagctggtggattcggtcctggatgtggtgcggaaggagtgtgaaaactgcgactgcctgcagggcttccagctgacccactcgctggggggcggcacgggctccggcatgggcacgttgctcatcagcaaggtgcgtgaggagtatcccgaccgcatcatgaacaccttcagcgtcgtgccctcacccaaggtgtcagacacggtggtggagccctacaacgccacgctgtccatccaccagctggtggagaacacggatgagacctactgcatcgacaacgaggcgctctacgacatctgcttccgcaccctcaagctggccacgcccacctacggggacctcaaccacctggtatcggccaccatgagcggagtcaccacctccttgcgcttcccgggccagctcaacgctgacctgcgcaagctggccgtcaacatggtgcccttcccgcgcctgcacttcttcatgcccggcttcgcccccctcacagcccggggcagccagcagtaccgggccctgaccgtgcccgagctcacccagcagatgttcgatgccaagaacatgatggccgcctgcgacccgcgccacggccgctacctgacggtggccaccgtgttccggggccgcatgtccatgaaggaggtggacgagcagatgctggccatccagagcaagaacagcagctacttcgtggagtggatccccaacaacgtgaaggtggccgtgtgtgacatcccgccccgcggcctcaagatgtcctccaccttcatcgggaacagcacggccatccaggagctgttcaagcgcatctccgagcagttcacggccatgttccggcgcaaggccttcctgcactggtacacgggcgagggcatggacgagatggagttcaccgaggccgagagcaacatgaacgacctggtgtccgagtaccagcagtaccaggacgccacggccgaggaagagggcgagatgtacgaagacgacgaggaggagtcggaggcccagggccccaagtgaagctgctcgcagctggagtgagaggcaggtggcggccggggccgaagccagcagtgtctaaacccccggagccatcttgctgccgacaccctgctttcccctcgccctagggctcccttgccgccctcctgcagtatttatggcctcgtcctccccacctaggccacgtgtgagctgctcctgtctctgtcttattgcagctccaggcctgacgttttacggttttgttttttactggtttgtgtttatattttcggggatacttaataaatctattgctgtcagataccctt

SEQ ID NO: 40 - protein, 378 aa, Homo sapiens
MDSVRSGAFGHLFRPDNFIFGQSGAGNNWAKGHYTEGAELVDSVLDVVRKECENCDCLQGFQLTHSLGGGTGSGMGTLLISKVREEYPDRIMNTFSVVPSPKVSDTVVEPYNATLSIHQLVENTDETYCIDNEALYDICFRTLKLATPTYGDLNHLVSATMSGVTTSLRFPGQLNADLRKLAVNMVPFPRLHFFMPGFAPLTARGSQQYRALTVPELTQQMFDAKNMMAACDPRHGRYLTVATVFRGRMSMKEVDEQMLAIQSKNSSYFVEWIPNNVKVAVCDIPPRGLKMSSTFIGNSTAIQELFKRISEQFTAMFRRKAFLHWYTGEGMDEMEFTEAESNMNDLVSEYQQYQDATAEEEGEMYEDDEEESEAQGPK

SEQ ID NO: 41 - DNA, 2388 bp, Homo sapiens
ttgcccgagttcctccgggaagcgctccgggtagattctgggggccgggagctgagatccctgggcggggagctggggaagggatggggctgaggacagcgaagaagagggggcttgggggaggggggaaatggaagagagaggagggaggaggtacaagaggaaggagagaagtgcggccagcttgctttctccagtcgggtggccgcggggacccgggcgacgtcggaggccctgccgggaacccaggctgcagcccccacccccgcgctgcgacacgccccccaccccttccggctcacacccccgcccacactcctgagtggtgcggtgcagcgtcggccgaggcagcagagccgaggagagcaggtccacacctctgcatccctgcaccaggactcaccaagacgcccatcctggaaaaggtcccccgtaagatggcagcaaaaactcccagcagtgaggagtctgggctgcccaaactgcccgtgcccccgctgcagcagaccctggccacgtacctgcagtgcatgcgacacttggtgtctgaggagcagttcaggaagagccaggccattgtgcagcagtttggggcccctggtggcctcggcgagaccctgcagcagaaactcctggagcggcaggagaagacagccaactgggtgtctgagtactggctgaatgacatgtatctcaacaaccgcctggccctgcctgtcaactccagccctgccgtgatctttgctcggcagcacttccctggcaccgatgaccagctgaggtttgcagccagcctcatctctggtgtactcagctacaaggccctgctggacagccactccattcccactgactgtgccaaaggccagctgtcagggcagcccctttgcatgaagcaatactatgggctcttctcctcctaccggctccccggccatacccaggacacgctggtggctcagaacagcagcatcatgccggagcctgagcacgtcatcgtagcctgctgcaatcagttctttgtcttggatgttgtcattaatttccgccgtctcagtgagggggatctgttcactcagttgagaaagatagtcaaaatggcttccaacgaggacgagcgtttgcctccaattggcctgctgacgtctgacgggaggagcgagtgggccgaggccaggacggtcctcgtgaaagactccaccaaccgggactcgctggacatgattgagcgctgcatctgccttgtatgcctggacgcgccaggaggcgtggagctcagcgacacccacagggcactccagctccttcacggcggaggctacagcaagaacggggccaatcgctggtacgacaagtccctgcagtttgtggtgggccgagacggcacctgcggtgtggtgtgcgaacactccccattcgatggcatcgtcctggtgcagtgcactgagcatctgctcaagcacatgacgcagagcagcaggaagctgatccgagcagactccgtcagcgagctccccgccccccggaggctgcggtggaaatgctccccggaaattcaaggccacttagcctcctcggcagaaaaacttcaacgaatagtaaagaaccttgacttcattgtctataagtttgacaactatgggaaaacattcattaagaagcagaaatgcagccctgatgccttcatccaggtggccctccagctggccttctacaggctccatcgaagactggtgcccacctacgagagcgcgtccatccgccgattccaggagggacgcgtggacaacatcagatcggccactccagaggcactggcttttgtgagagccgtgactgaccacaaggctgctgtgccagcttctgagaagcttctgctcctgaaggatgccatccgtgcccagactgcatacacagtcatggccataacagggatggccattgacaaccacctgctggcactgcgggagctggcccgggccatgtgcaaggagctgcccgagatgttcatggatgaaacctacctgatgagcaaccggtttgtcctctccactagccaggtgcccacaaccacggagatgttctgctgctatggtcctgtggtcccaaatgggtatggtgcctgctacaacccccagccagagaccatccttttctgcatctctagctttcacagctgcaaagagacttcttctagcaagtttgcaaaagctgtggaagaaagcctcattgacatgagagacctctgcagtctgctgccgcctactgagagcaagccattggcaacaaaggaaaaagccacgaggcccagccagggacaccaaccttgactcctgccactaggtttcacctcccaaacccagcctctagaacagccagaccctgcag

SEQ ID NO: 42 - protein, 630 aa, Homo sapiens
MAAKTPSSEESGLPKLPVPPLQQTLATYLQCMRHLVSEEQFRKSQAIVQQFGAPGGLGETLQQKLLERQEKTANWVSEYWLNDMYLNNRLALPVNSSPAVIFARQHFPGTDDQLRFAASLISGVLSYKALLDSHSIPTDCAKGQLSGQPLCMKQYYGLFSSYRLPGHTQDTLVAQNSSIMPEPEHVIVACCNQFFVLDVVINFRRLSEGDLFTQLRKIVKMASNEDERLPPIGLLTSDGRSEWAEARTVLVKDSTNRDSLDMIERCICLVCLDAPGGVELSDTHRALQLLHGGGYSKNGANRWYDKSLQFVVGRDGTCGVVCEHSPFDGIVLVQCTEHLLKHMTQSSRKLIRADSVSELPAPRRLRWKCSPEIQGHLASSAEKLQRIVKNLDFIVYKFDNYGKTFIKKQKCSPDAFIQVALQLAFYRLHRRLVPTYESASIRRFQEGRVDNIRSATPEALAFVRAVTDHKAAVPASEKLLLLKDAIRAQTAYTVMAITGMAIDNHLLALRELARAMCKELPEMFMDETYLMSNRFVLSTSQVPTTTEMFCCYGPVVPNGYGACYNPQPETILFCISSFHSCKETSSSKFAKAVEESLIDMRDLCSLLPPTESKPLATKEKATRPSQGHQP

SEQ ID NO: 43 - DNA, 2411 bp, Homo sapiens
aagagccgacgcgaggggaggggagcgcagcggcggggctaacgggcgggcaagcgggcgggcggcaacagcatgtccctcggccagcgcgggcggcctcttagcgcggcgggggctgctctgggcgcgccccgggcgaagtgcgcccagtctccggccccggcccctcggcgcgcccgacttcccggccgcccctgagcccagcagccgcgggtcccgggatcggctaagagtagctgcaacgcctcgccggacggagtcctttcctttcccgggacgctgggccatgagctccgcggccacctgaggcacaggggagtctgctcggccaggacagcctccccgaagtcccgtgccctcgcctctgcactgcgggacgccagcgctcggccctggcggaggcgtcctcggaagagcatcggggtgggggcatggaatccgcggaacctgcgggccaggcccgggcggcggccaccaagctgtcggaggctgtgggcgcggcgctgcaggagccccggcggcagaggcgcctggtgcttgttatcgtgtgcgtggcgctgttactggacaacatgctgtacatggtcatcgtgcccatagtgcccgactacatcgcccacatgcgcgggggcggcgagggccccacccggactcccgaggtgtgggagcccaccctgccgctgcccactccggccaatgccagcgcctacacggccaacacctcggcgtccccgacagctgcgtggccagcgggctcagcccttcggccccgctaccctacggagagcgaagacgtgaagatcggggtgctgtttgcttccaaggctatcctgcagctgctagtgaaccccttgagcgggcccttcatcgaccgcatgagctacgacgtgccgctgctgatcggcctgggcgtcatgttcgcctctacagtcctgttcgccttcgccgaggactacgccacgctgttcgcggcgcgcagcctgcagggcctgggctcagccttcgccgacacgtctggcatagccatgatcgccgataagtacccggaggagccggagcgcagtcgtgcactgggcgtggcgctggccttcattagcttcggaagcctagtggccccgcccttcgggggcatcctctatgagttcgccggcaagcgcgtgcccttcttggtgctagctgccgtgtcgctctttgacgcgctgttgctgctggcagtggccaaacccttctcggcggctgcacgggctcgggccaacctgccagtgggcactcccatccaccgcctcatgctagacccctacattgccgtggtggccggcgcgctcaccacctgtaacattcccctcgccttcctcgaacccaccattgccacgtggatgaagcatacgatggcggcttccgagtgggagatgggcatggcctggctgccggccttcgtgcctcatgtgctgggcgtctacctcaccgtgcgcctggcggcgcgctacccacacctgcagtggctgtacggcgcgcttgggctggctgtgatcggcgccagctcgtgcatcgtgcccgcctgccgctccttcgcgccgctagtggtctcactatgcggcctctgttttggcatagccctagtcgacacagcactgctgcccacgctcgccttcctggtggacgtgcgccatgtctcagtctatggcagcgtctacgccatcgccgacatctcctattcggtggcctacgcgctcgggcccatagtggcaggccacattgtgcactcgctgggctttgagcagctcagccttggcatgggactggccaacctgctctatgctcccgtcttgctgctgctccgcaacgtgggcctcctgacgcgctcccgttccgagcgcgatgtgctgcttgatgagccaccgcaaggtctgtacgatgcggtgcgcctgcgtgagcgtcctgtgtctggccaggacggcgagcctcgcagcccgcctggcccttttgatgcgtgcgaggacgactacaactactactacacccgcagctagcatccccactcctcctccagcccacccaaccgccttgggtcaagggggctgctctgcaagcccactggccagctctggctcagggcccacctcctccagcgagtaccccagccactcctcaaccttgacttctgcccaaatcccctccctgtgacccgttccatatccctttctctcttgtccaatggggcttggagcaccgaggccagcgaagccatcgcgctccttgcggaggtgaagaggaccctgagtccccacctgcggctcccctgtgtagagcctgcatctgtctgtccttccttccattgctcccagtgccaaacttgggccgctgcaccgcggcgcctccgcccaaatcaataaactgtgtctgtcccagga

SEQ ID NO: 44 - protein, 532 aa, Homo sapiens
MESAEPAGQARAAATKLSEAVGAALQEPRRQRRLVLVIVCVALLLDNMLYMVIVPIVPDYIAHMRGGGEGPTRTPEVWEPTLPLPTPANASAYTANTSASPTAAWPAGSALRPRYPTESEDVKIGVLFASKAILQLLVNPLSGPFIDRMSYDVPLLIGLGVMFASTVLFAFAEDYATLFAARSLQGLGSAFADTSGIAMIADKYPEEPERSRALGVALAFISFGSLVAPPFGGILYEFAGKRVPFLVLAAVSLFDALLLLAVAKPFSAAARARANLPVGTPIHRLMLDPYIAVVAGALTTCNIPLAFLEPTIATWMKHTMAASEWEMGMAWLPAFVPHVLGVYLTVRLAARYPHLQWLYGALGLAVIGASSCIVPACRSFAPLVVSLCGLCFGIALVDTALLPTLAFLVDVRHVSVYGSVYAIADISYSVAYALGPIVAGHIVHSLGFEQLSLGMGLANLLYAPVLLLLRNVGLLTRSRSERDVLLDEPPQGLYDAVRLRERPVSGQDGEPRSPPGPFDACEDDYNYYYTRS

SEQ ID NO: 45 - DNA, 2417 bp, Homo sapiens
cttgccattaggaaatctgtggacttctagcctgtgttttaaaccagccatgtttccttgtatatttccctacccgctgccccacatacccagcatgccgctgtggccaccatgtcctcaaagccttctgtctgtatcaggaatgtagtctgagactgccaggaagcaacaaggagagagaaacactaactagtcttcctttataacccattcatactctctggctgtccccaaccttcatagtctcctgcatccaaatgtcctctttggctcaaaaagtaggccaggcatggtggttcatgcctgtaatagcactttgggagactgaggtgggaggatcacttggggccaggagtttgagaccagcttgggcaacacagcgcaatctcgtctctactaaaaaaaaaaaaaaaaaaaaattagctgggcatgatggcatgctcctgtggtcccagctacttgggaggctgaggcaggaggatcacttggtcccaggagtttgaggcgacagtgagctaggatcgcaccactgcactccagcctgagtgacagagcaagaccctgtctctaaaaaaaattaaaatgaaagaccaggtgctgggattaaggaaacacaggtctgagggtctgagggaaggggcctgcctcccagggagtcaacatagatgttccccatgaacagggatttgactttggaggccaacctggcctggcctctgccctttatctcacactccctatccttggcccactgccagtccctgccttgtggcaaaggggccccaaaagaaaagctgcccttccccaaatgtaaggacccaggtacactttcacccgtggaaagcagtgtctgtcgagagtctgtttcctattaatacttatcaaagccatgtgcgagggaggtggtcagctgtcaatatgccttagtatgtttatatgagtttgttttgttctaaaatacccaaacagttctggtcaagcggggctatgcccgtctggcccaaaacacagtccgttattaacgagatggccctggcaggcgggaacaaatctgcctccatgcactgcttcctgtagtcttttagaaagtaactccaggacatcgaagtgcccagatttgactcctaagttctaggagactgtagcgcagggtctgtcaaccttagcactattggcatttggggctgggtaattctttcttgtgggggccgtcttgggtactgtaggaagctgagcagcattcctggcctccatccacaagatacctgtagcagtgtcctgccaacggtaacaatcaagtatgtcatcagacattgcccaatgtccccagggggcaacacccctctcttggacttcagggtcaagagaatctctgctggctaccccaggacttctcattatagatttcctggagcacgcagcagaaactttgcctagcccagtggttgtttccattatctgctgccaaagtgggatttgagggtgtccgggggagggggcatggggagggcagtatgctttcaaaaacccctcccaggccaggcgtggtggctcatgcctgtaatcacaggactttgggaggccgaggctggcagatcacttgaggctgggagttagagaccaacctggctaacatggcaaaacctcgtctctactaaaaatacaaaaatcagcccggcgtggtggcgggcatctgtaatcccatctactcgggaggctgaggcaggagaattacttgaacccaggaggcagaggctgcagtgagccgagatggcaccactgcactccagcttgttgacagaatgagaccctgtggaaaaaaaaaaaaaagccctcccatgccagaacagaggatggcagtctgtttcaataagacactgtgtccttggtgttggttctgattaagactcactgagatccagtgctcttgagctgggtctcagtcccctcccatgtcctgtgctctgccgccactgttttcattgttgtgttctcgttgtgattgttaagactcacactcctggctcagcagtggttttccagaaggcccaaagagcggtgccgggcaccccacgtcgcagtgtccgttccgggcttgggaagctggggaggtgggcagacctggtcgcatctcaccacacacacacacacacacacacacacacacgctgtcagaaactcggccgtcccccctacctctgagctctcaatgctgctaatctctgccaagtgtccctgtgctccagcaccttccttgaaggactgacgcccaccccacgctctttgcgaggttgtccaggctgtgtttgtcgcatgctcttcttctgtatagttctcatcttccaattttatgggattcaacaaaagcctattatgcttgtttgcattatggttacaatattaaaaagtggattcaa

SEQ ID NO: 46 - protein, 1434 aa, Homo sapiens; identical to SEQ ID NO: 10

SEQ ID NO: 47 - DNA, 2409 bp, Homo sapiens
gagattctgctttggagggtaaagtggggaagaaatcggattcccagaggtgaatcagctcctctcctacttgtgactagagggtggtggaggtaaggccttccagagcccatggcttcaggagagggtctctctccaggactgccaggctgctggaggacctgcccctacctgctgcatcgtcaggctcccacgctttgtccgtgatgcccccctaccccctcactctccccgtctccatggtcccgaccaggaagggaagccatcggtaccttctcaggtactttgtttctggatatcacgatgctgcgagttgcctaaccctccccctacctttatgagaggaattccttctccaggcccttgctgagattgtagagattgagtgctctggaccgcaaaagccaggctagtccttgtagggtgagcatggaattggaatgtgtcacagtggataagcttttagaggaactgaatccaaacattttctccagccggacattgaatgttgctacaaagggagccttgaagctttaacatggttcaggcccttggtgtgagagcccagggggaggacagcttgtctgctgctccaaatcacttagatctgattcctgttttgaaagtcctgccctgccttcctcctgcctgtagcccagcccatctaaatggaagctgggaattgcccctcacctcccctgtgtcctgtccagctgaagcttttgcagcactttacctctctgaaagccccagaggaccagagcccccagccttacctctcaacctgtcccctccactgggcagtggtggtcagtttttactgcaaaaaaaaaaaaagaaaaaagagaaagaaaaaaaagaatgaatgcaagctgatagctgagactgtgagactgtttttgtccactcttctgaatcactgccacttgggtcagggaccacagccattgccacccttggcccatctctctgcgtgcgtgccttgagcacacatataaaaagtgccatgtgcaattgtcttatcttttatgatctaggctttgcctagggatcactactccttaacgggctggctggggcaatgaggaaaagctcctttgctcctgtaaggccataagtggctgttaacagattttcaaatgcctgaagagattgctgagacctgctagagtcatatgttcggggaattaagtctttatcctagacaacaaggtacagatgcaaactgcagtgttattggagggtcaatcggcaaggatatgattatcccaaaatggagttcatcgaccctagctttcctttagattatatataaataaaagtgcagtcctcttctaatggccacagttggttttcttgtagcccagaaagtccaaattaaaggaaataaattcagttttatgttagccttccttggtgcatcagggtgtcagtggaaataggatcaggtggtgtgtgtgtgtgtgttttgtgtgtgtgtgtacacatgtgtttatatatacatgtgtgagggaaagtgtgtacatatatgtaggattgtaaccagacggaaaagaacgaggatctccagggtgtttgaatcagcaacagatttgtgttttctaacatgcatttagttggagaggcatggttctgtttgttttgttttgatctaatttgccattggaaataggtacagttacacagagaaggaagaaccaggaaagtgagatccatgaaactaaatgagcagctgtcagaatccagtgtggctgagcctacctagcttatgaaatctaacccagggttccctgagtccaagaccacttagattattaagattttgaacgtccagaggagtgaaaagtctgttttctgacgtaagccggagctgaggataaagccagaggccagtggattaggtgtatggaatgtggatggagagggcttgtgtgggatgtggccagggagtgggtgaggaaggccgcttctaaatggcctgtaaaaacttgagattggatagacgaaaggaaatggagaaattaaagaattggagaaactagttatctgtgttgctgactttgggacccatccaagactcctgcccttggggtgttccatggtggtttcttcctgcctgggcgccaccctttccccagttcaggccctccctggaggactagtttgtgtattggtatcctccccagtggacccaaaccagcgcatacttggtgtgtggagatgggagacaaaggacagatctaggagccttgaaggatcaccagccaccgaccctccatcagggccaactgggcaggaaagggaacattgcagacctgatttcccgacgatgtcaccctgtcctccctccttgcttcttgctctgctaactcaactctgccttcctctttttcattcttctactctgcc

SEQ ID NO: 48 - protein, 211 aa, Homo sapiens
MSLSSLCPVFSAAASSLQVHLLKDQLAAEAAARLEAQARVHQLLLQNKDMLQHISLLVKQVQELELKLSGQNAMGSQDSLLEITFRSGALPVLCDPTTPKPEDLHSPPLGAGLADFAHPAGSPLGRRDCLVKLECFRFLPPEDTPPPAQGEALLGGLELIKFRESGIASEYESNTDESEERDSWSQEELPRLLNVLQRQELGDGLDDEIAV

SEQ ID NO: 49 - DNA, 3279 bp, Homo sapiens
ctcttctcctggcgctcgcgtgcgagagggaactagcgagaacgaggaagcagctggaggtgacgccgggcagattacgcctgtcagggccgagccgagcggatcgctgggcgctgtgcagaggaaaggcgggagtgcccggctcgctgtcgcagagccgagcctgtttctgcgccggaccagtcgaggactctggacagtagaggccccgggacgaccgagctgatggcgtcttcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcaaggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatgctgcctggggaggtgggctgctcatgtccaggaagcaccgccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatacctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaatcatccttcgcagaacatgagtttatgggaatgccttttccctctggcactccagaacaaacctctatatgttgctgaaacacacaggccatttcattgagggaaaacataatatcttgaagaatattgttaaaaccttacttaaagcttgtttgttctagttagcaggaaatagtgttctttttaaaaagttgcacattaggaacagagtatatatgtacagttatacatacctctctctatatatacatgtatagtgagtgtggcttagtaatagatcacggcatgtttcccgctccaagagaattcactttaccttcagcagttaccgaggagctaaacatgctgccaaccagcttgtccaacaactccaggaaaactgtttttcaaaacgccatgtcctaggggccgagggaaatgctgttggtgagaatcgacctcactgtcagcgtttctccacctgaagtgatgatggatgagaaaaaacaccaccaaatgacaagtcacaccctccccattagtatcctgttaggggaaaatagtagcagagtcattgttacaggtgtactatggctgtatttttagagattaatttgtgtagattgtgtaaattcctgttgtctgaccttggtggtgggagggggagactatgtgtcatgatttcaatgattgtttaattgtaggtcaatgaaatatttgcttatttatattcagagatgtaccatgttaaagaggcgtcttgtattttcttcccatttgtaatgtatcttatttatatatgaagtaagttctgaaaactgtttatggtattttcgtgcatttgtgagccaaagagaaaagattaaaattagtgagatttgtatttatattagagtgcccttaaaataatgatttaagcattttactgtctgtaagagaattctaagattgtacataaagtcatatatatggaaatcctgttacttaaatagcatctgctcttctcttacgctctctgtctggctgtacgtctggtgttctcaatgcttttctagcaactgttggataataactagatctcctgtaattttgtagtagttgatgaccaatctctgtgactcgcttagctgaaacctaaggcaacatttccgaagaccttctgaagatctcagataaagtgaccaggctcacaactgtttttgaagaagggaaattcacactgtgcgttttagagtatgcaagaagaatataaataaataaaaatattctccatggagaatttgaacaaaa

SEQ ID NO: 50 - protein, 594 aa, Homo sapiens
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 51 - DNA, 2404 bp, Homo sapiens
acgcacgcgcgcgcagggccaagcccgaggcagctcgcccgcagctcgcactcgcaggcgacctgctccagtctccaaagccgatggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataataaccttgctcaccaagctgttccacttctctagagaacatgccctcagctaagccccctactgagaaacttcctttgagaattgtgcgacttcacaaaatgcaaggtgaacaccactttgtctctgagaacagacgttaccaattatggagtgtcaccagctgccaaaatcgtaggtgttggctctgctggtcactggagtagttgctactcttcagaatatggacaaagaaggcacaggtgtaaatatagtagcaggatgaggaacctcaaactgggtatcattttgcacgtgctcttctgttctcaaatgctaaatgcaaacactgtgtatttattagttaggtgtgccaaactaccgttcccaaattggtgtttctgaatgacatcaacattcccccaacattactccattactaaagacagaaaaaaataaaaacataaaatatacaaacatgtggcaacctgttcttcctaccaaatataaacttgtgtatgatccaagtattttatctgtgttgtctctctaaacccaaataaatgtgtaaatgtggacacatctc

SEQ ID NO: 52 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 53 - DNA, 2415 bp, Homo sapiens
ctaactccattatcttcactctttccttcttcctgccacctcatgcccattctctttactgtctagcatgctgaaagaaggaagtgatctaaatgccagcgtgttcagtggtaaatattagttggtgcaaaagaaaaaccatgattacttttgcactaacctaatagctttgcaaattttaagaacttgctttatgaagatattcggatatggattctccccaccccacatacttagacattgttcaaatatactacttttaaaaaaacaccttttcaaacagaattagcgttttgccaagtctggtattaatggaattgtacaggagctttgaaagttttcaaactttattaaactaaaaaaaaaaaatcgaaaatctctgtctgttccgcatagtatgcatttatttgacccctatttatcaatactatgatggggttttttttttttaaagaaaatttaagagtaggtaggtggattttaaaataatattttaaagaccttttatatctatatgtagcatttatagaaaaataaaaactaaaaatagaattgaattgtaacattatttaaggactgaagttttttttcttgtatcagtaagaaatacccaagaggctgggtgcagtgactcacacctgtaatcccagcactttgggaggctgcagtgggaggatcacatgacatcaggagtttgagaccagcttggccaacatagtgaaacgccgtctctattaaaaatacagaaaattagctgaatgtggtggcaggcgcctataattcctgctacttgggaggctgaggcaggagaattgcttgaacccaggaggcagaggttgcagtgagccaaacgttccactgcattccagcctggatgacaagagcgaaactccgtctcaaaaaaaaaaaaaaaaattgttatgcttagttccatggaaagactattctgaagctttaagtcttctttttctattttccatagtattgccccttccccacttcattgcttaactgtctctaaattttaatgataataatattttaaaggtcagataatgccattagaggcagagacaaacctggggaccaagctaatttctctgttattgagagcgctaaagacaggtgaactggagttttatttcctctgctagagtgaaataataccctctccaccaggcacaatggctcacacctgtaattccagcactttgggaggccaaggtgggcggatcacttgaggtcaggagttcgagaccagcctggccaacatggtgaaaccccatctctactaaaaatacaaaaattagccagccatggtggcatgctcctgtaatcccagctacttgggagactgaggtgggataatcacttgaacctgggaggcggaggttgcagtgagctgagattgtgccactgcactccagcctgggtgacagagcaagactccatctaaaaaacaaaaacaaaaacaaaaaaaccctctcaattggtttaataccacgatagaaaagataaatattttaggatggaatcttaaatatgtctgtccttttgtttcatatagttgaaatcaattcagtattgtttctacattaggtgtttcaaaaacagtcacctttgcaacagaagagctcttttgttgaaaatgattcacaatatatttcagttggaatgtcaggtggtatttctcttaccaacacctcacagaatctatgccaagctgctctaccaccaatgcaagtatttttttaaagctactaaaataaactttcttcaccagccaactctaatttggagacagtgtgcattggtgaaggacctcgtcagaataagtttgaatgtctactgaactaagaagaattttgtcgtttgggggagagaatagatggcatcagtccttcaattctgtaactgaagactccaattatagtagataagaattgtgtctagcaatttttaaacactgacagtccaaacaaaaatatttggtgaggaaggatgtcccatatttttgcttaaatatcataaacaaatatgagcatttactaattttttaaatggcattttgaaagattattcttatttacactctaaaattaaaggtgtactttatcttaagaaaatgatatattaaaaattcatattttaaaagataaaattggggaatttacagttattttgtgaatggcctttaaactatgatttgatctatatacaacttttcagaatacttttgattgtgtgttggacatatctaaaattaattttatctggcagaattaaacctaaatttatttgtataaaagagtcccctttctaaaattcatacagtgcccttgtattcatttatgcagtgtttataaatatttcaagttagattgtgagatattaataaatgatttatgctgtgaa

SEQ ID NO: 54 - protein, 630 aa, Homo sapiens
METTPLNSQKQLSACEDGEDCQENGVLQKVVPTPGDKVESGQISNGYSAVPSPGAGDDTRHSIPATTTTLVAELHQGERETWGKKVDFLLSVIGYAVDLGNVWRFPYICYQNGGGAFLLPYTIMAIFGGIPLFYMELALGQYHRNGCISIWRKICPIFKGIGYAICIIAFYIASYYNTIMAWALYYLISSFTDQLPWTSCKNSWNTGNCTNYFSEDNITWTLHSTSPAEEFYTRHVLQIHRSKGLQDLGGISWQLALCIMLIFTVIYFSIWKGVKTSGKVVWVTATFPYIILSVLLVRGATLPGAWRGVLFYLKPNWQKLLETGVWIDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHVWAKRRERFVLAVVITCFFGSLVTLTFGGAYVVKLLEEYATGPAVLTVALIEAVAVSWFYGITQFCRDVKEMLGFSPGWFWRICWVAISPLFLLFIICSFLMSPPQLRLFQYNYPYWSIILGYCIGTSSFICIPTYIAYRLIITPGTFKERIIKSITPETPTEIPCGDIRLNAV

SEQ ID NO: 55 - DNA, 2446 bp, Homo sapiens
aaggtataaggtgaggccataaacagaaaggtatgcaatgagagtagaggctgcagaaaaggggaggaaatagaatgtcagaaaggtcaagaagagtggtttaggactgagactgtaagtaatgtaaggattgccaaggaatggtttttatgcagagtcagctgtgtagcacagggagtaagcagtcctgttataaatgggtgaagctgaactccaaaatacaaacttatgtctctttaatttgcccctttcctccctctagtcactaaatgaagatgttcttcaagtttctgtctttgcacttctactctttttgccatctcttcatggggaatgtcacccagatacatgacttcagttcttattttgaatgactcctctatttgcagttataaccctgacatctttcttgaatttttttgccccttaattccaacgaactcctggatgtctatgtagatgtctcataatttaggccaatgctgtacaacttcatgtggcatcatccacatagaatgaataggggttgttcaatatgacagccctgccaagcatctcaaattgaacaggtctataaaggaactcagaatcacttgaatatcacagtccatcagcttcctttcccagtaatttaatttccattaatggtatattaaacatcgtattttcaatcatttttaggtttagtcagctacttaaaaataggctttttcttcacataaataaattgtataccatttaatgtgttttatttcaacagactaatagatcttttaacacttcttagtcctcaaaataaattattaaaaactttgtatttcatgtcatattatataaggaaatagaattttgaagaataattcatgctgttttaaatttttacattgtacattttaatagctaatcaactcttaagtatacatttgatggtaaatagatgctagctaatctagaatcatttgaaatattgattatctgatttatagtttggttgcaaggaaatctactgtaagcaaatcataatagatagcaaatcttagaacatgtagaaaaaaagaaattaaaaaaagcaaatcttagaacatgtagaaaaaggaaattaaaaaatagatagcaaataacacaaattatgcatactatatgtggttattttcatacaactttgataacggtgaagaatcatacttattgaattttaaaatgatgatgtttaaaatcactctatgactaaagcacatgagaataaatgttaaaggcacagccttataatccacaacttcagaacaatggaatttttttttttttttttttttttttttttttttttttttttttgagacggagtcttgctctgtcgcccaggctggagtgcagtggtgcgatcttggctcactgcaagctccacctcccaggttcacggcattctcctgcctctgtctcctgagtagctgggactacaggcgcccgccaccacgtccggctcattttttgtatttttagtagagacaaggtttcaccgtgttagccaggatggtttcaatctcctgacattgtgatctgcccacctcggcctcccaaagtgctgggattacaggcgtgagccactgcacccggccaaacattggaaatttttaatgatggatttcagttcttctcacctctgggtatttgtaatatttggtaaatcaggagtcctgagagtcttagaattttcatgcattttaaatatatgtcttcaaagcacctaggaatatgtaaatagttacccaaatgtgagaacagaattgaactaaaagcttcgtttataatttttttttttttttgagacagagtctcactctgttgcccaggctggagcgcagttgcgtgatttcagctcgctgcaacctgggactgcaggcgtgcttcaccatgccccgctaatttttgtttttttagtagagatggggtttcgccatgttggccaggctggtctcgaacttctgaccgcaggtgatccatccgcctcggcctcccaaagtgctgggattacaagcatgagccaccatgcccagccggttaataattcttaatcagtaaatcattattcaaagacatagttaatatcttcatgacttcttaaggatgctcaaaagctggtcttcagaatacctacatttctttatctacatattctgtcatttggagttatacaagtagtaatgacacaccaaaatgtaaataattattgttagcttttaagttggcttccaaattcaaaaaagaaaaaaatcctgaattctctaaaggaacatatgatagagtccatatgttaattcataggaagtgtttaaggtactatggtctattttggtctaatctttgttttactatgtaatatatatttatgatttacaagtttgttattcagtgggataataaatgaacacaaaatttatgttctcaa

SEQ ID NO: 56 - protein, 444 aa, Homo sapiens
MIEDNKENKDHSLERGRASLIFSLKNEVGGLIKALKIFQEKHVNLLHIESRKSKRRNSEFEIFVDCDINREQLNDIFHLLKSHTNVLSVNLPDNFTLKEDGMETVPWFPKKISDLDHCANRVLMYGSELDADHPGFKDNVYRKRRKYFADLAMNYKHGDPIPKVEFTEEEIKTWGTVFQELNKLYPTHACREYLKNLPLLSKYCGYREDNIPQLEDVSNFLKERTGFSIRPVAGYLSPRDFLSGLAFRVFHCTQYVRHSSDPFYTPEPDTCHELLGHVPLLAEPSFAQFSQEIGLASLGASEEAVQKLATCYFFTVEFGLCKQDGQLRVFGAGLLSSISELKHALSGHAKVKPFDPKITCKQECLITTFQDVYFVSESFEDAKEKMREFTKTIKRPFGVKYNPYTRSIQILKDTKSITSAMNELQHDLDVVSDALAKVSRKPSI

SEQ ID NO: 57 - DNA, 2360 bp, Homo sapiens
gcattgctcttcagcaccagggttctggacagcgccccaagcaggcagctgatcgcacgccccttcctctcaatctccgccagcgctgctactgcccctctagtaccccctgctgcagagaaagaatattacaccgggatccatgcagccagcaatgatgatgttttccagtaaatactgggcacggagagggttttccctggattcagcagtgcccgaagagcatcagctacttggcagctcaacactaaataaacctaactctggcaaaaatgacgacaaaggcaacaagggaagcagcaaacgtgaagctgctaccgaaagtggcaagacagcagttgttttctccttgaagaatgaagttggtggattggtaaaagcactgaggctctttcaggaaaaacgtgtcaacatggttcatattgaatccaggaaatctcggcgaagaagttctgaggttgaaatctttgtggactgtgagtgtgggaaaacagaattcaatgagctcattcagttgctgaaatttcaaaccactattgtgacgctgaatcctccagagaacatttggacagaggaagaagagctagaggatgtgccctggttccctcggaagatctctgagttagacaaatgctctcacagagttctcatgtatggttctgagcttgatgctgaccacccaggatttaaggacaatgtctatcgacagagaagaaagtattttgtggatgtggccatgggttataaatatggtcagcccattcccagggtggagtatactgaagaagaaactaaaacttggggtgttgtattccgggagctctccaaactctatcccactcatgcttgccgagagtatttgaaaaacttccctctgctgactaaatactgtggctacagagaggacaatgtgcctcaactcgaagatgtctccatgtttctgaaagaaaggtctggcttcacggtgaggccggtggctggatacctgagcccacgagactttctggcaggactggcctacagagtgttccactgtacccagtacatccggcatggctcagatcccctctacaccccagaaccagacacatgccatgaactcttgggacatgttccactacttgcggatcctaagtttgctcagttttcacaagaaataggtctggcgtctctgggagcatcagatgaagatgttcagaaactagccacgtgctatttcttcacaatcgagtttggcctttgcaagcaagaagggcaactgcgggcatatggagcaggactcctttcctccattggagaattaaagcacgccctttctgacaaggcatgtgtgaaagcctttgacccaaagacaacttgcttacaggaatgccttatcaccaccttccaggaagcctactttgtttcagaaagttttgaagaagccaaagaaaagatgagggactttgcaaagtcaattacccgtcccttctcagtatacttcaatccctacacacagagtattgaaattctgaaagacaccagaagtattgaaaatgtggtgcaggaccttcgcagcgacttgaatacagtgtgtgatgctttaaacaaaatgaaccaatatctggggatttgatgcctggaactatgttgttgccagcatgatctttttggggcttagcagcagttcagtcaatgtcatataacgcaaataaccttctgtgtcatggcttggctaataagcatgcaattccatatatctataccatcttgtaactcactgtgttagtatataaagcaccataagaaatccaatggcagataaccactcattgtatgaaataacgtattatgtttaaacatcttaaaaagatttgacattcctgcttagtgtccttaaccaaactgcatctagttaaaatttgtaacaaatagccctcttatgagtctcatttatgcccttttctttttcagatctaagcctttcctctgtgttcattagataaaatgaaaaaaagcagtgaagctgtttccattttcaatagtatcagtgttttcacgcattatttgagataaacccagaattgtaggaaacttcccatcacaataacaaaggttcaatattctatttcaaaaattgttgaggtaacacagcagttggaatgatttttaggttgagtatttacacaatgcaagaaaacacctttttacaaatggaattatgtaggttgcgttgaccttgtagaacctgagttatgacaagcttcctgaagtattttggaagatagtacttccggaaaggacattaggaaagactaaacagtggacaatcaatcttgggactatgaattttatgctggaataaagtaaattatcatgtt

SEQ ID NO: 58 - protein, 490 aa, Homo sapiens
MQPAMMMFSSKYWARRGFSLDSAVPEEHQLLGSSTLNKPNSGKNDDKGNKGSSKREAATESGKTAVVFSLKNEVGGLVKALRLFQEKRVNMVHIESRKSRRRSSEVEIFVDCECGKTEFNELIQLLKFQTTIVTLNPPENIWTEEEELEDVPWFPRKISELDKCSHRVLMYGSELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYIRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRSIENVVQDLRSDLNTVCDALNKMNQYLGI

SEQ ID NO: 59 - DNA, 2406 bp, Homo sapiens
ttcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 60 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 61 - DNA, 2429 bp, Homo sapiens
cagcttctgggggtagatctcaatttgcagtattcagacttctttttctttcttttacattcttttttctttctttctttctgccaactttgttttccagtgtttacaaggtgacaaatgtttgactttggttgtgtttaaatgtccgtgtaaaatagctgccttttattttttaaggtaacaaataccacctagaggtaggtaggatcatcccacgcttgctttagcacaggacaactttacaaaacatgattgtttacagctgctcttcccctcttttctgatctgcagtttttgcctgggtcccactcaggtgaaaatccatctcattctggaatggttttgcttttgaatttttggttatttttgtgtttctttgggggttagaccactttctgattagccgccacctgcctgcatctgtgaaaagggatctgctcccaggcgttctcacccttcttttgaaggactccttaggctttgttgaatgaagcagagaagattgtatagttggggctggtcttggtgaacacacattattaccccacacatcccctttgtgtagaaagccaaataaaatctatacataccatttccttttgagcccagaatctagatttgagcggaagagcatgtgtgcttcagggaattagtgtctttttttggaaatctgttgaagtaaagtaacatcggccttctgttcacttaggcagcatttatagaaacaaaagaagaaagaaacaacctactgtctggagtcataacacaactttcctggattggaaaccaagtgggggaaaaaatacagaaactttaagggggatgggaggggggggagaagggaaaagccagccctttgtatagaaattttgcttttttttccctcattctactttagaactgcaagcttgtgcactgtggatgcgtgaatattttagtgtgaaacgtgtttttgtcatagtattgaaataaaacttcaacatagtttggttgtggaaggtatagcagatagttcagaaaaaatattcaggaaacaaaaatcactcaaacggaatcgaagccttttaacaaagaaaatgaaatacagatgatgatgatgatgatgaagatgatgctaagtaaacagaaatcagtactccgcatgcgctcctctcctaaggtacaaagcagcaagaggttagggtggcaaggctgcctctgggtccattctgtgggccactctccccaacgttctgacacttctgcagtctgatcagtggcgatgctagattataatttcaaactgtgaagaataatggtcttgtcatttgctcaatgtggggttatgttgcattttctcagctcctggggatggaaatggaggatcccagaacacacagccctggcccctttgattctagggcctgcacagatctctggttcaaatgcacaggccctcagaatagaggaacatgaagagagatcttagagcacacagtagaatgtgagagcctgggtgtctgagaccgggagggcccagcagtgaggggcaggctcttctggtcaccaggctgttcagtggactcagttcttcatcttgtaatgtcgatggctttgccacaccaggccaagcccatgccataccttgtcaagactgtcaaagtggttgtggttaggtcaaactggttttggttctgatggttaggaagaaacaggtcagccctcagatcacctggcccgggacagctgaccccctagaaccctggctctgccattagctaggacctaagactctgcccacattttggtctgttctctcccattacacataggtttgtctcagcatgcaagagtttttcctttaaaaaaaaaaaaaaaaaaaaaaaaaaagcaatgctttctctaaaatcaaagagggagtcattttattccaagatgttttatcttttatgttaagagatcaaagcttataattttcttttttaatttttgaaggagggatcaactccagtttccaatgtctatgtgtctatgtgtgtatgtgccatacatatgtattcacatgaagaccggcatggccaagttctgctggaggagcactcaagtgtgacgagcagggccactggaccctgcagggctgtggtgtatatagtgcagctttggaggtggaactctattttcacacttttctatggagccttccgagtcccaggttttcacttgaggctgtctgtctggatggcggttttcagacctccattaacatccctacccagcattctgtacttcgggggccttctctcttgttataaaactttttaccaagtgaaacatcgataccacctttgtttccattctcactggtgtaaatactgagtactaactgagaattttgactttgcattctgtcggaatacttgtgttcaataaaaattgaaagaaaaaagcta

SEQ ID NO: 62 - protein, 848 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKTLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 63 - DNA, 1395 bp, Homo sapiens
gggccgccggctgccagcccagggcggggcggagccctacttctggccgaccgcgtaggcggtgcttgaacttagggctgcttgtggctgggcactcgcgcagaggccggcccgacgagccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaagtaagtacactgcaggctgtgcactctgtgccagaacaacagcagccctgctgtagatgcccattacttgggaagccagattgtactcatctgttggttgccttgtacagtaataagaagtaacatcagcagatatactaaccatagtgtattaagtaccaactgtgtgccatgcactgcactagccactctctgcatatcgtttcctggaaccctctcaatgagccttgcaggaggagtatcataccccttgcttagagacagcgcatatgagattcatagaagtcaagtgagatatcaaagtcacgttgctagtaagtgatgggctgggatttaaatctaggtctttggactccaagagttatactgttaaccactacaatacactccctccacctcccatcccagaaaaagttggatcagggatagggttcacttcaggagccagggtcagagctgggcctgtagtcacatataaaagataggacacaaacacaattagcagcagtgttggtttaataaaccgccgta

SEQ ID NO: 64 - protein, 248 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTK

SEQ ID NO: 65 - DNA, 7563 bp, Homo sapiens
agaagcggctcgaggctggaagaggatcttgggcgccgccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaa

SEQ ID NO: 66 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 67 - DNA, 1379 bp, Homo sapiens
tttggttccttgtggccacatttccagtacccagtagtcatctgtgccagggggttatccaggtacagaacattcccatcgttgcagaaggttctatcagctagcactgggttggacgacacttgccaagacgagctggctagaggatggttctccggacctggtcccacgtggttcccagctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactgaggccccgcagctctggccacagggacctctgcagtgccccctaagtgacccggacacttccgagggggccatcaccgcctgtgtatataacgtttccggtattactctgctacacgtagcctttttacttttggggttttgtttttgttctgaactttcctgttaccttttcagggctgacgtcacatgtaggtggcgtgtatgagtggagacgggcctgggtcttggggactggagggcaggggtccttctgccctggggtcccagggtgctctgcctgctcagccaggcctctcctgggagccactcgcccagagactcagcttggccaacttggggggctgtgtccacccagcccgcccgtcctgtgggctgcacagctcaccttgttccctcctgccccggttcgagagccgagtctgtgggcactctctgccttcatgcacctgtcctttctaacacgtcgccttcaactgtaatcacaacatcctgactccgtcatttaataaagaaggaacatcaggcatgctaccaggcctgtgcagtccctcag

SEQ ID NO: 68 - protein, 165 aa, Homo sapiens
MMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 69 - DNA, 2384 bp, Homo sapiens
gtgaatctgcgctaagctatgcagtctgctttttcttctcagctctggtagttcttcagaaatgtaccctccaggcacatccactattgcgagggtgagcacgaagggtgggagatgcccatgtcctcaaggcatcacttcctaaatccaaaagcatcggcaggagaaaggactggggacaaatactgtcccttcgggagtagggagggaacactgaggcccatccctggctccttccctaaaagtagagtaaaatggaagcgagcatcctgggattgggggcaagagggggaccgcagggtagctgtgggttccaactgctgtcagagtcagagaggcagccccaagccagcctccctgctttgccagggaatttgggggaggaaggtgacagctgcccagaggctgactcatctgatatttagcactgggtaggatgattgtttctgagcatttttcttaaaggcctcagatctaaattatgccaccggctcccactcttgctacctcccgtcaacttctctgccttgccttccacccctgtagttaccatacacagaggaggaggagctgtccttgtcccaggttgggaggctgacaaccccttagcaagatgctgccagcccagagctctccaaggggaggaacacccctgagactcaggcccctctccttcagccctgcttgggctgcaagcgccgtgccaaggaaaggcatcttggtgagaagagctgctgtgggggaagggagatcaaatgccagagaaatgtggggtgccccaccctcaggatagtaaaagagtatggaggtatttctggaaggaaatgagcggcactgtgtgaagcctcgcacctgtgtgacacttcctatggggtctttgtcacactctagtactatgtccctgaagagtttagcagccacactcttagaagggtgctgggagatggtgttgccctctgcagccatgtttaggggagcggaacctgaggcccacagtgggtgagattagctcaagaagccacagaggccaccagagggccacggacttcggaaaggagaagagaagaacagggcatcaggcctcacaacgcaaacctacccagagatgggcacagtggctcatgcctgtaatcccaccactttgggaagaggcggatcgcttgaggtcaggagttcgagactagcctcgaaaccctatctctactaaaaatacaaaaattagccaggcatggtggcctgcgcctgtaatcccagctactcaggaggctgaggcaggagaatcacttgaacccgggaggcggaggttgcagtgagccaagattgcaccactgcactccagcctgggagatagagtgagactccatctcaaaaaaataaaaaataaaataaacctacccggaatgaccatgctgaggactgggagcccgcagactttcagccacaggccgcgacagccgtgggtccctccctggtcaagtcagcaggccttgtggaggctgtggggtatctgtggtgactcaggtaattatagagggctggcccccagccctggttcctgtacacatgccccaaccccatccccatccactccctcgccagtcctaacctctttcctgggtccccccccttcagcacctaagtccatacctagggccgtggaattcccgctcaagagcaacagaagcccctctctgcacccccatttctggactggattgtccactgagacgcgcaatgtctgcatctctgacatctagaggcttcctcgggaagggcatggggatctccgtgagatgtggggactttcactggccaaccaagaaatctacacagcgtccggggacctgtgacacacatccctcccgcctcctcaacctgatgtccctctctgaatctgcagctttcgtgctgtgaaggtgtctttacatgtgaaacaaacaaacccaagtcaagagtaaatcatctcatttactagtgagaaaatgttggagctggagtccttcagagagtcctggccaggcaagagggccatcagctctcttctgctcaacaggggctctcagcctcaggacactctcaggcctggaatgtccccaacacactcaaggagaaacatgtcctgtgcagacccacaggaggcatctttgcccggcacaaggaagagctggggtcagtgggacctgtagatgtagacacatcatatggagggtgggtaggaccaatgtggcagcttcatggaggccaagtgtggctctgcaccaggaaggggctgtgatggctggaggtgcccagcagtgcaggcggggagtgcctggcagtggcgtggccaggtggaggccacctgtcaagtttgcaataaagcagtttcctgaatttggtgagaa

SEQ ID NO: 70 - protein, 161 aa, Homo sapiens
MNLAISIALLLTVLQVSRGQKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKCEGISLLAQNTSWLLLLLLSLSLLQATDFMSL

SEQ ID NO: 71 - DNA, 2421 bp, Homo sapiens
gcaatgtttatggtgaaaaatggcaacgggaccgcgtgcataatggccaacttctctgctgccttctcagtgaactacgacaccaagagtggccctaagaacatgacctttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcatgctgatccccatcgctgtgggtggtgccctggcggggctggtcctcatcgtcctcatcgcctacctcgtcggcaggaagaggagtcacgcaggctaccagactatctagcctggtgcacgcaggcacagcagctgcaggggcctctgttcctttctctgggcttagggtcctgtcgaaggggaggcacactttctggcaaacgtttctcaaatctgcttcatccaatgtgaagttcatcttgcagcatttactatgcacaacagagtaactatcgaaatgacggtgttaattttgctaactgggttaaatattttgctaactggttaaacattaatatttaccaaagtaggattttgagggtgggggtgctctctctgagggggtgggggtgccgctgtctctgaggggtgggggtgccgctgtctctgaggggtgggggtgccgctctctctgagggggtgggggtgccgctttctctgagggggtgggggtgccgctctctctgagggggtgggggtgctgctctctccgaggggtggaatgccgctgtctctgaggggtgggggtgccgctctaaattggctccatatcatttgagtttagggttctggtgtttggtttcttcattctttactgcactcagatttaagccttacaaagggaaagcctctggccgtcacacgtaggacgcatgaaggtcactcgtggtgaggctgacatgctcacacattacaacagtagagagggaacatcctaagacagaggaactccagagatgagtgtctggagcgcttcagttcagctttaaaggccaggacgggccacacgtggctggcggcctcgttccagtggcggcacgtccttgggcgtctctaatgtctgcagctcaagggctggcacttttttaaatataaaaatgggtgttatttttatttttatttgtaaagtgatttttggtcttctgttgacattcggggtgatcctgttctgcgctgtgtacaatgtgagatcggtgcgttctcctgatgttttgccgtggcttggggattgtacacgggaccagctcacgtaatgcattgcctgtaacaatgtaataaaaagcctctttctttttggggtgggccttgtccttctgtcagctaaaatgggagctcatgagagaaggacgtcagggaaacggggttgagggtggtctcggtgcagagagaagggtgtcagggaaacggggggtgagggtggtcttggtgccagacgtagggaatggtgttgggagtggcccgagtgcctggcacagttgtctggttcattcatgtaacatgataatttttaaatcattaaaaaaattacctttcatacaga

SEQ ID NO: 72 - protein, 417 aa, Homo sapiens
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMLIPIAVGGALAGLVLIVLIAYLVGRKRSHAGYQTI

SEQ ID NO: 73 - DNA, 3767 bp, Homo sapiens
aagaaagagccccgcccctagtcttatgactcgcactgaagcgccgattcctggcttttgcaaggctgtggtcggtggtcatcagtgctcttgacccaggtccagcgagccttttccctggtgttgcagctgttgttgtaccgccgccgtcgccgccgtcgccgcctgctctgcggggtcatggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagtacgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagctgaagaatgttctgctgactctgacctcaactttcttattcctgttgcagtgggtgtggccttgggcttccttataattgttgtctttatctcttatatgattggaagaaggaaaagtcgtactggttatcagtctgtgtaatcagttaaatctagtgtttgtttgtttttttcaattagaagttacgtttccattggctaaaagccaggacatgctgtgcaatagattgtttaagatatgcagactaacttcagtgagttcctagctaacttgggcatgagtacacttatttaagacaaaatatattaggaccaatttttttctgttttttttcttcctttgttaaagtataattaaaagaaaaattgtggcttagaattttttaagtaaataatgattttaagcccctggatccaattatgaaagcatttttgctgatgtgtaattttatatgttacagttacttatattttactactttgatgttatttgcaaaatcaaaggtgttaaagaatttaacttgcttcaggaaataaattcaagaacatagtggattcattttcattggtggcagacacgaaatttggttcatgataagacttcctttccccacctcctgatcagcattatttaaatctgtatttttctgttagttaagaaagaaatggcttcatgatattgtatttaatagcaaaagtttggctgtcttcttcattactgttaatagctactatattttaacaaggagatttctttttttgttgttgttgttctagagtttggaatatactgattatctcagacttgacatttatactgaaggatgaagtaagacctccagctttttttaaaaaaggtgttgatttggaacacctgtatgggttatggtttattaaggttatggtttagaaagtttttttccctcagagccttaacttgttaagaaggttcatttatcctgcactgaaaacaaaaactctatatactttgtttgtgtgcctcctgcactctcccattccctatgtgaatatgctctagttgatatttttaatatattgatttcttttttctcacagcaacaagtgcttactctagaggttagtgggccctgatatgtcatcagtcagatgcctgcctagccaaagctggactaagattattctgtacatttgttgatcttgatatagacttatatccctgtagggactgctaatggctccggcttctggagtaaggtactggagaccactcatccctgtgtctgcttgattggttcagctgttgaattgcccttttatttggaagcagtgttgaagttgtctagggttcaaatggctgctttgtacacctgtcattagtataaggcagatgtttattttatcaagctattttatctctacatttaactaaaaacaaaagttcccaaagatctgccttcacttcagaaattttttttggattaaaaaaattaagcctgaaccttaaataaagtgagttggttattcattccaaggattaagtcccaatctacctctcagcacaatgcagaagctcaccactgtattgctgccattaactcatgccagaaccctttgccaataactggaattacaaatttttgttaaagaaaatttatcaagatctttctttactgccttctctatatgtacatctcaaaaacatgtacatctcaaaaactggagtagaaagttagattgctcaactacaactcctctagaactctatagctctgacatacagattcacactctcctctatttgctaagtatgtaaagaatgttttcttttaaaatgttctcttttgagaacaactgcttatttgttataaaagcatttggttaaaatgatgtcatcataaaaaacagtggctttgtttcaatacatatttttgagatgattatctagaagccagattaataaaatcagcttgtgaccttgctaagcatataaactggaaattcagatacattcaaaattatgggttcatttaaaagtgttctaccttttgggtatgagactaatatcactaattcctcaatagttatcatggctctatcttaattaattagaaaatatgtgtgtttaattctttgagaattaaaatagagaatattaacagagggttaaaaactgcttcaactccaataagataaaggaagctcaaaatctatgagctgagtgttcaattagctttgcctactgagttcaattttatgtcaatacaacagtggatcagacagtacgactttgaactggtgaatgtaaacaattgtttttcacctaagctgctttggaagaactgatgcttgctgctaactaaagttttggatgtatcgatttagagaaccaattaatacctgcaaaataaagcatactgtggtacttctgtttgatctagtatgtgtgattttagattgatggattaaaaattaataaagatcatacattccataccaaaaaaaaaaaaaaaa

SEQ ID NO: 74 - protein, 411 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAEECSADSDLNFLIPVAVGVALGFLIIVVFISYMIGRRKSRTGYQSV

SEQ ID NO: 75 - DNA, 2389 bp, Homo sapiens
gctgaaactgaagaggacatgtcaaatattacagatccacagatgtgggattttgatgatctaaatttcactggcatgccacctgcagatgaagattacagcccctgtatgctagaaactgagacactcaacaagtatgttgtgatcatcgcctatgccctagtgttcctgctgagcctgctgggaaactccctggtgatgctggtcatcttatacagcagggtcggccgctccgtcactgatgtctacctgctgaacctggccttggccgacctactctttgccctgaccttgcccatctgggccgcctccaaggtgaatggctggatttttggcacattcctgtgcaaggtggtctcactcctgaaggaagtcaacttctacagtggcatcctgctgttggcctgcatcagtgtggaccgttacctggccattgtccatgccacacgcacactgacccagaagcgtcacttggtcaagtttgtttgtcttggctgctggggactgtctatgaatctgtccctgcccttcttccttttccgccaggcttaccatccaaacaattccagtccagtttgctatgaggtcctgggaaatgacacagcaaaatggcggatggtgttgcggatcctgcctcacacctttggcttcatcgtgccgctgtttgtcatgctgttctgctatggattcaccctgcgtacactgtttaaggcccacatggggcagaagcaccgagccatgagggtcatctttgctgtcgtcctcatcttcctgctttgctggctgccctacaacctggtcctgctggcagacaccctcatgaggacccaggtgatccaggagagctgtgagcgccgcaacaacatcggccgggccctggatgccactgagattctgggatttctccatagctgcctcaaccccatcatctacgccttcatcggccaaaattttcgccatggattcctcaagatcctggctatgcatggcctggtcagcaaggagttcttggcacgtcatcgtgttacctcctacacttcttcgtctgtcaatgtctcttccaacctctgaaaaccatcgatgaaggaatatctcttctcagaaggaaagaataaccaacaccctgaggttgtgtgtggaaggtgatctggctctggacaggcactatctgggttttggggggacgctataggatgtggggaagttaggaactggtgtcttcaggggccacaccaaccttctgaggagctgttgaggtacctccaaggaccggcctttgcacctccatggaaacgaagcaccatcattcccgttgaacgtcacatctttaacccactaactggctaattagcatggccacatctgagccccgaatctgacattagatgagagaacagggctgaagctgtgtcctcatgagggctggatgctctcgttgaccctcacaggagcatctcctcaactctgagtgttaagcgttgagccaccaagctggtggctctgtgtgctctgatccgagctcaggggggtggttttcccatctcaggtgtgttgcagtgtctgctggagacattgaggcaggcactgccaaaacatcaacctgccagctggccttgtgaggagctggaaacacatgttccccttgggggtggtggatgaacaaagagaaagagggtttggaagccagatctatgccacaagaaccccctttacccccatgaccaacatcgcagacacatgtgctggccacctgctgagccccaagtggaacgagacaagcagcccttagcccttcccctctgcagcttccaggctggcgtgcagcatcagcatccctagaaagccatgtgcagccaccagtccattgggcaggcagatgttcctaataaagcttctgttccgtgcttgtccctgtggaagtatcttggttgtgacagagtcaagggtgtgtgcagcattgttggctgttcctgcagtagaatgggggcagcacctcctaagaaggcacctctctgggttgaagggcagtgttccctggggctttaactcctgctagaacagtctcttgaggcacagaaactcctgttcatgcccatacccctggccaaggaagatccctttgtccacaagtaaaaggaaatgctcctccagggagtctcagcttcaccctgaggtgagcatcatcttctgggttaggccttgcctaggcatagccctgcctcaagctatgtgagctcaccagtccctccccaaatgctttccatgagttgcagttttttcctagtctgttttccctccttggagacagggccctgtcggtttattcactgtatgtccttggtgcctggagcctactaaatgctcaataaataatgatcacaggaatgaa

SEQ ID NO: 76 - protein, 350 aa, Homo sapiens
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 77 - DNA, 2446 bp, Homo sapiens
ctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtgatagaaagctgagtctccctaaggcatgtgtgaaacatactcatagatgttatgcaaaaaaaagtctatggccaggtatgcatggaaaatgtgggaattaagcaaaatcaagcaagcctctctcctgcgggacttaacgtgctcatgggctgtgtgatctcttcagggtggggtggtctctgataggtagcattttccagcactttgcaaggaatgttttgtagctctagggtatatatccgcctggcatttcacaaaacagcctttgggaaatgctgaattaaagtgaattgttgacaaatgtaaacattttcagaaatattcatgaagcggtcacagatcacagtgtcttttggttacagcacaaaatgatggcagtggtttgaaaaactaaaacagaaaaaaaaatggaagccaacacatcactcattttaggcaaatgtttaaacatttttatctatcagaatgtttattgttgctggttataagcagcaggattggccggctagtgtttcctctcatttccctttgatacagtcaacaagcctgaccctgtaaaatggaggtggaaagacaagctcaagtgttcacaacctggaagtgcttcgggaagaaggggacaatggcagaacaggtgttggtgacaattgtcaccaattggataaagcagctcaggttgtagtgggccattaggaaactgtcggtttgctttgatttccctgggagctgttctctgtcgtgagtgtctcttgtctaaacgtccattaagctgagagtgctatgaagacaggatctagaataatcttgctcacagctgtgctctgagtgcctagcggagttccagcaaacaaaatggactcaagagagatttgattaatgaatcgtaatgaagttggggtttattgtacagtttaaaatgttagatgtttttaattttttaaataaatggaatactttttttttttttttaaagaaagcaactttactgagacaatgtagaaagaagttttgttccgtttctttaatgtggttgaagagcaatgtgtggctgaagacttttgttatgaggagctgcagattagctaggggacagctggaattatgctggcttctgataattattttaaaggggtctgaaatttgtgatggaatcagattttaacagctctcttcaatgacatagaaagttcatggaactcatgtttttaaagggctatgtaaatatatgaacattagaaaaatagcaacttgtgttacaaaaatacaaacacatgttaggaaggtactgtcatgggctaggcatggtggctcacacctgtaatcccagcattttgggaagctaagatgggtggatcacttgaggtcaggagtttgagaccagcctggccaacatggcgaaacccctctctactaaaaatacaaaaatttgccaggcgtggtggcgggtgcctgtaatcccagctacttgggaggctgaggcaagagaatcgcttgaacccaggaggcagaggttgcagtgagccgagatcgtgccattgcactccagcctgggtgacaaagcgagactccatctcaaaaaaaaaaaaaaaaaaaaaggaaagaactgtcatgtaaacataccaacatgtttaaacctgacaatggtgttatttgaaactttatattgttcttgtaagctttaactatatctctctttaaaatgcaaaataatgtcttaagattcaaagtctgtatttttaaagcatggctttggctttgcaaaataaaaaatgtgttttgtacatgaa

SEQ ID NO: 78 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 79 - DNA, 2588 bp, Homo sapiens
aggctaaggtcagacactgacacttgcagttgtctttggtagtttttttgcactaacttcaggaaccagctcatgatctcaggatgtatggaaaaataatctttgtattactattgtcagaaattgtgagcatatcagcattaagtaccactgaggtggcaatgcacacttcaacttcttcttcagtcacaaagagttacatctcatcacagacaaatgatacgcacaaacgggacacatatgcagccactcctagagctcatgaagtttcagaaatttctgttagaactgtttaccctccagaagaggaaaccgagataacactcattatttttggggtgatggctggtgttattggaacgatcctcttaatttcttacggtattcgccgactgataaagaaaagcccatctgatgtaaaacctctcccctcacctgacacagacgtgcctttaagttctgttgaaatagaaaatccagagacaagtgatcaatgagaatctgttcaccaaaccaaatgtggaaagaacacaaagaagacataagacttcagtcaagtgaaaaattaacatgtggactggacactccaataaattatatacctgcctaagttgtacaatttcagaatgcaattttcattataatgagttccagtgactcaatgatggggaaaaaaatctctgctcattaatatttcaagataaagaacaaatgtttccttgaatgcttgcttttgtgtgttagcataatttttagaattgtttgagaattctgatccaaaactttagttgaattcatctacgtttgtttaatattaacttaacctattctattgtattataatgatgattctgtcaaatgaaaggcttgaaatacctagatgaagtttagattttcttcctattgtaaacttttgagtctggtttcattgttttaaataaattaaggggacactaaagtcctatcattcatttccttcattgctgaacaggcaagatataatattacatgaatgattactatattttgttcacactaataaagcttatgctcagaaatgccatacacacacacacacacacacacaaacacacacatttatcatttaatgcataaatcaacacaaaaggttttcccattaatatgaaatattacatatatataagtgccatatttaaaataatttgtctaacagtagaactgtgtcggagcactcactgaagcttgcattccactgaaagagttatttgtgtaagtagagtatccggagaaggaaaagaacttacgacctttctttataacagaaactcaactctaaattcaacaagatgtgcaaaccggacatgcaggtgaatattttaataggttactataaggttctcaattaaattctttaatctgtccagtcccagtttctcttattaataaaactttggaaattgctttaaaccatttaaaggaaatttctagatatagaaactaaggactgtgactatacagctgtcactcatttgtagtaaaacttaaaaagcaaaaacaaaaaacaaaaaagaccttcctgtgatactttatttccgaactaataaaaatctatatgactttttattattgtgtgataaccaagtaaatgttttctattttgcatattttcaggcatggtaacagaaatttaccttttaataaattaaaaaatctaaattttaacctacttgtatgttcggagagtgtttttgtactatattgactacttaaaatagagaatgagactaagaagggaacatttctgttgatacatgttttttaaaagtaattttaagagcattattaggttaattaatccaattaatgacccaaatgccaaggtaattttaaatttacatttttaataaaagcaacatgttgaaacaagagagggtgagattaacctttttgctaaagtaatttacaagtcaaagacaggaagagatcagagtgaatgtgccttcttaaccagagctacagaatttagtgaataattaaagtacaaactgctttgacctccttgaacttttccaagcaatttctctgtacttctatatatgaatgtcttagccaattttctgctactataacagaatacgacagactgggtaatttaaaaagaaaagaaatttattttcttcctagttctggaggctgggaaggcgaagggcatggcactgacatctgccttgtaactgatgagaaccttcttactgcatgataacaaagcagcaaggcaagcaaaagcgtaagatgaagagagaggaaatgaagccaaacacatcctttcatcagaagcccattccctctataaggcgttattacatttatgagaatggagtcctcatgacctaatcgtgaccttaaaggcccctcccaacactgttacaatggcaattaaatttcaacaaaggttccagaggtgacattcgaatcagcaatgaaattttcatagttaaatttggtattcgtgggggaagaaatgaccatttcccttgtatttttataattaaatcagcaaaatattgtaataaagaaatctttcctgtgaagataccatgacccca

SEQ ID NO: 80 - protein, 137 aa, Homo sapiens
MYGKIIFVLLLSEIVSISALSTTEVAMHTSTSSSVTKSYISSQTNDTHKRDTYAATPRAHEVSEISVRTVYPPEEETEITLIIFGVMAGVIGTILLISYGIRRLIKKSPSDVKPLPSPDTDVPLSSVEIENPETSDQ

SEQ ID NO: 81 - DNA, 1611 bp, Homo sapiens
gagaagccaatcagtgtcgtcgcggtcgctgttctaaagtccgcacgcacccaccgggactcagattctccccagacgccgaggatggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagagcaccccacccccatgtccaccatgaccctcttcccacgctgacctgtgctccctctccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaattaatggaacgcatgagaaccttccagagtcca

SEQ ID NO: 82 - protein, 365 aa, Homo sapiens
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV